SEQUENCE LISTING (275 sequences)

SEQ ID NO: 1 - DNA, 500 bp, artificial
  Sequence of the first arm of the HRPL/CFA6 dsRNA used to concomitantly target HrpL and Cfa6 genes of Pto DC3000
atgaacaagcgctacggcaacaacttgccgggatcctgttgcgcgctgagcaaccggatcgactggacgtcaccttcgcgcagcacgccatctggcaaaaaggccgccgttaatcgtttccaatatttctgcaggagtctgcccgtgaccaccttcaccgtgcccctgattctgtcggatgcaagccccacggcactcgccgctaccgcgcaacgtctgcgcgccgagcacgagacgcgctccttcgccgctgatgactgacatcaccgtgcccttccagctcggaatgcacttcgtcttcccagctttcctgatacggctgacgatacattttgcggaagtgattgcggatcaggttcagcgcgatgccacacagccaggtctgcggtttgctggcatgttgaaacttgtgctcgttacgcagggcttcaagaaacacgcactggagaatgtcatccacatcatcagggttcatcacccgcttttggataaacgccctg

SEQ ID NO: 2 - DNA, 1410 bp, artificial
  Sequence of the CHSA intron used to generate all the inverted repeat from the present invention
ggcgcgcccaatcgatgatttaaatgtgtaagaatttcttatgttacattattacattcaacgttttatcttaattggctcttcatttgattgaaatttgacaattatttcttgtttttttttttgtcacactctttttgggttggggtggccgacgaattgtgggaaggtagaaagaggggaggacttttgttatactccattagtaattactgtttccgtttcaatttatgtgacaatatttcctttttagtcggttccaaaagaaaatgtcagcattataaacaatttaattttgaaattacaattttgccattaataaaatgatttacaaccacaaaagtatctatgagcctgtttgggtgggcttataagcagcttattttaagtggcttataagtcaaaaagtgacattttttgagaagttagaaaatcctaacttctcaaaaagtagcttttaagccacttatgacttataagtccaaaaatttttaagttaccaaacatatattaatgggtttataagcttataagccacttttaagctcacccaaacgggttctatgtctcactttagactacaaattttaaaagtcttcatttatttcttaatctccgtggcgagtaaaactataacacataaagtgaaacggagggaataagatggagtcataaactaatccaaatctatactctctccgttaatttgttttttagtttgatttggtacattaataaaacagatttttcgaaggttataaacacagacagatgtttcccagcgagctagcaaaattccaagatttctgtcgaaaattcgtgtgtttctagctagtacttgatgttatctttaaccttttagtaattttttgtccttttctttctatttttcatcttacaatgaattatgagcaagttccttaagtagcatcacacgtgagatgttttttatgatattgactaaatccaatctttaccattccttaactagtaaaatacaacacatgttaattgatacattgcttaacactgaggttagaaaattttagaaattagttgtccaaatgctttgaaattagaaatctttaatcccttatttttttttaaaatgttttttctcactccaaagaaagagaaactgacatgaaagctcaaaagatcatgaatcttactaactttgtggaactaaatgtacatcagaatgtttctgacatgtgaaaatgaaagctcttaattttcttcttttatttattgagggtttttgcatgctatgcattcaatttgagtactttaaagcacctataaacacttacttacacttgccttggagtttatgttttagtgttttcttcacatcttttttggtcaatttgcaggtatttggatcctaggtgagtctagaggcgcgcc

SEQ ID NO: 3 - DNA, 500 bp, artificial
  Sequence of the second arm of the HRPL/CFA6 dsRNA used to concomitantly target HrpL and Cfa6 genes of Pto DC3000
cagggcgtttatccaaaagcgggtgatgaaccctgatgatgtggatgacattctccagtgcgtgtttcttgaagccctgcgtaacgagcacaagtttcaacatgccagcaaaccgcagacctggctgtgtggcatcgcgctgaacctgatccgcaatcacttccgcaaaatgtatcgtcagccgtatcaggaaagctgggaagacgaagtgcattccgagctggaagggcacggtgatgtcagtcatcagcggcgaaggagcgcgtctcgtgctcggcgcgcagacgttgcgcggtagcggcgagtgccgtggggcttgcatccgacagaatcaggggcacggtgaaggtggtcacgggcagactcctgcagaaatattggaaacgattaacggcggcctttttgccagatggcgtgctgcgcgaaggtgacgtccagtcgatccggttgctcagcgcgcaacaggatcccggcaagttgttgccgtagcgcttgttcat

SEQ ID NO: 4 - DNA, 250 bp, artificial
  Sequence of the first arm of the CFA6-A dsRNA used to target the Cfa6 gene of Pto DC3000
atgaacaagcgctacggcaacaacttgccgggatcctgttgcgcgctgagcaaccggatcgactggacgtcaccttcgcgcagcacgccatctggcaaaaaggccgccgttaatcgtttccaatatttctgcaggagtctgcccgtgaccaccttcaccgtgcccctgattctgtcggatgcaagccccacggcactcgccgctaccgcgcaacgtctgcgcgccgagcacgagacgcgctccttcgccg

SEQ ID NO: 5 - DNA, 250 bp, artificial
  Sequence of the second arm of the CFA6-A dsRNA used to target the Cfa6 gene of Pto DC3000
cggcgaaggagcgcgtctcgtgctcggcgcgcagacgttgcgcggtagcggcgagtgccgtggggcttgcatccgacagaatcaggggcacggtgaaggtggtcacgggcagactcctgcagaaatattggaaacgattaacggcggcctttttgccagatggcgtgctgcgcgaaggtgacgtccagtcgatccggttgctcagcgcgcaacaggatcccggcaagttgttgccgtagcgcttgttcat

SEQ ID NO: 6 - DNA, 472 bp, artificial
  Sequence of the first arm of the CFA6-B dsRNA used to target the Cfa6 gene of Pto DC3000
atgaacaagcgctacggcaacaacttgccgggatcctgttgcgcgctgagcaaccggatcgactggacgtcaccttcgcgcagcacgccatctggcaaaaaggccgccgttaatcgtttccaatatttctgcaggagtctgcccgtgaccaccttcaccgtgcccctgattctgtcggatgcaagccccacggcactcgccgctaccgcgcaacgtctgcgcgccgagcacgagacgcgctccttcgccgaatggcaagtattctgccgacagcaactgacacgcgaccatgccgaataccgcgcggccatcttagccaccgatcaagccagcctactcaaagggctggatatgctggcccgcggccgagcctcgcgccatctggtgctgggccgcgccgaccagttgcgacagccggtactggtgtttccaggccaaggaccgctatggccgcggatgactaccggactga

SEQ ID NO: 7 - DNA, 472 bp, artificial
  Sequence of the second arm of the CFA6-B dsRNA used to target the Cfa6 gene of Pto DC3000
tcagtccggtagtcatccgcggccatagcggtccttggcctggaaacaccagtaccggctgtcgcaactggtcggcgcggcccagcaccagatggcgcgaggctcggccgcgggccagcatatccagccctttgagtaggctggcttgatcggtggctaagatggccgcgcggtattcggcatggtcgcgtgtcagttgctgtcggcagaatacttgccattcggcgaaggagcgcgtctcgtgctcggcgcgcagacgttgcgcggtagcggcgagtgccgtggggcttgcatccgacagaatcaggggcacggtgaaggtggtcacgggcagactcctgcagaaatattggaaacgattaacggcggcctttttgccagatggcgtgctgcgcgaaggtgacgtccagtcgatccggttgctcagcgcgcaacaggatcccggcaagttgttgccgtagcgcttgttcat

SEQ ID NO: 8 - DNA, 250 bp, artificial
  Sequence of the first arm of the HRPL-A dsRNA used to target the HrpL gene of Pto DC3000
ctgatgactgacatcaccgtgcccttccagctcggaatgcacttcgtcttcccagctttcctgatacggctgacgatacattttgcggaagtgattgcggatcaggttcagcgcgatgccacacagccaggtctgcggtttgctggcatgttgaaacttgtgctcgttacgcagggcttcaagaaacacgcactggagaatgtcatccacatcatcagggttcatcacccgcttttggataaacgccctg

SEQ ID NO: 9 - DNA, 250 bp, artificial
  Sequence of the second arm of the HRPL-A dsRNA used to target the HrpL gene of Pto DC3000
cagggcgtttatccaaaagcgggtgatgaaccctgatgatgtggatgacattctccagtgcgtgtttcttgaagccctgcgtaacgagcacaagtttcaacatgccagcaaaccgcagacctggctgtgtggcatcgcgctgaacctgatccgcaatcacttccgcaaaatgtatcgtcagccgtatcaggaaagctgggaagacgaagtgcattccgagctggaagggcacggtgatgtcagtcatcag

SEQ ID NO: 10 - DNA, 348 bp, artificial
  Sequence of the first arm of the HRPL-B dsRNA used to target the HrpL gene of Pto DC3000
ctgatgactgacatcaccgtgcccttccagctcggaatgcacttcgtcttcccagctttcctgatacggctgacgatacattttgcggaagtgattgcggatcaggttcagcgcgatgccacacagccaggtctgcggtttgctggcatgttgaaacttgtgctcgttacgcagggcttcaagaaacacgcactggagaatgtcatccacatcatcagggttcatcacccgcttttggataaacgccctgagcatctgaatctgatcggccgtcatttggcgaataccggcggacgaagatggctgacggggctgggttgagtcgaggatcacaatcttctgaaacat

SEQ ID NO: 11 - DNA, 348 bp, artificial
  Sequence of the second arm of the HRPL-B dsRNA used to target the HrpL gene of Pto DC3000
atgtttcagaagattgtgatcctcgactcaacccagccccgtcagccatcttcgtccgccggtattcgccaaatgacggccgatcagattcagatgctcagggcgtttatccaaaagcgggtgatgaaccctgatgatgtggatgacattctccagtgcgtgtttcttgaagccctgcgtaacgagcacaagtttcaacatgccagcaaaccgcagacctggctgtgtggcatcgcgctgaacctgatccgcaatcacttccgcaaaatgtatcgtcagccgtatcaggaaagctgggaagacgaagtgcattccgagctggaagggcacggtgatgtcagtcatcag

SEQ ID NO: 12 - DNA, 360 bp, artificial
  Sequence of the first arm of the HRCC dsRNA used to target the HrcC gene of Pto DC3000
gaaggcgtggttctggttcgtggtccggccaaatacgtggagtttgtgcgcgactacagcaagaaagtcgaaaagcccgacgagaaggccgacaagcaagatgttgtcgtgctgccactcaaatacgccaacgcggctgatcggactattcgctaccgtgaccagcagttagtggtggccggtgtcgccagtattcttcaagagctgctggaaagccgttcgcgtggcgaaagcattgacagcgtgaacctgttgccggggcagggcagcagtgttgccaacagcacaggtgtcgcggccgccggcctgccttacaacctgggctccaatggtatcgatacgggagcactgcaacagggc

SEQ ID NO: 13 - DNA, 360 bp, artificial
  Sequence of the second arm of the HRCC dsRNA used to target the HrcC gene of Pto DC3000
gccctgttgcagtgctcccgtatcgataccattggagcccaggttgtaaggcaggccggcggccgcgacacctgtgctgttggcaacactgctgccctgccccggcaacaggttcacgctgtcaatgctttcgccacgcgaacggctttccagcagctcttgaagaatactggcgacaccggccaccactaactgctggtcacggtagcgaatagtccgatcagccgcgttggcgtatttgagtggcagcacgacaacatcttgcttgtcggccttctcgtcgggcttttcgactttcttgctgtagtcgcgcacaaactccacgtatttggccggaccacgaaccagaaccacgccttc

SEQ ID NO: 14 - DNA, 400 bp, artificial
  Sequence of the first arm of the AvrPto/AvrPtoB dsRNA used to target the AvrPto and AvrPtoB genes of Pto DC3000
atgggaaatatatgtgtcggcggatccaggatggcccatcaggtgaactccccagaccgagttagtaacaactcgggtgacgaagataacgtaacgtccagtcaactgctgagcgtcagacatcaacttgcggagtctgctggtgtaccaagagatcagcatgaatttgttagtaaccaagcacctcaaagcctgagaaaatggcgggtatcaatagagcgggaccatcgggcgcttattttgttggccacacagaccccgagccagtatcggggcaagcacacggatccggcagcggcgccagctcctcgaacagtccgcaggttcagccgcgaccctcgaatactcccccgtcgaacgcgcccgcaccgccgccaaccggacgtgagaggctttcacg

SEQ ID NO: 15 - DNA, 400 bp, artificial
  Sequence of the second arm of the AvrPto/AvrPtoB dsRNA used to target the AvrPto and AvrPtoB genes of Pto DC3000
cgtgaaagcctctcacgtccggttggcggcggtgcgggcgcgttcgacgggggagtattcgagggtcgcggctgaacctgcggactgttcgaggagctggcgccgctgccggatccgtgtgcttgccccgatactggctcggggtctgtgtggccaacaaaataagcgcccgatggtcccgctctattgatacccgccattttctcaggctttgaggtgcttggttactaacaaattcatgctgatctcttggtacaccagcagactccgcaagttgatgtctgacgctcagcagttgactggacgttacgttatcttcgtcacccgagttgttactaactcggtctggggagttcacctgatgggccatcctggatccgccgacacatatatttcccat

SEQ ID NO: 16 - DNA, 752 bp, artificial
  Sequence of the first arm of the CYP51 dsRNA used to target the FgCYP51A, FgCYP51B and FgCYP51C genes of Fusarium graminearum
cagcaagtttgacgagtccctggccgctctctaccacgacctcgatatgggcttcacccccatcaacttcatgcttcactgggcccctctcccctggaaccgtaagcgcgaccacgcccagcgcactgttgccaagatctacatggacactatcaaggagcgccgcgccaagggcaacaacgaatccgagcatgacatgatgaagcaccttatgaactctcggtccattgacaatccccgtctttggtagcgatgtcgtatacgattgtcccaactcgaagctcatggaacaaaagaagtttgtcaagtttggccttacgcaaaaagcactcgagtcacacgtccagttaatcgagcgagaggttcttgactacgtcgaaactgatccatccttttctggcagaactagcaccatcgatgtccccaaggcaatggctgagataacaatctttactgcctcacgttctttgcagggtgaggaagttcggagaaaactcactgccgagtttgctgcattggaagcaccgtacaatatggcatcgacccgtacgcttttttcttcgactgcagagataaatacggcgactgctttacctttattctccttggcaaatcaacgactgtctttcttggtcccaagggcaatgactttatcctcaacggcaaacacgccgatctcaacgccgaggacgtttatgggaaacttaccacgcccgtgtttggtgaggaggttgtttatgactgctccaatg

SEQ ID NO: 17 - DNA, 752 bp, artificial
  Sequence of the second arm of the CYP51 dsRNA used to target the FgCYP51A, FgCYP51B and FgCYP51C genes of Fusarium graminearum
cattggagcagtcataaacaacctcctcaccaaacacgggcgtggtaagtttcccataaacgtcctcggcgttgagatcggcgtgtttgccgttgaggataaagtcattgcccttgggaccaagaaagacagtcgttgatttgccaaggagaataaaggtaaagcagtcgccgtatttatctctgcagtcgaagaaaaaagcgtacgggtcgatgccatattgtacggtgcttccaatgcagcaaactcggcagtgagttttctccgaacttcctcaccctgcaaagaacgtgaggcagtaaagattgttatctcagccattgccttggggacatcgatggtgctagttctgccagaaaaggatggatcagtttcgacgtagtcaagaacctctcgctcgattaactggacgtgtgactcgagtgctttttgcgtaaggccaaacttgacaaacttcttttgttccatgagcttcgagttgggacaatcgtatacgacatcgctaccaaagacggggattgtcaatggaccgagagttcataaggtgcttcatcatgtcatgctcggattcgttgttgcccttggcgcggcgctccttgatagtgtccatgtagatcttggcaacagtgcgctgggcgtggtcgcgcttacggttccaggggagaggggcccagtgaagcatgaagttgatgggggtgaagcccatatcgaggtcgtggtagagagcggccagggactcgtcaaacttgctg

SEQ ID NO: 18 - DNA, 667 bp, artificial
  Sequence of the first arm of the HRPG/HRPB/HRCC dsRNA used to target concomitantly the HrpG, HrpB and HrcC genes of Ralstonia species
cgcatgcctggccgtgcagaacgtggaatgtgtccggttcgacgatgccctgacgctgttgcgtgcacggcgtaccgaggcattcagtctgctgctgatcgatgcccagcagttccgcagcgcgggacagctggtgctgtcctggcgcgagtgcaacgccgacatgtgctggccgacgctggtgttcggccagttcgcagtgaagaagcggaagaaggcttccgcctggcgcagcgcctgatccgccattcggatgacgcgcgatcgctacgccgcggcggcgagctgcttctcgcgcatggccgaagacgatggcgcgacctggacccagcaggtcgagggcctgatcggcccgcggctggctggccaccatcgatctgatcatctacgttttcgccgtgcaggccggcatccgctgctccaaccgcctgctcgagcatgcgttctggcaatcggccgaaatgggcatctcgacgcaggtcgagggcagcgtgacgggctcgttcaacgagacgccgcagaagtttctcgaccgcatggccggcacgttcggctttgcgtggtactacgacggcgcggtgctgcgcgtgaccagcgccaacgaagcgcagagcgccaccatcgcgctgacccgcgcctcgaccgcgcaggtcaagcgggcgctgac

SEQ ID NO: 19 - DNA, 667 bp, artificial
  Sequence of the second arm of the HRPG/HRPB/HRCC dsRNA used to target concomitantly the HrpG, HrpB and HrcC genes of Ralstonia species
gtcagcgcccgcttgacctgcgcggtcgaggcgcgggtcagcgcgatggtggcgctctgcgcttcgttggcgctggtcacgcgcagcaccgcgccgtcgtagtaccacgcaaagccgaacgtgccggccatgcggtcgagaaacttctgcggcgtctcgttgaacgagcccgtcacgctgccctcgacctgcgtcgagatgcccatttcggccgattgccagaacgcatgctcgagcaggcggttggagcagcggatgccggcctgcacggcgaaaacgtagatgatcagatcgatggtggccagccagccgcgggccgatcaggccctcgacctgctgggtccaggtcgcgccatcgtcttcggccatgcgcgagaagcagctcgccgccgcggcgtagcgatcgcgcgtcatccgaatggcggatcaggcgctgcgccaggcggaagccttcttccgcttcttcactgcgaactggccgaacaccagcgtcggccagcacatgtcggcgttgcactcgcgccaggacagcaccagctgtcccgcgctgcggaactgctgggcatcgatcagcagcagactgaatgcctcggtacgccgtgcacgcaacagcgtcagggcatcgtcgaaccggacacattccacgttctgcacggccaggcatgcg

SEQ ID NO: 20 - DNA, 705 bp, artificial
  Sequence of the first arm of the HRPB/HRCC/TssB/XpsR dsRNA used to target concomitantly the HrpB, HrcC, XpsR and TssB genes of Ralstonia species
gcgcgatcgctacgccgcggcggcgagctgcttctcgcgcatggccgaagacgatggcgcgacctggacccagcaggtcgagggcctgatcggcccgcggctggctggccaccatcgatctgatcatctacgttttcgccgtgcaggccggcatccgctgctccaaccgcctgctcgagcatgcgttctggcaatcggccgaaatgggcatctcgacgcaggtcgagggcagcgtgacgggctcgttcaacgagacgccgcagaagtttctcgaccgcatggccggcacgttcggctttgcgtggtactacgacggcgcggtgctgcgcgtgaccagcgccaacgaagcgcagagcgccaccatcgcgctgacccgcgccggccgcgcgtgcggagacccatcggcggccgcagagcgaatgcgactggaagcattactttgcggacctgctctatgcgccgaacggcgccgagttcaagctcagcctgtttccgctgcccgcgcagcagatcggcgacacgccctggtcgcgggcattccgcgggcagccggcgatgcttgacaaccaatccgccgcgctcgcgcagagcgcaccggcagccgagtccgtcaacctgctcgacgagatcgtcgagcagagccgcgtcgccaagtcggacgccgagcacgcgcgcgccaaggacatcatcggcgagctggtcaac

SEQ ID NO: 21 - DNA, 705 bp, artificial
  Sequence of the second arm of the HRPB/HRCC/TssB/XpsR dsRNA used to target concomitantly the HrpB, HrcC, XpsR and TssB genes of Ralstonia species
gttgaccagctcgccgatgatgtccttggcgcgcgcgtgctcggcgtccgacttggcgacgcggctctgctcgacgatctcgtcgagcaggttgacggactcggctgccggtgcgctctgcgcgagcgcggcggattggttgtcaagcatcgccggctgcccgcggaatgcccgcgaccagggcgtgtcgccgatctgctgcgcgggcagcggaaacaggctgagcttgaactcggcgccgttcggcgcatagagcaggtccgcaaagtaatgcttccagtcgcattcgctctgcggccgccgatgggtctccgcacgcgcggccggcgcgggtcagcgcgatggtggcgctctgcgcttcgttggcgctggtcacgcgcagcaccgcgccgtcgtagtaccacgcaaagccgaacgtgccggccatgcggtcgagaaacttctgcggcgtctcgttgaacgagcccgtcacgctgccctcgacctgcgtcgagatgcccatttcggccgattgccagaacgcatgctcgagcaggcggttggagcagcggatgccggcctgcacggcgaaaacgtagatgatcagatcgatggtggccagccagccgcgggccgatcaggccctcgacctgctgggtccaggtcgcgccatcgtcttcggccatgcgcgagaagcagctcgccgccgcggcgtagcgatcgcgc

SEQ ID NO: 22 - DNA, 633 bp, artificial
  Sequence of the first arm of the HRPG/HRPX/RsmA dsRNA used to target concomitantly the HrpG, HrpX, and RsmA genes of Xanthomonas campestris pv. campestris
gtgatggacgccgctgcgaatgaccgccaaggatcggcattcgtactgacgcaggacgcgcggctggcatcacagatcaacgccagcctggcgactctggtcccagaggtcaccatcttttcagacgaactcgaattgctgcgctgcctgcgccactcgccgtgcgagcttctggttttcgatgcccattgcgtggcatcggacgacagtatgatcctttcgacctacttcgcagcgatctctgcgttgtcttacgcagaccgtcttccgatctatacgagcaggatgcttgttggtgcttggccacagggtctgcagcacctccagcaacgtcgcgaggggcaggcagctccggatggcagcgacgacgaagtcgcattgctgggcgccagcgccgatgccttgttgattctggagtatatgttgatcctcactcgccgagtaggcgaaaccctgatgatcggcgacttggtcaccgtgaccgtgctcggcgtcaagggaaatcaggtgcgcattggcattgacgcgcctaaggatgttgccgtgcatcgcgaagaaatctatcagcgcatccagcgcggtgacgagccggttgcgtccggtgcgcatcacaacgacgattgttcggactga

SEQ ID NO: 23 - DNA, 633 bp, artificial
  Sequence of the second arm of the HRPG/HRPX/RsmA dsRNA used to target concomitantly the HrpG, HrpX, and RsmA genes of Xanthomonas campestris pv. campestris
tcagtccgaacaatcgtcgttgtgatgcgcaccggacgcaaccggctcgtcaccgcgctggatgcgctgatagatttcttcgcgatgcacggcaacatccttaggcgcgtcaatgccaatgcgcacctgatttcccttgacgccgagcacggtcacggtgaccaagtcgccgatcatcagggtttcgcctactcggcgagtgaggatcaacatatactccagaatcaacaaggcatcggcgctggcgcccagcaatgcgacttcgtcgtcgctgccatccggagctgcctgcccctcgcgacgttgctggaggtgctgcagaccctgtggccaagcaccaacaagcatcctgctcgtatagatcggaagacggtctgcgtaagacaacgcagagatcgctgcgaagtaggtcgaaaggatcatactgtcgtccgatgccacgcaatgggcatcgaaaaccagaagctcgcacggcgagtggcgcaggcagcgcagcaattcgagttcgtctgaaaagatggtgacctctgggaccagagtcgccaggctggcgttgatctgtgatgccagccgcgcgtcctgcgtcagtacgaatgccgatccttggcggtcattcgcagcggcgtccatcac

SEQ ID NO: 24 - DNA, 676 bp, artificial
  Sequence of the first arm of the RpoB/RpoC/FusA dsRNA used to target concomitantly the RpoB, RpoC and FusA genes of Pto DC3000 and Pseudomonas syringae CC440
aacacgtatccgcaaggactttagcaagttgccggacgtaatggatgtgccgtatctcttggccatccagctggattcgtatcgcgaattcctgcaggcgggagcgaccaaagatcagttccgcgacgtcggtctgcatgcagccttcaaatccgttttcccgatcatcagctactccggcaatgctgcgctggagtatgtaggttatcgcttgggcgagatcttgaaagacctactgaatttgctgaaaaaccagggtcaagtcgaagagttcgacgcaatccgtatcggacttgcatcgcctgagatgatccgctcttggtcgttcggtgaagttaaaaagccggaaaccatcaactaccgtacgttcaagcctgagcgtgacggtctgttctgcgccaagatctttggtccggtaaaagattacgagtgcctgtgcggtaagtacaaggcatatggctcgtactactccgattagccgttaccgtaacatcggtatcgtcgctcacgtggatgctggtaaaaccaccaccaccgagcgcgtccttttttacaccggcaaaagccacaaaatgggcgaggtgcatgatggcgccgcgaccacggactggatggttcaggagcaggagcgtggtattaccattacttctgctgctatcactgccttttggcggct

SEQ ID NO: 25 - DNA, 676 bp, artificial
  Sequence of the second arm of the RpoB/RpoC/FusA dsRNA used to target concomitantly the RpoB, RpoC and FusA genes of Pto DC3000 and Pseudomonas syringae CC440
tcaggccaaaaggcagtgatagcagcagaagtaatggtaataccacgctcctgctcctgaaccatccagtccgtggtcgcggcgccatcatgcacctcgcccattttgtggcttttgccggtgtaaaaaaggacgcgctcggtggtggtggttttaccagcatccacgtgagcgacgataccgatgttacggtaacggctaatcggagtagtacgagccatatgccttgtacttaccgcacaggcactcgtaatcttttaccggaccaaagatcttggcgcagaacagaccgtcacgctcaggcttgaacgtacggtagttgatggtttccggctttttaacttcaccgaacgaccaagagcggatcatctcaggcgatgcaagtccgatacggattgcgtcgaactcttcgacttgaccctggtttttcagcaaattcagtaggtctttcaagatctcgcccaagcgataacctacatactccagcgcagcattgccggagtagctgatgatcgggaaaacggatttgaaggctgcatgcagaccgacgtcgcggaactgatctttggtcgctcccgcctgcaggaattcgcgatacgaatccagctggatggccaagagatacggcacatccattacgtccggcaacttgctaaagtccttgcggatacgctgc

SEQ ID NO: 26 - DNA, 681 bp, artificial
  Sequence of the first arm of the SecE/RpoA/RplQ dsRNA used to target concomitantly the SecE, RpoA and RplQ genes of Pto DC3000 and Pseudomonas syringae CC440
aacatgaatcccaaggctgaagcatcagactctcgctttgatttgctgaaatggctcctggtagtcattttggtagtcgtgggtgttgtcggtaatcagtattactctgctgagccgatcctgtaccgtgttctcgctctccttgtgattgccgcagcagctgcatttgtagcgctgcagactggcaagggcaaagctttcttcgttttggcgaaagaagcgcgtgcagagatgatctgcagatttcggtaaatgagttcctgacaccccgccacattgatgtgcaggttgtcagtccaacccgcgccaaaatcactctcgagcctctcgagcgtggtttcggccataccctgggcaacgcgcttcgccgcattttgttgtcctccatgcccggctgtgcagtagtcgaggccgagattgacggtgtactccatgagtacagcgccatcgaaggtgtagcatatgcgtcatcgtaaaagtggacgtcacctgagccgtaccagctctcaccgtaaagccatgtttcaaaacatggcggtgtcgctgttcgagcacgagctgatcaaaactaccctgccgaaagccaaggaactgcgccgcgttgccgagccgctgatcaccctggccaaggaagacagtgttgctaaccgtcgtctggctttcgaccgtactcgttcggct

SEQ ID NO: 27 - DNA, 681 bp, artificial
  Sequence of the second arm of the SecE/RpoA/RplQ dsRNA used to target concomitantly the SecE, RpoA and RplQ genes of Pto DC3000 and Pseudomonas syringae CC440
tcaggaacgagtacggtcgaaagccagacgacggttagcaacactgtcttccttggccagggtgatcagcggctcggcaacgcggcgcagttccttggctttcggcagggtagttttgatcagctcgtgctcgaacagcgacaccgccatgttttgaaacatggctttacggtgagagctggtacggctcaggtgacgtccacttttacgatgacgcatatgctacaccttcgatggcgctgtactcatggagtacaccgtcaatctcggcctcgactactgcacagccgggcatggaggacaacaaaatgcggcgaagcgcgttgcccagggtatggccgaaaccacgctcgagaggctcgagagtgattttggcgcgggttggactgacaacctgcacatcaatgtggcggggtgtcaggaactcatttaccgaaatctgcagatcatctctgcacgcgcttctttcgccaaaacgaagaaagctttgcccttgccagtctgcagcgctacaaatgcagctgctgcggcaatcacaaggagagcgagaacacggtacaggatcggctcagcagagtaatactgattaccgacaacacccacgactaccaaaatgactaccaggagccatttcagcaaatcaaagcgagagtctgatgcttcagccttgggattcactgc

SEQ ID NO: 28 - DNA, 718 bp, artificial
  Sequence of the first arm of the NadHb/NadHd/NadHe dsRNA used to target concomitantly the NadHb, NadHd and NadHe genes of Xanthomonas species
aacagaagaagggagcgctggaatgggagtgattcagaccctggatcgtctgatgaccaacccgatgccggaaggccgggtcgaagacatcctgcgcccggaaggcgaaaacccgttgctcgaaaagggctacgtgaccaccagcgtcgatgcgctgttgaactgggcgcgtacaggttcgatgtggccgatgacctttggtctggcctgctgtgcagtcgagatgatgcagatcgtgagtgagtaccgccaggcaaccgatgccttcgcgagcaatcctgtggaaagcaagcaggaaatccgcaattacacgatgaacttcggcccgcagcatcctgcggcgcacggcgtattgcgcctgatcctggaaatggacggcgaaaccgtggtgcgtgccgacccgcatatcggcctgctgcaccgtggcaccgagaagctggccgagtccaagccgttcaaccagtcggttccgtacagcatcgacgggtaatttcgaagcggcgcgcgacgtcgatccgcaggtagtgctgagcgacaagacgcgcgcgcacatcgatcattggctgagcaagttcccgcccgaccgcaagcgttcggccgtgttgcagggtctgcatgccgcgcaggaacagaaccagggttggttgaccgacgagctgatcgtgggcgtggccaagtatctggagctgccgccggtgtgggcctacgaagtggggct

SEQ ID NO: 29 - DNA, 718 bp, artificial
  Sequence of the second arm of the NadHb/NadHd/NadHe dsRNA used to target concomitantly the NadHb, NadHd and NadHe genes of Xanthomonas species
tcagccacttcgtaggcccacaccggcggcagctccagatacttggccacgcccacgatcagctcgtcggtcaaccaaccctggttctgttcctgcgcggcatgcagaccctgcaacacggccgaacgcttgcggtcgggcgggaacttgctcagccaatgatcgatgtgcgcgcgcgtcttgtcgctcagcactacctgcggatcgacgtcgcgcgccgcttcgaaattacccgtcgatgctgtacggaaccgactggttgaacggcttggactcggccagcttctcggtgccacggtgcagcaggccgatatgcgggtcggcacgcaccacggtttcgccgtccatttccaggatcaggcgcaatacgccgtgcgccgcaggatgctgcgggccgaagttcatcgtgtaattgcggatttcctgcttgctttccacaggattgctcgcgaaggcatcggttgcctggcggtactcactcacgatctgcatcatctcgactgcacagcaggccagaccaaaggtcatcggccacatcgaacctgtacgcgcccagttcaacagcgcatcgacgctggtggtcacgtagcccttttcgagcaacgggttttcgccttccgggcgcaggatgtcttcgacccggccttccggcatcgggttggtcatcagacgatccagggtctgaatcactcccattccagcgctcccttcttcctgc

SEQ ID NO: 30 - DNA, 677 bp, artificial
  Sequence of the first arm of the DnaA/DnaE1/DnaE2 dsRNA used to target concomitantly the DnaA, DnaE1 and DnaE2 genes of Xanthomonas species
aacaatgcttggccccgctgtctggaacgtctcgaagctgaattcccgcccgaggatgtccacacctggttgaaacccctgcaggccgaagatcgcggcgacagcatcgtgctgtacgcgccgaacgccttcattgttgagcaggtccgcgagcgatacctgccgcgcatccgcgagttgctggcatatttcgccggcaatggcgaggtggcgctggcggtcggctcccgtcgatcgcgcgaagccttcctcaagccgttggcccaggtggtcaatgtcgtcgaacggcgtcagacattgccggtactggcgaacttgctggtgcaggtgaacaacggccagctgtcgctgacggggaccgacctggaagtcgaaatgatctcgcgcaccatggtcgaggacgcccaggacggcgaaaccacgatcccggcgcgcaagctgttcgacatcctggcatatgtccacttcccgcttcgtccatctgcacgtccacaccgagttctcgttggcggattccaccatccgcgtgcccgagaaaccggatcaggctgacccgaaaaaagccaagcaggccaacctgctgagccgcgcggtcgaactcgacttgcccgcgctggcggtcaccgacctgaacaacctgttcgccctggtcaagttctacaaggccgccgaaggct

SEQ ID NO: 31 - DNA, 677 bp, artificial
  Sequence of the second arm of the DnaA/DnaE1/DnaE2 dsRNA used to target concomitantly the DnaA, DnaE1 and DnaE2 genes of Xanthomonas species
tcagttcggcggccttgtagaacttgaccagggcgaacaggttgttcaggtcggtgaccgccagcgcgggcaagtcgagttcgaccgcgcggctcagcaggttggcctgcttggcttttttcgggtcagcctgatccggtttctcgggcacgcggatggtggaatccgccaacgagaactcggtgtggacgtgcagatggacgaagcgggaagtggacatatgccaggatgtcgaacagcttgcgcgccgggatcgtggtttcgccgtcctgggcgtcctcgaccatggtgcgcgagatcatttcgacttccaggtcggtccccgtcagcgacagctggccgttgttcacctgcaccagcaagttcgccagtaccggcaatgtctgacgccgttcgacgacattgaccacctgggccaacggcttgaggaaggcttcgcgcgatcgacgggagccgaccgccagcgccacctcgccattgccggcgaaatatgccagcaactcgcggatgcgcggcaggtatcgctcgcggacctgctcaacaatgaaggcgttcggcgcgtacagcacgatgctgtcgccgcgatcttcggcctgcaggggtttcaaccaggtgtggacatcctcgggcgggaattcagcttcgagacgttccagacagcggggccaagcatctgc

SEQ ID NO: 32 - DNA, 717 bp, artificial
  GFP reporter sequence contained in the GFPpPNpt plasmid
atgagtaagggggaggagctctttacaggggtggtacccatactcgtcgagctcgacggggacgtgaacggtcagaagttttcggtaagcggggaaggggagggggacgcgacgtatgggaagctgacactcaagttcatatgtacaacaggaaaactgccggtcccttggcccacgctcgttacaacattaacatacggggtgcagtgtttctcgaggtatccggaccacatgaagcaacacgatttctttaaaagcgcaatgccagaggggtacgttcaagagaggacgattttctataaggacgatggtaattataaaacccgggcggaggttaaattcgagggggacacactggtgaacaggatagaattgaaggggatagacttcaaggaggacggcaacattcttggacacaaaatggaatacaactataactcacataatgtatacatcatggcagacaaaccaaagaatggaatcaaagttaacttcaaaattagacacaacattaaagatggaagcgttcaattagcagaccattatcaacaaaatactccaattggcgatggccctgtccttttaccagacaaccattacctgtccacacaatctgccctttccaaagatcccaacgaaaagagagatcacatgatccttcttgagtttgtaacagctgctgggattacacatggcatggatgaactatacaaataa

SEQ ID NO: 33 - DNA, 21 bp, artificial
  Primer sequence of Cfa6-Forward used for LMW Northern Blot
caacaacttgccgggatcctg

SEQ ID NO: 34 - DNA, 22 bp, artificial
  Primer sequence of Cfa6-Reverse used for LMW Northern Blot
ggcttgcatccgacagaatcag

SEQ ID NO: 35 - DNA, 21 bp, artificial
  Primer sequence of HrpL-Forward used for LMW Northern Blot
cacttcgtcttcccagctttc

SEQ ID NO: 36 - DNA, 24 bp, artificial
  Primer sequence of HrpL-Forward used for LMW Northern Blot
tttatccaaaagcgggtgatgaac

SEQ ID NO: 37 - DNA, 21 bp, artificial
  Primer sequence of miR159 probe used for LMW Northern Blot
aaacctaacttccctcgagat

SEQ ID NO: 38 - DNA, 19 bp, artificial
  Primer sequence of GyrA-Fwd used for RT-qPCR
aactgctgggtgagtacca

SEQ ID NO: 39 - DNA, 19 bp, artificial
  Primer sequence of GyrA-Rev used for RT-qPCR
gagctcttcgcggatcact

SEQ ID NO: 40 - DNA, 20 bp, artificial
  Primer sequence of CFA6-Fwd used for RT-qPCR
gtcttcatctttcccggtca

SEQ ID NO: 41 - DNA, 20 bp, artificial
  Primer sequence of CFA6-Rev used for RT-qPCR
gtctcgatctggtcgatggt

SEQ ID NO: 42 - DNA, 22 bp, artificial
  Primer sequence of HrpL-Fwd used for RT-qPCR
cgagtcattcaggccattgatt

SEQ ID NO: 43 - DNA, 22 bp, artificial
  Primer sequence of HrpL-Rev used for RT-qPCR
gtttcctgataattgccgtcca

SEQ ID NO: 44 - DNA, 20 bp, artificial
  Primer sequence of ProC-Fwd used for RT-qPCR
cgcagatgatgaaaagcgtc

SEQ ID NO: 45 - DNA, 19 bp, artificial
  Primer sequence of ProC-Rev used for RT-qPCR
agtcaggctggcacaggtg

SEQ ID NO: 46 - DNA, 20 bp, artificial
  qPCR primer RpoB-Fwd for bacterial transcript RpoB
gtaggtctggtccgtgttga

SEQ ID NO: 47 - DNA, 20 bp, artificial
  Primer sequence of RpoB-Rev used for RT-qPCR
gcaagtaatctcggacagcg

SEQ ID NO: 48 - DNA, 20 bp, artificial
  Primer sequence of Cyp3-Forward used for LMW Northern Blot
atccgagcatgacatgatga

SEQ ID NO: 49 - DNA, 20 bp, artificial
  Primer sequence of Cyp3-Reverse used for LMW Northern Blot
gtacgggtcgatgccatatt

SEQ ID NO: 50 - DNA, 21 bp, artificial
  Probe preparation for northern blot analysis U6
aggggccatgctaatcttctc

SEQ ID NO: 51 - DNA, 22 bp, artificial
  Primer sequence of Tomato Ubi-Fwd used for RT-qPCR
ggacggacgtactctagctgat

SEQ ID NO: 52 - DNA, 20 bp, artificial
  Primer sequence of Tomato Ubi-Rev used for RT-qPCR
agctttcgacctcaagggta

SEQ ID NO: 53 - DNA, 20 bp, artificial
  Primer sequence of Pto GFP-Fwd used for RT-qPCR
tggaagcgttcaactagcag

SEQ ID NO: 54 - DNA, 20 bp, artificial
  Primer sequence of Pto GFP-Rev used for RT-qPCR
aaagggcagattgtgtggac

SEQ ID NO: 55 - DNA, 20 bp, artificial
  Primer sequence of IR-CFA6/HRPL-Fwd used for RT-qPCR
gttcatcacccgcttttgga

SEQ ID NO: 56 - DNA, 20 bp, artificial
  Primer sequence of IR-CFA6/HRPL-Rev used for RT-qPCR
cccctctttctaccttccca

SEQ ID NO: 57 - DNA, 21 bp, artificial
  Primer sequence of Ath-Ubi-Fwd used for RT-qPCR
tgaagtcgtgagacagcgttg

SEQ ID NO: 58 - DNA, 20 bp, artificial
  Primer sequence of Ath-Ubi -Rev used for RT-qPCR
gggctttctcattgttggtc

SEQ ID NO: 59 - DNA, 35 bp, artificial
  HRPL-pDON207-F for Cloning of WT HRPL and mut HRPL in pDON207-attB1/B2
aaaaagcaggcttcatgtttcagaagattgtgatc

SEQ ID NO: 60 - DNA, 34 bp, artificial
  HRPL-pDON207-Rev for Cloning of WT HRPL and mut HRPL in pDON207-attB1/B2
agaaagctgggtcggcgaacgggtcgatttgctg

SEQ ID NO: 61 - DNA, 21 bp, artificial
  Primer dcl2-1-WT-fwd for genotyping dcl2-1 allele
tgaatcatctggaagaggtgg

SEQ ID NO: 62 - DNA, 19 bp, artificial
  Primer dcl2-1-mut-fwd for genotyping dcl2-1 allele
attttgccgatttcggaac

SEQ ID NO: 63 - DNA, 21 bp, artificial
  Primer dcl2-1-WT-Rev for genotyping dcl2-1 allele
tgaatcatctggaagaggtgg

SEQ ID NO: 64 - DNA, 21 bp, artificial
  Primer dcl3-1-fwd for genotyping dcl3-1 allele
acaggtaaccttgccatgttg

SEQ ID NO: 65 - DNA, 21 bp, artificial
  Primer dcl3-1-Rev for genotyping dcl3-1 allele
tggaaaagtttgctacaacgg

SEQ ID NO: 66 - DNA, 22 bp, artificial
  primer LBa1
tggttcacgtagtgggccatcg

SEQ ID NO: 67 - DNA, 23 bp, artificial
  Primer dcl4-2-G8605 Fwd for genotyping dcl4-2 allele
ggctgcacagctgatgattacaa

SEQ ID NO: 68 - DNA, 27 bp, artificial
  Primer dcl4-2-G8605 Rev for genotyping dcl4-2 allele
gccgctcgagatcatcagcaaaggaat

SEQ ID NO: 69 - DNA, 28 bp, artificial
  Primer GABI-8474-LP
ataataacgctgcggacatctacatttt

SEQ ID NO: 70 - DNA, 19 bp, artificial
  Northern blot analysis IR-HHR Fwd Primer
ggcatcacagatcaacgcc

SEQ ID NO: 71 - DNA, 18 bp, artificial
  Northern blot analysis IR-HHR Rev Primer
actgtcgtccgatgccac

SEQ ID NO: 72 - DNA, 20 bp, artificial
  RT-qPCR LuxA Fwd
cggagtttggtttgcttggt

SEQ ID NO: 73 - DNA, 20 bp, artificial
  RT-qPCR LuxA Rev
caagttggcgtactggatgg

SEQ ID NO: 74 - DNA, 20 bp, artificial
  RT-qPCR LuxB Fwd
gcggaggaagcttgcttatt

SEQ ID NO: 75 - DNA, 20 bp, artificial
  RT-qPCR LuxB Rev
tgatattcaaccgggcgatt

SEQ ID NO: 76 - DNA, 35 bp, artificial; identical to SEQ ID NO: 59
  HRPL-pDON207-Fwd

SEQ ID NO: 77 - DNA, 34 bp, artificial; identical to SEQ ID NO: 60
  HRPL-pDON207-Rev

SEQ ID NO: 78 - DNA, 37 bp, artificial
  T7 Fwd CFA6/HRPL
taatacgactcactatagggagatgaacaagcgctac

SEQ ID NO: 79 - DNA, 37 bp, artificial
  T7 Rev CFA6/HRPL
taatacgactcactatagggagcagggcgtttatcca

SEQ ID NO: 80 - DNA, 37 bp, artificial
  T7 Fwd CYP51
taatacgactcactatagggagcagcaagtttgacga

SEQ ID NO: 81 - DNA, 37 bp, artificial
  T7 Rev CYP51
taatacgactcactatagggaggcagcaaactcggca

SEQ ID NO: 82 - DNA, 43 bp, artificial
  T7 Fwd Dc3000_FusA
taatacgactcactatagggagatggctcgtactactccgatt

SEQ ID NO: 83 - DNA, 43 bp, artificial
  T7 Rev Dc3000_FusA
taatacgactcactatagggaggccaaaaggcagtgatagcag

SEQ ID NO: 84 - DNA, 42 bp, artificial
  T7 Fwd Dc3000_SecE
taatacgactcactatagggagtgaatcccaaggctgaagca

SEQ ID NO: 85 - DNA, 42 bp, artificial
  T7 Rev Dc3000_SecE
taatacgactcactatagggagatctctgcacgcgcttcttt

SEQ ID NO: 86 - DNA, 43 bp, artificial
  T7 Fwd Dc3000_GyrB
taatacgactcactatagggagatgagcgagaacaacacgtac

SEQ ID NO: 87 - DNA, 43 bp, artificial
  T7 Rev Dc3000_GyrB
taatacgactcactatagggagccgtatggatggtgatgctga

SEQ ID NO: 88 - DNA, 213 bp, artificial
  First strand XC_RS06155  XC_1225
atgcatcgacatgttcatgagcccgaaatcctagcagacccggcagccttcgaagctgccgaacattggcggcaacgcgatcttgcggtggtgtggcacccctgcacccagatgcgcgagcacccgcacacactacccctggtgccgatcgcacgtggcgacggcgcctggctgatcggccacgatggccgccactatctggatgcggtcagc

SEQ ID NO: 89 - DNA, 213 bp, artificial
  Second strand XC_RS06155  XC_1225
gctgaccgcatccagatagtggcggccatcgtggccgatcagccaggcgccgtcgccacgtgcgatcggcaccaggggtagtgtgtgcgggtgctcgcgcatctgggtgcaggggtgccacaccaccgcaagatcgcgttgccgccaatgttcggcagcttcgaaggctgccgggtctgctaggatttcgggctcatgaacatgtcgatgcat

SEQ ID NO: 90 - DNA, 189 bp, artificial
  First strand XC_RS02265 = XC_0447
atgagcgttgacgctgccgcgccgacttccgcctccactccgccgcaaccgcctgcgcctccccagttccccaccttgctcggccacccccggccgctgtggatgctgttcatgaccgaattctgggaacgctttgcgttctacggcattcgttgggcgctggtgctgtacatcgtggcgcagttcttc

SEQ ID NO: 91 - DNA, 189 bp, artificial
  Second strand XC_RS02265 = XC_0447
gaagaactgcgccacgatgtacagcaccagcgcccaacgaatgccgtagaacgcaaagcgttcccagaattcggtcatgaacagcatccacagcggccgggggtggccgagcaaggtggggaactggggaggcgcaggcggttgcggcggagtggaggcggaagtcggcgcggcagcgtcaacgctcat

SEQ ID NO: 92 - DNA, 196 bp, artificial
  First strand XC_RS18260 = XC_3609
atgagcgatgtccttccgatcattctttccggcggctccggcacgcgcctgtggccgttgtcgcgcgaatcgtacccgaagcagttcctgccgctggtcggcgagcacagcatgctgcaggccacctggctgcgctccgctccggtggcggcgcacgcacccatcgtggtggccaacgaagagcaccgcttcatgg

SEQ ID NO: 93 - DNA, 196 bp, artificial
  Second strand XC_RS18260 = XC_3609
ccatgaagcggtgctcttcgttggccaccacgatgggtgcgtgcgccgccaccggagcggagcgcagccaggtggcctgcagcatgctgtgctcgccgaccagcggcaggaactgcttcgggtacgattcgcgcgacaacggccacaggcgcgtgccggagccgccggaaagaatgatcggaaggacatcgctcat

SEQ ID NO: 94 - DNA, 189 bp, artificial
  First strand XC_RS11930 = XC_2375
gagccgtcgcatcattccctgcctggacgtgcgcgatggccgcgtggtcaagggcgtcaagttccgcgaccacatcgacatgggcgacatcgtcgagttggcgatgcgctaccgcgaccagggcgcggacgagttggtgttctacgacattggcgccagcccggaagggcgttcggtggactatgcgtg

SEQ ID NO: 95 - DNA, 189 bp, artificial
  Second strand XC_RS11930 = XC_2375
cacgcatagtccaccgaacgcccttccgggctggcgccaatgtcgtagaacaccaactcgtccgcgccctggtcgcggtagcgcatcgccaactcgacgatgtcgcccatgtcgatgtggtcgcggaacttgacgcccttgaccacgcggccatcgcgcacgtccaggcagggaatgatgcgacggctc

SEQ ID NO: 96 - DNA, 196 bp, artificial
  First strand XC_RS17005 = XC_3357
atggcaaagactcatgaaatcaaggtcgagcgccgcgcagacgaggggaagggtgcgagccgccgcctgcgtcacgctggcgtcattccggccatcgtttacggtggcgaactcgagccggtcagcatccagctcaaccacgagcagatctggcttgcgcagcagaacgagtggttctactcgtcgatcctcgacc

SEQ ID NO: 97 - DNA, 196 bp, artificial
  First strand XC_RS17005 = XC_3357
ggtcgaggatcgacgagtagaaccactcgttctgctgcgcaagccagatctgctcgtggttgagctggatgctgaccggctcgagttcgccaccgtaaacgatggccggaatgacgccagcgtgacgcaggcggcggctcgcacccttcccctcgtctgcgcggcgctcgaccttgatttcatgagtctttgccat

SEQ ID NO: 98 - DNA, 44 bp, artificial
  T7 Fwd XC_RS06155  XC_1225
taatacgactcactatagggagatgcatcgacatgttcatgagc

SEQ ID NO: 99 - DNA, 42 bp, artificial
  T7 Rev XC_RS06155  XC_1225
taatacgactcactatagggaggctgaccgcatccagatagt

SEQ ID NO: 100 - DNA, 40 bp, artificial
  T7 Fwd XC_RS02265 = XC_0447
taatacgactcactatagggagatgagcgttgacgctgcc

SEQ ID NO: 101 - DNA, 43 bp, artificial
  T7 Rev XC_RS02265 = XC_0447
taatacgactcactatagggaggaagaactgcgccacgatgta

SEQ ID NO: 102 - DNA, 41 bp, artificial
  T7 Fwd XC_RS18260 = XC_3609
taatacgactcactatagggagatgagcgatgtccttccga

SEQ ID NO: 103 - DNA, 40 bp, artificial
  T7 Rev XC_RS18260 = XC_3609
taatacgactcactatagggagccatgaagcggtgctctt

SEQ ID NO: 104 - DNA, 40 bp, artificial
  T7 Fwd XC_RS11930 = XC_2375
taatacgactcactatagggaggagccgtcgcatcattcc

SEQ ID NO: 105 - DNA, 41 bp, artificial
  T7 Rev XC_RS11930 = XC_2375
taatacgactcactatagggagcacgcatagtccaccgaac

SEQ ID NO: 106 - DNA, 43 bp, artificial
  T7 Fwd XC_RS17005 = XC_3357
taatacgactcactatagggagatggcaaagactcatgaaatc

SEQ ID NO: 107 - DNA, 42 bp, artificial
  T7 Rev XC_RS17005 = XC_3357
taatacgactcactatagggagggtcgaggatcgacgagtag

SEQ ID NO: 108 - DNA, 632 bp, artificial
  first strand IT13 (P. aeruginosa)
gtgtccgtggaactttggcagcagtgcgtggatcttctccgcgatgagctgccgtcccaacaattcaacacctggatccgtcccttgcaggtcgaagccgaaggcgacgaattgcgtgtgtatgcacccaaccgtttcgtcctcgattgggtgaacgagaaatacctcggtcggcttctggaactgctcggtgaacgcgggagggtcagttgatcatgcatttcaccattcaacgcgaagccctgttgaaaccgctgcaactggtcgccggcgtcgtggaacgccgccagacattgccggttctctccaacgtcctgctggtggtcgaaggccagcaactgtcgctgaccggcaccgacctcgaggtcgagctggttggtcgcgtggtactggaagatgccgccgaacccggcgagatcaccgcatatgagcgagaacaacacgtacgactcttccagcatcaaggtgctgaaggggctggatgccgtacgcaagcgccccggcatgtacatcggcgacaccgacgatggcaccggtctgcaccacatggtgttcgaggtggtggataactccatcgacgaagcgctggccggttactgcagcgaaatcagcatcaccatccatacggggct

SEQ ID NO: 109 - DNA, 632 bp, artificial
  second strand IT13 (P. aeruginosa)
tcagccgtatggatggtgatgctgatttcgctgcagtaaccggccagcgcttcgtcgatggagttatccaccacctcgaacaccatgtggtgcagaccggtgccatcgtcggtgtcgccgatgtacatgccggggcgcttgcgtacggcatccagccccttcagcaccttgatgctggaagagtcgtacgtgttgttctcgctcatatgcggtgatctcgccgggttcggcggcatcttccagtaccacgcgaccaaccagctcgacctcgaggtcggtgccggtcagcgacagttgctggccttcgaccaccagcaggacgttggagagaaccggcaatgtctggcggcgttccacgacgccggcgaccagttgcagcggtttcaacagggcttcgcgttgaatggtgaaatgcatgatcaactgaccctcccgcgttcaccgagcagttccagaagccgaccgaggtatttctcgttcacccaatcgaggacgaaacggttgggtgcatacacacgcaattcgtcgccttcggcttcgacctgcaagggacggatccaggtgttgaattgttgggacggcagctcatcgcggagaagatccacgcactgctgccaaagttccacggacac

SEQ ID NO: 110 - DNA, 803 bp, artificial
  first strand IT14 (P. aeruginosa)
tgaaagacttgcttaatctgttgaaaaaccagggtcaaatcgaagagttcgatgccatccgtattggcctggcttcgcccgagatgattcgttcctggtctttcggcgaagttaaaaagccggaaaccatcaactaccgtaccttcaagccggagcgcgacggcctgttctgcgccaagatcttcggcccggtgaaggactacgagtgcctgtgcggcaagtacaagcgcctcaagcaccgcggtgtgatctgcgagaagtgatcatgaatgccaaggcagaagccaaagaatcacgttttgatctcttgaaatggctcttggttgccgttctggttgtggttgccgttgtgggcaatcagtacttttcggctcaaccaatcctgtatcgcgttctcggtattctcgttctggcggtgatcgctgccttcctggctctgcaaacggccaaggggcaggccttctttagtcttgctaaggaagcgcgcgtcgagattcgcaaggtcgtatggccgagtcgtcaagaaacaactcagaccacgctgatcggcatatggctttcgaattgccgccgctgccttacgaaaagaacgcccttgagccgcacatttccgcagaaaccctggaataccaccacgacaagcaccacaacacctacgtggtgaacctgaacaacctgatcccgggtaccgagttcgaaggcaagagcctcgaagagatcgtcaagagctcctccggcggcatcttcaacaacgccgcccaggtgtggaaccacaccttctactggaactgcctgagccggct

SEQ ID NO: 111 - DNA, 803 bp, artificial
  second strand IT14 (P. aeruginosa)
tcagggctcaggcagttccagtagaaggtgtggttccacacctgggcggcgttgttgaagatgccgccggaggagctcttgacgatctcttcgaggctcttgccttcgaactcggtacccgggatcaggttgttcaggttcaccacgtaggtgttgtggtgcttgtcgtggtggtattccagggtttctgcggaaatgtgcggctcaagggcgttcttttcgtaaggcagcggcggcaattcgaaagccatatgccgatcagcgtggtctgagttgtttcttgacgactcggccatacgaccttgcgaatctcgacgcgcgcttccttagcaagactaaagaaggcctgccccttggccgtttgcagagccaggaaggcagcgatcaccgccagaacgagaataccgagaacgcgatacaggattggttgagccgaaaagtactgattgcccacaacggcaaccacaaccagaacggcaaccaagagccatttcaagagatcaaaacgtgattctttggcttctgccttggcattcatgatcacttctcgcagatcacaccgcggtgcttgaggcgcttgtacttgccgcacaggcactcgtagtccttcaccgggccgaagatcttggcgcagaacaggccgtcgcgctccggcttgaaggtacggtagttgatggtttccggctttttaacttcgccgaaagaccaggaacgaatcatctcgggcgaagccaggccaatacggatggcatcgaactcttcgatttgaccctggtttttcaacagattaagcaagtctttca

SEQ ID NO: 112 - DNA, 684 bp, artificial
  first strand IT16 (P. aeruginosa)
atgtcccagcctttgctccgcgccctgtttgccccttccagtcgttcgtacgttcccgccgtccttctcagcctggccctcggcatccaggcggcgcacgccgaaaacagcggcgggaacgccttcgtcccggccggcaaccagcaggaggcgcactggacgatcaacctcaaggatgccgacatccgcgaattcatcgaccagatttccgaaatcaccggcgagaccttgatcatggcgcagatattcaaccccaacccggggaataccctcgataccgtggccaatgccctgaaggagcaggccaacgcagcgaacaaggacgtcaacgacgcgatcaaggccttgcaggggaccgacaatgccgacaacccggcgctgctggccgagctgcaacacaagatcaacaagtggtcggtcatctacaacatcaactcgacggtgacccgtgcgcgcatatgcgccgcctgctgatcggcggactgctggcgctgctgcctggcgcggtcttgcgcgcgcagccgctggactggcccagcctgccttacgactatgtggcgcagggcgaaagcctgcgcgacgtgctggccaacttcggcgccaactacgatgcctcggtgatcgtcagtgacaaggtcaacgaccaggtcagcggtcgcttcgacctggaaagcccgcagggct

SEQ ID NO: 113 - DNA, 684 bp, artificial
  Second strand IT16 (P. aeruginosa)
tcagctgcgggctttccaggtcgaagcgaccgctgacctggtcgttgaccttgtcactgacgatcaccgaggcatcgtagttggcgccgaagttggccagcacgtcgcgcaggctttcgccctgcgccacatagtcgtaaggcaggctgggccagtccagcggctgcgcgcgcaagaccgcgccaggcagcagcgccagcagtccgccgatcagcaggcggcgcatatgcgcgcacgggtcaccgtcgagttgatgttgtagatgaccgaccacttgttgatcttgtgttgcagctcggccagcagcgccgggttgtcggcattgtcggtcccctgcaaggccttgatcgcgtcgttgacgtccttgttcgctgcgttggcctgctccttcagggcattggccacggtatcgagggtattccccgggttggggttgaatatctgcgccatgatcaaggtctcgccggtgatttcggaaatctggtcgatgaattcgcggatgtcggcatccttgaggttgatcgtccagtgcgcctcctgctggttgccggccgggacgaaggcgttcccgccgctgttttcggcgtgcgccgcctggatgccgagggccaggctgagaaggacggcgggaacgtacgaacgactggaaggggcaaacagggcgcggagcaaaggctgggacat

SEQ ID NO: 114 - DNA, 643 bp, artificial
  first strand IT18 (P. aeruginosa)
atgtcccagcctttgctccgcgccctgtttgccccttccagtcgttcgtacgttcccgccgtccttctcagcctggccctcggcatccaggcggcgcacgccgaaaacagcggcgggaacgccttcgtcccggccggcaaccagcaggaggcgcactggacgatcaacctcaaggatgccgacatccgcgaattcatcgaccagattgatcatgcaaggagccaaatctcttggccgaaagcagataacgtcttgtcattggaacattccaactttcgaatacagggtaaacaaggaagagggcgtatatgttctgctcgagggcgaactgaccgtccaggacatcgattccactttttgcctggcgcctggcgagttgcttttcgtccgccgcggaagctatgtcgtaagtaccaagggaaaggacagccgaatacgcatatgccacgttgtagtacccgttccgcccagcgcctgtctccgctgttcctggtcctgtcgctggccgtcctcggcctggcgccgccggtccatccggcacctgccgagactgccgccgcgcaagaggaagagcagtggacgatcaacatgaaggatgccgagatcggcgacttcatcgagcaggtatcgagcatcagcggct

SEQ ID NO: 115 - DNA, 643 bp, artificial
  second strand IT18 (P. aeruginosa)
tcaggctgatgctcgatacctgctcgatgaagtcgccgatctcggcatccttcatgttgatcgtccactgctcttcctcttgcgcggcggcagtctcggcaggtgccggatggaccggcggcgccaggccgaggacggccagcgacaggaccaggaacagcggagacaggcgctgggcggaacgggtactacaacgtggcatatgcgtattcggctgtcctttcccttggtacttacgacatagcttccgcggcggacgaaaagcaactcgccaggcgccaggcaaaaagtggaatcgatgtcctggacggtcagttcgccctcgagcagaacatatacgccctcttccttgtttaccctgtattcgaaagttggaatgttccaatgacaagacgttatctgctttcggccaagagatttggctccttgcatgatcaatctggtcgatgaattcgcggatgtcggcatccttgaggttgatcgtccagtgcgcctcctgctggttgccggccgggacgaaggcgttcccgccgctgttttcggcgtgcgccgcctggatgccgagggccaggctgagaaggacggcgggaacgtacgaacgactggaaggggcaaacagggcgcggagcaaaggctgggacat

SEQ ID NO: 116 - DNA, 637 bp, artificial
  first strand IT21 (Shigella flexneri)
aatgatcaaggcgacggacagaaaactggtagtaggactggaaattggtaccgcgaaggttgccgctttagtaggggaagttctgcccgacggtatggtcaatatcattggcgtgggcagctgcccgtcacgtggtatggataaaggtggggtgaacgacctcgaatccgtggtcaagtgcgtacaacgcgccattgaccaggcagaattgatggcaggatctttgtggttggcgtgtttcagcttgaggttggaaatcccgtgacggtaacgttgctcaagggtttcgcggttggtggcggtaacatccagatcacgcagcaagccgtcgtgaatgccgtaggcccagccgtgaatggtaactttctgcccgcgtttccacgctgattgcataatggtggagtggcccaggttatacacctgcattggctgaaattaccgcatccctggtaaaagagctgcgtgagcgtactggcgcaggcatgatggattgcaaaaaagcactgactgaagctaacggcgacatcgagctggcaatcgaaaacatgcgtaagtccggtgctattaaagcagcgaaaaaagcaggcaacgttgctgctgacggcgtgatcaaaaccaaaatcgacggcaactggct

SEQ ID NO: 117 - DNA, 636 bp, artificial
  second strand IT21 (Shigella flexneri)
tcagagttgccgtcgattttggttttgatcacgccgtcagcagcaacgttgcctgcttttttcgctgctttaatagcaccggacttacgcatgttttcgattgccagctcgatgtcgccgttagcttcagtcagtgcttttttgcaatccatcatgcctgcgccagtacgctcacgcagctcttttaccagggatgcggtaatttcagccaatgcaggtgtataacctgggccactccaccattatgcaatcagcgtggaaacgcgggcagaaagttaccattcacggctgggcctacggcattcacgacggcttgctgcgtgatctggatgttaccgccaccaaccgcgaaacccttgagcaacgttaccgtcacgggatttccaacctcaagctgaaacacgccaaccacaaagatcctgccatcaattctgcctggtcaatggcgcgttgtacgcacttgaccacggattcgaggtcgttcaccccacctttatccataccacgtgacgggcagctgcccacgccaatgatattgaccataccgtcgggcagaacttcccctactaaagcggcaaccttcgcggtaccaatttccagtcctactaccagttttctgtccgtcgccttgatcat

SEQ ID NO: 118 - DNA, 686 bp, artificial
  first strand IT26 (Shigella flexneri)
taccgggggtaccactacgccttcacgcggcgcttcaggattcggcgctggcagattcatcaacttcgccagaatgctcgccagtttcaggcgcatttccggacgacggacgatcatgtcgatcgcgcctttctcgatcaggaattcactgcgctggaatctaggcggcagtttttcgcgaacggtctgttcgataacacgcggaccggcaaagccgattaacgcttgatctttcttgatgtgcttcatcagacgcttacgtttacgcatctgggttggcgtcagggtgttacgcttgttcgcatacgggttttccccttctttgaactgaatacgaatcggcgatcccattacgtccagcgatttgcggaagtagttcatcaagtagcgcttgtaggaatcaggcaggtctttcacctgattaccgtgaatcaccacaatcggcggggcattttttatcgtcaaccaatgggctggcgtcgtgttctgcttcgatctcttcagcaggaagtggggcgggttcagcgtctggcgtaacaaaggtttcggtagatactgccagcggctggccaattttcgtgacagacaggctttccagttgctcaaccagattcactttacccggtgcaaacaggttgataacggtggaaccgagtttaaagcgacccatttcctggcctttggct

SEQ ID NO: 119 - DNA, 686 bp, artificial
  second strand IT26 (Shigella flexneri)
tcagaaaggccaggaaatgggtcgctttaaactcggttccaccgttatcaacctgtttgcaccgggtaaagtgaatctggttgagcaactggaaagcctgtctgtcacgaaaattggccagccgctggcagtatctaccgaaacctttgttacgccagacgctgaacccgccccacttcctgctgaagagatcgaagcagaacacgacgccagcccattggttgacgataaaaaatgccccgccgattgtggtgattcacggtaatcaggtgaaagacctgcctgattcctacaagcgctacttgatgaactacttccgcaaatcgctggacgtaatgggatcgccgattcgtattcagttcaaagaaggggaaaacccgtatgcgaacaagcgtaacaccctgacgccaacccagatgcgtaaacgtaagcgtctgatgaagcacatcaagaaagatcaagcgttaatcggctttgccggtccgcgtgttatcgaacagaccgttcgcgaaaaactgccgcctagattccagcgcagtgaattcctgatcgagaaaggcgcgatcgacatgatcgtccgtcgtccggaaatgcgcctgaaactggcgagcattctggcgaagttgatgaatctgccagcgccgaatcctgaagcgccgcgtgaaggcgtagtggtacccccggta

SEQ ID NO: 120 - DNA, 687 bp, artificial
  first strand IT27 (Shigella flexneri)
aatgacggttagctcaggcaatgaaactttgactatcgatgaagggcaaattgcttttatagagcgaaatatacaaataaacgtctccataaaaaaatctgatagcattaatccatttgagattataagccttgacagaaatttattattaagcattattagaataatggaaccaatttattcatttcaacactcctattctgaggagaaaagggggttagatcatggtggatttgtgcaacgacttgttaagtataaaggaaggccaaaagaaagagtttacactccattctggtaataaagtttcctttatcaaagccaagattcctcataaaaggatccaagatttaaccttcgtcaaccaaaaaacgaatgtacgcgatcaagaatccctaacagaagaatcactagccgatatcataaaaactataaagctacaacaattcttccctggcatggtagcggtgctgaccataacggtgatggtggtgaggctgttacaggagacaatctgtttataataaatggagaaattatttcaggtggacatggtggcgatagttatagtgatagtgatggggggaatggaggtgatgccgtcacaggagtcaatctacccataatcaacaaagggactatttccggtggtaatggaggtaacaattatggtgagggtgatggcgggct

SEQ ID NO: 121 - DNA, 687 bp, artificial
  second strand IT27 (Shigella flexneri)
tcagcgccatcaccctcaccataattgttacctccattaccaccggaaatagtccctttgttgattatgggtagattgactcctgtgacggcatcacctccattccccccatcactatcactataactatcgccaccatgtccacctgaaataatttctccatttattataaacagattgtctcctgtaacagcctcaccaccatcaccgttatggtcagcaccgctaccatgccagggaagaattgttgtagctttatagtttttatgatatcggctagtgattcttctgttagggattcttgatcgcgtacattcgttttttggttgacgaaggttaaatcttggatccttttatgaggaatcttggctttgataaaggaaactttattaccagaatggagtgtaaactctttcttttggccttcctttatacttaacaagtcgttgcacaaatccaccatgatctaacccccttttctcctcagaataggagtgttgaaatgaataaattggttccattattctaataatgcttaataataaatttctgtcaaggcttataatctcaaatggattaatgctatcagatttttttatggagacgtttatttgtatatttcgctctataaaagcaatttgcccttcatcgatagtcaaagtttcattgcctgagctaaccgtcatt

SEQ ID NO: 122 - DNA, 217 bp, artificial
  First strand FusA  (Shigella flexneri)
atgatcaaggcgacggacagaaaactggtagtaggactggaaattggtaccgcgaaggttgccgctttagtaggggaagttctgcccgacggtatggtcaatatcattggcgtgggcagctgcccgtcacgtggtatggataaaggtggggtgaacgacctcgaatccgtggtcaagtgcgtacaacgcgccattgaccaggcagaattgatggcag

SEQ ID NO: 123 - DNA, 217 bp, artificial
  Second strand FusA (Shigella flexneri)
ctgccatcaattctgcctggtcaatggcgcgttgtacgcacttgaccacggattcgaggtcgttcaccccacctttatccataccacgtgacgggcagctgcccacgccaatgatattgaccataccgtcgggcagaacttcccctactaaagcggcaaccttcgcggtaccaatttccagtcctactaccagttttctgtccgtcgccttgatcat

SEQ ID NO: 124 - DNA, 200 bp, artificial
  First strand Can (Shigella flexneri)
tttgtggttggcgtgtttcagcttgaggttggaaatcccgtgacggtaacgttgctcaagggtttcgcggttggtggcggtaacatccagatcacgcagcaagccgtcgtgaatgccgtaggcccagccgtgaatggtaactttctgcccgcgtttccacgctgattgcataatggtggagtggcccaggttatacacct

SEQ ID NO: 125 - DNA, 200 bp, artificial
  Second strand Can (Shigella flexneri)
aggtgtataacctgggccactccaccattatgcaatcagcgtggaaacgcgggcagaaagttaccattcacggctgggcctacggcattcacgacggcttgctgcgtgatctggatgttaccgccaccaaccgcgaaacccttgagcaacgttaccgtcacgggatttccaacctcaagctgaaacacgccaaccacaaa

SEQ ID NO: 126 - DNA, 207 bp, artificial
  First strand tsf (Shigella flexneri)
tggctgaaattaccgcatccctggtaaaagagctgcgtgagcgtactggcgcaggcatgatggattgcaaaaaagcactgactgaagctaacggcgacatcgagctggcaatcgaaaacatgcgtaagtccggtgctattaaagcagcgaaaaaagcaggcaacgttgctgctgacggcgtgatcaaaaccaaaatcgacggcaact

SEQ ID NO: 127 - DNA, 207 bp, artificial
  Second strand tsf (Shigella flexneri)
agttgccgtcgattttggttttgatcacgccgtcagcagcaacgttgcctgcttttttcgctgctttaatagcaccggacttacgcatgttttcgattgccagctcgatgtcgccgttagcttcagtcagtgcttttttgcaatccatcatgcctgcgccagtacgctcacgcagctcttttaccagggatgcggtaatttcagcca

SEQ ID NO: 128 - DNA, 227 bp, artificial
  First strand accD (Shigella flexneri)
taccgggggtaccactacgccttcacgcggcgcttcaggattcggcgctggcagattcatcaacttcgccagaatgctcgccagtttcaggcgcatttccggacgacggacgatcatgtcgatcgcgcctttctcgatcaggaattcactgcgctggaatctaggcggcagtttttcgcgaacggtctgttcgataacacgcggaccggcaaagccgattaacgctt

SEQ ID NO: 129 - DNA, 227 bp, artificial
  Second strand accD (Shigella flexneri)
aagcgttaatcggctttgccggtccgcgtgttatcgaacagaccgttcgcgaaaaactgccgcctagattccagcgcagtgaattcctgatcgagaaaggcgcgatcgacatgatcgtccgtcgtccggaaatgcgcctgaaactggcgagcattctggcgaagttgatgaatctgccagcgccgaatcctgaagcgccgcgtgaaggcgtagtggtacccccggta

SEQ ID NO: 130 - DNA, 217 bp, artificial
  First strand der(Shigella flexneri)
tttcttgatgtgcttcatcagacgcttacgtttacgcatctgggttggcgtcagggtgttacgcttgttcgcatacgggttttccccttctttgaactgaatacgaatcggcgatcccattacgtccagcgatttgcggaagtagttcatcaagtagcgcttgtaggaatcaggcaggtctttcacctgattaccgtgaatcaccacaatcggcggg

SEQ ID NO: 131 - DNA, 217 bp, artificial
  Second strand der(Shigella flexneri)
cccgccgattgtggtgattcacggtaatcaggtgaaagacctgcctgattcctacaagcgctacttgatgaactacttccgcaaatcgctggacgtaatgggatcgccgattcgtattcagttcaaagaaggggaaaacccgtatgcgaacaagcgtaacaccctgacgccaacccagatgcgtaaacgtaagcgtctgatgaagcacatcaagaaa

SEQ ID NO: 132 - DNA, 230 bp, artificial
  First strand psd(Shigella flexneri)
tttttatcgtcaaccaatgggctggcgtcgtgttctgcttcgatctcttcagcaggaagtggggcgggttcagcgtctggcgtaacaaaggtttcggtagatactgccagcggctggccaattttcgtgacagacaggctttccagttgctcaaccagattcactttacccggtgcaaacaggttgataacggtggaaccgagtttaaagcgacccatttcctggccttt

SEQ ID NO: 133 - DNA, 230 bp, artificial
  Second strand psd(Shigella flexneri)
aaaggccaggaaatgggtcgctttaaactcggttccaccgttatcaacctgtttgcaccgggtaaagtgaatctggttgagcaactggaaagcctgtctgtcacgaaaattggccagccgctggcagtatctaccgaaacctttgttacgccagacgctgaacccgccccacttcctgctgaagagatcgaagcagaacacgacgccagcccattggttgacgataaaaa

SEQ ID NO: 134 - DNA, 229 bp, artificial
  First strand VirB(Shigella flexneri)
atggtggatttgtgcaacgacttgttaagtataaaggaaggccaaaagaaagagtttacactccattctggtaataaagtttcctttatcaaagccaagattcctcataaaaggatccaagatttaaccttcgtcaaccaaaaaacgaatgtacgcgatcaagaatccctaacagaagaatcactagccgatatcataaaaactataaagctacaacaattcttccctg

SEQ ID NO: 135 - DNA, 229 bp, artificial
  Second strand VirB(Shigella flexneri)
cagggaagaattgttgtagctttatagtttttatgatatcggctagtgattcttctgttagggattcttgatcgcgtacattcgttttttggttgacgaaggttaaatcttggatccttttatgaggaatcttggctttgataaaggaaactttattaccagaatggagtgtaaactctttcttttggccttcctttatacttaacaagtcgttgcacaaatccaccat

SEQ ID NO: 136 - DNA, 220 bp, artificial
  First strand VirF(Shigella flexneri)
aatgacggttagctcaggcaatgaaactttgactatcgatgaagggcaaattgcttttatagagcgaaatatacaaataaacgtctccataaaaaaatctgatagcattaatccatttgagattataagccttgacagaaatttattattaagcattattagaataatggaaccaatttattcatttcaacactcctattctgaggagaaaagggggtta

SEQ ID NO: 137 - DNA, 220 bp, artificial
  Second strand VirF(Shigella flexneri)
taacccccttttctcctcagaataggagtgttgaaatgaataaattggttccattattctaataatgcttaataataaatttctgtcaaggcttataatctcaaatggattaatgctatcagatttttttatggagacgtttatttgtatatttcgctctataaaagcaatttgcccttcatcgatagtcaaagtttcattgcctgagctaaccgtcatt

SEQ ID NO: 138 - DNA, 226 bp, artificial
  First strand icsA(Shigella flexneri)
ggtagcggtgctgaccataacggtgatggtggtgaggctgttacaggagacaatctgtttataataaatggagaaattatttcaggtggacatggtggcgatagttatagtgatagtgatggggggaatggaggtgatgccgtcacaggagtcaatctacccataatcaacaaagggactatttccggtggtaatggaggtaacaattatggtgagggtgatggcg

SEQ ID NO: 139 - DNA, 226 bp, artificial
  Second strand icsA(Shigella flexneri)
cgccatcaccctcaccataattgttacctccattaccaccggaaatagtccctttgttgattatgggtagattgactcctgtgacggcatcacctccattccccccatcactatcactataactatcgccaccatgtccacctgaaataatttctccatttattataaacagattgtctcctgtaacagcctcaccaccatcaccgttatggtcagcaccgctacc

SEQ ID NO: 140 - DNA, 203 bp, artificial
  First strand spa47 (Shigella flexneri)
gagctatacaaaattgctcactcaattatcttttcctaatagaatctcggggccaatcttggaaacaagtcttagcgatgtttcgattggtgagatttgtaacattcaggctggaattgaaagtaatgaaattgttgcaagagctcaggttgtaggatttcatgatgaaaaaacaatattaagcttgattggaaattctcgtg

SEQ ID NO: 141 - DNA, 203 bp, artificial
  Second strand spa47 (Shigella flexneri)
cacgagaatttccaatcaagcttaatattgttttttcatcatgaaatcctacaacctgagctcttgcaacaatttcattactttcaattccagcctgaatgttacaaatctcaccaatcgaaacatcgctaagacttgtttccaagattggccccgagattctattaggaaaagataattgagtgagcaattttgtatagctc

SEQ ID NO: 142 - DNA, 240 bp, artificial
  First strand mukB (Shigella flexneri)
attgaacgcggtaaatttcgctcactgacgctgattaactggaacggcttttttgcccgaacttttgaccttgacgagctggtcacgacgctttccggcggtaacggggcgggtaaatccaccacgatggcggcgttcgttacggcgctgatccccgacctgaccctgctgcatttccgtaacactacggaagccggggccaccagcggttcgcgcgataaaggtctgcacggtaagctg

SEQ ID NO: 143 - DNA, 240 bp, artificial
  Second strand mukB (Shigella flexneri)
cagcttaccgtgcagacctttatcgcgcgaaccgctggtggccccggcttccgtagtgttacggaaatgcagcagggtcaggtcggggatcagcgccgtaacgaacgccgccatcgtggtggatttacccgccccgttaccgccggaaagcgtcgtgaccagctcgtcaaggtcaaaagttcgggcaaaaaagccgttccagttaatcagcgtcagtgagcgaaatttaccgcgttcaat

SEQ ID NO: 144 - DNA, 200 bp, artificial
  First strand ybiT(Shigella flexneri)
gtttccagtaacgtcaccatgcagttcggcagtaagccgttgtttgaaaacatttccgtcaaattcggcggcggcaaccgttacggcctgattggcgcgaacggtagtggtaaatccacctttatgaagattctcggcggcgaccttgagccgacgctgggtaacgtttccctcgatcccaacgagcgcattggtaagct

SEQ ID NO: 145 - DNA, 200 bp, artificial
  Second strand ybiT(Shigella flexneri)
agcttaccaatgcgctcgttgggatcgagggaaacgttacccagcgtcggctcaaggtcgccgccgagaatcttcataaaggtggatttaccactaccgttcgcgccaatcaggccgtaacggttgccgccgccgaatttgacggaaatgttttcaaacaacggcttactgccgaactgcatggtgacgttactggaaac

SEQ ID NO: 146 - DNA, 33 bp, artificial
  Pak_dnaaC_Fw
aggtctcaggctgtgtccgtggaactttggcag

SEQ ID NO: 147 - DNA, 30 bp, artificial
  Pak_dnaaC_Rv
aggtctcagatcaactgaccctcccgcgtt

SEQ ID NO: 148 - DNA, 32 bp, artificial
  Pak_dnanC_Fw
aggtctcagatcatgcatttcaccattcaacg

SEQ ID NO: 149 - DNA, 28 bp, artificial
  Pak_dnanC_Rv
aggtctcaatgcggtgatctcgccgggt

SEQ ID NO: 150 - DNA, 33 bp, artificial
  Pak_gyrbC_Fw
aggtctcagcatatgagcgagaacaacacgtac

SEQ ID NO: 151 - DNA, 33 bp, artificial
  Pak_gyrbC_Rv
aggtctcactgaccgtatggatggtgatgctga

SEQ ID NO: 152 - DNA, 33 bp, artificial
  Pak_dnaaB_Fw
aggtctcaaacagtgtccgtggaactttggcag

SEQ ID NO: 153 - DNA, 33 bp, artificial
  Pak_gyrbB_Rv
aggtctcaagccccgtatggatggtgatgctga

SEQ ID NO: 154 - DNA, 33 bp, artificial
  Pak_gyrbD_Fw
aggtctcatcagccgtatggatggtgatgctga

SEQ ID NO: 155 - DNA, 33 bp, artificial
  Pak_dnaaD_Rv
aggtctcagcaggtgtccgtggaactttggcag

SEQ ID NO: 156 - DNA, 37 bp, artificial
  Pak_rpocC_Fw
aggtctcaggcttgaaagacttgcttaatctgttgaa

SEQ ID NO: 157 - DNA, 32 bp, artificial
  Pak_rpocC_Rv
aggtctcagatcacttctcgcagatcacaccg

SEQ ID NO: 158 - DNA, 32 bp, artificial
  Pak_seceC_Fw
aggtctcagatcatgaatgccaaggcagaagc

SEQ ID NO: 159 - DNA, 32 bp, artificial
  Pak_seceC_Rv
aggtctcaatgccgatcagcgtggtctgagtt

SEQ ID NO: 160 - DNA, 31 bp, artificial
  Pak_sodbC_Fw
aggtctcagcatatggctttcgaattgccgc

SEQ ID NO: 161 - DNA, 32 bp, artificial
  Pak_sodbC_Rv
aggtctcactgaggctcaggcagttccagtag

SEQ ID NO: 162 - DNA, 37 bp, artificial
  Pak_rpocB_Fw
aggtctcaaacatgaaagacttgcttaatctgttgaa

SEQ ID NO: 163 - DNA, 32 bp, artificial
  Pak_sodbB_Rv
aggtctcaagccggctcaggcagttccagtag

SEQ ID NO: 164 - DNA, 32 bp, artificial
  Pak_sodbD_Fw
aggtctcatcagggctcaggcagttccagtag

SEQ ID NO: 165 - DNA, 37 bp, artificial
  Pak_rpocD_Rv
aggtctcagcagtgaaagacttgcttaatctgttgaa

SEQ ID NO: 166 - DNA, 31 bp, artificial
  Pak_xcpqC_Fw
aggtctcaggctatgtcccagcctttgctcc

SEQ ID NO: 167 - DNA, 34 bp, artificial
  Pak_xcpqC2_Rv
aggtctcagatcaatctggtcgatgaattcgcgg

SEQ ID NO: 168 - DNA, 32 bp, artificial
  Pak_pscfC_Fw
aggtctcagatcatggcgcagatattcaaccc

SEQ ID NO: 169 - DNA, 27 bp, artificial
  Pak_pscfC_Rv
aggtctcaatgcgcgcacgggtcaccg

SEQ ID NO: 170 - DNA, 27 bp, artificial
  Pak_psccC_Fw
aggtctcagcatatgcgccgcctgctg

SEQ ID NO: 171 - DNA, 29 bp, artificial
  Pak_psccC_Rv
aggtctcactgactgcgggctttccaggt

SEQ ID NO: 172 - DNA, 31 bp, artificial
  Pak_xcpqB_Fw
aggtctcaaacaatgtcccagcctttgctcc

SEQ ID NO: 173 - DNA, 29 bp, artificial
  Pak_psccB_Rv
aggtctcaagccctgcgggctttccaggt

SEQ ID NO: 174 - DNA, 29 bp, artificial
  Pak_psccD_Fw
aggtctcatcagctgcgggctttccaggt

SEQ ID NO: 175 - DNA, 31 bp, artificial
  Pak_xcpqD_Rv
aggtctcagcagatgtcccagcctttgctcc

SEQ ID NO: 176 - DNA, 32 bp, artificial
  Sf_ftsaB_Fw
aggtctcaaacaatgatcaaggcgacggacag

SEQ ID NO: 177 - DNA, 32 bp, artificial
  Sf_ftsaB_Rv
aggtctcagatcctgccatcaattctgcctgg

SEQ ID NO: 178 - DNA, 32 bp, artificial
  Sf_canB_Fw
aggtctcagatctttgtggttggcgtgtttca

SEQ ID NO: 179 - DNA, 33 bp, artificial
  Sf_canB_Rv
aggtctcaatgcaggtgtataacctgggccact

SEQ ID NO: 180 - DNA, 32 bp, artificial
  Sf_tsfB_Fw
aggtctcagcattggctgaaattaccgcatcc

SEQ ID NO: 181 - DNA, 32 bp, artificial
  Sf_tsfB_Rv
aggtctcaagccagttgccgtcgattttggtt

SEQ ID NO: 182 - DNA, 32 bp, artificial
  Sf_tsfD_Fw
aggtctcatcagagttgccgtcgattttggtt

SEQ ID NO: 183 - DNA, 32 bp, artificial
  Sf_ftsaD_Rv
aggtctcagcagatgatcaaggcgacggacag

SEQ ID NO: 184 - DNA, 31 bp, artificial
  Sf_accDB_Fw
aggtctcaaacataccgggggtaccactacg

SEQ ID NO: 185 - DNA, 32 bp, artificial
  Sf_accDB_Rv
aggtctcagatcaagcgttaatcggctttgcc

SEQ ID NO: 186 - DNA, 36 bp, artificial
  Sf_derB_Fw
aggtctcagatctttcttgatgtgcttcatcagacg

SEQ ID NO: 187 - DNA, 32 bp, artificial
  Sf_derB_Rv
aggtctcaatgccccgccgattgtggtgattc

SEQ ID NO: 188 - DNA, 34 bp, artificial
  Sf_psdB_Fw
aggtctcagcattttttatcgtcaaccaatgggc

SEQ ID NO: 189 - DNA, 32 bp, artificial
  Sf_psdB_Rv
aggtctcaagccaaaggccaggaaatgggtcg

SEQ ID NO: 190 - DNA, 32 bp, artificial
  Sf_psdD_Fw
aggtctcatcagaaaggccaggaaatgggtcg

SEQ ID NO: 191 - DNA, 31 bp, artificial
  Sf_accdD_Rv
aggtctcagcagtaccgggggtaccactacg

SEQ ID NO: 192 - DNA, 32 bp, artificial
  Sf_virfB_Fw
aggtctcaaacaaatgacggttagctcaggca

SEQ ID NO: 193 - DNA, 33 bp, artificial
  Sf_virfB_Rv
aggtctcagatctaacccccttttctcctcaga

SEQ ID NO: 194 - DNA, 33 bp, artificial
  Sf_virbB_Fw
aggtctcagatcatggtggatttgtgcaacgac

SEQ ID NO: 195 - DNA, 35 bp, artificial
  Sf_virbB_Rv
aggtctcaatgccagggaagaattgttgtagcttt

SEQ ID NO: 196 - DNA, 32 bp, artificial
  Sf_icsaB_Fw
aggtctcagcatggtagcggtgctgaccataa

SEQ ID NO: 197 - DNA, 32 bp, artificial
  Sf_icsaB_Rv
aggtctcaagcccgccatcaccctcaccataa

SEQ ID NO: 198 - DNA, 32 bp, artificial
  Sf_icsaD_Fw
aggtctcatcagcgccatcaccctcaccataa

SEQ ID NO: 199 - DNA, 31 bp, artificial
  Sf_virfD_Rv
ggtctcagcagaatgacggttagctcaggca

SEQ ID NO: 200 - DNA, 44 bp, artificial
  T7 polymerase Pak_dnaA_Fwd
ataatacgactcactatagggaggtgtccgtggaactttggcag

SEQ ID NO: 201 - DNA, 40 bp, artificial
  T7 polymerase Pak_dnaA_Rev
taatacgactcactatagggagaactgaccctcccgcgtt

SEQ ID NO: 202 - DNA, 42 bp, artificial
  T7 polymerase Pak_dnaN_Fwd
taatacgactcactatagggagatgcatttcaccattcaacg

SEQ ID NO: 203 - DNA, 38 bp, artificial
  T7 polymerase Pak_dnaN_Rev
taatacgactcactatagggagggtgatctcgccgggt

SEQ ID NO: 204 - DNA, 43 bp, artificial; identical to SEQ ID NO: 86
  T7 polymerase Pak_gyrB_Fwd

SEQ ID NO: 205 - DNA, 43 bp, artificial; identical to SEQ ID NO: 87
  T7 polymerase Pak_gyrB_Rev

SEQ ID NO: 206 - DNA, 47 bp, artificial
  T7 polymerase Pak_rpoC_Fwd
taatacgactcactatagggagtgaaagacttgcttaatctgttgaa

SEQ ID NO: 207 - DNA, 42 bp, artificial
  T7 polymerase Pak_rpoC_Rev
taatacgactcactatagggagacttctcgcagatcacaccg

SEQ ID NO: 208 - DNA, 42 bp, artificial
  T7 polymerase Pak_secE_Fwd
taatacgactcactatagggagatgaatgccaaggcagaagc

SEQ ID NO: 209 - DNA, 42 bp, artificial
  T7 polymerase Pak_secE_Rev
taatacgactcactatagggagcgatcagcgtggtctgagtt

SEQ ID NO: 210 - DNA, 41 bp, artificial
  T7 polymerase Pak_sodB_Fwd
taatacgactcactatagggagatggctttcgaattgccgc

SEQ ID NO: 211 - DNA, 42 bp, artificial
  T7 polymerase Pak_sodB_Rev
taatacgactcactatagggagggctcaggcagttccagtag

SEQ ID NO: 212 - DNA, 42 bp, artificial
  T7 polymerase sf_ftsA_Fwd
taatacgactcactatagggagatgatcaaggcgacggacag

SEQ ID NO: 213 - DNA, 42 bp, artificial
  T7 polymerase sf_ftsA_Rev
taatacgactcactatagggagctgccatcaattctgcctgg

SEQ ID NO: 214 - DNA, 42 bp, artificial
  T7 polymerase sf_Can_Fwd
taatacgactcactatagggagtttgtggttggcgtgtttca

SEQ ID NO: 215 - DNA, 43 bp, artificial
  T7 polymerase sf_Can_Rev
taatacgactcactatagggagaggtgtataacctgggccact

SEQ ID NO: 216 - DNA, 42 bp, artificial
  T7 polymerase sf_tsf_Fwd
taatacgactcactatagggagtggctgaaattaccgcatcc

SEQ ID NO: 217 - DNA, 42 bp, artificial
  T7 polymerase sf_tsf_Rev
taatacgactcactatagggagagttgccgtcgattttggtt

SEQ ID NO: 218 - DNA, 41 bp, artificial
  T7 polymerase sf_accD_Fwd
taatacgactcactatagggagtaccgggggtaccactacg

SEQ ID NO: 219 - DNA, 42 bp, artificial
  T7 polymerase sf_accD_Rev
taatacgactcactatagggagaagcgttaatcggctttgcc

SEQ ID NO: 220 - DNA, 46 bp, artificial
  T7 polymerase sf_der_Fwd
taatacgactcactatagggagtttcttgatgtgcttcatcagacg

SEQ ID NO: 221 - DNA, 42 bp, artificial
  T7 polymerase sf_der_Rev
taatacgactcactatagggagcccgccgattgtggtgattc

SEQ ID NO: 222 - DNA, 44 bp, artificial
  T7 polymerase sf_psd_Fwd
taatacgactcactatagggagtttttatcgtcaaccaatgggc

SEQ ID NO: 223 - DNA, 42 bp, artificial
  T7 polymerase sf_psd_Rev
taatacgactcactatagggagaaaggccaggaaatgggtcg

SEQ ID NO: 224 - DNA, 42 bp, artificial
  T7 polymerase sf_virF_Fwd
taatacgactcactatagggagaatgacggttagctcaggca

SEQ ID NO: 225 - DNA, 43 bp, artificial
  T7 polymerase sf_virF_Rev
taatacgactcactatagggagtaacccccttttctcctcaga

SEQ ID NO: 226 - DNA, 43 bp, artificial
  T7 polymerase sf_virB_Fwd
taatacgactcactatagggagatggtggatttgtgcaacgac

SEQ ID NO: 227 - DNA, 45 bp, artificial
  T7 polymerase sf_virB_Rev
taatacgactcactatagggagcagggaagaattgttgtagcttt

SEQ ID NO: 228 - DNA, 42 bp, artificial
  T7 polymerase sf_icsA_Fwd
taatacgactcactatagggagggtagcggtgctgaccataa

SEQ ID NO: 229 - DNA, 42 bp, artificial
  T7 polymerase sf_icsA_Rev
taatacgactcactatagggagcgccatcaccctcaccataa

SEQ ID NO: 230 - DNA, 47 bp, artificial
  T7 polymerase sf_spa47_Fwd
taatacgactcactatagggaggagctatacaaaattgctcactcaa

SEQ ID NO: 231 - DNA, 43 bp, artificial
  T7 polymerase sf_spa47_Rev
taatacgactcactatagggagcacgagaatttccaatcaagc

SEQ ID NO: 232 - DNA, 42 bp, artificial
  T7 polymerase sf_mukB_Fwd
taatacgactcactatagggagattgaacgcggtaaatttcg

SEQ ID NO: 233 - DNA, 42 bp, artificial
  T7 polymerase sf_mukB_Rev
taatacgactcactatagggagcagcttaccgtgcagacctt

SEQ ID NO: 234 - DNA, 44 bp, artificial
  T7 polymerase sf_ybiT_Fwd
taatacgactcactatagggaggtttccagtaacgtcaccatgc

SEQ ID NO: 235 - DNA, 41 bp, artificial
  T7 polymerase sf_ybiT_Rev
taatacgactcactatagggagagcttaccaatgcgctcgt

SEQ ID NO: 236 - DNA, 20 bp, artificial
  PAK-dnaA-qF
aaatacctcggtcggcttct

SEQ ID NO: 237 - DNA, 18 bp, artificial
  PAK-dnaA-qR
gtgggtctgcgatgggac

SEQ ID NO: 238 - DNA, 18 bp, artificial
  PAK-dnaN-qF
ggtcgcgtggtactggaa

SEQ ID NO: 239 - DNA, 20 bp, artificial
  PAK-dnaN-qR
ttctgctcttcgacacggat

SEQ ID NO: 240 - DNA, 20 bp, artificial
  PAK-gyrB-qF
cagcatcaccatccatacgg

SEQ ID NO: 241 - DNA, 20 bp, artificial
  PAK-gyrB-qR
ggaggacggtcatgatcact

SEQ ID NO: 242 - DNA, 20 bp, artificial
  PAK-rpoC-qF
gtgtgatctgcgagaagtgc

SEQ ID NO: 243 - DNA, 20 bp, artificial
  PAK-rpoC-qR
agcgacttcaggaaccagat

SEQ ID NO: 244 - DNA, 20 bp, artificial
  PAK-secE-qF
tatggccgagtcgtcaagaa

SEQ ID NO: 245 - DNA, 20 bp, artificial
  PAK-secE-qR
gaaaccaaccaacccagcag

SEQ ID NO: 246 - DNA, 21 bp, artificial
  PAK-sodB-qF
ggaaccacaccttctactgga

SEQ ID NO: 247 - DNA, 20 bp, artificial
  PAK-sodB-qR
cggaagtcttggtgaactct

SEQ ID NO: 248 - DNA, 500 bp, Artificial
  Sequence of the first arm of the LuxA/LuxB dsRNA used to target concomitantly the LuxA and LuxB genes of Pto DC3000
atgaaatttggaaactttttgcttacataccaacctccccaattttctcaaacagaggtaatgaaacgtttggttaaattaggtcgcatctctgaggagtgtggttttgataccgtatggttactggagcatcatttcacggagtttggtttgcttggtaacccttatgtcgctgctgcatatttacttggcgcgactaaaaaattgaatgtaggaactgccgctattgttcttcccacagcccatccagatgaaatttggattgttcttccttaacttcatcaattcaacaactgttcaagaacaaagtatagttcgcatgcaggaaataacggagtatgttgataagttgaattttgaacagattttagtgtatgaaaatcatttttcagataatggtgttgtcggcgctcctctgactgtttctggttttctgctcggtttaacagagaaaattaaaattggttcattaaatcacatcattacaactcatcatcctg

SEQ ID NO: 249 - DNA, 500 bp, Artificial
  Sequence of the second arm of the LuxA/LuxB dsRNA used to target concomitantly the LuxA and LuxB genes of Pto DC3000
caggatgatgagttgtaatgatgtgatttaatgaaccaattttaattttctctgttaaaccgagcagaaaaccagaaacagtcagaggagcgccgacaacaccattatctgaaaaatgattttcatacactaaaatctgttcaaaattcaacttatcaacatactccgttatttcctgcatgcgaactatactttgttcttgaacagttgttgaattgatgaagttaaggaagaacaatccaaatttcatctggatgggctgtgggaagaacaatagcggcagttcctacattcaattttttagtcgcgccaagtaaatatgcagcagcgacataagggttaccaagcaaaccaaactccgtgaaatgatgctccagtaaccatacggtatcaaaaccacactcctcagagatgcgacctaatttaaccaaacgtttcattacctctgtttgagaaaattggggaggttggtatgtaagcaaaaagtttccaaatttcat

SEQ ID NO: 250 - DNA, 750 bp, artificial
  Sequence of the first arm of the LptH/LolA/TolB dsRNA used to concomitantly target LptH, LolA and TolB genes genes from P. aeruginosa
atgaggttcgttaataccctcccccttattttcggtctgactgccgccctcggcagctccatggccttggccctgccttcggaccgcgaacaaccgatccgcgtccaggccgacagcgccgaactggacgacaagcagggcgtcgcggtctaccgcggcgacgtcgtggtgacccagggcagcaccaagctgaccggcaacaccgtgaccctgaagcaggacaagaacggcgacatcgaggtcgtgacctatgcgactgatccgcacgttgttcgttgccgccctggccatgggcgcttcgctggcccatgccgacgacagcgccgccgtccagcgcctgaccggcctgctgaacaaggcccagaccctgaccgcgcgtttttcccagctgaccctggacggtagcggcacgcgcctgcaggaaaccgccggccaactgagcctgaagcggccgggactgttccgctggcataccgatgcgccgaacgagcaattgctcagtgagtaccctgattcgcattgcgctgttcgccctggccctgatggcaggtgctgcgcaggctgccgacccgctggtgatctccagcggtaacgaccgtgccattcccatcgccgtggtgccgttcggcttccaggggggcaacgtgttgccggaagacatgtccaatatcatcggcaacgacctgcgcaactccggctacttcgagccgctgccccggcagaacatgatcagccagccggcgcaggcca

SEQ ID NO: 251 - DNA, 750 bp, artificial
  Sequence of the second arm of the LptH/LolA/TolB dsRNA used to concomitantly target LptH, LolA and TolB genes from P. aeruginosa
tggcctgcgccggctggctgatcatgttctgccggggcagcggctcgaagtagccggagttgcgcaggtcgttgccgatgatattggacatgtcttccggcaacacgttgcccccctggaagccgaacggcaccacggcgatgggaatggcacggtcgttaccgctggagatcaccagcgggtcggcagcctgcgcagcacctgccatcagggccagggcgaacagcgcaatgcgaatcagggtactcactgagcaattgctcgttcggcgcatcggtatgccagcggaacagtcccggccgcttcaggctcagttggccggcggtttcctgcaggcgcgtgccgctaccgtccagggtcagctgggaaaaacgcgcggtcagggtctgggccttgttcagcaggccggtcaggcgctggacggcggcgctgtcgtcggcatgggccagcgaagcgcccatggccagggcggcaacgaacaacgtgcggatcagtcgcataggtcacgacctcgatgtcgccgttcttgtcctgcttcagggtcacggtgttgccggtcagcttggtgctgccctgggtcaccacgacgtcgccgcggtagaccgcgacgccctgcttgtcgtccagttcggcgctgtcggcctggacgcggatcggttgttcgcggtccgaaggcagggccaaggccatggagctgccgagggcggcagtcagaccgaaaataagggggagggtattaacgaacctcat

SEQ ID NO: 252 - DNA, 750 bp, artificial
  Sequence of the first arm of the LpxA/LpxD/TolB dsRNA used to concomitantly target LpxA, LpxD and TolB genes from P. aeruginosa
atgagtttgatcgatcctcgcgccatcatcgatccgagcgccaggctggcggctgacgtccaggtgggcccgtggtccatcgtcggggcggaagtggagatcggtgaagggacggtgatcggtccgcacgtggtgctcaagggccccacgaagatcggcaagcacaaccgcatctaccagttttccagcgtcggcgaggatactcccgacctgaaatacaagggcgagccgacgcgcctggtgatcggtgatgatgagtaccttgtcctacaccctgggccagctggccgcgcatgtcggcgcggaagtccgtggcgacgcggacctgccaatccagggcctggcgaccttgcaggaggccggaccggctcagctgagcttcctggccaacccgcagtatcgcaagtacctgcccgaaagccgtgccggcgccgtcctgctgacggctgccgacgccgacgggttcgccggtaccgcgctggtggtggccaacccctaccgtgagtaccctgattcgcattgcgctgttcgccctggccctgatggcaggtgctgcgcaggctgccgacccgctggtgatctccagcggtaacgaccgtgccattcccatcgccgtggtgccgttcggcttccaggggggcaacgtgttgccggaagacatgtccaatatcatcggcaacgacctgcgcaactccggctacttcgagccgctgccccggcagaacatgatcagccagccggcgcaggcca

SEQ ID NO: 253 - DNA, 750 bp, artificial
  Sequence of the second arm of the LpxA/LpxD/TolB dsRNA used to concomitantly target LpxA, LpxD and TolB genes from P. aeruginosa
tggcctgcgccggctggctgatcatgttctgccggggcagcggctcgaagtagccggagttgcgcaggtcgttgccgatgatattggacatgtcttccggcaacacgttgcccccctggaagccgaacggcaccacggcgatgggaatggcacggtcgttaccgctggagatcaccagcgggtcggcagcctgcgcagcacctgccatcagggccagggcgaacagcgcaatgcgaatcagggtactcacggtaggggttggccaccaccagcgcggtaccggcgaacccgtcggcgtcggcagccgtcagcaggacggcgccggcacggctttcgggcaggtacttgcgatactgcgggttggccaggaagctcagctgagccggtccggcctcctgcaaggtcgccaggccctggattggcaggtccgcgtcgccacggacttccgcgccgacatgcgcggccagctggcccagggtgtaggacaaggtactcatcatcaccgatcaccaggcgcgtcggctcgcccttgtatttcaggtcgggagtatcctcgccgacgctggaaaactggtagatgcggttgtgcttgccgatcttcgtggggcccttgagcaccacgtgcggaccgatcaccgtcccttcaccgatctccacttccgccccgacgatggaccacgggcccacctggacgtcagccgccagcctggcgctcggatcgatgatggcgcgaggatcgatcaaactcat

SEQ ID NO: 254 - DNA, 750 bp, artificial
  Sequence of the first arm of the secE/dnaN/gyrB dsRNA used to concomitantly target secE, dnaN and gyrB genes from P. aeruginosa
atgaatgccaaggcagaagccaaagaatcacgttttgatctcttgaaatggctcttggttgccgttctggttgtggttgccgttgtgggcaatcagtacttttcggctcaaccaatcctgtatcgcgttctcggtattctcgttctggcggtgatcgctgctttcctggctctgcaaacggccaaggggcaggccttctttagtcttgctaaggaagcgcgcgtcgagattcgcaaggtcgtatggccgaatgcatttcaccattcaacgcgaagccctgttgaaaccgctgcaactggtcgccggcgtcgtggaacgccgccagacattgccggttctctccaacgtcctgctggtggtcgaaggccagcaactgtcgctgaccggcaccgacctcgaagtcgagctggttggtcgcgtggtactggaagatgccgccgaacccggcgagatcaccgtaccggcgcgcaagctgatggacatctgcaagagcctgccgaatgagcgagaacaacacgtacgactcttccagcatcaaggtgctgaaggggctggatgccgtacgcaagcgccccggcatgtacatcggcgacaccgacgatggcaccggtctgcaccacatggtgttcgaggtggtggataactccatcgacgaagcgctggccggttactgcagcgaaatcagcatcaccatccatacggatgagtcgatcactgtccgcgacaatggacgcggtattccggtggata

SEQ ID NO: 255 - DNA, 750 bp, ARTIFICIAL
  Sequence of the second arm of the secE/dnaN/gyrB dsRNA used to concomitantly target secE, dnaN and gyrB genes from P. aeruginosa
tatccaccggaataccgcgtccattgtcgcggacagtgatcgactcatccgtatggatggtgatgctgatttcgctgcagtaaccggccagcgcttcgtcgatggagttatccaccacctcgaacaccatgtggtgcagaccggtgccatcgtcggtgtcgccgatgtacatgccggggcgcttgcgtacggcatccagccccttcagcaccttgatgctggaagagtcgtacgtgttgttctcgctcattcggcaggctcttgcagatgtccatcagcttgcgcgccggtacggtgatctcgccgggttcggcggcatcttccagtaccacgcgaccaaccagctcgacttcgaggtcggtgccggtcagcgacagttgctggccttcgaccaccagcaggacgttggagagaaccggcaatgtctggcggcgttccacgacgccggcgaccagttgcagcggtttcaacagggcttcgcgttgaatggtgaaatgcattcggccatacgaccttgcgaatctcgacgcgcgcttccttagcaagactaaagaaggcctgccccttggccgtttgcagagccaggaaagcagcgatcaccgccagaacgagaataccgagaacgcgatacaggattggttgagccgaaaagtactgattgcccacaacggcaaccacaaccagaacggcaaccaagagccatttcaagagatcaaaacgtgattctttggcttctgccttggcattcat

SEQ ID NO: 256 - DNA, 1050 bp, artificial
  Sequence of the first arm of the XcpQ/ExsA/PcrV/LasR/RhlR/VqsM/RmsA dsRNA used to concomitantly target XcpQ, ExsA, PcrV, LasR, RhlR, VqsM and RmsA genes from P. aeruginosa
atgtcccagcctttgctccgcgccctgtttgccccttccagtcgttcgtacgttcccgccgtccttctcagcctggccctcggcatccaggcggcgcacgccgaaaacagcggcgggaacgccttcgtcccggccggcaaccagcaggagatgcaaggagccaaatctcttggccgaaagcagataacgtcttgtcattggaacattccaactttcgaatacagggtaaacaaggaagagggcgtatatgttctgctcgagggcgaactgaccgtccaggacatcgattccactttttgcatggaagtcagaaaccttaatgccgctcgcgagctgttcctggacgagctcctggccgcgtcggcggcgcctgccagtgccgagcaggaggaactgctggccctgttgcgcagcgagcggatcgtgctggcccacgccggccagccgctgatggccttggttgacggttttcttgagctggaacgctcaagtggaaaattggagtggagcgccatcctgcagaagatggcgagcgaccttggattctcgaagatcctgttcggcctgttgcctaaggacagccaggactacgagaacgccatgaggaatgacggaggctttttgctgtggtgggacggtttgcgtagcgagatgcagccgatccacgacagccagggcgtgttcgccgtcctggaaaaggaagtgcggcgcctgggcttcgattactacgcctatggcgtgcgccacacgatggtgaccgaacacatatttgattttgaatggcgccctctgcagaactatcttcgtgatcgtgatatcgcaataccaccctttgcggcagaacaagagtcaagaagtgacgcactttacaactggatagtcagcaaggggtattcaggaatgctgattctgactcgtcgggtcggagagaccctgatggtaggtgacgacgtcaccgtgacggtactgggtgtcaaagggaaccaggtgcgcatcggcgtcaacgcgccgaaggaagtcgccgtacaccgggaggaaatttaccagcgc

SEQ ID NO: 257 - DNA, 1050 bp, artificial
  Sequence of the second arm of the XcpQ/ExsA/PcrV/LasR/RhlR/VqsM/RmsA dsRNA used to concomitantly target XcpQ, ExsA, PcrV, LasR, RhlR, VqsM and RmsA genes from P. aeruginosa
gcgctggtaaatttcctcccggtgtacggcgacttccttcggcgcgttgacgccgatgcgcacctggttccctttgacacccagtaccgtcacggtgacgtcgtcacctaccatcagggtctctccgacccgacgagtcagaatcagcattcctgaataccccttgctgactatccagttgtaaagtgcgtcacttcttgactcttgttctgccgcaaagggtggtattgcgatatcacgatcacgaagatagttctgcagagggcgccattcaaaatcaaatatgtgttcggtcaccatcgtgtggcgcacgccataggcgtagtaatcgaagcccaggcgccgcacttccttttccaggacggcgaacacgccctggctgtcgtggatcggctgcatctcgctacgcaaaccgtcccaccacagcaaaaagcctccgtcattcctcatggcgttctcgtagtcctggctgtccttaggcaacaggccgaacaggatcttcgagaatccaaggtcgctcgccatcttctgcaggatggcgctccactccaattttccacttgagcgttccagctcaagaaaaccgtcaaccaaggccatcagcggctggccggcgtgggccagcacgatccgctcgctgcgcaacagggccagcagttcctcctgctcggcactggcaggcgccgccgacgcggccaggagctcgtccaggaacagctcgcgagcggcattaaggtttctgacttccatgcaaaaagtggaatcgatgtcctggacggtcagttcgccctcgagcagaacatatacgccctcttccttgtttaccctgtattcgaaagttggaatgttccaatgacaagacgttatctgctttcggccaagagatttggctccttgcatctcctgctggttgccggccgggacgaaggcgttcccgccgctgttttcggcgtgcgccgcctggatgccgagggccaggctgagaaggacggcgggaacgtacgaacgactggaaggggcaaacagggcgcggagcaaaggctgggacat

SEQ ID NO: 258 - DNA, 750 bp, artificial
  Sequence of the first arm of the XcpQ/PscF/PscC dsRNA used to concomitantly target XcpQ, PscF and PscC genes from P. aeruginosa
atgtcccagcctttgctccgcgccctgtttgccccttccagtcgttcgtacgttcccgccgtccttctcagcctggccctcggcatccaggcggcgcacgccgaaaacagcggcgggaacgccttcgtcccggccggcaaccagcaggaggcgcactggacgatcaacctcaaggatgccgacatccgcgaattcatcgaccagatttccgaaatcaccggcgagaccttcgtcgtcgacccgcgggtcatcagatcttctgcaggatgccttgcatcaggtcgcgcagcgcacgggtcaccgtcgagttgatgttgtagatgaccgaccacttgttgatcttgtgttgcagctcggccagcagcgccgggttgtcggcattgtcggtcccctgcaaggccttgatcgcgtcgttgacgtccttgttcgctgcgttggcctgctccttcagggcattggccacggtatcgagggtattccccgggttggggttgaatatcatgcgccgcctgctgatcggcggactgctggcgctgctgcctggcgcggtcttgcgcgcgcagccgctggactggcccagcctgccttacgactatgtggcgcagggcgaaagcctgcgcgacgtgctggccaacttcggcgccaactacgatgcctcggtgatcgtcagcgacaaggtcaacgaccaggtcagcggtcgcttcgacctggaaagcccgcaggccttcctgcagttgatggcctccctct

SEQ ID NO: 259 - DNA, 750 bp, artificial
  Sequence of the second arm of the XcpQ/PscF/PscC dsRNA used to concomitantly target XcpQ, PscF and PscC genes from P. aeruginosa
agagggaggccatcaactgcaggaaggcctgcgggctttccaggtcgaagcgaccgctgacctggtcgttgaccttgtcgctgacgatcaccgaggcatcgtagttggcgccgaagttggccagcacgtcgcgcaggctttcgccctgcgccacatagtcgtaaggcaggctgggccagtccagcggctgcgcgcgcaagaccgcgccaggcagcagcgccagcagtccgccgatcagcaggcggcgcatgatattcaaccccaacccggggaataccctcgataccgtggccaatgccctgaaggagcaggccaacgcagcgaacaaggacgtcaacgacgcgatcaaggccttgcaggggaccgacaatgccgacaacccggcgctgctggccgagctgcaacacaagatcaacaagtggtcggtcatctacaacatcaactcgacggtgacccgtgcgctgcgcgacctgatgcaaggcatcctgcagaagatctgatgacccgcgggtcgacgacgaaggtctcgccggtgatttcggaaatctggtcgatgaattcgcggatgtcggcatccttgaggttgatcgtccagtgcgcctcctgctggttgccggccgggacgaaggcgttcccgccgctgttttcggcgtgcgccgcctggatgccgagggccaggctgagaaggacggcgggaacgtacgaacgactggaaggggcaaacagggcgcggagcaaaggctgggacat

SEQ ID NO: 260 - DNA, 750 bp, artificial
  Sequence of the first arm of the ExoS/ExsA/Vrf dsRNA used to concomitantly target ExoS, ExsA and Vrf genes from P. aeruginosa
atgcatattcaatcgcttcagcagagtccgtctttcgccgtcgaattgcaccaggccgccagtgggcgtttgggacagattgaggcccgccaggtcgccacccccagtgaagcgcagcagttggcccagcgccaggacgcgccgaagggtgaggggctgctcgctcgcttgggtgcggcactcgtgcgtcccttcgtggcgatcatggactggctgggcaagctgttgggctcccacgcccgcaccggccatgcaaggagccaaatctcttggccgaaagcagataacgtcttgtcattggaacattccaactttcgaatacagggtaaacaaggaagagggcgtatatgttctgctcgagggcgaactgaccgtccaggacatcgattccactttttgcctggcgcctggcgagttgcttttcgtccgccgcggaagctatgtcgtaagtaccaagggaaaggacagccgaatactctggattccattatctgcccagtatggtagctattacccacacacccaaactcaaacacctagacaagctgctcgcacactgtcaccgccgccgctacaccgcaaagagcaccatcatctatgccggcgatcgctgcgaaacgctgttcttcatcatcaagggttcggtcaccatcctcatcgaggacgacgacggccgcgaaatgatcatcggctacctcaacagcggtgatttcttcggcgagctgggattgttcgaaaaggaaggcagcg

SEQ ID NO: 261 - DNA, 750 bp, artificial
  Sequence of the second arm of the ExoS/ExsA/Vrf dsRNA used to concomitantly target ExoS, ExsA and Vrf genes from P. aeruginosa
cgctgccttccttttcgaacaatcccagctcgccgaagaaatcaccgctgttgaggtagccgatgatcatttcgcggccgtcgtcgtcctcgatgaggatggtgaccgaacccttgatgatgaagaacagcgtttcgcagcgatcgccggcatagatgatggtgctctttgcggtgtagcggcggcggtgacagtgtgcgagcagcttgtctaggtgtttgagtttgggtgtgtgggtaatagctaccatactgggcagataatggaatccagagtattcggctgtcctttcccttggtacttacgacatagcttccgcggcggacgaaaagcaactcgccaggcgccaggcaaaaagtggaatcgatgtcctggacggtcagttcgccctcgagcagaacatatacgccctcttccttgtttaccctgtattcgaaagttggaatgttccaatgacaagacgttatctgctttcggccaagagatttggctccttgcatggccggtgcgggcgtgggagcccaacagcttgcccagccagtccatgatcgccacgaagggacgcacgagtgccgcacccaagcgagcgagcagcccctcacccttcggcgcgtcctggcgctgggccaactgctgcgcttcactgggggtggcgacctggcgggcctcaatctgtcccaaacgcccactggcggcctggtgcaattcgacggcgaaagacggactctgctgaagcgattgaatatgcat

SEQ ID NO: 262 - DNA, 750 bp, artificial
  Sequence of the first arm of the ExoU/ExsA/Vrf dsRNA used to concomitantly target ExoU, ExsA and Vrf genes from P. aeruginosa
atgcatatccaatcgttgggggctactgcctcctcgctgaatcaggagcctgtcgaaaccccgtcgcaggcagcgcataagtccgccagcttgcgtcaggaaccttcagggcaaggtctcggggttgccctaaagagcacgccgggaatactttccgggaagttgccggaaagcgttagcgacgtgcgtttcagcagtccccaagggcaaggggagtcccgtactctgactgactcggcagggccgcggcatgcaaggagccaaatctcttggccgaaagcagataacgtcttgtcattggaacattccaactttcgaatacagggtaaacaaggaagagggcgtatatgttctgctcgagggcgaactgaccgtccaggacatcgattccactttttgcctggcgcctggcgagttgcttttcgtccgccgcggaagctatgtcgtaagtaccaagggaaaggacagccgaatactctggattccattatctgcccagtatggtagctattacccacacacccaaactcaaacacctagacaagctgctcgcacactgtcaccgccgccgctacaccgcaaagagcaccatcatctatgccggcgatcgctgcgaaacgctgttcttcatcatcaagggttcggtcaccatcctcatcgaggacgacgacggccgcgaaatgatcatcggctacctcaacagcggtgatttcttcggcgagctgggattgttcgaaaaggaaggcagcg

SEQ ID NO: 263 - DNA, 750 bp, artificial
  Sequence of the second arm of the ExoU/ExsA/Vrf dsRNA used to concomitantly target ExoU, ExsA and Vrf genes from P. aeruginosa
cgctgccttccttttcgaacaatcccagctcgccgaagaaatcaccgctgttgaggtagccgatgatcatttcgcggccgtcgtcgtcctcgatgaggatggtgaccgaacccttgatgatgaagaacagcgtttcgcagcgatcgccggcatagatgatggtgctctttgcggtgtagcggcggcggtgacagtgtgcgagcagcttgtctaggtgtttgagtttgggtgtgtgggtaatagctaccatactgggcagataatggaatccagagtattcggctgtcctttcccttggtacttacgacatagcttccgcggcggacgaaaagcaactcgccaggcgccaggcaaaaagtggaatcgatgtcctggacggtcagttcgccctcgagcagaacatatacgccctcttccttgtttaccctgtattcgaaagttggaatgttccaatgacaagacgttatctgctttcggccaagagatttggctccttgcatgccgcggccctgccgagtcagtcagagtacgggactccccttgcccttggggactgctgaaacgcacgtcgctaacgctttccggcaacttcccggaaagtattcccggcgtgctctttagggcaaccccgagaccttgccctgaaggttcctgacgcaagctggcggacttatgcgctgcctgcgacggggtttcgacaggctcctgattcagcgaggaggcagtagcccccaacgattggatatgcat

SEQ ID NO: 264 - DNA, 750 bp, artificial
  Sequence of the first arm of the LasR/RhlR/VqsM dsRNA used to concomitantly target LasR, RhlR and VqsM genes from P. aeruginosa
atggccttggttgacggttttcttgagctggaacgctcaagtggaaaattggagtggagcgccatcctgcagaagatggcgagcgaccttggattctcgaagatcctgttcggcctgttgcctaaggacagccaggactacgagaacgccttcatcgtcggcaactacccggccgcctggcgcgagcattacgaccgggctggctacgcgcgggtcgacccgacggtcagtcactgtacccagagcgtacatgaggaatgacggaggctttttgctgtggtgggacggtttgcgtagcgagatgcagccgatccacgacagccagggcgtgttcgccgtcctggaaaaggaagtgcggcgcctgggcttcgattactacgcctatggcgtgcgccacacgattcccttcacccggccgaagaccgaggtccatggcacctatcccaaggcctggctggagcgataccagatgcagaactacggggccgtggatccggcgaatggtgaccgaacacatatttgattttgaatggcgccctctgcagaactatcttcgtgatcgtgatatcgcaataccaccctttgcggcagaacaagagtcaagaagtgacgcactttacaactggatagtcagcaaggggtattcaggaaaagaaggactgaatcttggaacttattaccatatatctgattatggcgtgataggattggcgttgctatgcgccgagaacgtaggtgatattttgaaag

SEQ ID NO: 265 - DNA, 750 bp, artificial
  Sequence of the second arm of the LasR/RhlR/VqsM dsRNA used to concomitantly target LasR, RhlR and VqsM genes from P. aeruginosa
ctttcaaaatatcacctacgttctcggcgcatagcaacgccaatcctatcacgccataatcagatatatggtaataagttccaagattcagtccttcttttcctgaataccccttgctgactatccagttgtaaagtgcgtcacttcttgactcttgttctgccgcaaagggtggtattgcgatatcacgatcacgaagatagttctgcagagggcgccattcaaaatcaaatatgtgttcggtcaccattcgccggatccacggccccgtagttctgcatctggtatcgctccagccaggccttgggataggtgccatggacctcggtcttcggccgggtgaagggaatcgtgtggcgcacgccataggcgtagtaatcgaagcccaggcgccgcacttccttttccaggacggcgaacacgccctggctgtcgtggatcggctgcatctcgctacgcaaaccgtcccaccacagcaaaaagcctccgtcattcctcatgtacgctctgggtacagtgactgaccgtcgggtcgacccgcgcgtagccagcccggtcgtaatgctcgcgccaggcggccgggtagttgccgacgatgaaggcgttctcgtagtcctggctgtccttaggcaacaggccgaacaggatcttcgagaatccaaggtcgctcgccatcttctgcaggatggcgctccactccaattttccacttgagcgttccagctcaagaaaaccgtcaaccaaggccat

SEQ ID NO: 266 - DNA, 686 bp, artificial
  Sequence of the first arm of the GacA/RmsA/MvfR dsRNA used to concomitantly target GacA, RmsA and MvfR genes from P. aeruginosa
gtgattaaggtgctggtggtcgacgaccacgatctggtacgcaccggtattacccgcatgctggccgacatcgaaggcttgcaagtggtcggccaggccgactgcggtgaagactgtctgaaactggcccgcgaactgaagccggatgtcgtcctgatggacgtgaagatgcccggtatcggcggcctggaggcgacccgcaagctgctgcgcagccagcccgacatcaaggtcgtggtagtcaccgtctatgctgattctgactcgtcgggtcggagagaccctgatggtaggtgacgacgtcaccgtgacggtactgggtgtcaaagggaaccaggtgcgcatcggcgtcaacgcgccgaaggaagtcgccgtacaccgggaggaaatttaccagcgcatccagaaagagaaagatcaagagccaaaccattaaatgcctattcataacctgaatcacgtgaacatgttcctccaggtcatcgcctccggttcgatttcctccgctgcgcggatcctgcgcaagtcgcacaccgcggtcagctcggcggtcagcaacctggaaatcgacctgtgcgtggagctggtccgtcgggacggctacaaggtcgaacccaccgagcaggcgcttcgcctgatcccttacatgcgcagcctgctgaactaccagcagctgatcggcgaca

SEQ ID NO: 267 - DNA, 686 bp, artificial
  Sequence of the second arm of the GacA/RmsA/MvfR dsRNA used to concomitantly target GacA, RmsA and MvfR genes from P. aeruginosa
tgtcgccgatcagctgctggtagttcagcaggctgcgcatgtaagggatcaggcgaagcgcctgctcggtgggttcgaccttgtagccgtcccgacggaccagctccacgcacaggtcgatttccaggttgctgaccgccgagctgaccgcggtgtgcgacttgcgcaggatccgcgcagcggaggaaatcgaaccggaggcgatgacctggaggaacatgttcacgtgattcaggttatgaataggcatttaatggtttggctcttgatctttctctttctggatgcgctggtaaatttcctcccggtgtacggcgacttccttcggcgcgttgacgccgatgcgcacctggttccctttgacacccagtaccgtcacggtgacgtcgtcacctaccatcagggtctctccgacccgacgagtcagaatcagcatagacggtgactaccacgaccttgatgtcgggctggctgcgcagcagcttgcgggtcgcctccaggccgccgataccgggcatcttcacgtccatcaggacgacatccggcttcagttcgcgggccagtttcagacagtcttcaccgcagtcggcctggccgaccacttgcaagccttcgatgtcggccagcatgcgggtaataccggtgcgtaccagatcgtggtcgtcgaccaccagcaccttaatcac

SEQ ID NO: 268 - DNA, 750 bp, artificial
  Sequence of the first arm of the VirF/VirB/IcsA dsRNA used to concomitantly target VirF, VirB and IcsA genes from Shigella flexneri
atgatggatatgggacataaaaacaaaatagatataaaggttcgcttgcataactatattattttatatgcaaaaaggtgttcaatgacggttagctcaggcaatgaaactttgactatcgatgaagggcaaattgcttttatagagcgaaatatacaaataaacgtctccataaaaaaatctgatagcattaatccatttgagattataagccttgacagaaatttattattaagcattattagaataaatggtggatttgtgcaacgacttgttaagtataaaggaaggccaaaagaaagagtttacactccattctggtaataaagtttcctttatcaaagccaagattcctcataaaaggatccaagatttaaccttcgtcaaccaaaaaacgaatgtacgcgatcaagaatccctaacagaagaatcattagccgatatcataaaaactataaagctacaacaattcttccctgtaataggaagggagattgatgcccttgggagctggtgaagatggaatggatgcgtggtatataacttcttccaacccctctcatgcatctagaactaagctacggattaactctgatattatgattagcgcaggtcatggtggtgctggtgataataatgatggtaatagttgtggcggtaatggtggtgactctattaccggatctgacttgtctataatcaatcaaggcatgattcttggtggtagcggcggtagcggtgctgaccata

SEQ ID NO: 269 - DNA, 750 bp, artificial
  Sequence of the second arm of the VirF/VirB/IcsA dsRNA used to concomitantly target VirF, VirB and IcsA genes from Shigella flexneri
tatggtcagcaccgctaccgccgctaccaccaagaatcatgccttgattgattatagacaagtcagatccggtaatagagtcaccaccattaccgccacaactattaccatcattattatcaccagcaccaccatgacctgcgctaatcataatatcagagttaatccgtagcttagttctagatgcatgagaggggttggaagaagttatataccacgcatccattccatcttcaccagctcccaagggcatcaatctcccttcctattacagggaagaattgttgtagctttatagtttttatgatatcggctaatgattcttctgttagggattcttgatcgcgtacattcgttttttggttgacgaaggttaaatcttggatccttttatgaggaatcttggctttgataaaggaaactttattaccagaatggagtgtaaactctttcttttggccttcctttatacttaacaagtcgttgcacaaatccaccatttattctaataatgcttaataataaatttctgtcaaggcttataatctcaaatggattaatgctatcagatttttttatggagacgtttatttgtatatttcgctctataaaagcaatttgcccttcatcgatagtcaaagtttcattgcctgagctaaccgtcattgaacacctttttgcatataaaataatatagttatgcaagcgaacctttatatctattttgtttttatgtcccatatccatcat

SEQ ID NO: 270 - DNA, 800 bp, artificial
  Sequence of the first arm of the fnbA/clfA/clfB/spa dsRNA used to concomitantly target fnbA, clfA, clfB and spa genes from S. aureus
atgggacaagacaaagaagctgcagcatcagaacaaaagacaactacagtagaagaaaatgggaattcagctactgataataaaacaagtgaaacacaaacaactgcaactaacgttaatcatatagaagaaactcaatcatataacgcaacagtaacagaacaaccgtcaaacgcaacacaagtaacaactgaagaagcatgaatatgaagaaaaaagaaaaacacgcaattcggaaaaaatcgattggcgtggcttcagtgcttgtaggtacgttaatcggttttggactactcagcagtaaagaagcagatgcaagtgaaaatagtgttacgcaatctgatagcgcaagtaacgaaagcaaaagtaatgattcaagtagcgttagtgctgcacctaattacgctttttctttatgatcttgcttgcgttttctaaacaatagtaatgatcctaataatgccatcattgcaccaaataaagttgcatttgtgttttcgctcttatctcctgtttctggtaaagcatcagttttgtgttgttttgataccttattagaatggtttacttcacctttaggatttgatggtgctttctgttttatagttcgcgacgacgtccagctaataacgctgcacctaaggctaatgataatccaccaaatacagttgtaccgatgaatggattttcttcaccagtttctggtaatgcttgagctttgttagcatctgcatggtttgctggttgcttcttatcaacaacaagttcttgaccaggtttgatcatgtttttatcagcta

SEQ ID NO: 271 - DNA, 800 bp, artificial
  Sequence of the second arm of the fnbA/clfA/clfB/spa dsRNA used to concomitantly target fnbA, clfA, clfB and spa genes from S. aureus
tagctgataaaaacatgatcaaacctggtcaagaacttgttgttgataagaagcaaccagcaaaccatgcagatgctaacaaagctcaagcattaccagaaactggtgaagaaaatccattcatcggtacaactgtatttggtggattatcattagccttaggtgcagcgttattagctggacgtcgtcgcgaactataaaacagaaagcaccatcaaatcctaaaggtgaagtaaaccattctaataaggtatcaaaacaacacaaaactgatgctttaccagaaacaggagataagagcgaaaacacaaatgcaactttatttggtgcaatgatggcattattaggatcattactattgtttagaaaacgcaagcaagatcataaagaaaaagcgtaattaggtgcagcactaacgctacttgaatcattacttttgctttcgttacttgcgctatcagattgcgtaacactattttcacttgcatctgcttctttactgctgagtagtccaaaaccgattaacgtacctacaagcactgaagccacgccaatcgattttttccgaattgcgtgtttttcttttttcttcatattcatgcttcttcagttgttacttgtgttgcgtttgacggttgttctgttactgttgcgttatatgattgagtttcttctatatgattaacgttagttgcagttgtttgtgtttcacttgttttattatcagtagctgaattcccattttcttctactgtagttgtcttttgttctgatgctgcagcttctttgtcttgtcccat

SEQ ID NO: 272 - DNA, 800 bp, artificial
  Sequence of the first arm of the lukF-PV/lukS-PV/lukE/lukD dsRNA used to concomitantly target lukF-PV, lukS-PV, lukE and lukD genes from S. aureus
ttagctcataggatttttttccttagattgagtatctattaatttaactgtatgattttcccaatcaacttcataaattgatgtatgagttgctctattttcatctttataattattacctatccagtgaagttgattccaaaagtttgtatatctatccatttctctttgataagtaacagtaattttcgatttttttgtcaattatgtcctttcactttaatttcatgagttttccagttcacttcatatttaactgtgtaatttctgtttacaaatgcgttgtgtattctagatccttctaaataactattgccatagtgtgttgttcttctagtagcatgagtaacatccatatttctgccatacgttatttcaaattcacttgtatctcctgagcatgtttaagaaaaaaatgttagctgcaagtttgtcagtaggactgattgcacctttagcatctccgattcaagaatctagagcaaatactaatattgaaaatattggtgatggtgctgaagtaatcaaacgtacggaggatgtaagtagtaagaaatggggcgttactcaaaatgtccaattcgactttgtaaaagataaatgaaaattgaaaaattaggcaaatcatcagttgcttcatcaattgcactgcttttgctatcgaatacagttgatgcagctcaaaatatcacacctaaaagagagaaaaaagtagatgacaaaatcactttatacaaaacaacagcaacatctgataatgataaattgaatatttttcaaattttaacgtttaatttcat

SEQ ID NO: 273 - DNA, 800 bp, artificial
  Sequence of the second arm of the lukF-PV/lukS-PV/lukE/lukD dsRNA used to concomitantly target lukF-PV, lukS-PV, lukE and lukD genes from S. aureus
atgaaattaaacgttaaaatttgaaaaatattcaatttatcattatcagatgttgctgttgttttgtataaagtgattttgtcatctacttttttctctcttttaggtgtgatattttgagctgcatcaactgtattcgatagcaaaagcagtgcaattgatgaagcaactgatgatttgcctaatttttcaattttcatttatcttttacaaagtcgaattggacattttgagtaacgccccatttcttactacttacatcctccgtacgtttgattacttcagcaccatcaccaatattttcaatattagtatttgctctagattcttgaatcggagatgctaaaggtgcaatcagtcctactgacaaacttgcagctaacatttttttcttaaacatgctcaggagatacaagtgaatttgaaataacgtatggcagaaatatggatgttactcatgctactagaagaacaacacactatggcaatagttatttagaaggatctagaatacacaacgcatttgtaaacagaaattacacagttaaatatgaagtgaactggaaaactcatgaaattaaagtgaaaggacataattgacaaaaaaatcgaaaattactgttacttatcaaagagaaatggatagatatacaaacttttggaatcaacttcactggataggtaataattataaagatgaaaatagagcaactcatacatcaatttatgaagttgattgggaaaatcatacagttaaattaatagatactcaatctaaggaaaaaaatcctatgagctaa

SEQ ID NO: 274 - DNA, 800 bp, artificial
  Sequence of the first arm of the HlgB/hla/tsst-1/atl dsRNA used to concomitantly target HlgB, hla, tsst-1 and atl genes from S. aureus
atgaaaatgaataaattagtcaaatcatccgttgctacatctatggcattattattactttctggtactgctaatgctgaaggtaaaataacaccagtcagcgtaaaaaaagtcgatgacaaagttactttatacaaaacaacagccacagcagattctgataaatttaaaatttcacagattttaacatttaatttcatttaatttgtcatttcttctttttcccaatcgattttatatctttctgaagaacgatctatccatttatctttagtattggtacctttccaatttgttgaagtccagtgcaattggtagtcatcacgaactcgttcgtatattacatctatatttgtttgttgtttggatgcttttctatccatagtaataactgtagcgaatgaataaaaaattactaatgaatttttttatcgtaagccctttgttgcttgcgacaatcgctacagattttacccctgttcccttatcatctaatcaaataatcaaaactgcaaaagcatctacaaacgataatataaaggatttgctagactggtatagtagtgggtctgacacttttacaaatagtgaagttttagattatttatattgtgggatgtcgaagtatttgccgacttcgccaattttatcatagtagcctttgatgattttagcattgatgttagcccaatctacatctgtagcatattggtgtgttcctggatgtgcaggattccatctcattttgtaaagtgtattttgaccagcttttacatatgagttgccgatgaatttagcac

SEQ ID NO: 275 - DNA, 800 bp, artificial
  Sequence of the second arm of the HlgB/hla/tsst-1/atl dsRNA used to concomitantly target HlgB, hla, tsst-1 and atl genes from S. aureus
gtgctaaattcatcggcaactcatatgtaaaagctggtcaaaatacactttacaaaatgagatggaatcctgcacatccaggaacacaccaatatgctacagatgtagattgggctaacatcaatgctaaaatcatcaaaggctactatgataaaattggcgaagtcggcaaatacttcgacatcccacaatataaataatctaaaacttcactatttgtaaaagtgtcagacccactactataccagtctagcaaatcctttatattatcgtttgtagatgcttttgcagttttgattatttgattagatgataagggaacaggggtaaaatctgtagcgattgtcgcaagcaacaaagggcttacgataaaaaaattcattagtaattttttattcattcgctacagttattactatggatagaaaagcatccaaacaacaaacaaatatagatgtaatatacgaacgagttcgtgatgactaccaattgcactggacttcaacaaattggaaaggtaccaatactaaagataaatggatagatcgttcttcagaaagatataaaatcgattgggaaaaagaagaaatgacaaattaaatgaaattaaatgttaaaatctgtgaaattttaaatttatcagaatctgctgtggctgttgttttgtataaagtaactttgtcatcgactttttttacgctgactggtgttattttaccttcagcattagcagtaccagaaagtaataataatgccatagatgtagcaacggatgatttgactaatttattcattttcat

(19) United States
(12) Patent Application Publication
(10) Pub. No.: US 2022/0288230 A1
(43) Pub. Date: Sep. 15, 2022

(54) RNA-BASED THERAPEUTIC METHODS TO PROTECT ANIMALS AGAINST PATHOGENIC BACTERIA AND / OR PROMOTE BENEFICIAL EFFECTS OF SYMBIOTIC AND COMMENSAL BACTERIA

(71) Applicants: CENTRE NATIONAL DE LA RECHERCHE SCIENTIFIQUE (CNRS), PARIS (FR); ECOLE NORMALE SUPERIEURE, PARIS (FR); INSERM (Institut National de la Sante et de la Recherche Medicale, PARIS (FR)

(72) Inventors: Lionel NAVARRO, ANTONY (FR); Meenu SINGLA RASTOGI, Meerut (IN)

(21) Appl. No.: 17/636,225

(22) PCT Filed: Aug. 19, 2020
(86) PCT No.: PCT/EP2020/073231

§ 371 Date: Feb. 17, 2022

(30) Foreign Application Priority Data
Aug. 19, 2019 (EP) PCT/EP2019/072170

(51) Int. Cl. A61K 48/00; A61K 31/7088; A61P 31/04; C12N 15/113; C12N 15/82; C12N 15/85; C12Q 1/686

(52) U.S. Cl. CPC A61K 48/00; A61K 31/7088; A61P 31/04; C12N 15/113; C12N 15/8281; C12N 15/85; C12Q 1/686; C12N 2310/10

(57) ABSTRACT

The invention relates to a method to inhibit gene expression in bacteria, which is referred to here as Antibacterial Gene Silencing (AGS). In particular embodiments, the method is used to protect plants and animals against pathogenic bacteria by targeting pathogenicity factors and/or essential genes in a sequence-specific manner via small non-coding RNAs. The method can also be used to enhance beneficial effects and/or growth of symbiotic or commensal bacteria. The invention involves the exogenous delivery of small RNA entities onto bacteria, either in the form of RNA extracts or embedded into plant extracellular vesicles (EVs), so as to reduce bacterial growth, survival and/or pathogenicity. The invention also describes a method to identify in a rapid, reliable and cost-effective manner, small RNAs that possess antibacterial activity and that have the potential to be further developed as anti-infective agents. In addition, the latter method is instrumental to rapidly characterize any gene from any bacterial species.

### SUMMARY OF THE INVENTION

The invention relates to a method to inhibit gene expression in bacteria, which is referred to here as Antibacterial Gene Silencing (AGS). In particular embodiments, the method is used to protect plants and animals against pathogenic bacteria by targeting pathogenicity factors and/or essential genes in a sequence-specific manner via small non-coding RNAs. The method can also be used to enhance beneficial effects and/or growth of symbiotic or commensal bacteria. The invention involves the exogenous delivery of small RNA entities onto bacteria, either in the form of RNA extracts or embedded into plant extracellular vesicles (EVs), so as to reduce bacterial growth, survival and/or pathogenicity. The invention also describes a method to identify in a rapid, reliable and cost-effective manner, small RNAs that possess antibacterial activity and that have the potential to be further developed as anti-infective agents. In addition, the latter method is instrumental to rapidly characterize any gene from any bacterial species.

### PRIOR ART DESCRIPTION

Overview of the Plant Immune System

The first layer of the plant immune system involves the recognition of Pathogen- or Microbe-Associated Molecular Patterns (PAMPs or MAMPs), which are conserved microbial signatures that are sensed by surface-localized Pattern-Recognition Receptors (PRRs) (1). Upon ligand binding, these receptors initiate a complex phosphorylation cascade at the PRR complex that leads to PAMP-triggered immunity (PTI) (1). To enable disease, pathogens secrete effectors that suppress PTI (2). For instance, the Gram-negative bacterium Pseudomonas syringae pv. tomato strain DC3000 (Pto DC3000) injects 36 type-III secreted effectors into plant cells to dampen PTI (3). This bacterium also produces coronatine (COR), a phytotoxin that is essential for pathogenicity (4). Plants have evolved disease resistance (R) proteins that can perceive the presence of pathogen effectors to trigger a host counter-counter defense (5). Most R proteins belong to the nucleotide-binding domain (NBD), leucine-rich repeat (NLR) superfamily, which are also present in animals (2, 5). They recognize, directly or indirectly, pathogen effectors and mount Effector-triggered immunity (ETI), a potent immune response that significantly overlaps with PTI, although with a stronger amplitude (6, 7).

Post-Transcriptional Gene Silencing (PTGS) Controls Host-Pathogen Interactions

PTGS is a conserved post-transcriptional gene regulatory mechanism that has been extensively characterized as a natural antiviral defense response in plants by targeting and degrading viral transcripts (8). The core mechanism of RNA silencing or RNA interference (RNAi) in plants involves the recognition and processing of double-stranded RNAs (dsRNAs) by RNase III enzyme DICER-LIKE (DCL) proteins leading to the production of 20-25 nt long short interfering RNA (siRNA) duplexes. These siRNA duplexes associate with an Argonaute (AGO) protein, the central component of the RNA-induced silencing complex (RISC). Subsequent strand separation on the AGO protein forms a mature RISC composed of AGO and a single-stranded RNA, the guide strand, while the passenger strand is degraded. The guide small RNA directs AGO-RISC onto sequence complementary mRNA targets, leading to their endonucleolytic cleavage and/or translational inhibition. During the last decade, several endogenous short interfering RNAs (siRNAs) and microRNAs (miRNAs) were additionally found to orchestrate PTI and ETI responses against non-viral pathogens (9), implying a key role of PTGS in the regulation of the plant immune system.

In plants, mobile small RNAs can trigger non-cell autonomous silencing in adjacent cells as well as in distal tissues (10). They are notably important to prime antiviral defense ahead of the infection front (10). Non-cell autonomous silencing is also critical for the translocation of silencing signals between plant cells and their interacting non-viral pathogenic, parasitic or symbiotic organisms—excluding bacteria, which have not been shown to be targeted by this process (11). This natural cross-kingdom regulatory mechanism has been notably recently characterized in plant-fungal interactions (12-17). For instance, specific plant miRNAs were found to be exported into the hyphae of the fungal pathogen Verticillium dahliae to trigger silencing of virulence factors (14, 17). On the other hand, endogenous B. cinerea small RNAs can be exported into plant cells to silence plant defense genes (16), highlighting bi-directional cross-kingdom RNAi between plant and fungal pathogens. Although very little is known about the mechanisms of small RNA/dsRNA trafficking between host cells and fungal cells, the presence of numerous vesicles in the extrahaustorial matrix suggests that they may transfer silencing signals between the two organisms (18). Consistent with this hypothesis, two recent studies provide evidence that plant extracellular vesicles (EVs) are essential to deliver antifungal small RNAs into B. cinerea cells as well as anti-oomycete small RNAs into Phytophthora capsici cells (17, 19).

Cross-Kingdom RNAi can be Exploited to Confer Protection Against Eukaryotic Pathogens Possessing a Canonical RNA Silencing Machinery

The biological relevance of cross-kingdom RNAi has been initially demonstrated by expressing dsRNAs bearing homologies to vital or pathogenicity factors from a given parasite or pest provided that they possess a canonical RNAi machinery (e.g. functional DCL and AGO proteins). So far, this Host-Induced Gene Silencing (HIGS) technology has been successfully used to protect plants from invasion and predation of insects, nematodes, oomycetes, fungi and parasitic plants (WO 2012/155112, WO 2012/155109, CA 2 799 453, EP 2 405 013, US 2013/177539, 15, 20, 21)). For example, HIGS confers full protection against Fusarium graminearum and B. cinerea and this phenomenon is fully recapitulated by spraying relevant exogenous dsRNAs or siRNAs into wild type plants prior fungal infections (15, 20, 21). The latter phenomenon is referred to as Spray-Induced Gene Silencing (SIGS) and is reminiscent of ‘environmental RNAi’, a process involving the uptake of RNAs from the environment initially described in Caenorhabditis elegans and in some insects (21, 22). HIGS/SIGS is thus considered as a powerful complement, or even sometimes an alternative, to conventional breeding or genetic engineering designed to introduce R genes or PAMP receptors in agriculturally relevant crops (5, 23, 24). Furthermore, this technology provides a more durable and environmental friendly plant protection solution that will likely contribute to a reduced use of agrochemicals, which can have, in some instances, significant impact on human health and on the environment.

Current Limitation of HIGS SIGS Technologies

HIGS/SIGS technologies are limited by the fact that they have only been shown to be functional against plant pathogens and parasites that possess a canonical RNA silencing machinery. For example, SIGS against F. graminearum relies at least in part on the uptake of dsRNAs and further processing by the fungal DICER-LIKE 1 protein (21). So far, there is no example of HIGS/SIGS directed against plant pathogens that do not possess a canonical RNAi machinery such as the bacterial pathogens that are used here by the inventors and that do not contain canonical eukaryotic-like RNA silencing factors in their genomes, as explained in the review of S. Ghag, 2017 (22). That is why, as of today, RNA-based silencing technologies have not been exploited to protect plants from bacterial pathogens. This is a considerable limitation because bacterial pathogens have a major impact on agricultural food quality and production, which results in significant economic losses worldwide. This is for instance the case of bacterial pathogens such as Pseudomonas, Ralstonia, Xylella, Xanthomonas, which cause infections of a broad range of cultivated plants (25). Alongside phytopathogenic bacteria, animal pathogenic bacteria also represent a major threat for human and animal health. In 2009, a joint report from the European Medicines Agency and European Centre for Disease Prevention and Control highlighted this concern. They have for instance estimated that about 25,000 patients, out of the 400,000 patients infected with multidrug-resistant (MDR) bacteria, die each year in the EU due to antibiotic-resistant bacterial strains, and this number is expected to increase due to the rise of such MDR bacteria. This results in extra healthcare costs, which represent €1.5 billion economic losses annually (65-66). Furthermore, there is emerging evidence indicating that untreated cultivated plants such as raw vegetables are vehicles for the transmission of human food borne infections (26-29). As an example, drug-resistant Salmonella and Shigella were recovered in lettuce and green peppers grown from different outlets in Addis Ababa (Ethiopa) and thus represent a source of inoculum for consumers (26).

Some authors have speculated that it could be possible to affect bacterial growth by contacting bacterial cells with long dsRNAs. For example, WO 2006/046148 proposes to control the proliferation of pests that can take-up long dsRNA fragments (>80 base pairs), among which, supposedly, bacteria. Yet, WO 2006/046148's inventors did not provide any experimental evidence that bacteria are sensitive to such long RNA fragments (their examples only disclose the effect of dsRNAs on nematodes). On the contrary, the present inventors herein demonstrate that bacteria are not sensitive to long dsRNAs, indicating that the hypothesis raised by WO 2006/046148's inventors is not valid when targeting prokaryotic cells.

Purpose of the Invention

In the present invention, the authors show here, for the first time, that plant small RNAs can efficiently inhibit the expression of genes from bacterial phytopathogens in a sequence-specific manner, a phenomenon referred to here as “Antibacterial Gene Silencing” (AGS). This regulatory mechanism was notably shown to operate within two different Gram-negative phytopathogenic bacterial species, indicating that plant small RNAs can be taken-up by bacterial cells despite the presence of a cell wall comprising an intricate double membrane structure (the bacterial inner and outer membranes). This is an unexpected result, since it has never been shown in the past that plant small RNAs can penetrate through the bacterial phospholipid bilayer or be passively or actively transported inside pathogenic bacterial cells. Furthermore, this phenomenon was not restricted to plant pathogenic bacteria, because the inventors additionally demonstrated that plant small RNAs can trigger AGS in a typical Gram-negative human pathogenic bacterium, highlighting the widespread potential of the invention.

Yet, despite all these prejudices, the Inventors' discoveries demonstrate that it is in fact possible to direct silencing of any bacterial gene, e.g. virulence factors, essential genes or artificial reporter genes, by contacting bacterial cells with small RNAs bearing sequence homologies to one or multiple bacterial target genes. These small RNAs can be stably expressed by said plant cells to protect them against one or multiple bacterial pathogens.

Alternatively, they can be exogenously administrated on the surface or within plant tissues or animal tissues that will encounter the targeted pathogenic bacterium, thereby dampening its pathogenicity and growth. Thus, contrary to what was thought so far, small RNA-directed silencing can be used to efficiently knock-down gene expression from plant and animal bacterial pathogens that do not possess eukaryotic-like RNA silencing machinery and that even possess a double membrane.

This unexpected sensitivity of bacterial cells to exogenously delivered small RNAs can be used purposely in antibacterial applications, and a vast number of treatments can be envisaged to reduce survival, pathogenicity and/or growth of plant and animal bacterial pathogens.

Finally, the Inventors have employed an in vitro-based assay to identify in a rapid, reliable and cost-effective manner, small RNAs with antibacterial activity. Therefore, it is anticipated that the present invention will be extensively employed to (i) protect plants and animals against bacterial pathogens, (ii) enhance the beneficial effect and/or the growth of commensal and symbiotic bacteria, and (iii) characterize the function of any genes in any bacterial species.

### DETAILED DESCRIPTION OF THE INVENTION

Overview

In the results below, the Inventors show that AGS is an efficient technology to enhance protection towards bacterial infections by targeting—individually or concomitantly—key genes required for bacterial pathogenicity. They have notably constitutively expressed in Arabidopsis stable transgenic plants small RNAs bearing homologies to two major virulence factors from the Gram-negative bacterium Pto DC3000, namely Cfa6 and HrpL, and found a significantly lower virulence and growth of this bacterial pathogen when contacted with plant cells expressing these small RNAs. An enhanced protection against Xanthomonas campestris pv. campestris (Xcc), which is the causal agent of black rot, one of the most devastating diseases of crucifer crops, was also observed in Arabidopsis transgenic plants expressing small RNAs against the virulence factors HrpG, HrpX and RsmA. These data demonstrate that AGS can be employed to protect plants against unrelated agriculturally relevant phytopathogens.

They have also shown that the reduced virulence observed in Arabidopsis transgenic plants expressing anti-Cfa6 and anti-HrpL siRNAs is associated with a specific decrease in the expression of the two-targeted virulence factors in Pto DC3000. This in vivo antibacterial gene silencing phenomenon was not only found to be effective against these endogenous stress-responsive virulence genes but also against heterologous reporter genes expressed constitutively from Pto DC3000 genome. These findings therefore highlight that, despite its lack of canonical eukaryotic-like RNA silencing machinery, bacterial cells are actually sensitive to the action of plant-encoded small RNAs. They also provide evidence that artificial small RNAs produced in plants can induce gene silencing in extracellular bacterial pathogens, indicating that the small RNAs must be exported from host cells to bacterial cells, through a mechanism implicating different populations of extracellular plant small RNAs (see below).

Strikingly, this silencing effect has been observed not only on genetically modified plants so as to stably express the small RNAs bearing homology to Cfa6 and HrpL genes, but also on WT plants pre-treated with total RNAs containing anti-Cfa6 and anti-HrpL siRNAs and subsequently inoculated with Pto DC3000. Intriguingly, the inventors have also discovered that in vitro synthesized double-stranded small RNAs directed against either the genes from Pto DC3000 or the Gram-negative human pathogenic bacterium Pseudomonas aeruginosa were also competent for AGS. These findings further support the fact that small RNAs can reach the cytoplasm of bacteria, despite the presence of a double membrane, and trigger gene silencing in various prokaryotic cells, despite the absence of a canonical eukaryotic-like RNAi machinery.

In addition, by generating recombinant bacteria expressing a small RNA resilient version of HrpL that contains as many silent mutations as possible in the region that is targeted by small RNAs (which were designed to alter the binding of small RNAs with the HrpL mRNA but to produce the same protein sequence), the Inventors showed that the silencing of HrpL was no longer effective. In addition, they observed that the virulence of this recombinant bacterium was unaltered upon exogenous application of total RNAs containing effective anti-HrpL small RNAs. These findings provide thus compelling experimental evidence that small RNAs directed against the HrpL gene are causal for both AGS and the dampening of bacterial pathogenicity.

The Inventors went on to further investigate which RNA entities are responsible for the observed AGS phenomenon in response to exogenous total RNAs carrying antibacterial RNAs. Interestingly, by separating small RNA and long RNA species from total RNAs extracted from transgenic plants expressing a chimeric hairpin that target both the Cfa6 and HrpL genes, they showed that the exogenous delivery of the small RNA fraction onto plants triggered the antibacterial effect, while treatment with the long RNA fraction was ineffective. In addition, the inventors showed that total RNA extracts from a IR-CFA6/HRPL reference line, which was mutated in DCL2, DCL3 and DCL4 genes and thus impaired in the biosynthesis of anti-Cfa6 and anti-HrpL siRNAs, were not effective in triggering AGS nor pathogenesis reduction. Collectively, these findings provide compelling evidence that small RNAs, but not their long dsRNA precursors (unless they are processed into small RNAs in planta), are the RNA entities that are causal for AGS. This is a major distinction from environmental RNAi previously reported in C. elegans and plant herbivores, which specifically relies on long dsRNAs (30-36), or in the eukaryotic filamentous pathogens B. cinerea and F. graminearum, which is triggered by either dsRNAs or siRNAs (15, 21).

Importantly, the Inventors additionally demonstrate that exogenous application of total RNAs containing effective small RNAs against Cfa6 and HrpL genes can efficiently reduce Pto DC3000 growth and pathogenicity in the agriculturally relevant plant Solanum lycopersicum (tomato), which is the natural host of this bacterium. Therefore, it is anticipated that this RNA-based biocontrol approach can be exploited to confer—with a high sequence-based selectivity-protection against a wide range of bacterial pathogens. It can also be predicted that applying small RNAs bearing sequence homologies to virulence factors and/or essential genes on the surface of (or within) various tissues of either plants or animals will significantly reduce bacterial infection. Furthermore, this method can be easily designed to control multiple bacterial pathogens by concomitantly targeting essential genes and/or virulence factors from various plant or animal bacterial pathogens. AGS therefore represents a novel environmental friendly RNAi-based technology to protect both plants and animals against bacterial diseases.

In the results below, the inventors have also investigated the possible role of EVs in the trafficking of plant small RNAs towards bacterial cells. They have discovered at least two populations of EVs possessing antibacterial activities, one of large size, which were fully active in dampening bacterial pathogenesis, and another one of smaller EVs, which were moderately less active. Furthermore, they showed that these antibacterial small RNAs are protected from micrococcal nuclease (Mnase) digestion when embedded within these EVs, highlighting the potential of plant EVs for future disease management strategies in field conditions and in RNA-based therapeutics. Intriguingly, the inventors have additionally discovered that apoplastic EV-free antibacterial small RNAs, which were not associated with proteins, were also fully active in dampening pathogenesis. These novel small RNA species are referred to here as Extracellular Free Small RNAs or “efsRNAs”, and were sensitive to Mnase digestion. The inventors therefore concluded that the apoplast of IR-CFA6/HRPL transgenic plants is composed of at least three populations of functional antibacterial small RNAs, which are either embedded in large EVs, in smaller EVs, or in a free form.

The Inventors have also transiently expressed small RNAs using well-established Agrobacterium-mediated transient transformation of tobacco leaves, followed by the in vitro incubation of corresponding candidate antibacterial siRNAs with bacterial cells. This approach was notably useful to determine that siRNAs directed against the HrpL gene were equally efficient in preventing Pto DC3000-induced stomatal reopening as compared to siRNAs targeting Cfa6 and HrpL genes concomitantly. Furthermore, the inventors have demonstrated that the in vitro synthesis of small RNAs is an easy, rapid and reliable approach to screen for candidate small RNAs triggering antibacterial effects, such as bacterial gene silencing and the suppression of bacterial-induced stomatal reopening. They have also coupled the in vitro small RNA synthesis approach with a droplet-based microfluidic system to show that siRNAs directed against the conserved genes, GyrB or FusA from Pto DC3000 can drastically alter bacterial growth in vitro, thereby identifying novel bactericidal agents. It is therefore anticipated that such transient tobacco- or in vitro-based synthesis of candidate small RNAs, followed by incubation of corresponding small RNAs with bacterial cells, will be extensively employed in the future by academic laboratories and industrials to identify small RNAs having strong effects on bacterial gene expression and/or on specific phenotypes (e.g. bacterial growth, survival, metabolic activities). It is also anticipated that the AGS technology described herein will be widely used to characterize the function of bacterial genes through a novel RNA-based reverse genetic approach. This method was, for instance, instrumental to demonstrate for the first time a role for HrpL in Pto DC3000-induced stomatal reopening, as well as a role for GyrB and FusA in the survival or fitness of Pto DC3000. Finally, because tobacco plants are already used by industrials to produce high yields of recombinant proteins or vesicle-like particles in a cost-effective manner (cf. EP2610345 from Medicago Inc.), they will likely be exploited to produce candidate small RNAs, particularly within EVs in which they will be well protected from nuclease degradation, for future RNA-based biocontrol applications in crops.

The above findings, along with the fact that long dsRNAs expressed from mammalian cells are known to trigger potent antiviral interferon response (37), which is not the case in plant cells, prompted the inventors to assess whether plants could be exploited as bioreactors for small RNA production against animal pathogenic bacteria. For this end, they have transiently expressed specific inverted repeat constructs in tobacco leaves using Agrobacterium-mediated transient transformation and further incubated corresponding RNA extracts (containing antibacterial small RNAs) with cells of the human pathogenic bacterium P. aeruginosa. By doing so, they discovered that these plant small RNAs were indeed capable of triggering AGS of both artificial reporter genes as well as some endogenous housekeeping genes in P. aeruginosa. They have notably shown that plant RNA extracts containing siRNAs against multiple essential genes triggered reduced growth of a P. aeruginosa strain in in vitro conditions. In addition, the inventors have performed proof-of-concept experiments demonstrating that the in vitro synthesis of antibacterial small RNAs coupled with a droplet-based microfluidic system is also a suitable approach to rapidly identify candidate small RNAs possessing bactericidal activities. This was notably the case of anti-SecE siRNAs, which triggered a significant reduction in the in vitro growth of P. aeruginosa. It is therefore anticipated that such transient tobacco- or in vitro-based synthesis of candidate small RNAs, followed by incubation of corresponding small RNAs with the target bacterial cells, will be extensively used by academic laboratories and industrials to identify small RNAs that can interfere with bacterial gene expression and/or on specific phenotypes from animal pathogenic or beneficial bacteria. This approach will for instance be instrumental to identify small RNAs that can efficiently silence antibiotic resistance genes and that will be further used to restore antibiotic sensitivity when co-administered with a given antibiotic. It is also anticipated that the AGS technology described herein will be exploited to characterize the function of bacterial genes from animal pathogenic and beneficial bacteria. This approach was for example instrumental to provide evidence for a role of SecE, DnaN and GyrB genes as fitness determinants of P. aeruginosa, thereby validating previous reports (38-40). Finally, because tobacco plants are already used by industrials to produce high yields of recombinant proteins or vesicle-like particles for pharmaceutical applications (cf. EP2610345 from Medicago Inc.), they will likely be exploited to produce anti-infective small RNAs, particularly within EVs in which they will be protected from nuclease degradation, for future RNA-based therapeutics. The use of plant EVs for small RNA delivery and therapeutic applications is particularly attractive because these natural vesicles do not usually induce cytotoxic effects in mammalian cells, which can be the case of synthetic nanoparticles (41).

Based on all these discoveries, the present Inventors propose a method to inhibit the expression of at least one gene in bacteria, said method comprising either:

i) introducing into at least one plant cell at least one functional interfering RNA molecule (iRNA) targeting specifically at least one bacterial gene, said iRNA being able to induce sequence-specific silencing of said gene(s) in bacteria carrying said gene(s), or

ii) delivering small RNAs, e.g., extracellular vesicles or apoplastic fluids containing same, or extracellular free RNAs, on plant or animal tissues prior to and/or after bacterial infection, or

iii) delivering small RNAs, e.g., extracellular vesicles or apoplastic fluids containing same, or extracellular free RNAs, directly on bacterial cells.

In one embodiment, this method allows the targeting of one or multiple bacterial gene(s) by expressing iRNA molecules (precursors of siRNAs and miRNAs) in plant cells, ii) recovering the apoplastic fluid (APF) of said plant cells, iii) delivering the small RNAs present in said APF on animal tissues, within animals (e.g. organs, body fluids) or on bacterial cells. This approach will have major impact on public health, especially in the management of bacterial infections.

More precisely, this technology will provide a way to control bacterial infections in plants and animals, and therefore reduce antibiotic treatments without having a negative effect on beneficial bacteria or on the environment due to the high sequence-based selectivity of this approach.

Furthermore, this strategy will provide more durable disease resistance, which is not the case with some conventional treatments.

Finally, it can be anticipated that the herein described technology will also be useful to control the expression of genes from beneficial bacteria in order to enhance their multiplication and/or their beneficial effects for the host animals.

Besides these advantages, the proposed method is cost-effective and relatively easy to industrialize. Indeed, the process of designing and producing effective artificial iRNAs (such as siRNAs) against bacterial genes only takes a few weeks when transiently expressed from N. benthamiana leaves or even a single day when synthesized in vitro. Furthermore, it is relatively easy to redesign and produce de novo artificial iRNAs upon appearance of siRNA-resistant bacteria. Finally, it is possible to produce iRNAs directed against either a specific bacterial species or against a vast range of pathogenic bacterial strains thereby providing targeted or broad-spectrum treatments depending on the RNA-based therapeutics desired.

The present method/use can be performed either in vivo or in vitro. By “in vitro”, it is herein meant that the steps of the claimed methods or uses are conducted using biological components (e.g., bacterial cells) that have been isolated from their usual host organisms (skin, mucosa, stool, etc.) or that are directly grown in in vitro media (in the absence of their host organisms). This is the case when the small RNAs of the invention are contacted directly with the bacterial cells.

By “in vivo” or “in planta”, it is herein meant that the steps of the claimed methods or uses are conducted using whole organisms, for example whole individuals.

When small RNAs of the invention are contacted directly with bacterial cells that contain tissues in their surroundings, notably to trigger silencing of virulence factors within bacterial cells, the present method/use is said to be performed as a “semi-in vivo” assay.

Useful Precursors of the Small RNAs of the Invention

The present invention targets the use of at least one functional interfering RNA (iRNA) for inhibiting the expression of at least one gene in a bacterial cell.

As used herein, the term “functional interfering RNA” (functional iRNA) refers to a RNA molecule capable of inducing the process of sequence-specific silencing of at least one bacterial gene(s), especially in bacteria cells. In particular, said functional interfering RNA molecule can be either i) a small interfering RNA, well-known in the art as small or short interfering RNA (siRNA) molecule (simplex or duplex), or a precursor thereof, or ii) a microRNA (miRNA) molecule (simplex or duplex) or a precursor thereof.

The term “precursor of siRNA” or “siRNA precursor” herein refers to an RNA molecule which can be directly or indirectly processed into siRNA duplex(es) in plants (or plant extracts). Examples of siRNA precursors that can be directly processed include long double-stranded RNA (long dsRNA), while examples of siRNA precursors that can be indirectly processed include long single-stranded RNA (long ssRNA) that can be used as template for the production of processable long dsRNAs.

The term “precursor of miRNA” or “miRNA precursor” herein refers to an RNA molecule which can be processed into miRNA duplex(es) in plants (or plant extracts). Examples of miRNA precursors include primary miRNA precursors (pri-miRNAs) and pre-miRNAs, comprising a hairpin loop.

Of note, plasmids or vectors and other DNA constructs or viral vectors encoding said precursor molecules are also encompassed in the definition of “functional interfering iRNA”.

For targeting multiple genes in a bacterium, the method of the invention can use i) a mixture of several different iRNAs which altogether target multiple bacterial genes of interest or ii) a chimeric iRNA targeting several different bacterial genes of interest or iii) a mixture of any of these chimeric iRNAs.

In one particular embodiment, the method/use of the invention comprises the introduction of one or several functional iRNAs into eukaryotic cells (e.g., plant cells) as precursors, to produce in planta the small RNAs (such as siRNAs or miRNAs) that can be further formulated and used to prevent bacteria infection.

In a more particular embodiment, the functional iRNAs of the invention are long single-stranded RNA molecules (named hereafter as “long ssRNAs”). Such long ssRNA may be produced by a plant transgene, converted into long dsRNA molecules by plant RNA-dependent RNA polymerases, and further processed into siRNAs by plant DCL proteins.

Alternatively, long ssRNA may be produced by a plant RNA virus and further converted into long dsRNA molecules either during viral replication (as replicative intermediates) and/or through the action of plant RNA-dependent RNA polymerases. The resulting viral dsRNA is subsequently processed into siRNAs by plant DCL proteins, which subsequently trigger sequence-specific silencing through a process referred to as Virus-Induced Gene Silencing (VIGS) (11).

As used herein, the term “long ssRNA” designates single-stranded structures containing a single-strand of at least 50 bases, more preferably of 80 to 7000 bases. Long ssRNAs may contain 80 to 7000 bases when produced by a plant transgene, but preferably contain 80 to 2000 bases when produced by a plant recombinant RNA virus.

In a more particular embodiment, the functional iRNAs of the invention are long double-stranded RNA molecules (named hereafter as “long dsRNAs”) that act as siRNA precursor and can be processed into siRNAs, in planta, thanks to DCL proteins and other small RNA biogenesis factors encoded by plant genomes.

As used herein, the term “long dsRNA” designates double-stranded structures containing a first (sense strand) and a second (antisense) strand of at least 50 base pairs, more preferably of 80 to 7000 base pairs.

In plants or plant cells, long dsRNAs can be processed into small RNA duplexes. Such long dsRNAs are advantageously chimeric dsRNA, i.e., they bear sequence homologies to multiple bacterial genes (see below).

In one embodiment, the functional iRNA of the invention is a long dsRNA that is cleavable by DCL proteins in plant cells so as to generate siRNAs.

The long dsRNAs of the invention can be generated from a hairpin structure, through sense-antisense transcription constructs, through an artificial sense transcript construct further used as a substrate by plant RNA-dependent RNA polymerases, or through VIGS. More precisely, they may comprise bulges, loops or wobble base pairs to modulate the activity of the dsRNA molecule so as to mediate efficient RNA interference in bacterial cells. The complementary sense and antisense regions of the long dsRNA molecule of the invention may be connected by means of nucleic acid based or non-nucleic acid based linker(s). The long dsRNA of the invention may also comprise one duplex structure and one loop structure to form a symmetric or asymmetric hairpin secondary structure.

Therefore, in one embodiment, the functional iRNA of the invention is a long (at least 50 base pairs, more preferably of 80 to 400 base pairs, 100 to 200 base pairs, 125 to 175 base pairs, in particular about 150 base pairs) dsRNA comprising a hairpin such as miRNA precursors.

As demonstrated in the examples of the present application, the introduction of dsRNA into plant eukaryotic cells induces a sequence-specific silencing of the bacterial gene(s) in the bacteria cells through the action of small RNAs but not long dsRNAs (example 6 & FIG. 7). This means that bacterial cells are only sensitive to AGS when they are directly contacted by small RNA entities. Contacting bacteria directly with precursors of small RNAs (long dsRNAs) will have no silencing effect since these prokaryotic cells do not possess canonical eukaryotic-like RNAi machinery to process them properly into functional antibacterial iRNAs.

Small RNAs of the Invention

As a matter of fact, it is possible to inhibit the expression of bacterial genes directly in bacterial cells by contacting them with small RNA species whose size is shorter than 50 base pairs (FIG. 8 & FIG. 10).

Therefore, in another preferred embodiment, the functional iRNAs of the invention are small RNAs such as siRNAs or miRNAs. These small RNAs have a short size, which is less than 50 base pairs, preferably comprised between 15 and 30 base pairs, more preferably between 19 and 27 base pairs, even more preferably between 20 and 25 base pairs.

These small RNAs can be formulated in pharmaceutical or cosmetical compositions, e.g., into topic composition or into sprayable liquid compositions (see below). In this case, the said compositions containing the said small RNAs can be administered directly to tissues or to bacteria.

In one particularly preferred embodiment, the functional iRNA of the invention is a “siRNA”, which designates either a “siRNA duplex” or a “siRNA simplex”.

More specifically, the term “siRNA duplex” designates double-stranded structures or duplex molecules containing a first (sense strand) and a second (antisense) strand of at least 15 base pairs, preferably of at least 19 base pairs; preferably, said antisense strand comprises a region of at least 15 contiguous nucleotides that are complementary to a transcript of the targeted gene. These siRNA duplexes can be produced from long dsRNA precursors that are processed by plant DCL proteins. They can also be de novo chemically synthesized, as disclosed below. They have a short size, which is less than 50 base pairs, preferably comprised between 15 and 30 base pairs, more preferably between 19 and 27 base pairs, even more preferably between 20 and 25 base pairs.

A shown in the experimental part below (Example 9 and FIG. 10), the small RNAs of the invention are efficient when they are under double-stranded structure. It has been demonstrated with in vitro de novo synthesized siRNA duplexes, and it is thought that the biological effect observed with plant extracts is at least in part due to these siRNA duplexes secreted by the plants. Therefore, in a preferred embodiment, the iRNAs of the invention are double-stranded small RNAs.

As used herein, the term “siRNA simplex” or “mature siRNA” designates simplex molecules (also known as “single-stranded” molecules) that originate from the siRNA duplex but have been matured in the RISC machinery of a plant cell and are loaded in an AGO protein and/or associated with other RNA-binding proteins. They can also be de novo chemically synthesized, as disclosed below. They have a short size, which is less than 50 bases, preferably between 15 and 30 bases, more preferably between 19 and 27 bases, even more preferably between 20 and 25 bases.

In another embodiment, the functional iRNA of the invention is a “miRNA”, which designates either a “miRNA duplex” or a “miRNA simplex”. In a preferred embodiment, the iRNAs of the invention are double-stranded miRNAs.

More specifically, the term “miRNA duplex” designates double-stranded structures or duplex molecules containing a first (sense strand) and a second (antisense) strand of at least 15 base pairs, preferably of at least 19 base pairs; preferably, said antisense strand comprises a region of at least 15 contiguous nucleotides that are complementary to a transcript of the targeted gene. These miRNA duplexes may also contain bulges. These miRNA duplexes can be produced from miRNA precursors that are processed by plant DCL proteins. They can also be de novo chemically synthesized, as disclosed below. As the duplex siRNAs, they have short size which is less than 50 base pairs, preferably comprised between 15 and 30 base pairs, more preferably between 19 and 27 base pairs, even more preferably between 20 and 25 base pairs.

As used herein, the term “miRNA simplex” or “mature miRNA” designates simplex molecules (also known as “single-stranded” molecules) that originate from the miRNA duplex but have been matured in the RISC machinery of a plant cell and are loaded in an AGO protein and/or associated with other RNA-binding proteins. They can also be de novo chemically synthesized, as disclosed below. As the simplex siRNAs, they have a short size which is less than 50 bases, preferably comprised between 15 and 30 bases, more preferably between 19 and 27 bases, even more preferably between 20 and 25 bases.

Methods to design iRNAs such as long dsRNAs/siRNA/miRNA are available in the art and can be used to obtain the sequence of long dsRNAs, siRNA and miRNA having these properties.

The inventors herein show (Example 9, FIG. 10) that it is possible to use artificial in vitro synthetized double-stranded siRNAs in order to (i) inhibit bacterial gene expression (ii) dampen bacterial pathogenicity, and (iii) trigger bactericidal effects in vitro (see FIG. 10).

The invention encompasses the use of synthetic, semi-synthetic or recombinant iRNAs comprising ribonucleotides only or both deoxyribonucleotides and ribonucleotides. The invention also encompasses the use of modified iRNA molecules comprising one or more modifications, which increase resistance to nuclease degradation in vivo and/or improve cellular stability (e.g. small RNA 3′ end methylation, locked nucleic acid (LNA)), uptake by bacterial cells (e.g. peptide carriers) or silencing efficacy within bacterial cells. The iRNAs of the invention may include nucleotides, which are modified at the sugar, phosphate, and/or base moiety, and/or modifications of the 5′ or 3′ end(s), or the inter-nucleotidic linkage.

Chemically synthesized dsRNA molecules as defined in the invention may be assembled from two distinct oligonucleotides, which are synthesized separately. Alternatively, both strands of the RNA duplex or RNA precursor molecule may be synthesized in tandem using a cleavable linker, for example a succinyl-based linker. Alternatively, the RNA precursor molecules of the invention may be expressed (in vitro or in planta) from transcription units inserted into DNA or RNA vectors known to those skilled in the art and commercially available. It is noteworthy that the latter approach can include the transcription of transgenes expressing long double-stranded fold-back structures, sense-antisense transcripts through promoters located from each part of the transgene and in opposite orientation, miRNA precursors, primary miRNA transcript, or sense transcripts that can, in some instances (e.g. targeted by endogenous or exogenous 22 nt long miRNAs) be used as substrates by the plant RNA-dependent RNA polymerases to generate dsRNAs.

The iRNA molecules of the invention, in particular the small RNAs of the invention, preferably decrease the level of expression of the targeted bacterial gene(s) by at least 30%, preferably by at least 60%, more preferably by at least 80%, in bacteria carrying said gene(s). The silencing of the bacterial gene(s) can be assessed at the RNA or protein level, by methods well-known in the art, for example by real time quantitative RT-PCR (RT-qPCR), Northern Blot, FACS, Immunohistological analyses or Western Blot analyses.

In the context of the invention, the silencing of the bacterial gene(s) by artificial iRNA molecules, which may be partial or total, should be sufficient to produce the desired effect on the bacteria, such as for example to reduce bacterial pathogenicity or infectivity of said bacteria in an organism.

In a preferred embodiment, the small RNA of the invention has a size comprised between 15 and 30 base pairs and inhibits specifically at least one bacterial gene selected from the group consisting of: LptH, LolA, ToiB, LpxA, LpxD, XcpQ, PcrV, PcrR, Vrf, dnaA, dnaN, gyrB, rpoC, secE, sodB, ExoS, ExoU, exsA, LasR, RhlR, MvfR, VqsM, GacA, RsmA, VirF, VirB, IcsA, fnbA, clfA, cpfB, spa, atl, lukF-PV, lukS-PV, lukE, lukD, HlgB, hla, tsst-1, PscC, PscJ, PscN, VirB1, VirD4, TssM, TssJ, TssB TssC, TssE, VgrG, Hcp, DotC, DotD, DotF, DotG, DotH, LuxS, Luxl/LuxR, AroA, LysC, CysH, GalU, PbpA, PbpB, PbpC, Sigma70, Sigma 54, Arc, Ptr, Nor, Mep, Cme, TEM, SHV, GES, mexX, mexA, VIM, NDM, AmpC, VIM-1, VIM-2, VIM-3, VIM-5, Case, OXA-28, OXA-14, OXA-19, OXA-145, PER-1, TEM-116, GES-9, FtsZ, FtsA, FtsN, FtsK, FtsI, FtsW, ZipA, ZapA, TolA, TolB, TolQ, TolR, Pal, MinCD, MreB and Mld.

Targeted Bacteria

The use/method of the invention is useful for silencing genes in any type of bacteria (pathogenic or non-pathogenic; Gram-positive or Gram-negative), including beneficial bacteria known to be associated with animal organisms.

In a preferred embodiment, said targeted bacteria are human pathogenic bacteria.

Non-limitative examples of pathogenic bacteria, which can be targeted using the use/method of the invention include:

Actinomyces israelii, Bacillus anthracis, Bacillus cereus, Bacteroides fragilis, Bordetella pertussis, Borrelia sp. (burgdorferi, garinii, afzelii, recurrentis, crocidurae, duttonii, hermsii etc.), Brucella sp. (abortus, canis, melitensis, suis), Campylobacter jejuni, Chlamydia sp. (pneumoniae, trachomatis), Chlamydophila psittaci, Clostridium sp. (botulinum, difficile, perfringens, tetani), Corynebacterium diphtheriae, Ehrlichia sp. (canis, chaffeensis), Enterococcus (faecalis, faecium), Escherichia coli O157:H7, Francisella tularensis, Haemophilus influenza, Helicobacter pylori, Klebsiella pneumoniae, Legionella pneumophila, Leptospira sp., Listeria monocytogenes, Mycobacterium sp. (leprae, tuberculosis), Mycoplasma pneumoniae, Neisseria (gonorrhoeae, meningitidis), Pseudomonas aeruginosa, Porphyromonas gingivalis, Nocardia asteroides, Rickettsia rickettsii, Salmonella sp. (typhi, typhimurium), Shigella sp. (sonnei, dysenteriae), Staphylococcus (aureus, epidermidis, saprophyticus), Streptococcus sp. (agalactiae, mutans, pneumoniae, pyogenes, viridans), Tannerella forsythia, Treponema pallidum, Vibrio cholerae, and Yersinia pestis.

In a preferred embodiment, the target pathogenic bacteria do not belong to the Staphylococcus genus. In particular, it is not Staphylococcus aureus.

As shown in the EXAMPLES below, the use/method of the invention is particularly efficient in Gram-negative bacteria such as Pseudomonas aeruginosa (FIGS. 11 and 12). These results are surprising since these bacteria are known to contain a double-membrane that should impair small RNAs to enter the bacterial cytoplasm. The results show that a concentration of 5 ng/μL of the iRNAs of the invention significantly reduce the growth of the targeted bacteria in in in vitro conditions.

In a preferred embodiment, the use/method of the invention is useful for silencing genes in pathogenic Gram-negative bacteria, for example proteobacteria including Escherichia coli (E. coli), Salmonella, Shigella, or other Enterobacteriaceae, Pseudomonas, Moraxella, Helicobacter, Stenotrophomonas, Bdellovibrio, acetic acid bacteria, Legionella, etc. Medically relevant gram-negative bacilli include a multitude of species. Some of them cause primarily respiratory problems (Klebsiella pneumoniae, Legionella pneumophila, Pseudomonas aeruginosa), primarily urinary problems (Escherichia coli, Proteus mirabilis, Enterobacter cloacae, Serratia marcescens), and primarily gastrointestinal problems (Helicobacter pylori, Salmonella enteritidis, Salmonella typhi, Shigella flexneri, Shigella sonnei, Shigella dysenteriae, Shigella boydii). The iRNAs of the invention can be used to limit or prevent an infection of any of these bacteria.

In a particular embodiment, the method of the invention uses functional iRNA(s) targeting one or multiple genes of beneficial bacteria (e.g., commensal or symbiotic bacteria). The purpose of this particular embodiment is to promote the beneficial effects of said bacteria. In this particular embodiment, the targeted bacterial genes are factors that, when silenced, promote the replication of the targeted bacterial cells or a pathway that is beneficial for the host and that positively regulate the production of a beneficial compound (e.g. an hormone), secondary metabolites that (i) alter the survival/pathogenicity of surrounding pathogens or competitors, (ii) activate host defense responses (e.g. antimicrobial peptide production), (iii) facilitate the uptake of nutrients from the environment, (iv) enhance the tolerance of the host organism to abiotic stress conditions etc. Silencing of such bacterial target genes would thus lead to an increased growth rate of the host organism and/or several other possible beneficial effects for the host organism.

In such an embodiment, the iRNAs of the invention should have sequence homologies with beneficial bacterial genes but no sequence homology to pathogenic bacterial genomes, with the host genome or with other genomes of host colonizers and/or mammals that feed on the host organism.

Non-limitative examples of beneficial (commensal or symbiotic) bacteria, which can be targeted with the method of the invention include:

Actinomyces naeslundii, Veillonella dispar, Faecalibacterium prausnitzii, Enterobacteriaceae, Bacteroides thetaiotaomicron, Escherichia coli K12, Bifidobacterium sp. (longum, bifidum, adolescentis, dentium, breve, themophilum), Eggerthella lenta, Bacteroides sp. (xylanisolvens, thetaiotaomicron, fragilis, vulgatus, salanitronis), Parabacteroides distasonis, Faecalibacterium prausnitzii, Ruminococcus sp. (bromii, champanellensis, SR1/5), Streptococcus (parasanguinis, salivarius, thermophilus, suis, pyogenes, anginosus), Lactococcus (lactis, garvieae), Enterococcus (faecium, faecalis, casseliflavus, durans, hirae, Melissococcus plutonius, Tetragenococcus halophilus, Lactobacillus sp. (casei, ruminis, delbrueckii, buchneri, reuteri, fermentum, pentosus, amylovorus, salivarius), Pediococcus (pentosaceus, claussenii), Leuconostoc (mesenteroides, lactis, carnosum, gelidum, citreum), Weissella (thailandensis, koreensis), Oenococcus oeni, Paenibacillus sp. (terrae, polymyxa, mucilaginosus, Y412MC10), Thermobacillus composti, Brevibacillus brevis, Bacillus (amyloliquefaciens, subtilis, licheniformis, atrophaeus, weihenstephanensis, cereus, thuringiensis, coagulans, megaterium, selenitireducens), Geobacillus thermodenitrificans, Lysinibacillus sphaericus, Halobacillus halophilus, Listeria sp., Streptomyces sp., Eubacterium (rectale, eligens, siraeum), Clostridium saccharolyticum, and butyrate-producing bacterium (SS3/4 and SSC/2).

Targeted Bacterial Genes

The iRNA of the invention should have a sufficient sequence homology with at least one bacterial gene in order to induce sequence-specific silencing of said at least one gene. In addition, to prevent unwanted off-target effects, the sequence homology of the dsRNAs, miRNAs or small RNA species of the invention with the eukaryotic host genome or other genomes of beneficial bacteria, host colonizers and/or mammals that feed on the host organism should be quasi inexistent (if not absent).

The iRNA of the invention is able to inhibit the expression of at least one bacterial gene.

According to the invention, the term “bacterial gene” refers to any gene in bacteria including (natural) protein-coding genes or non-coding genes, present naturally in bacteria and artificial genes introduced in bacteria by recombinant DNA technology. Said target bacterial genes are either specific to a given bacterial species or conserved across multiple bacterial species. Preferably, it shares no homology with any gene of the eukaryotic host genome, host colonizers and/or mammals that feed on the host organism. This avoids collateral effects on the plant host, beneficial bacteria associated with the host, host colonizers and/or mammals that feed on the host organism.

In a preferred embodiment, said at least one bacterial gene is a bacterial virulence factor or an essential gene for bacteria or an antibiotic resistance gene.

As used herein, the term “essential gene for bacteria” refers to any bacterial gene that is essential for bacterial cell viability. These genes are absolutely required to maintain bacteria alive, provided that all nutrients are available. It is thought that the absolutely required number of essential genes for bacteria is about 250-500 in number. The identification of such essential genes from unrelated bacteria is now becoming relatively easily accessible through the use of transposon sequencing approaches. These essential genes encode proteins to maintain a central metabolism, replicate DNA, ensure proper cell division, translate genes into proteins, maintain a basic cellular structure, and mediate transport processes into and out of the cell (42). This is the case of GyrB, DnaN or SecE genes, whose silencing were found to impair the growth of P. aeruginosa in vitro (FIG. 12). In the context of the invention, the iRNAs of the invention can also for example target the essential genes LptH, LolA, TolB, LpxA, LpxD, dnaA, dnaN, gyrB, rpoC, secE and sodB.

As used therein, the term “virulence gene” refers to any bacterial gene that has been shown to play a critical role for at least one of the following activity: pathogenicity, disease development, colonization of a specific host tissues or host cell environment, etc. All these activities help the bacteria to grow and/or promote disease symptoms in the host, although they are not essential for their survival in vitro.

In the context of the invention, the iRNAs of the invention target for example structural genes of secretion systems including the type II or III secretion system (e.g. PscC, PscJ, PscN, XcpQ, PcrV; PcrR), structural genes of the type IV secretion system (e.g. VirB1, VirD4), structural genes of the type VI secretion system (e.g. TssM, TssJ, TssB TssC, TssE, VgrG, Hcp), genes of the dot/icm system (DotC, DotD, DotF, DotG and DotH), transcriptional regulators or type III secreted effectors (ExoS, ExoU, exsA, VirF, VirB), the Vrf gene encoding the cAMP-dependent DNA-binding protein, adhesins (e.g. IcsA), quorum sensing-related genes (e.g. LasR, RhlR, MvfR, VqsM, LuxS, Luxl LuxR), essential genes involved in amino acid synthesis (AroA, LysC, CysH, GalU), transpeptidases (PbpA, PbpB, PbpC), genes encoding the GAC signaling-related components (GacA, RsmA), genes encoding components of bacterial transcriptional machinery (e.g. sigma 70, sigma 54), genes encoding structural components of bacterial cell walls (peptidoglycan biosynthesis genes), genes encoding surface bound proteins (fnbA, clfA, clfB, spa, atl), leukotoxins (lukF-PV, lukS-PV, lukE, lukD, HlgB), the alpha hemolysin hla, and the toxic shock syndrome toxin-1 tsst-1, genes that are critical for cell division (e.g. FtsZ, FtsA, FtsN, FtsK, FtsI, FtsW), structural homologs of actin (e.g. MreB, Mbl), other crucial genes such as ZipA, ZapA, TolA, TolB, TolQ, TolR, Pal, MinCD, actin-related genes (MreB and Mld), antibiotic targets in general (see The Comprehensive Antibiotic Resistance Database or “CARD”, 2017, a biological database that collects and organizes reference information on antimicrobial resistance genes, proteins and phenotypes, and covers all types of drug classes and resistance mechanisms and structures) (67) etc. for preventing or treating diseases caused by bacterial pathogens in human or non-human animals.

The iRNAs of the invention can also inhibit the expression of an antibiotic resistance gene in order to render the bacteria sensitive to said antibiotic treatment.

These antibiotic resistance genes are for example: bacterial efflux pump genes (Arc, Ptr, Nor, Mep, Cme types), genes of the four molecular classes of beta-lactamases: class A (e.g. TEM, SHV, GES types), class B (e.g. metallo beta-lactamases VIM, NDM), class C (e.g. AmpC type), class D (OXA type). Non-limitative examples of antibiotic resistance genes include: VIM-1, VIM-2, VIM-3, VIM-5, CasE, OXA-28, OXA-14, OXA-19, OXA-145, PER-1, TEM-116, and GES-9, as well as other vital genes that lead to lethality of the bacterium when these genes are deleted or inactivated in the microorganism and those listed in the The Comprehensive Antibiotic Resistance Database 2017 (or CARD 2017) (67). These target genes can also encode major virulence determinants of bacterial pathogens such as components required for the assembly of bacterial secretion system, transcriptional activators of bacterial effectors/toxins, quorum sensing receptors and other well-characterized pathogenicity factors from the bacterial pathogen that is targeted.

In a preferred embodiment, said virulence factor gene or bacterial viability gene or antibiotic resistant gene is therefore chosen in the group consisting of: LptH, LolA, TolB, LpxA, LpxD, XcpQ, PcrV, PcrR, Vrf, dnaA, dnaN, gyrB, rpoC, secE, sodB, ExoS, ExoU, exsA, LasR, RhlR, MvfR, VqsM, GacA, RsmA, VirF, VirB, IcsA, fnbA, clfA, cpfB, spa, atl, lukF-PV, lukS-PV, lukE, lukD, HlgB, hla, tsst-1, mexX, mexA and ampC.

In another preferred embodiment, said virulence factor gene or bacterial viability gene or antibiotic resistant gene is therefore chosen in the group consisting of: PscC, PscJ, PscN, VirB1, VirD4, TssM, TssJ, TssB TssC, TssE, VgrG, Hcp, DotC, DotD, DotF, DotG, DotH, LuxS, Luxl LuxR, AroA, LysC, CysH, GalU, PbpA, PbpB, PbpC, Pigma70, Sigma 54, Arc, Ptr, Nor, Mep, Cme, TEM, SHV, GES, VIM, NDM, mexX, mexA, AmpC, VIM-1, VIM-2, VIM-3, VIM-5, Case, OXA-28, OXA-14, OXA-19, OXA-145, PER-1, TEM-116, GES-9, FtsZ, FtsA, FtsN, FtsK, FtsI, FtsW, ZipA, ZapA, TolA, TolB, TolQ, TolR, Pal, MinCD, MreB and Mid.

The iRNAs of the invention are able to significantly affect the expression of any of these genes, in any pathogenic or commensal bacteria, preferably in pathogenic Gram-negative bacteria.

In this embodiment, the iRNAs have advantageously sequence homologies with essential genes for the viability or virulence genes from bacterial pathogen species but no sequence homology with commensal bacteria genomes. Such advantageous embodiment of the method avoids collateral effects on the commensal bacteria present in the host.

Particular useful sequences targeting some of these genes are provided in the EXAMPLES below, and in the SEQ ID NO: 108-145 and in SEQ ID NO:248-275. Each of these nucleotide constructs is also an aspect of the invention.

The iRNAs of the invention are for example the duplex small RNAs having the sequence SEQ ID NO: 108-109 (sequences of the first and second strand, concomitantly targeting the DnaA, DnaN and GyrB genes of P. aeruginosa), SEQ ID NO: 110-111 (sequences of the first and second strands, concomitantly targeting the RpoC, SecE and SodB genes of P. aeruginosa), SEQ ID NO: 112-113 (sequences of the first and second strands, concomitantly targeting the XcpQ, PscF and PscC genes of P. aeruginosa), SEQ ID NO: 114-115 (sequences of the first and second strands, concomitantly targeting the XcpQ, ExsA and HphA genes of P. aeruginosa), SEQ ID NO: 116-117 (sequences of the first and second strands, concomitantly targeting the FtsA, Can and Tsf genes of Shigella flexneri), SEQ ID NO: 118-119 (sequences of the first and second strands, concomitantly targeting the AccD, Der and Psd genes of Shigella flexneri), SEQ ID NO: 120-121 (sequences of the first and second strands, concomitantly targeting the VirF, VirB and IcsA gene of Shigella flexneri), SEQ ID NO: 122-123 (sequences of the first and second strands, targeting the FusA gene of Shigella flexneri), SEQ ID NO: 124-125 (sequences of the first and second strands, targeting the Can gene of Shigella flexneri), SEQ ID NO: 126-127 (sequences of the first and second strands, targeting the Tsf gene of Shigella flexneri), SEQ ID NO: 128-129 (sequences of the first and second strands, targeting the AccD gene of Shigella flexneri), SEQ ID NO: 130-131 (sequences of the first and second strands, targeting the Der gene of Shigella flexneri), SEQ ID NO: 132-133 (sequences of the first and second strands, targeting the Psd gene of Shigella flexneri), SEQ ID NO: 134-135 (sequences of the first and second strands, targeting the VirB gene of Shigella flexneri), SEQ ID NO: 136-137 (sequences of the first and second strands, targeting the VirF gene of Shigella flexneri), SEQ ID NO: 138-139 (sequences of the first and second strands, targeting the IcsA gene of Shigella flexneri), SEQ ID NO: 140-141 (sequences of the first and second strands, targeting the Spa47 gene of Shigella flexneri), SEQ ID NO: 142-143 (sequences of the first and second strands, targeting the MukB gene of Shigella flexneri), SEQ ID NO: 144-145, sequences of the first and second strands, targeting the YbiT gene of Shigella flexneri).

The iRNA of the invention can also be, for example, any of the duplex small RNAs having the sequence SEQ ID NO: 248-249 (sequences of the first and second strand, concomitantly targeting the LuxA and LuxB genes from P. aeruginosa), SEQ ID NO: 250-251 (sequences of the first and second strand, concomitantly targeting the LptH, LolA and TolB genes from P. aeruginosa), SEQ ID NO: 252-253 (sequences of the first and second strands, concomitantly targeting the LpxA, LpxD and TolB genes from P. aeruginosa), SEQ ID NO: 254-255 (sequences of the first and second strands, concomitantly targeting the secE, dnaN and gyrB genes from P. aeruginosa), SEQ ID NO: 256-257 (sequences of the first and second strands, concomitantly targeting the XcpQ, exsA, PcrV, LasR, RhlR, VqsM and RmsA genes of P. aeruginosa), SEQ ID NO: 258-259 (sequences of the first and second strands, concomitantly targeting the XcpQ, PscF and PscC genes of P. aeruginosa), SEQ ID NO: 260-261 (sequences of the first and second strands, concomitantly targeting the ExoS, exsA and Vrf genes from P. aeruginosa), SEQ ID NO: 262-263 (sequences of the first and second strands, concomitantly targeting the ExoU, exsA and Vrf genes from P. aeruginosa), SEQ ID NO: 264-265 (sequences of the first and second strands, targeting the LasR, RhlR and VqsM genes from P. aeruginosa), SEQ ID NO: 266-267 (sequences of the first and second strands, targeting the GacA, RmsA and MvfR genes from P. aeruginosa), SEQ ID NO: 268-269 (sequences of the first and second strands, targeting the VirF, VirB and IcsA genes of Shigella flexneri), SEQ ID NO: 270-271 (sequences of the first and second strands, targeting the fnbA, clfA, clfB and spa genes of S. aureus), SEQ ID NO: 272-273 (sequences of the first and second strands, targeting the lukF-PV, lukS-PV, lukE and lukD genes from S. aureus), SEQ ID NO: 274-275 (sequences of the first and second strands, targeting the HlgB, hla, tsst-1 and atl genes from S. aureus). In another preferred embodiment, the iRNAs of the invention target genes that negatively regulate the survival of beneficial (commensal/symbiotic) bacteria, or genes that prevent their invasion in and association with the host, or genes negatively controlling their carbohydrate metabolism and uptake (knocking-down such gene resulting in an increased bacterial titer).

The iRNAs of the invention share advantageously sequence homologies with any of these essential genes or virulence genes or antibiotic resistance genes from the targeted bacterial pathogen species.

As used herein, the term “sequence homology” refers to sequences that have sequence similarity, i.e., a sufficient degree of identity or correspondence between nucleic acid sequences. In the context of the invention, two nucleotide sequences share “sequence homology” when at least about 80%, alternatively at least about 81%, alternatively at least about 82%, alternatively at least about 83%, alternatively at least about 84%, alternatively at least about 85%, alternatively at least about 86%, alternatively at least about 87%, alternatively at least about 88%, alternatively at least about 89%, alternatively at least about 90%, alternatively at least about 91%, alternatively at least about 92%, alternatively at least about 93%, alternatively at least about 94%, alternatively at least about 95%, alternatively at least about 96%, alternatively at least about 97%, alternatively at least about 98%, alternatively at least about 99% of the nucleotides are similar.

Conversely, nucleotide sequences that have “no sequence homology” are nucleotide sequences that have a degree of identity of less than about 10%, alternatively of less than about 5%, alternatively of less than 2%.

Preferably, the similar or homologous nucleotide sequences are identified by using the algorithm of Needleman and Wunsch. Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide residue matches and an identical percent sequence identity when compared to the corresponding alignment generated by GAP Version 10.

Of note, the iRNAs of the invention do not inhibit genes that are expressed in eukaryotic cells, or in fungi, insects, pests or other plant-infecting pathogens. Specifically, the iRNAs of the invention do not inhibit the expression of oncogenes that have bacterial origin and are inserted into other genomes. More precisely, the iRNAs of the invention do not inhibit the expression of the oncogenes iiaM and ipt of the Agrobacterium tumefaciens bacteria.

Chimeric Silencing Elements

For protecting plants against diseases caused by several bacterial pathogens, the method of the invention advantageously uses functional iRNAs carrying sequence homologies with more than one bacterial genes (hereafter referred to as “chimeric iRNAs”). These chimeric iRNAs preferably share homology with at least two, three, four, or more bacterial essential genes and/or virulence factors, such as those described above.

In a preferred embodiment, the iRNA of the invention is a chimeric iRNA inhibiting at least one gene encoding a virulence factor or an essential gene of bacterial cells as defined above, together with at least one other gene encoding a virulence factor or an essential gene of other pathogens or parasites known to be sensitive to HIGS. It can be also a gene required for the biosynthesis of toxic secondary metabolites from non-bacterial pathogens or plant parasites.

In another preferred embodiment, the method of the invention uses: (i) one or more iRNAs targeting a widespread sequence region of an essential or virulence gene that is conserved in a large set of bacterial pathogens or (ii) one or more iRNAs targeting genes that are essential or virulence factors from unrelated bacterial pathogens. Such particular embodiment of the method confers broad-spectrum protection towards multiple bacterial pathogens. The iRNAs of the invention are advantageously long dsRNAs, miRNAs and/or siRNA as defined above.

In a particular embodiment, the method of the invention further comprises introducing into the plants one or more dsRNAs targeting one or multiple genes of parasite(s) that are different from bacteria, such as viruses, fungi, oomycetes, insects or nematodes. In this embodiment, the iRNAs are directed to an essential gene or to a virulence gene of the parasite(s). The one or more iRNAs targeting the genes of the parasite(s) is/are advantageously delivered concomitantly or co-expressed with the iRNA targeting the bacterial gene(s). In another particular embodiment, the method of the invention comprises contacting bacteria with small RNAs targeting one or multiple genes of parasite(s) that are different from bacteria, such as viruses, fungi, oomycetes, insects or nematodes. In this embodiment, the small RNAs are directed to an essential gene or to a virulence gene of the parasite(s). The one or more small RNAs targeting the genes of the parasite(s) is/are advantageously delivered concomitantly or co-expressed with the small RNA targeting the bacterial gene(s).

Such methods are useful for concomitant prevention or treatment of diseases caused by bacterial pathogens and other parasites. They can be carried out using chimeric iRNAs carrying sequence homologies with bacterial but also other pathogenic/parasitic genes, as proposed above, or a cocktail of iRNA molecules, some bearing homologies to bacterial genes and other bearing homologies to genes from other pathogens/parasites.

Vectors for Producing the Small RNAs of the Invention

In one preferred embodiment, the long and small RNAs of the invention are isolated as extracellular free RNA molecules that are used directly on production plant cells and on target bacterial cells, respectively.

Layered Double Hydroxide (LDH) clay nanosheets, which are non-toxic and degradable, can also be used to carry antibacterial dsRNAs. They have already been successfully employed to deliver antiviral dsRNAs and were found to confer viral protection for a period of at least 20 days (43).

In another preferred embodiment, the long RNAs of the invention are encoded by recombinant DNA constructs that facilitate the introduction into a plant cell and/or facilitate the expression of long RNAs in said plant cell. Said recombinant constructs can be a plasmid or a vector, which may be commercially available. It is preferably a plant expression vector as described below.

In another aspect, the present invention therefore relates to a plant recombinant DNA vector (or “DNA construct”) or a plant viral vector comprising a polynucleotide sequence encoding at least one functional interfering RNA (iRNA) inhibiting the expression of at least one bacterial gene, wherein said polynucleotide sequence is expressible in eukaryotic cells.

Said functional iRNA is as defined above, either a short or long dsRNA, a long ssRNA, a siRNA or miRNA, preferably said functional iRNA is a long dsRNA, a long ssRNA, a siRNA or a miRNA.

Said at least one bacterial gene is preferably an essential or a virulence bacterial gene or an antibiotic resistance gene as defined above.

In an embodiment, the vector is a DNA vector. Said DNA vector comprises advantageously a transcription unit comprising: a transcription initiation region, a transcription termination region, and the polynucleotide encoding the iRNA of the invention, wherein said polynucleotide sequence is operably linked to said initiation and termination regions in a manner that allows the expression of the iRNA molecule in the eukaryotic cell.

In a preferred embodiment, said eukaryotic cell is a plant cell that is able to express high amounts of iRNAs, such as N. benthamiana leaves that are well-adapted for Agrobacterium-mediated transient transformation.

The DNA vector of the invention may encode one or both strands of the iRNA molecule of the invention, or a single self-complementary strand that self-hybridizes into a dsRNA duplex. The transcription initiation region may be from a promoter for a eukaryotic RNA polymerase II or III (pol II or III) including viral promoters active in plant cells such as the CaMV 35S promoter, since transcripts from these promoters are expressed at high levels in all cells of the plant organisms. A large choice of promoters suitable for expression of heterologous genes in plant cells are available in the art. They can be obtained for instance from plant viruses. They include constitutive promoters, i.e. promoters which are active in most tissues and cells and under most environmental conditions, as well as tissue-specific or cell-specific promoters which are active only or mainly in certain tissues or certain cell types, and inducible promoters that are activated in response to chemical stimuli. Organ or tissue specific promoters that can additionally be used in the present invention for plant protection against bacterial pathogens include in particular promoters that are active in tissues/cell types that are relevant for the entry and the propagation of bacterial pathogens, for example in hydathodes, guard cells, xylem parenchyma cells and cells surrounding the base of trichomes.

Said transcription termination region is preferably recognized by a eukaryotic RNA polymerase, more preferably by Pol II or Pol III. For example, said transcription termination can be a TTTTT sequence.

Large numbers of DNA vectors suitable for dsRNA molecule expression are known to those having skill in the art and commercially available. The selection of suitable vectors and the methods for inserting DNA constructs therein are well known. The recombinant vectors capable of stably expressing the dsRNA molecules can be transformed in planta, and persist in target cells. The choice of the vector depends on the intended host and on the intended method of transformation of said host.

In an embodiment, the vector is a viral vector, preferably a plant viral vector. Said viral vectors is preferably selected from various plant RNA viruses (e.g. Tobacco mosaic virus, Tobacco rattle virus, Potato virus X, Barley stripe mosaic virus, Tomato bushy shunt virus), which can be used to produce high amount of small RNAs by plant cells through VIGS (11). Here also, the choice of the viral vector depends on the intended host and on the intended method of infection of said host. For VIGS, the tobacco plants Nicothiana benthamiana can be used.

The present invention also encompasses recombinant DNA vectors or viral vectors including one or more marker genes, which allows selecting the transformed host cells.

In a preferred embodiment, the DNA or viral vector of the invention comprises a polynucleotide sequence encoding two, three, or four functional interfering RNA (iRNA) genes as defined above, therefore being able to inhibit two, three, or four different bacterial genes. The skilled person can identify the best combinations of iRNA by conventional means. Combinations of more than four targeted genes are also encompassed within the present invention.

In one embodiment, the DNA vector of the invention comprises at least one of the sequences SEQ ID NO: 108-145, and 248-249 and 250-275, preferably at least one of the sequences SEQ ID NO: 108-145, more preferably the sequences systems: SEQ ID NO: 108-109 (sequences of the first and second strand, concomitantly targeting the DnaA, DnaN and GyrB genes of P. aeruginosa), SEQ ID NO: 110-111 (sequences of the first and second strands, concomitantly targeting the RpoC, SecE and SodB genes of P. aeruginosa), SEQ ID NO: 112-113 (sequences of the first and second strands, concomitantly targeting the XcpQ, PscF and PscC genes of P. aeruginosa), SEQ ID NO: 114-115 (sequences of the first and second strands, concomitantly targeting the XcpQ, ExsA and HphA genes of P. aeruginosa), SEQ ID NO: 116-117 (sequences of the first and second strands, concomitantly targeting the FtsA, Can and Tsf genes of Shigella flexneri), SEQ ID NO: 118-119 (sequences of the first and second strands, concomitantly targeting the AccD, Der and Psd genes of Shigella flexneri), SEQ ID NO: 120-121 (sequences of the first and second strands, concomitantly targeting the VirF, VirB and IcsA genes of Shigella flexneri), SEQ ID NO: 122-123 (sequences of the first and second strands, targeting the FusA gene of Shigella flexneri), SEQ ID NO: 124-125 (sequences of the first and second strands, targeting the Can gene of Shigella flexneri), SEQ ID NO: 126-127 (sequences of the first and second strands, targeting the Tsf gene of Shigella flexneri), SEQ ID NO: 128-129 (sequences of the first and second strands, targeting the AccD gene of Shigella flexneri), SEQ ID NO: 130-131 (sequences of the first and second strands, targeting the Der gene of Shigella flexneri), SEQ ID NO: 132-133 (sequences of the first and second strands, targeting the Psd gene of Shigella flexneri), SEQ ID NO: 134-135 (sequences of the first and second strands, targeting the VirB gene of Shigella flexneri), SEQ ID NO: 136-137 (sequences of the first and second strands, targeting the VirF gene of Shigella flexneri), SEQ ID NO: 138-139 (sequences of the first and second strands, targeting the IcsA gene of Shigella flexneri), SEQ ID NO: 140-141 (sequences of the first and second strands, targeting the spa47 gene of Shigella flexneri), SEQ ID NO: 142-143 (sequences of the first and second strands, targeting the MukB gene of Shigella flexneri), SEQ ID NO: 144-145 (sequences of the first and second strands, targeting the YbiT gene of Shigella flexneri), SEQ ID NO: 248-249 (sequences of the first and second strands, targeting the LuxA and LuxB genes of Pto DC3000 and P. aeruginosa), SEQ ID NO: 250-251 (sequences of the first and second strand, concomitantly targeting the LptH, LolA and TolB genes from P. aeruginosa), SEQ ID NO: 252-253 (sequences of the first and second strands, concomitantly targeting the LpxA, LpxD and TolB genes from P. aeruginosa), SEQ ID NO: 254-255 (sequences of the first and second strands, concomitantly targeting the secE, dnaN and gyrB genes from P. aeruginosa), SEQ ID NO: 256-257 (sequences of the first and second strands, concomitantly targeting the XcpQ, ExsA, PcrV, LasR, RhlR, VqsM and RmsA genes of P. aeruginosa), SEQ ID NO: 258-259 (sequences of the first and second strands, concomitantly targeting the XcpQ, PscF and PscC genes of P. aeruginosa), SEQ ID NO: 260-261 (sequences of the first and second strands, concomitantly targeting the ExoS, exsA and Vrf genes from P. aeruginosa), SEQ ID NO: 262-263 (sequences of the first and second strands, concomitantly targeting the ExoU, exsA and Vrf genes from P. aeruginosa), SEQ ID NO: 264-265 (sequences of the first and second strands, targeting the LasR, RhlR and VqsM genes from P. aeruginosa), SEQ ID NO: 266-267 (sequences of the first and second strands, targeting the GacA, RmsA and MvfR genes from P. aeruginosa), SEQ ID NO: 268-269 (sequences of the first and second strands, targeting the VirF, VirB and IcsA genes of Shigella flexneri), SEQ ID NO: 270-271 (sequences of the first and second strands, targeting the fnbA, clfA, clfB and spa genes of S. aureus), SEQ ID NO: 272-273 (sequences of the first and second strands, targeting the lukF-PV, lukS-PV, lukE and lukD genes from S. aureus), SEQ ID NO: 274-275 (sequences of the first and second strands, targeting the HlgB, hla, tsst-1 and atl genes from S. aureus).

The DNA vector of the invention can be prepared by conventional methods known in the art. For example, it can be produced by amplification of a nucleic sequence by PCR or RT-PCR, by screening genomic DNA libraries by hybridization with a homologous probe, or else by total or partial chemical synthesis. The recombinant vectors can be introduced into host cells by conventional techniques, which are known in the art.

In Vitro Antibiotic Methods and Uses of the Invention

In another aspect, the present invention relates to an in vitro method for inhibiting the expression of at least one gene in a target bacterial cell, said method comprising the step of contacting said target bacterial cell with one or more of the small RNAs of the invention or with compositions comprising same. In the case of virulence factors that are transcriptionally activated in the contact of host cells, specific medium will be used (e.g. minimal media).

In other words, the present invention relates to the in vitro use of small RNAs or of a composition comprising small RNAs, for inhibiting the expression of at least one gene in a target bacterial cell, wherein said target bacterial cell is contacted directly with said small RNA or with said composition.

Preferably, said small RNA is a single-stranded or double-stranded siRNA or a single-stranded or double-stranded miRNA duplex. More preferably, said small or long RNA inhibits the expression of at least one gene encoding a virulence factor or of an essential gene or of an antibiotic resistance gene if said bacterial cell is pathogenic, or inhibits the expression of at least one gene encoding a repressor of growth or of a negative regulator of a pathway that is useful for the host if said bacterial cell is beneficial.

Preferably, said composition contains plant extracts obtained from producer plant cells that have been contacted with at least one long dsRNA that is specific to at least one gene of said bacterial cell. More preferably, said composition contains extracellular vesicles recovered from said plant extracts, or extracellular free RNAs secreted by said plant extracts, apoplastic fluid from the said plant extracts, or nanoparticles complexed with said small RNAs. Said producer plant cells are for example chosen in the group consisting of: Tobacco (e.g. Nicotiana excelsior, Nicotiana excelsiana, Nicotiana benthamiana, Nicotiana tobaccum); Taro (Colocasia esculenta); Giger (Zingiber officinale), Arabidopsis (e.g. Arabidopsis thaliana); Tomato (e.g. Lycopersicon esculentum or Solanum lycopersicum); Potato (Solanum tuberosum); Rice (Oryza sativa); Maize (Zea mays); Barley (Hordeum vulgare); Wheat (e.g. Triticum aestivum, Triticum durum), Cottonseed, Cotton, Bean, Banana/plantain, Sorghum, Pea, Sweet potatoes, Soybeans, Cabbage, Cassava, Onion, Melon, Oats, Peanut, Sunflower, Palm oil, Rye, Citrus, Wheat, Peppers, Yams, Olives, Grapes, Sesame, Sugarcane, Sugarbeet, Pea and Coffee, Orange trees, Apple trees, Citrus trees, Olive trees, etc.

By “inhibiting the expression of at least one gene”, it is herein meant that the expression of said gene is reduced, i.e., the mRNA or protein levels of the target sequence is statistically lower than the mRNA level or protein level of the same target sequence in appropriate control bacteria which is exposed to control small RNAs targeted unrelated genes (e.g. fungal genes). In particular, reducing the mRNA polynucleotide level and/or the polypeptide level of the target gene in a bacteria according to the invention results in reaching less than 60%, less than 50%, less than 40%, less than 30%, less than 20%, less than 10%, or less than 5% of the mRNA polynucleotide level, or the level of the polypeptide encoded thereby, of the same target sequence in an appropriate control bacterium. Methods to assay the expression level of the RNA transcript, the expression level of the polypeptide encoded by the targeted gene, or the activity of said polynucleotide or polypeptide are well-known in the art.

In this aspect, any type of bacteria can be targeted. Pathogenic bacteria that infect animal (including human) hosts, or beneficial (e.g. symbiotic or commensal) bacteria that provide a beneficial effect for animal (including human) host can be targeted, as described above.

In one embodiment, this method is of particular interest for inhibiting or limiting the pathogenicity and growth of pathogenic bacteria in a sample. It is also useful for killing pathogenic bacterial cells in a sample.

In another embodiment, this method can also be used for promoting the replication of beneficial bacteria by inhibiting genes that negatively regulate directly or indirectly bacterial growth, as mentioned above.

In another embodiment, it is also possible to use this method for restoring the sensitivity of bacterial cells to an antibiotic compound by targeting a gene that is involved in the bacterial resistance to said antibiotic compound.

Phytotherapeutic Methods and Uses of the Invention

According to the invention, the one or more iRNAs of the invention is/are introduced into plant cells by using the standard methods mentioned above for expressing nucleic acids. A variety of methods for genetic transformation of eukaryotic cells are available in the art for many plant species. By way of non-limitative examples, one can perform projectile bombardment, virus-mediated transformation, Agrobacterium-mediated transformation, and the like. Electroporation is not included.

The term “introduced” in the context of inserting a nucleic acid into a cell, means “transfection” or “transformation” or “transduction” and includes reference to the incorporation of a nucleic acid into a eukaryotic cell where the nucleic acid may be stably incorporated into the genome of the cell (e.g., chromosome, plasmid), or transiently expressed (e.g., transient delivery of a gene construct via Agrobacterium tumefaciens).

The expression of the iRNAs of the invention in the host plant cell may be transient or stable. Stable expression refers in particular to the preparation of transgenic plants using conventional techniques.

Said iRNA will be processed into siRNA or miRNA duplexes by using the plant Dicer-like enzymes and other small RNA processing factors. Said small RNAs duplexes and/or mature small RNA guides (i.e. loaded into AGOs) are thereafter translocated in the extracellular medium, or at the surface of the plant cells, where they might encounter the bacterial cells.

As demonstrated in the examples below (examples 4 and 5 and FIGS. 4-6), the growth and the virulence of bacterial cells is decreased when placed in contact with the plant cells of the invention in conditions where the mature iRNAs of the invention are secreted.

In one aspect, the present invention relates to a method for treating target plants against a bacterial infection, said method comprising the step of introducing into at least one cell of said target plant a long dsRNA molecule targeting specifically a virulence bacterial gene or an essential bacterial gene or an antibacterial resistance gene.

This method is particularly useful to avoid the contamination of edible plants by humans and animals. By blocking the growth or survival of bacteria present onto plants by the treatment of the invention, this will avoid contamination of animals and humans by ingestion of the contaminated, infected plants.

This method is particularly useful for preventing plants to be infected by pathogenic animal bacteria, such as Shigella, Salmonella, Listeria, Brucella, Escherichia coli and, consequently, for preventing their consumers to get subsequently infected.

In another aspect, the present invention therefore relates to an RNA-based biocontrol method for treating plants against bacterial infection, said method comprising the step of delivering small RNAs, or a plant extract containing such small RNAs or a composition comprising these small RNAs (e.g. total RNAs extracted from plant cells or tissue stably or transiently expressing these small RNA entities, extracellular vesicles containing same, or nanoparticles coupled with said RNAs) on plant tissues prior to and/or after bacterial infection by a human or animal pathogenic bacterium, such as Actinomyces israelii, Bacillus anthracis, Bacillus cereus, Bacteroides fragilis, Bordetella pertussis, Borrelia sp. (burgdorferi, garinii, afzelii, recurrentis, crocidurae, duttonii, hermsii etc.), Brucella sp. (abortus, canis, melitensis, suis), Campylobacter jejuni, Chlamydia sp. (pneumoniae, trachomatis), Chlamydophila psittaci, Clostridium sp. (botulinum, difficile, perfringens, tetani), Corynebacterium diphtheriae, Ehrlichia sp. (canis, chaffeensis), Enterococcus (faecalis, faecium), Escherichia coli O157:H7, Francisella tularensis, Haemophilus influenza, Helicobacter pylori, Klebsiella pneumoniae, Legionella pneumophila, Leptospira sp., Listeria monocytogenes, Mycobacterium sp. (leprae, tuberculosis), Mycoplasma pneumoniae, Neisseria (gonorrhoeae, meningitidis), Pseudomonas aeruginosa, Porphyromonas gingivalis, Nocardia asteroides, Rickettsia rickettsii, Salmonella sp. (typhi, typhimurium), Shigella sp. (sonnei, dysenteriae), Staphylococcus (aureus, epidermidis, saprophyticus), Streptococcus sp. (agalactiae, mutans, pneumoniae, pyogenes, viridans), Tannerella forsythia, Treponema pallidum, Vibrio cholerae, or Yersinia pestis.

Preferably, said bacterium is a Gram-negative bacterium as explained above.

Preferably, said composition contains plant extracts obtained from plant cells that express or have been contacted with at least one long dsRNA that is specific to said at least one virulence or essential or antibiotic resistance bacterial gene of said pathogenic bacterium. More preferably, said composition contains extracellular vesicles recovered from said plant extracts, or extracellular free small RNAs secreted by said plant cells, or nanoparticle coupled small RNAs. Even more preferably, said composition is a liquid sprayable composition.

In this aspect, the bacterial cells are eventually contacted directly with small RNAs (i.e., siRNAs or miRNAs) that will be able to cross the bacterial double-membrane in case of Gram-negative bacteria and reach the cytosol of bacterial cells where the targeted gene(s) will be silenced in a sequence-specific manner, thereby resulting in the dampening of bacterial pathogenicity (see examples 5-7 & FIGS. 4-6 & FIGS. 9-10).

As used herein, the term “small RNAs” designates the small RNAs carrying the inhibiting activity of the iRNAs of the invention. Specifically, they are siRNAs or miRNAs (duplexes or simplexes) that share at least 80% sequence homology with at least one bacterial gene, preferably with at least one bacterial virulence or an essential gene, more preferably with at least one of the genes cited above. These small RNAs generally comprise no more than 40 base pairs. Preferably, they contain between 18 and 30 base pairs, more preferably between 18 and 25 base pairs. More preferably, said small RNAs specifically inhibit at least one of the bacterial essential or virulence gene defined above.

Preferably, these small RNAs are double-stranded siRNAs, as disclosed above.

Another aspect of the invention relates to the use of at least one iRNA or a vector containing this iRNA, as defined above, as a phytotherapeutic agent. Preferably, said iRNA or vector is used for treating a disease caused by a pathogenic bacterium in plants or for preventing a bacterial infection in plants.

In one embodiment, this phytotherapeutic iRNA is a short or long dsRNA, a siRNA duplex or a miRNA duplex, a siRNA simplex or a miRNA simplex, as defined above. In yet another embodiment, the iRNA targets bacterial genes and genes of other non-bacterial pathogens or parasites, as defined above, for concomitant prevention or treatment of diseases caused by bacterial pathogens and other pathogens/parasites in plants. All the embodiments proposed above for the iRNAs, the vectors, and the transformation methods are herewith encompassed and do not need to be repeated.

In the context of a phytosanitary technical problem induced by animal pathogenic bacteria, said small RNAs can be delivered to the plant tissues by various means (e.g., by spray). They can be embedded within microspheres, nanoparticles, liposomes or natural exosomes. Preferred formulations are disclosed below.

Transgenic Plants Producing the Small RNAs of the Invention

The plant cells transformed with the iRNAs of the invention and able to generate the small RNAs of the invention are hereafter designated as “plant cells of the invention” or “host cells of the invention”. They contain at least one iRNA (preferably a long RNA) containing at least one sequence targeting specifically a bacterial gene, e.g., a virulence or essential bacterial gene, or a DNA construct or vector as defined above.

Plants that have been stably transformed with a transgene encoding the long RNAs may be supplied as seed, reproductive material, propagation material, or cell culture material which does not actively express the long RNA but has the capability to do so.

If they are only used for producing the small RNAs of the invention, they can be called “producer plant cells”. If they will beneficiate from the antibacterial effect conferred by the produced small RNAs, they can also be called “target plants”. Both types of plants (the producers and the target ones) are recombinant cells expressing and producing the small RNAs of the invention. Producer plants can be target plants, as plants secreting the small RNAs of the invention can be used for ornamental/food purposes.

The term “plant” herein encompasses a plant cell, a plant tissue, a plant part, a whole plant, ancestors and progenies thereof. A plant part may be any part or organ of the plant and includes for example seed, fruit, stem, leaf, shoot, flower, anther, root, tuber and petiole. The term “plant” also encompasses suspension cultures, embryos, meristematic, regions, callus tissue, gametophytes, sporophytes, pollen and microspores. It refers to all plants including ferns and trees.

In another aspect, the present invention relates to an isolated plant cell or to a transgenic plant stably or transiently expressing at least one functional iRNA of the invention. It also relates to an isolated plant cell containing a DNA or viral vector of the invention. Said plant cell may be a genetically modified cell obtained by transformation with said DNA vector.

Examples of transformation processes are Agrobacterium-mediated transformation or shot-gun-mediated transformation.

All the embodiments proposed above for the plant cells, the iRNAs, the vectors, and the transformation methods are herewith encompassed and do not need to be repeated.

Methods to generate such transgenic plants are disclosed in the example part below. They contain the step of:

i) transforming a plant cell with a DNA vector expressing at least one functional interfering RNA of the invention, or

ii) infecting a plant cell with a plant virus, preferably an plant RNA virus, expressing at least one functional interfering RNA of the invention, for a sufficient time (typically 3-4 days for a tobacco plant) for the plant cell to stably or transiently express a significant amount of small RNAs.

By “significant amount”, it is herein meant an amount that has been shown to have an antibacterial effect in a test such as described above. This significant amount is preferably comprised between 10 and 30 ng/μl of total RNAs containing the effective small RNAs of the invention.

In particular, said transgenic plant is capable of host-induced gene silencing of a bacteria, and contains an expressible iRNA, capable of down-regulating or suppressing the expression of at least one gene of a bacteria, wherein the plant expresses mature small RNAs. As demonstrated by the inventors, said small RNAs are capable of propagating across or crossing the double membrane of the targeted bacteria.

In another aspect, the present invention relates to a target transgenic plant stably or transiently expressing the mature small RNAs of the invention. In one embodiment, said target transgenic plant contains the DNA vector of the invention. In one preferred embodiment, said target plant is Rice, Maize, Barley, Cottonseed, Cotton, Bean, Banana/Plantain, Sorghum, Pea, Sweet potatoes, Soybeans, Cabbage, Cassava, Potato, Tomato, Onion, Melon, Oats, Peanut, Sunflower, Palm oil, Rye, Citrus, Wheat, Peppers, Yams, Olives, Grapes, Taro, Tobacco, Sesame, Sugarcane, Sugarbeet, Pea and Coffee, Orange trees, Apple trees, Citrus trees, and Olive trees. All the embodiments proposed above for the iRNAs, the vectors, and the transformation techniques are herewith encompassed and do not need to be repeated.

In another aspect, the present invention relates to a transgenic plant stably or transiently expressing the iRNAs of the invention. In one embodiment, said transgenic producer plant contains the DNA vector of the invention. In one preferred embodiment, said producer plant is Tobacco (e.g. Nicotiana excelsior, Nicotiana excelsiana, Nicotiana benthamiana, Nicotiana tobaccum); Taro (Colocasia esculenta); Giger (Zingiber officinale), Arabidopsis (e.g. Arabidopsis thaliana); Tomato (e.g. Lycopersicon esculentum or Solanum lycopersicum); Potato (Solanum tuberosum); Rice (Oryza sativa); Maize (Zea mays); Barley (Hordeum vulgare); Wheat (e.g. Triticum aestivum, Triticum durum), Cottonseed, Cotton, Bean, Banana/Plantain, Sorghum, Pea, Sweet potatoes, Soybeans, Cabbage, Cassava, Onion, Melon, Oats, Peanut, Sunflower, Palm oil, Rye, Citrus, Wheat, Peppers, Yams, Olives, Grapes, Sesame, Sugarcane, Sugarbeet, Pea and Coffee, Orange trees, Apple trees, Citrus trees, Olive trees, etc. Preferred producer plants are Tobacco, Taro and Giger. In a preferred embodiment, said producer plant is a Tobacco plant such as Nicotiana benthamiana, Nicotiana excelsior or Nicotiana excelsiana.

Probiotic Methods and Uses of the Invention

In another aspect, this method can also be used for promoting the replication of beneficial (commensal) bacteria by inhibiting genes that negatively regulate directly or indirectly bacterial growth, as mentioned above.

The present invention therefore relates to a small RNA having a length comprised between 15 and 30 base pairs and inhibiting specifically the expression of at least one bacterial gene, for use for promoting beneficial effects of beneficial commensal or symbiotic bacteria in a subject in need thereof, wherein said small RNA is administered orally, topically or systemically to said subject.

Preferably, said beneficial commensal or symbiotic bacteria are chosen in the group consisting of: Actinomyces naeslundii, Veillonella dispar, Faecalibacterium prausnitzii, Enterobacteriaceae, Bacteroides thetaiotaomicron, Escherichia coli K12, Bifidobacterium sp. (longum, bifidum, adolescentis, dentium, breve, themophilum), Eggerthella lenta, Bacteroides sp. (xylanisolvens, thetaiotaomicron, fragilis, vulgatus, salanitronis), Parabacteroides distasonis, Faecalibacterium prausnitzii, Ruminococcus sp. (bromii, champanellensis, SR1/5), Streptococcus (parasanguinis, salivarius, thermophilus, suis, pyogenes, anginosus), Lactococcus (lactis, garvieae), Enterococcus (faecium, faecalis, casseliflavus, durans, hirae, Melissococcus plutonius, Tetragenococcus halophilus, Lactobacillus sp. (casei, ruminis, delbrueckii, buchneri, reuteri, fermentum, pentosus, amylovorus, salivarius), Pediococcus (pentosaceus, claussenii), Leuconostoc (mesenteroides, lactis, carnosum, gelidum, citreum), Weissella (thailandensis, koreensis), Oenococcus oeni, Paenibacillus sp. (terrae, polymyxa, mucilaginosus, Y412MC10), Thermobacillus composti, Brevibacillus brevis, Bacillus (amyloliquefaciens, subtilis, licheniformis, atrophaeus, weihenstephanensis, cereus, thuringiensis, coagulans, megaterium, selenitireducens), Geobacillus thermodenitrificans, Lysinibacillus sphaericus, Halobacillus halophilus, Listeria sp., Streptomyces sp., Eubacterium (rectale, eligens, siraeum), Clostridium saccharolyticum, and butyrate-producing bacterium (SS3/4 and SSC 2).

Restoring Antibiotic Sensitivity with iRNAs of the Invention

In another aspect, the inventors propose to use this method for restoring the sensitivity of bacterial cells to an antibiotic compound by targeting a gene that is involved in the bacterial resistance to said antibiotic compound.

By “antibiotic compound”, it is meant a compound that is used or proposed for killing bacteria. Classical antibiotic compounds that are used in the therapeutic field are for example copper-based bactericides or secondary metabolites derived from macro- and micro-organisms. These include but are not restricted to Aminoglycosides, Carbapenems, Ceftazidime (3rd generation), Cefepime (4th generation), Ceftobiprole (5th generation), Ceftolozane tazobactam, Fluoroquinolones, Piperacillin tazobactam, Ticarcillin clavulanic acid, Amikacin, Gentamicin, Kanamycin, Neomycin, Netilmicin, Tobramycin, Paromomycin, Streptomycin, Spectinomycin, Geldenamycin, herbimycin, Rifaximin, Ertapenem, Doripenem, Imipenem, Meropenem, Cefadroxil, Cefazolin, Cephradine, Cephapirin, Cephalothin, Cefalexin, Cefaclor, Cefoxitin, Cefotetan, Cefamandole, Cefinetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cephalosporins, Cefepime, Cephalosporins, Ceftaroline fosamil, Ceftobiprole, Glycopeptides, Teicoplanin, Vancomycin, Telavancin, Dalbavancin, Oritavancin, Lincosamides(Bs), Clindamycin, Lincomycin, Lipopeptide, Daptomycin, Macrolides(Bs), Azithromycin, Clarithromycin, Erythromycin, Roxithromycin, Telithromycin, Spiramycin, Fidaxomicin, Monobactams, Aztreonam, Nitrofurans, Furazolidone, Nitrofurantoin(Bs), Oxazolidinones (Bs), Linezolid, Posizolid, Radezolid, Torezolid, Penicillins, Amoxicillin, Ampicillin, Azlocillin, Dicloxacillin, Flucloxacillin, Mezlocillin, Methicillin, Nafcillin, Oxacillin, Penicillin G, Penicillin, Piperacillin, Temocillin, Ticarcillin, Penicillin combinations, Amoxicillin clavulanate, Ampicillin sulbactam, Piperacillin tazobactam, Ticarcillin clavulanate, Polypeptides, Bacitracin, Colistin, Polymyxin B, Quinolones Fluoroquinolones, Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, Temafloxacin, Sulfonamides(Bs), Mafenide, Sulfacetamide, Sulfadiazine, Silver sulfadiazine, Sulfadimethoxine, Sulfamethizole, Sulfamethoxazole, Sulfanilimide (archaic), Sulfasalazine, Sulfisoxazole, Trimethoprim-Sulfamethoxazole (Co-trimoxazole) (TMP-SMX), Sulfonamidochrysoidine (archaic), Tetracyclines(Bs), Demeclocycline, Doxycycline, Metacycline, Minocycline, Oxytetracycline, Tetracycline, Clofazimine, Dapsone, Capreomycin, Cycloserine, Ethambutol(Bs), Ethionamide, Isoniazid, Pyrazinamide, Rifampicin, Rifabutin, Rifapentine, Streptomycin, Arsphenamine, Chloramphenicol(Bs), Fosfomycin, Fusidic acid, Metronidazole, Mupirocin, Platensimycin, Quinupristin Dalfopristin, Thiamphenicol, Tigecycline(Bs), Tinidazole, Trimethoprim(Bs) etc.

Preferably, the target bacteria are then chosen in the group consisting of:

Actinomyces israelii, Bacillus anthracis, Bacillus cereus, Bacteroides fragilis, Bordetella pertussis, Borrelia sp. (burgdorferi, garinii, afzelii, recurrentis, crocidurae, duttonii, hermsii etc.), Brucella sp. (abortus, canis, melitensis, suis), Campylobacter jejuni, Chlamydia sp. (pneumoniae, trachomatis), Chlamydophila psittaci, Clostridium sp. (botulinum, difficile, perfringens, tetani), Corynebacterium diphtheriae, Ehrlichia sp. (canis, chaffeensis), Enterococcus (faecalis, faecium), Escherichia coli O157:H7, Francisella tularensis, Haemophilus influenza, Helicobacter pylori, Klebsiella pneumoniae, Legionella pneumophila, Leptospira sp., Listeria monocytogenes, Mycobacterium sp. (leprae, tuberculosis), Mycoplasma pneumoniae, Neisseria (gonorrhoeae, meningitidis), Pseudomonas aeruginosa, Porphyromonas gingivalis, Nocardia asteroides, Rickettsia rickettsii, Salmonella sp. (typhi, typhimurium), Shigella sp. (sonnei, dysenteriae), Staphylococcus (aureus, epidermidis, saprophyticus), Streptococcus sp. (agalactiae, mutans, pneumoniae, pyogenes, viridans), Tannerella forsythia, Treponema pallidum, Vibrio cholerae, Yersinia pestis etc.

The amount of plant small RNAs to be used typically depends on the number of bacteria and on the type of bacteria that are targeted. This amount can be comprised between 10 and 30 ng/μl of total RNAs containing the effective small RNAs.

Therapeutic Methods of the Invention

In another aspect, the present invention relates to an RNA-based therapeutics method for treating animals against bacterial infection, said method comprising the step of delivering small RNAs (de novo synthetized or purified from plant extracts), or a plant extract containing such small RNAs or a composition comprising these small RNAs (e.g. total RNAs extracted from plant cells or tissue stably or transiently expressing these small RNA entities, extracellular vesicles from said plant cells, apoplastic fluid from said plant cells, extracellular free small RNAs from said plants, or nanoparticles coupled to said small RNAs) on (or within) animal tissues, prior to and/or after bacterial infection.

Preferably, said animal is of the genus: Homo sapiens, Canis lupus, Felis catus, Equus caballus, Bos taurus, Ovis aries, Capra hircus, Sus scrofa, Gallus gallus, Meleagris gallopavo, Anser anser, Anas platyrhynchos, Oryctolagus cuniculus. It can be a healthy animal hosting beneficial bacteria, or a sick animal already infected by a pathogenic bacteria.

More preferably, said animal is a human being.

It can be a healthy human hosting beneficial bacteria, or a sick human already infected by a pathogenic bacteria.

In this aspect, the bacterial cells are contacted directly with small RNAs (i.e., siRNAs or miRNAs) that will be able to cross the bacterial double-membrane in the case of Gram-negative bacteria and reach the cytosol of bacterial cells where the targeted gene(s) will be silenced in a sequence-specific manner, thereby resulting in the dampening of bacterial pathogenicity. For instance, an amount of 5 ng/μL of small RNAs has been shown to be able to significantly reduce bacterial growth in vitro.

As used herein, the term “small RNAs” designates the small RNAs carrying the inhibiting activity of the iRNAs of the invention. Specifically, they are siRNAs or miRNAs (duplexes or simplexes) that share at least 80% sequence homology with at least one bacterial gene, preferably with at least one bacterial virulence or an essential gene, more preferably with at least one of the genes cited above. These small RNAs generally comprise no more than 40 base pairs. Preferably, they contain between 18 and 25 base pairs. More preferably, said small RNAs specifically inhibit at least one of the bacterial essential or virulence gene defined above.

The treatment method of the invention includes oral, topic and systemic administration of the small RNAs of the invention. Nasal and intravenous administration can also be contemplated.

Another aspect of the invention relates to the use of at least one small RNA as defined above, as a cosmetic or therapeutic agent. Preferably, said small RNA is used for treating a disease caused by a pathogenic bacterium or for preventing a bacterial infection.

In one embodiment, this small RNA targets bacterial genes and genes of other non-bacterial pathogens or parasites, as defined above, for concomitant prevention or treatment of diseases caused by bacterial pathogens and other pathogens/parasites. All the embodiments proposed above for the iRNAs, the vectors, and the transformation methods are herewith encompassed and do not need to be repeated.

Another aspect of the invention relates to the use of at least one small RNA as defined above, or therapeutic compositions containing same (as disclosed below), for preparing a medicament intended to treat a disease caused by a pathogenic bacterium, or to prevent a bacterial infection.

In one embodiment, when the small RNA targets bacterial genes and genes of other non-bacterial pathogens or parasites, as defined above, said medicament can concomitantly treat or prevent diseases caused by bacterial pathogens and other pathogens/parasites.

The present invention also encompasses therapeutic or cosmetic methods involving the use of an effective amount of the small RNAs defined above.

By “effective amount”, it is herein meant an amount that has been shown to have an antibacterial effect in a test such as described in the examples below. This amount is preferably comprised between 10 and 30 ng/μl of total RNAs containing the effective small RNAs of the invention. When synthetic RNAs are used, this amount is more preferably comprised between 0.1 and 10 ng/μl of small RNAs of the invention.

In these therapeutic/cosmetic methods, the small RNAs of the invention can be formulated in a liquid solution, in a spray, in a pill, in a cream, or as a powder.

The small RNAs of the invention can be also advantageously coupled/associated/fused to nanoparticles that are known to convey small RNAs efficiently in vivo. Any nanoparticle-mediated systemic delivery of siRNA can be used for treating animals (including humans), as soon as its toxicity is controlled or absent. A number of systems has been proposed, and used in clinical trials, as described in (44).

To deliver the small RNAs of the invention systemically into animals, it is also possible to couple them to nanoparticle systems, as disclosed in (44). These can be silicon- or metal- or carbon-based nanoparticles having a size comprised between 50 nm and 500 nm, dendrimers, polymers, cyclodextrins, lipid-based nanoparticles, liposomes, hydrogels, or semiconductor nanocrystals, as disclosed in Table 3 of (44). As explained in this review, all of these delivery systems have been proved to efficiently transfer siRNAs in vivo.

Lipidic nanoparticles are herein preferred, as they have been recently approved in human therapy by the FDA.

Therapeutic Compositions of the Invention

In the context of public health, the small RNAs of the invention or the compositions comprising same can be delivered to the animal tissues by various means (orally, topically, systemically, etc.). In a particular embodiment, they can be embedded within microspheres, liposomes or natural EVs, in order to be protected from deleterious agents. They can also be coupled to nanoparticles. They can also be incorporated as naked iRNA molecules directly in the compositions.

In another aspect, the present invention therefore relates to therapeutic compositions containing, as active principle, the small RNAs of the invention. In particular, it relates to therapeutic compositions containing a significant amount of siRNAs or miRNAs inhibiting the expression of at least one bacterial gene, preferably inhibiting the expression of one essential or of one virulence bacterial gene or of one antibiotic resistance bacterial gene.

The small RNAs contained in the therapeutic compositions of the invention may be synthetic or may be obtained from plants, plant tissues or plant cells stably or transiently expressing said small RNAs, as thoroughly disclosed above.

In particular, plants, plant tissues or plant cells stably or transiently transformed by a DNA vector of the invention or infected by a viral vector of the invention will produce small RNAs.

A therapeutic composition of the invention may thus comprise either total RNAs of plants, plant tissues or plant cells stably or transiently expressing the small RNAs of interest, or a purified small RNA fraction of the total RNAs, or de novo synthesized small RNAs.

By “significant amount”, it is herein meant an amount that has been shown to have an antibacterial effect in a test such as described in the examples below. This amount is preferably comprised between 10 and 30 ng/μl of total RNAs containing the effective small RNAs of the invention. When purified or synthetic RNAs are used, this amount is more preferably comprised between 0.1 and 10 ng/μl of small RNAs of the invention.

The silencing element of the invention can be added in an external composition such as a spray or a cream or a pill.

Preferably, it is embedded within microspheres, liposomes or natural exosomes, in order to be protected from deleterious agents or coupled to nanoparticles, as disclosed below.

The therapeutic compositions of the invention can also comprise cells (such as crude plant cell extracts), containing the active antibacterial small RNAs. Compositions comprising a mixture of cell extracts, some cell extracts from plant cells expressing at least one iRNA of the invention, are also encompassed. In other embodiments, the therapeutic compositions of the invention do not contain any cell.

In one embodiment, the composition of the invention is applied externally to an animal tissue (i.e., by spraying the composition or by applying a lotion, a gel, a cream on said tissue), to protect the individual from bacterial infection.

The composition of the invention can be applied on any tissue that can be in contact with bacteria. This tissue is preferably chosen in the group consisting of: skin, hair, mucosa, nail, gut, wound, eyes, etc.

The therapeutic compositions of the invention can be formulated in a suitable and/or environmentally acceptable carrier. Such carriers can be any material that the individual to be treated can tolerate. Furthermore, the carrier must be such that the composition remains effective at controlling the bacteria infection. Examples of such carriers include water, saline, Ringer's solution, dextrose or other sugar solutions, Hank's solution, and other aqueous physiologically balanced salt solutions, phosphate buffer, bicarbonate buffer and Tris buffer.

These compositions may furthermore contain a surface-active agent, an inert carrier, a preservative, a humectant, a feeding stimulant, an attractant, an encapsulating agent, a binder, an emulsifier, a dye, a UV protectant, a buffer, a flow agent, etc. It can also contain other active principles, such as insecticides, fungicides, bactericides, nematicides, molluscicides or acaracides. These agents can be combined with carriers, surfactants or adjuvants customarily employed in the art of formulation or other components to facilitate product handling and application. Suitable carriers and adjuvants can be solid or liquid and correspond to the substances ordinarily employed in formulation technology, e.g., natural or regenerated mineral substances, solvents, dispersants, wetting agents, tackifiers, or binders.

In a preferred embodiment, the composition of the invention is a liquid sprayable composition. It can then easily be applied on tissues or on clothes or on any material that can be in contact with pathogenic bacteria, as a preventing measure or as a treatment to get rid of the bacteria infection. It can also be easily inhaled for preventing nasally acquired infections.

In another preferred embodiment, the composition of the invention is formulated as a pill that can be easily swallowed by animal and humans.

In another preferred embodiment, the composition of the invention is formulated as a cream, lotion, or gel, that can conveniently be applied on skin or hair tissues.

More generally, it is possible to add the small RNAs of the invention (or the EVs comprising same) in cosmetic products in order to prevent bacterial infection to occur.

In another preferred embodiment, the composition of the invention is formulated in a pill, for example in a slow release pill, that can conveniently be swallowed to act on gut mucosa or other internal tissues.

Extracellular Vesicles Comprising the Small RNAs of the Invention

In a preferred embodiment, the small RNAs of the invention or their precursors are contained within natural Extracellular Vesicles (EVs) or in artificial vesicles in which they will be protected from the action of RNases. As a matter of fact, these vesicles are not toxic for the treated animal (especially in human) and can protect efficiently the small RNAs contained herein.

A number of studies have now been published, highlighting the important protective role of EVs in the delivery of small RNAs to plant eukaryotic pathogens (17, 19).

The present inventors herein show that the delivery of small RNAs from plant to bacteria also occurred, at least partially, through EVs secreted by the transgenic plants (FIG. 9B).

The compositions of the invention therefore preferably contain EVs that have been secreted by the transgenic plants of the invention and that contain the mature small RNAs of the invention.

EVs have heterogeneous size diameters (45, 46). They contain cytosolic and membrane proteins derived from the parental cells (45-48). They also contain functional mRNAs, long non-coding RNAs, miRNA precursors and mature miRNAs and siRNAs (17, 19, 49, 50).

Purification of EVs can be performed by various methods, the most common and most preferred of which being differential ultracentrifugation (45, 46).

More particularly, it is possible to obtain EVs from plant cells by filtration and differential centrifugation steps as previously described (45, 46). Briefly, leaves are vacuum infiltrated with classical buffers used to collect apoplastic wash fluid (e.g. pH 6 MES buffer) and further centrifuged at low speed (46). The apoplastic wash fluid is further collected, filtered and centrifuged successfully as recently described (46). A population of plant EVs, in a size range of approximately 50 to 300 nm in Arabidopsis (with a median at 150 nm) can be recovered at a centrifugation speed of 40,000 g from apoplastic fluid (46). Smaller EVs, in a size range of approximately 10-20 nm in Arabidopsis, can also be recovered by exerting differential ultracentrifugation from apoplastic fluid at centrifugation speed at 40,000 g followed by another one at 100,000 g on the supernatant obtained in the previous step (46). Plant EVs can be also concentrated using dedicated columns (e.g. Amicon Ultra-15 Centrifugal Filters Ultracel 30K), and resuspended in dedicated buffer so that they can be subsequently used for incubation with bacterial cells (in vitro assay) or exogenously applied on plant surface (in planta assay) prior or after bacterial infections.

Apoplastic Fluids Containing EV-Free Small RNAs

The composition of the invention may also contain apoplastic EV-free small RNAs secreted by the transgenic plants of the invention and that are not associated with proteins. These small RNA species are referred to here as Extracellular Free Small RNAs or “efsRNAs”.

These small RNA species can be obtained by recovering the supernatant from either a differential ultracentrifugation of apoplastic fluid involving a 100,000 g centrifugation speed or the supernatant from a differential ultracentrifugation of apoplastic fluid involving a 40,000 g followed by a 100,000 g centrifugation speed.

The resulting supernatant can be mixed in dedicated buffer or used directly for incubation with bacterial cells (in vitro assay) or exogenously applied on plant surface (in planta assay) prior or after bacterial infections.

These EV fractions are advantageously kept or supplied in frozen form or in freeze-dried or lyophilized powder form, under which they maintain their high functionality.

Combination Products of the Invention

The compositions of the invention may be applied simultaneously or in succession with other compounds.

In particular, the compositions of the invention may be applied with antibiotic compounds, especially when the iRNAs they carry target an antibiotic resistance gene.

In this case, the composition of the invention may be supplied as a “kit of parts”, comprising the silencing element of the invention (the small RNAs defined above) and the corresponding bactericidal compound in a separate container.

In a further aspect, the present invention therefore relates to a pharmaceutical kit containing:

a) a small interfering RNA (siRNA) having a length comprised between 15 and 30 base pairs and inhibiting specifically an antibiotic resistance gene, or a therapeutic composition containing same, as disclosed above, and

b) an antibiotic compound.

The present invention also targets the use of such pharmaceutical kit for treating and/or preventing a bacterial infection in a subject in need thereof and treating methods using same.

In another aspect, the present invention relates to a combination product comprising:

a) a small interfering RNA (si RNA or miRNA) having a length comprised between 15 and 30 base pairs and inhibiting specifically an antibiotic resistance gene, or a therapeutic composition comprising same, as disclosed above, and

b) an antibiotic compound,

for use for simultaneous, separated or staggered use for preventing and/or treating a bacterial infection in a subject in need thereof.

In a preferred embodiment, said siRNA or miRNA is administered before said antibiotic compound, preferably one week before, more preferably one day before.

In these kits and products, said antibiotic resistance gene is preferably chosen from: VIM-1, VIM-2, VIM-3, VIM-5, CasE, OXA-28, OXA-14, OXA-19, OXA-145, PER-1, TEM-116, and GES-9.

In these kits and products, said antibiotic compound is preferably chosen from: Aminoglycosides, Carbapenems, Ceftazidime (3rd generation), Cefepime (4th generation), Ceftobiprole (5th generation), Ceftolozane tazobactam, Fluoroquinolones, Piperacillin tazobactam, Ticarcillin clavulanic acid, Amikacin, Gentamicin, Kanamycin, Neomycin, Netilmicin, Tobramycin, Paromomycin, Streptomycin, Spectinomycin, Geldenamycin, herbimycin, Rifaximin, Ertapenem, Doripenem, Imipenem, Meropenem, Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cephalosporins, Cefepime, Cephalosporins, Ceftaroline fosamil, Ceftobiprole, Glycopeptides, Teicoplanin, Vancomycin, Telavancin, Dalbavancin, Oritavancin, Lincosamides(Bs), Clindamycin, Lincomycin, Lipopeptide, Daptomycin, Macrolides(Bs), Azithromycin, Clarithromycin, Erythromycin, Roxithromycin, Telithromycin, Spiramycin, Fidaxomicin, Monobactams, Aztreonam, Nitrofurans, Furazolidone, Nitrofurantoin(Bs), Oxazolidinones(Bs), Linezolid, Posizolid, Radezolid, Torezolid, Penicillins, Amoxicillin, Ampicillin, Azlocillin, Dicloxacillin, Flucloxacillin, Mezlocillin, Methicillin, Nafcillin, Oxacillin, Penicillin G, Penicillin, Piperacillin, Temocillin, Ticarcillin, Penicillin combinations, Amoxicillin/clavulanate, Ampicillin/sulbactam, Piperacillin/tazobactam, Ticarcillin clavulanate, Polypeptides, Bacitracin, Colistin, Polymyxin B, Quinolones Fluoroquinolones, Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, Temafloxacin, Sulfonamides(Bs), Mafenide, Sulfacetamide, Sulfadiazine, Silver sulfadiazine, Sulfadimethoxine, Sulfamethizole, Sulfamethoxazole, Sulfanilimide (archaic), Sulfasalazine, Sulfisoxazole, Trimethoprim-Sulfamethoxazole (Co-trimoxazole) (TMP-SMX), Sulfonamidochrysoidine (archaic), Tetracyclines(Bs), Demeclocycline, Doxycycline, Metacycline, Minocycline, Oxytetracycline, Tetracycline, Clofazimine, Dapsone, Capreomycin, Cycloserine, Ethambutol(Bs), Ethionamide, Isoniazid, Pyrazinamide, Rifampicin, Rifabutin, Rifapentine, Streptomycin, Arsphenamine, Chloramphenicol(Bs), Fosfomycin, Fusidic acid, Metronidazole, Mupirocin, Platensimycin, Quinupristin Dalfopristin, Thiamphenicol, Tigecycline(Bs), Tinidazole, Trimethoprim(Bs) etc.

Preferably, said subject is an animal of the genus: Homo sapiens, Canis lupus, Felis catus, Equus caballus, Bos taurus, Ovis aries, Capra hircus, Sus scrofa, Gallus gallus, Meleagris gallopavo, Anser anser, Anas platyrhynchos, Oryctolagus cuniculus. It can be a healthy animal hosting beneficial bacteria, or a sick animal already infected by pathogenic bacterium preferably a Gram-negative bacterium.

More preferably, said animal is a human being.

It can be a healthy human hosting beneficial bacteria, or a sick human already infected by a pathogenic bacterium, preferably a Gram-negative bacterium.

Screening System of the Invention

In one specific embodiment, the methods of the invention can be also used as tools for experimental research, particularly in the field of functional genomics. Down-regulating bacterial genes with small RNAs can be indeed used to study gene function, in an analogous approach to what has been described in the art for the nematode worm C. elegans and also Drosophila melanogaster. This approach is particularly useful against bacteria that cannot be cultured in vitro.

Assays based on targeted down- or up-regulation of specific bacterial genes, leading to a measurable phenotype, provide new tools for identifying anti-bacterial agents.

The Inventors have indeed further developed assays to identify candidate small RNAs having antibacterial activity prior to in planta assays (the latters are more time-consuming for the experimentalist). As demonstrated in FIG. 8C/D, this system can rely on the transient expression of small RNAs using well-established Agrobacterium-mediated transient transformation of tobacco leaves. It can be followed by the incubation of corresponding candidate siRNAs with bacterial cells (in the presence of plant tissues/extracts in the proximity of bacterial cells or in in vitro media such as minimal media mimicking the host environment, which are known to trigger the expression of virulence factors).

In another aspect, the present invention relates to in vitro screening methods allowing the rapid, reliable and cost-effective identification of functional iRNAs having an antibacterial activity, said method comprising the steps of:

a) expressing in plant cells at least one long dsRNA, whose cognate siRNAs inhibit at least one bacterial gene,

b) contacting said plant cells with a lysis buffer,

c) incubating said plant cell lysates or RNA extracts thereof, with bacterial cells, and

d) assessing the viability, growth, metabolic activity, of said bacterial cells.

Step d) can be performed by assessing the expression/activity of reporters (e.g. reporters of bacterial replication, of general stress response, cell division etc.), metabolic activity (e.g. exogenous delivery of the fluorescent marker resazurin that is commonly used to monitor bacterial respiratory activity, redox balance indicator and viability), growth (e.g. expression of fluorescent reporter driven by a constitutive promoter that is either chromosomally integrated or encoded from a plasmid), the expression of the gene that is targeted by small RNAs (e.g. RT-qPCR analysis, Western Blot analyses, expression of a reporter gene fused to the targeted gene or the region of the gene that is targeted by small RNAs) of said bacterial cells.

Stable or transient expression of the antibacterial small RNAs can be used, as disclosed above. For transient expression, said plant cells are preferably tobacco leaves cells that can be easily and efficiently transformed with exogenous constructs through Agrobacterium-mediated transient transformation. All the embodiments proposed above for the production of iRNAs, the vectors, the host cells, the targeted genes, the bacteria and the transformation technics are herewith encompassed and do not need to be repeated.

It is also possible to use the apoplastic fluid of the plant cells, containing the secreted molecules and EVs (in association with the effective small RNAs), to contact the bacterial cells in step b). The apoplastic fluid can be recovered by any conventional means such as vacuum infiltration and centrifugation that are commonly used by those skilled in the art. Concentration of EVs can be also further performed using dedicated columns (e.g. Amicon Ultra-15 Centrifugal Filters Ultracel 30K), according to manufacturer instructions.

The method of the invention may contain a final step e) comparing the viability, growth, metabolic or gene reporter activities of bacterial cells incubated with the said apoplastic fluid or said small RNAs with the ones of the same bacterial cells but in the absence of the apoplastic wash fluid or said small RNAs or, preferentially, in the presence of apoplastic wash fluid—from plants expressing control small RNAs—or control small RNAs targeting unrelated genes such as the fungal genes CYP51 from F. graminearum as used in the present invention.

It is anticipated that this screening system will be exploited in the future to select, and eventually produce, efficient antibacterial small RNAs, that can be incorporated into therapeutic compositions or agents.

The present inventors also developed systems that are not related to plant production of small RNAs, by using rapid in vitro synthesis of double-stranded small RNAs targeting bacterial genes (FIG. 10). As proof-of-concept experiments, the inventors have demonstrated that in vitro synthesized anti-Cfa6 and anti-HrpL siRNAs triggered bacterial gene silencing as well as suppression of Pto DC3000-induced stomatal reopening to the same extent as total RNAs derived from IR-CFA6 HRPL transgenic plants (FIG. 10B/C, FIG. 6A). Furthermore, they have shown that in vitro-synthesized siRNAs directed against Pto DC3000 FusA and GyrB genes possess strong bactericidal effects, thereby preventing the growth of Pto DC3000 in in vitro conditions (FIG. 10D/E). In addition, using the same methodology, they showed that in vitro-synthesized siRNAs directed against P. aeruginosa SecE, GyrB and DnaN genes triggered also a reduced growth of P. aeruginosa in in vitro conditions (FIG. 12).

They therefore propose an in vitro method to identify candidate genes that affect the proliferation of human pathogenic bacterial cells, said method comprising the steps of:

a) generating small RNAs inhibiting the expression of at least one bacterial gene,

b) incubating said small RNAs with bacterial cells, and

c) assessing the viability, growth, metabolic activity of said bacterial cells, as disclosed above.

In particular, the present invention relates to an in vitro method to identify candidate genes involved in bacterial antibiotic resistance, said method comprising the steps of:

a) incubating bacterial cells with a small RNA having a length comprised between 15 and 30 base pairs and inhibiting specifically at least one bacterial gene,

b) incubating said small RNA treated bacterial cells with an antibiotic compound,

c) assessing the viability, growth, metabolic activity, of said small RNA treated bacterial cells in the presence of the antibiotic compound, and compare same with the viability, growth, metabolic activity, of said small RNA treated bacterial cells in the absence of the antibiotic compound.

In a preferred embodiment, said candidate gene is involved in bacterial antibiotic resistance if the viability, growth, metabolic activity, of said small RNA treated bacterial cells in the presence of the antibiotic compound is lower than the viability, growth, metabolic activity, of said small RNA treated bacterial cells in the absence of the antibiotic compound.

In addition to the above arrangements, the invention also comprises other arrangements, which will emerge from the description that follows, which refers to exemplary embodiments of the subject of the present invention, with reference to the attached drawings and Table of sequences in which:

TABLE I
Sequence details on the tools used in the examples

| SEQ ID NO: | Name/details |
|---|---|
| 1 | Sequence of the first arm of the CFA6/HRPL dsRNA used |
| | to concomitantly target HrpL and Cfa6 genes of Pto DC3000 |
| 2 | Sequence of the CHSA intron used to generate all |
| | the inverted repeat from the present invention |
| 3 | Sequence of the second arm of the CFA6/HRPL dsRNA used |
| | to concomitantly target HrpL and Cfa6 genes of Pto DC3000 |
| 4 | Sequence of the first arm of the CFA6-A dsRNA |
| | used to target the Cfa6 gene of Pto DC3000 |
| 5 | Sequence of the second arm of the CFA6-A dsRNA |
| | used to target the Cfa6 gene of Pto DC3000 |
| 6 | Sequence of the first arm of the CFA6-B dsRNA |
| | used to target the Cfa6 gene of Pto DC3000 |
| 7 | Sequence of the second arm of the CFA6-B dsRNA |
| | used to target the Cfa6 gene of Pto DC3000 |
| 8 | Sequence of the first arm of the HRPL-A dsRNA |
| | used to target the HrpL gene of Pto DC3000 |
| 9 | Sequence of the second arm of the HRPL-A dsRNA |
| | used to target the HrpL gene of Pto DC3000 |
| 10 | Sequence of the first arm of the HRPL-B dsRNA |
| | used to target the HrpL gene of Pto DC3000 |
| 11 | Sequence of the second arm of the HRPL-B dsRNA |
| | used to target the HrpL gene of Pto DC3000 |
| 12 | Sequence of the first arm of the HRCC dsRNA |
| | used to target the HrcC gene of Pto DC3000 |
| 13 | Sequence of the second arm of the HRCC dsRNA |
| | used to target the HrcC gene of Pto DC3000 |
| 14 | Sequence of the first arm of the AvrPto/AvrPtoB dsRNA used |
| | to target the AvrPto and AvrPtoB genes of Pto DC3000 |
| 15 | Sequence of the second arm of the AvrPto/AvrPtoB dsRNA used |
| | to target the AvrPto and AvrPtoB genes of Pto DC3000 |
| 16 | Sequence of the first arm of the CYP51 dsRNA used to target the FgCYP51A, FgCYP51B |
| | and FgCYP51C genes of Fusarium grammearum |
| 17 | Sequence of the second arm of the CYP51 dsRNA used to target the FgCYP51A, |
| | FgCYP51B and FgCYP51C genes of Fusarium graminearum |
| 18 | Sequence of the first arm of the HRPG/HRPB/HRCC dsRNA used to target concomitantly |
| | the HrpG, HrpB and HrcC genes of Ralstonia species |
| 19 | Sequence of the second arm of the HRPG/HRPB/HRCC dsRNA used to target |
| | concomitantly the HrpG, HrpB and HrcC genes of Ralstonia species |
| 20 | Sequence of the first arm of the HRPB/HRCC/TssB/XpsR dsRNA used to target |
| | concomitantly the HrpB, HrcC, XpsR and TssB genes ofRalstonia species |
| 21 | Sequence of the second arm of the HRPB/HRCC/TssB/XpsR dsRNA used to target |
| | concomitantly the HrpB, HrcC, XpsR and TssB genes of Ralstonia species |
| 22 | Sequence of the first arm of the HRPG/HRPXRsmA dsRNA used to target concomitantly |
| | the HrpG, HrpX and RsmA genes of Xanthomonas campestris pv. campestris |
| 23 | Sequence of the second arm of the HRPG/HRPX/RsmA dsRNA used to target |
| | concomitantly the HrpG, HrpX and RsmA genes of Xanthomonas campestris pv. |
| | campestris |
| 24 | Sequence of the first arm of the RpoB/RpoC/FusA dsRNA used to target concomitantly the |
| | RpoB, RpoC and FusA genes of Pto DC3000 and Pseudomonas svnngae CC440 |
| 25 | Sequence of the second arm of the RpoB/RpoC/FusA dsRNA used to target concomitantly |
| | the RpoB, RpoC and FusA genes of Pto DC3000 and Pseudomonas svnngae CC440 |
| 26 | Sequence of the first arm of the SecE/RpoA/RplQ dsRNA used to target concomitantly the |
| | SecE, RpoA and RplQ genes of Pto DC3000 and Pseudomonas svringae CC440 |
| 27 | Sequence of the second arm of the SecE/RpoA/RplQ dsRNA used to target concomitantly |
| | the SecE, RpoA and RplQ genes of Pto DC3000 and Pseudomonas syringae CC440 |
| 28 | Sequence of the first arm of the NadHb/NadHd/NadHe dsRNA used to target |
| | concomitantly the NadHb, NadHd and NadHe genes of Xanthomonas species |
| 29 | Sequence of the second arm of the NadHb/NadHd/NadHe dsRNA used to target |
| | concomitantly the NadHb, NadHd and NadHe genes of Xanthomonas species |
| 30 | Sequence of the first arm of the DnaA/DnaE1/DnaE2 dsRNA used to target concomitantly |
| | the DnaA, DnaE1 and DnaE2 genes of Xanthomonas species |
| 31 | Sequence of the second arm of the DnaA/DnaE1/DnaE2 dsRNA used to target |
| | concomitantly the DnaA, DnaE1 and DnaE2 genes of Xanthomonas species |
| 32 | GFP reporter sequence contained in the GFPpPNpt plasmid |
| 33 | Primer sequence of Cfa6-Forward used for LMW Northern Blot |
| 34 | Primer sequence of Cfa6-Reverse used for LMW Northern Blot |
| 35 | Primer sequence of HrpL-Forward used for LMW Northern Blot |
| 36 | Primer sequence of HrpL-Reverse used for LMW Northern Blot |
| 37 | Primer sequence of miR159 probe used for LMW Northern Blot |
| 38 | Primer sequence of GyM-Fwd used for RT-qPCR |
| 39 | Primer sequence of GyrA-Rev used for RT-qPCR |
| 40 | Primer sequence of CFI6-Fwd used for RT-qPCR |
| 41 | Primer sequence of CFA6-Rev used for RT-qPCR |
| 42 | Primer sequence of HrpL-Fwd used for RT-qPCR |
| 43 | Primer sequence of HrpL-Rev used for RT-qPCR |
| 44 | Primer sequence of FroC-Fwd used for RT-qPCR |
| 45 | Primer sequence of ProC-Rev used for RT-qPCR |
| 46 | Primer sequence of RpoB-Fwd used for RT-qPCR |
| 47 | Primer sequence of RpoB-Rev used for RT-qPCR |
| 48 | Primer sequence of Cyp3-Forward used for LMW Northern Blot |
| 49 | Primer sequence of Cyp3-Reverse used for LMW Northern Blot |
| 50 | Probe preparation for northern blot analysis: U6 |
| 51 | Primer sequence of Tomato Ubi-Fwd used for RT-qPCR |
| 52 | Primer sequence of Tomato Ubi-Rev used for RT-qPCR |
| 53 | Primer sequence of Pto GFP-Fwd used for RT-qPCR |
| 54 | Primer sequence of Pto GFP-Rev used for RT-qPCR |
| 55 | Primer sequence of IR-CFA6/HRPL-Fwd used for RT-qPCR |
| 56 | Primer sequence of IR-CFA6/HRPL-Rev used for RT-qPCR |
| 57 | Primer sequence of IR-CFA6/HRPL-Fwd used for RT-qPCR |
| 58 | Primer sequence of Ath-Ubi -Rev used for RT-qPCR |
| 59 | HRPL-pDON207-Fwd for Cloning of WT HRPL and mut HRPL in pDON207-attB1/B2 |
| 60 | HRPL-pDON207-Rev for Cloning of WT HRPL and mut HRPL in pDON207-attB1/B2 |
| 61 | Primer dcl2-1-WT-fwd for genotyping dcl2-1 allele |
| 62 | Primer dcl2-1-mut-fwd for genotyping dcl2-1 allele |
| 63 | Primer dcl2-1-WT-Rev for genotyping dcl2-1 allele |
| 64 | Primer dcl3-1-fwd for genotyping dcl3-1 allele |
| 65 | Primer dcl3-1-Rev for genotyping dcl3-1 allele |
| 66 | Primer LBal |
| 67 | Primer dcl-4-2-G8605 Fwd for genotyping dcl4-2 allele |
| 68 | Primer dcl-4-2-G8605 Rev for genotyping dcl4-2 allele |
| 69 | Primer GABI-8474-LP |
| 70 | Northern blot analysis IR-HHR Fwd Primer |
| 71 | Northern blot analysis IR-HHR Rev Primer |
| 72 | RT-qPCR LuxA Fwd |
| 73 | RT-qPCR LuxA Rev |
| 74 | RT-qPCR LuxB Fwd |
| 75 | RT-qPCR LuxB Rev |
| 76 | HRPL-pDON207-Fwd |
| 77 | HRPL-pDON207-Rev |
| 78 | T7 Fwd CFA6/HRPL |
| 79 | T7 Rev CFA6/HRPL |
| 80 | T7 Fwd CYP51 |
| 81 | T7 Rev CYP51 |
| 82 | T7 Fwd Dc3000_FusA |
| 83 | T7 Rev Dc3000_FusA |
| 84 | T7 Fwd Dc3000_SecE |
| 85 | T7 Rev Dc3000_SecE |
| 86 | T7 Fwd Dc3000_GyrB |
| 87 | T7 Rev Dc3000_GyrB |
| 88 | First strand XC_RS06155 : XC_1225 |
| 89 | Second strand XC_RS06155 : XC_1225 |
| 90 | First strand XC_RS02265 = XC_0447 |
| 91 | Second strand XC_RS02265 = XC_0447 |
| 92 | First strand XC_RS18260 = XC_3609 |
| 93 | Second strand XC_RS18260 = XC_3609 |
| 94 | First strand XC_RS11930 = XC_2375 |
| 95 | Second strand XC_RS11930 = XC_2375 |
| 96 | First strand XC_RS17005 = XC_3357 |
| 97 | Second strand XC_RS17005 = XC_3357 |
| 98 | T7 Fwd XC_RS06155 : XC_1225 |
| 99 | T7 Rev XC_RS06155 : XC_1225 |
| 100 | T7 Fwd XC_RS02265 = XC_0447 |
| 101 | T7 Rev XC_RS02265 = XC_0447 |
| 102 | T7 Fwd XC_RS18260 = XC_3609 |
| 103 | T7 Rev XC_RS18260 = XC_3609 |
| 104 | T7 Fwd XC_RS11930 = XC_2375 |
| 105 | T7 Rev XC_RS11930 = XC_2375 |
| 106 | T7 Fwd XC_RS17005 = XC_3357 |
| 107 | T7 Rev XC_RS17005 = XC_3357 |
| 108 | first strand IT13 (P. aeruginosa) |
| 109 | second strand IT13 (P. aeruginosa) |
| 110 | first strand IT14 (P. aeruginosa) |
| 111 | second strand IT14 (P. aeruginosa) |
| 112 | first strand IT16 (P. aeruginosa) |
| 113 | Second strand IT16 (P. aeruginosa) |
| 114 | first strand IT18 (P. aeruginosa) |
| 115 | second strand IT18 (P. aeruginosa) |
| 116 | first strand IT21 (Shigella flexneri) |
| 117 | second strand IT21 (Shigella flexneri) |
| 118 | first strand IT26 (Shigella flexneri) |
| 119 | second strand IT26 (Shigella flexneri) |
| 120 | first strand IT27 (Shigella flexneri) |
| 121 | second strand IT27 (Shigella flexneri) |
| 122 | First strand FusA (Shigella flexneri) |
| 123 | Second strand FusA (Shigella flexneri) |
| 124 | First strand Can (Shigella flexneri) |
| 125 | Second strand Can (Shigella flexneri) |
| 126 | First strand tsf (Shigella flexneri) |
| 127 | Second strand tsf (Shigella flexneri) |
| 128 | First strand accD (Shigella flexneri) |
| 129 | Second strand accD (Shigella flexneri) |
| 130 | First strand der(Shigella flexneri) |
| 131 | Second strand der(Shigella flexneri) |
| 132 | First strand psd(Shigella flexneri) |
| 133 | Second strand psd(Shigella flexneri) |
| 134 | First strand VirB(Shigella flexneri) |
| 135 | Second strand VirB(Shigella flexneri) |
| 136 | First strand VirF(Shigella flexneri) |
| 137 | Second strand VirF (Shigella flexneri) |
| 138 | First strand IcsA (Shigella flexneri) |
| 139 | Second strand IcsA (Shigella flexneri) |
| 140 | First strand Spa47 (Shigella flexneri) |
| 141 | Second strand Spa47 (Shigella flexneri) |
| 142 | First strand MukB (Shigella flexneri) |
| 143 | Second strand MukB (Shigella flexneri) |
| 144 | First strand YbiT (Shigella flexneri) |
| 145 | Second strand YbiT (Shigella flexneri) |
| 146 | Pak_dnaaC_Fw |
| 147 | Pak_dnaaC_Rv |
| 148 | Pak_dnanC_Fw |
| 149 | Pak_dnanC_Rv |
| 150 | Pak_gyrbC_Fw |
| 151 | Pak_gyrbC_Rv |
| 152 | Pak_dnaaB_Fw |
| 153 | Pak_gyrbB_Rv |
| 154 | Pak_gyrbD_Fw |
| 155 | Pak_dnaaD_Rv |
| 156 | Pak_rpocC_Fw |
| 157 | Pak_rpocC_Rv |
| 158 | Pak_seceC_Fw |
| 159 | Pak_seceC_Rv |
| 160 | Pak_sodbC_Fw |
| 161 | Pak_sodbC_Rv |
| 162 | Pak_rpocB_Fw |
| 163 | Pak_sodbB_Rv |
| 164 | Pak_sodbD_Fw |
| 165 | Pak_rpocD_Rv |
| 166 | Pak_xcpqC_Fw |
| 167 | Pak_xcpqC2_Rv |
| 168 | Pak_pscfC_Fw |
| 169 | Pak_pscfC_Rv |
| 170 | Pak_psccC_Fw |
| 171 | Pak_psccC_Rv |
| 172 | Pak_xcpqB_Fw |
| 173 | Pak_psccB_Rv |
| 174 | Pak_psccD_Fw |
| 175 | Pak_xcpqD_Rv |
| 176 | Sf_ftsaB_Fw |
| 177 | Sf_ftsaB_Rv |
| 178 | Sf_canB_Fw |
| 179 | Sf_canB_Rv |
| 180 | Sf_tsfB_Fw |
| 181 | Sf_tsfB_Rv |
| 182 | Sf_tsfD_Fw |
| 183 | Sf_ftsaD_Rv |
| 184 | Sf_accDB_Fw |
| 185 | Sf_accDB_Rv |
| 186 | Sf_derB_Fw |
| 187 | Sf_derB_Rv |
| 188 | Sf_psdB_Fw |
| 189 | Sf_psdB_Rv |
| 190 | Sf_psdD_Fw |
| 191 | Sf_accdD_Rv |
| 192 | Sf_virfB_Fw |
| 193 | Sf_virfB_Rv |
| 194 | Sf_virbB_Fw |
| 195 | Sf_virbB_Rv |
| 196 | Sf_icsaB_Fw |
| 197 | Sf_icsaB_Rv |
| 198 | Sf_icsaD_Fw |
| 199 | Sf_virfD_Rv |
| 200 | T7 polymerase Pak_DnaA_Fwd |
| 201 | T7 polymerase Pak_DnaA_Rev |
| 202 | T7 polymerase Pak_DnaN_Fwd |
| 203 | T7 polymerase Pak_DnaN_Rev |
| 204 | T7 polymerase Pak_GyrF_Fwd |
| 205 | T7 polymerase Pak_GyrB_Rev |
| 206 | T7 polymerase Pak_RpoC_Fwd |
| 207 | T7 polymerase Pak_RpoC Rev |
| 208 | T7 polymerase Pak_SecF_Fwd |
| 209 | T7 polymerase Pak_SecE_Rev |
| 210 | T7 polymerase Pak_SodB_Fwd |
| 211 | T7 polymerase Pak_SodB_Rev |
| 212 | T7 polymerase sf_FtsA_Fwd |
| 213 | T7 polymerase sf_FtsA_Rev |
| 214 | T7 polymerase sf_Can_Fwd |
| 215 | T7 polymerase sf_Can_Rev |
| 216 | T7 polymerase sf_Fsf_Fwd |
| 217 | T7 polymerase sf_Fsf_Rev |
| 218 | T7 polymerase sf_AccF_Fwd |
| 219 | T7 polymerase sf_AccD_Rev |
| 220 | T7 polymerase sf_Der_Fwd |
| 221 | T7 polymerase sf_Der_Rev |
| 222 | T7 polymerase sf_Psd_Fwd |
| 223 | T7 polymerase sf_Psd_Rev |
| 224 | T7 polymerase sf_VirF_Fwd |
| 225 | T7 polymerase sf_VirF_Rev |
| 226 | T7 polymerase sf_FzrB_Fwd |
| 227 | T7 polymerase sf_FzrB_Rev |
| 228 | T7 polymerase sf_IcsA_Fwd |
| 229 | T7 polymerase sf_IcsA_Rev |
| 230 | T7 polymerase sf_Spa47_Fwd |
| 231 | T7 polymerase sf_Spa47_Rev |
| 232 | T7 polymerase sf_MukB_Fwd |
| 233 | T7 polymerase sf_MukB_Rev |
| 234 | T7 polymerase sf_YbiT_Fwd |
| 235 | T7 polymerase sf_YbiT_Rev |
| 236 | PAK-DnaA-qF |
| 237 | PAK-DnaN-qR |
| 238 | PAK-DnaN-qF |
| 239 | PAK-DnaN-qR |
| 240 | PAK-GyrB-qF |
| 241 | PAK-GyrB-qR |
| 242 | PAK-RpoC-qF |
| 243 | PAK-RpoC-qR |
| 244 | PAK-SecE-qF |
| 245 | PAK-SecE-qR |
| 246 | PAK-SodB-qF |
| 247 | PAK-SodB-qR |
| 248 | Sequence of the first arm of the LuxA/LuxB dsRNA |
| | used to target concomitantly the LuxA and LuxB genes |
| 249 | Sequence of the second arm of the LuxA/LuxB dsRNA |
| | used to target concomitantly the LuxA and LuxB genes |
| 250 | Sequence of the first arm of the LptH/LolA/TolB dsRNA used to concomitantly target |
| | LptH, LolA and TolB genes genes from P. aeruginosa |
| 251 | Sequence of the second arm of the LptH/LolA/TolB dsRNA used to concomitantly target |
| | LptH, LolA and TolB genes from P. aeruginosa |
| 252 | Sequence of the first arm of the LpxA/LpxD/TolB dsRNA used to concomitantly target |
| | LpxA, LpxD and TolB genes from P. aeruginosa |
| 253 | Sequence of the second arm of the LpxA/LpxD/TolB dsRNA used to concomitantly target |
| | LpxA, LpxD and TolB genes from P. aeruginosa |
| 254 | Sequence of the first arm of the secE/dnaN/gyrB dsRNA used to concomitantly target secE, |
| | dnaN and gyrB genes from P. aeruginosa |
| 255 | Sequence of the second arm of the secE/dnaN/gyrB dsRNA used to concomitantly target |
| | secE, dnaN and gyrB genes from P. aeruginosa |
| 256 | Sequence of the first arm of the XcpQ/ExsA/PcrV/LasR/RhlR/VqsM/RmsA dsRNA used to |
| | concomitantly target XcpQ, ExsA, PcrV, LasR, RhlR, VqsM and RmsA genes from P. |
| | aeruginosa |
| 257 | Sequence of the second arm of XhsXcpQ/ExsA/PcrV/LasR/RhlR/VqsM/RmsA dsRNA used |
| | to concomitantly targetXcpQ, ExsA, PcrV, LasR, RhlR, VqsM and RmsA genes from P. |
| | aeruginosa |
| 258 | Sequence of the first arm of the XcpQ/PscF/PscC dsRNA used to concomitantly target |
| | XcpQ, PscF and PscC genes from P. aeruginosa |
| 259 | Sequence of the second arm of the XcpQ/PscF/PscC dsRNA used to concomitantly target |
| | XcpQ, PscF and PscC genes from P. aeruginosa |
| 260 | Sequence of the first arm of the ExoS/exsA/Vrf dsRNA used to concomitantly target ExoS, |
| | ExsA and Vrf genes from P. aeruginosa |
| 261 | Sequence of the second arm of the ExoS/exsA/Vrf dsRNA used to concomitantly target |
| | ExoS, ExsA and Vrf genes from P. aeruginosa |
| 262 | Sequence of the first arm of the ExoU/exsA/Vrf dsRNA used to concomitantly target ExoU, |
| | ExsA and Vrf genes from P. aeruginosa |
| 263 | Sequence of the second arm of the ExoU/exsA/Vrf dsRNA used to concomitantly target |
| | ExoU, ExsA and Vrf genes from P. aeruginosa |
| 264 | Sequence of the first arm of the LasR/RhlR/VqsM dsRNA used to concomitantly target |
| | LasR, RhlR and VqsM genes from P. aeruginosa |
| 265 | Sequence of the second arm of the LasR/RhlR/VqsM dsRNA used to concomitantly target |
| | LasR, RhlR and VqsM genes from P. aeruginosa |
| 266 | Sequence of the first arm of the GacA/RmsA/MvfR dsRNA used to concomitantly target |
| | GacA, RmsA and MvfR genes from P. aeruginosa |
| 267 | Sequence of the second arm of the GacA/RmsA/MvfR dsRNA used to concomitantly target |
| | GacA, RmsA and MvfR genes from P. aeruginosa |
| 268 | Sequence of the first arm of the VirF/VirB/IcsA dsRNA used to concomitantly target VirF, |
| | VirB and IcsA genes from Shigella flexneri |
| 269 | Sequence of the second arm of the VirF/VirB/IcsA dsRNA used to concomitantly target |
| | VirF, VirB and IcsA genes from Shigella flexneri |
| 270 | Sequence of the first arm of the fnbA/clfA/clfB/spa dsRNA used to concomitantly target |
| | fnbA, clfA, clfB and spa genes from S. aureus |
| 271 | Sequence of the second arm of the fnbA/clfA/clfB/spa dsRNA used to concomitantly target |
| | fnbA, clfA, clfB and spa genes from S. aureus |
| 272 | Sequence of the first arm of the lukF-PV/lukS-PV/lukE/lukD dsRNA used to concomitantly |
| | target lukF-PV, lukS-PV, lukE and lukD genes from S. aureus |
| 273 | Sequence of the second arm of the lukF-PV/lukS-PV/lukE/lukD dsRNA used to |
| | concomitantly target lukF-PV, lukS-PV, lukE and lukD genes from S. aureus |
| 274 | Sequence of the first arm of the HlgB/hla/tsst-1/atl dsRNA used to concomitantly target |
| | HlgB, hla, tsst-1 and atl genes from S. aureus |
| 275 | Sequence of the second arm of the HlgB/hla/tsst-1/atl dsRNA used to concomitantly target |
| | HlgB, hla, tsst-1 and atl genes from S. aureus |

### FIGURE LEGENDS

FIG. 1. Phenotypical and molecular characterization of Arabidopsis transgenic plants expressing the inverted repeat IR-CFA6/HRPL in both untreated and bacterial challenged conditions
A. Schematic representation of the Pto DC3000 genes Cfa6 and HrpL. The 250 bp regions of Cfa6 (1-250 nt) and HrpL (99-348 nt) genes were used to generate the chimeric hairpin construct under the control of the constitutive 35S promoter.
B. Representative pictures of five-week old Col-0 plants and of independent homozygous transgenic plants expressing the 35Sₚᵣₒ:IR-CYP51 (Control vector: CV) or the 35Sₚᵣₒ:IR-CFA6/HRPL construct.
C. Accumulation level of anti-Cfa6 and anti-HrpL siRNAs detected by low molecular weight Northern blot analysis of the Arabidopsis plants depicted in B. U6 was used as a loading control.
D. Pto DC3000 HrpL mRNA accumulation is significantly decreased on IR-CFA6/HRPL-infected plants compared to Col-0- and CV-infected plants. Arabidopsis plants depicted in B. were dip-inoculated with Pto DC3000 WT strain and at 3 days post-infection (dpi), bacterial transcript levels of ProC, Cfa6 and HrpL were monitored by quantitative RT-PCR analysis. These mRNA levels are quantified relative to the level of bacterial GyrA transcript. Error bars indicate the standard deviations of mRNA values obtained in three independent experiments. Statistically significant differences were assessed using ANOVA test (ns: p-value>0.05; *: p-value<0.05, **: p-value<0.01, ***: p-value<0.001).

FIG. 2. Phenotypical and molecular characterization of Arabidopsis transgenic plants expressing the inverted repeat IR-LuxA/LuxB in both untreated and bacterial challenged conditions
A. Schematic representation of the luxCDABE operon inserted into Pto DC3000 WT genome. The 250 bp regions of luxA (1-250 nt) and luxB (1-250 nt) genes were used to generate the chimeric hairpin construct under the control of the constitutive 35S promoter.
B. Accumulation level of anti-LuxA/LuxB detected by low molecular weight Northern blot analysis of the Arabidopsis transgenic plants. U6 was used as a loading control.
C. A significant impact on the luminescence of Pto DC3000 luciferase (Pto Luc) was observed in the transgenic lines expressing the IR-LuxA LuxB as compared to Col-0 upon infection. The two independent transgenic lines of IR-LuxA LuxB #18 and #20, along with Col-0 were syringe-infiltrated with Pto Luc at a concentration of 10⁶ cfu/ml and the luminescence was measured at 24 hours-post infiltration.
D. The in planta growth of Pto DC3000 is unaltered in IR-LuxA LuxB transgenic plants compared to Col-0 plants. Leaf discs from the plants used in C. were grinded and plated in a serial dilution to count Pto Luc for each condition at 24 hours post-infection.

FIG. 3. Arabidopsis transgenic plants expressing the IR-CFA6/HRPL construct suppress Pto DC3000-induced stomatal reopening
A. The Pto Δcfa6 and ΔhrpL strains, but not the ΔhrcC strain, were impaired in their ability to reopen stomata and these phenotypes were rescued upon addition of exogenous COR. Sections of unpeeled leaves of Col-0 plants were incubated with mock solution (water) or Pto DC3000 WT, Δcfa6, ΔhrpL or ΔhrcC strains for 3 hours. Stomata aperture was assessed by measuring the width and length using ImageJ software.
B. Pto DC3000 WT no longer induced stomatal reopening in Arabidopsis transgenic lines overexpressing the IR-CFA6/HRPL hairpin. Stomatal aperture measurement was conducted in Col-0 and 35Sₚᵣₒ:IR-CFA6/HRPL #4, #5, #10 transgenic lines infected with Pto WT strain as described in A.
C. The Pto DC3000-induced stomatal reopening response was unaltered in CV compared to Col-0 plants. Stomatal aperture measurement was conducted in Col-0 and CV plants infected with Pto WT strain as described in A.

Note: For all these experiments, n=number of stomata analyzed per condition and statistical significance was assessed using the ANOVA test (ns: p-value>0.05; ****: p-value<0.0001).

FIG. 4. Arabidopsis transgenic plants expressing the IR-CFA6/HRPL construct exhibit a reduced vascular spreading and growth of Pto DC3000 in adult leaves
A. IR-CFA6/HRPL #4, #5 and #10-infected plants exhibit reduced vascular spreading of Pto WT compared to Col-0- and CV-infected plants. Plants were wound-inoculated in midveins with Pto WT-GFP and Col-0 was wound-inoculated with PtoΔcfa6-GFP. GFP fluorescence signal was observed under UV light and pictures were taken at 3 days post-infection (dpi). To index the spreading of bacteria from the inoculation sites, GFP fluorescence was observed under UV light. When the bacteria propagated away from any of the three inoculation sites, it was indexed as propagation with 4 corresponding to the highest propagation index. Pictures from three biological replicates were taken into consideration.
B. Representative picture of infected leaves of conditions used in A. are depicted. White circles indicate the site of wound-inoculation in the leaf midvein.
C. IR-CFA6/HRPL #4, #5 and #10 transgenic lines exhibit a significantly reduced Pto WT titer when compared to Col-0 and CV-infected plants. Col-0, CV and IR-CFA6/HRPL #4, #5 and #10 plants were dip-inoculated with Pto WT and Col-0 plants were dip-inoculated with the PtoΔcfa6-GFP strain. Bacterial titers were monitored at 2 days post-infection (dpi). Four leaves from three plants per condition and from three independent experiments (n) were considered for the comparative analysis.
D. IR-CFA6/HRPL #4, #5 and #10 transgenic plants exhibit reduced water-soaking symptoms in comparison to Col-0 and CV plants. Representative leaf pictures of water-soaking symptoms were taken 24 hours after dip-inoculation.

Note: For all the above experiments, statistical significance was assessed using the two-way ANOVA test (ns: p-value>0.05; *: p-value<0.05; **: p-value<0.01; ***: p-value<0.001; ****: p-value<0.0001).

FIG. 5. Phenotypical characterization of Arabidopsis transgenic plants expressing the inverted repeat IR-HRPG/HRPX/RSMA in both untreated and Xanthomonas campestris pv. campestris challenged conditions
A. IR-HRPG/HRPX/RSMA #1- and #6-infected plants exhibit reduced vascular spreading of the virulent XccΔXopAC (GUS/GFP) strain compared to Col-0-infected plants. Plants were wound-inoculated in midveins with XccΔXopAC (GUS/GFP) at OD=0.01. GFP fluorescence signal was observed under UV light and pictures were taken at 3 days post-infection (dpi). The indexing was done as described in 4A.
B. Representative picture of infected leaves of conditions used in B. are depicted. White circles indicate the site of wound-inoculation in the leaf midvein.

FIG. 6. Exogenously delivered total RNAs from IR-CFA6/HRPL transgenic plants reduce Pto DC3000 pathogenicity when applied on the surface of wild type Arabidopsis and tomato leaves
A. In vitro AGS assay showing that total RNA extract from CFA6/HRPL #4 plants triggers silencing of both Cfa6 and HrpL genes. Pto WT cells were incubated in vitro for 4 and 8 hours with 20 ng/μl of total RNAs from CV or IR-CFA6/HRPL #4 plants. Significant reduction of the bacterial transcripts Cfa6 and HrpL was observed by RT-qPCR at both the timepoints, while accumulation of ProC and RpoB transcripts remained unaffected. GyrA was used as an internal control to quantify the accumulation of bacterial transcripts. Error bars indicate the standard deviations of values from three independent experiments.
B. The ability of Pto WT to reopen stomata was altered upon exogenous application of total RNAs extract from IR-CFA6/HRPL plants compared to CV plants. Col-0 leaves were treated for 1 hour with water or 20 ng/μl of total RNAs extracted from CV or IR-CFA6/HRPL #4 plants and were incubated with Pto WT for 3 hours. Stomatal aperture was measured and analyzed as described in FIG. 3A.
C. Treatment with IR-CFA6/HRPL, but not with CV, total RNAs compromised the ability of Pto DC3000 to multiply in the apoplast of leaves when compared to pretreatment with CV total RNAs. Col-0 leaves were treated with 20 ng/μl of total RNAs from CV or IR-CFA6/HRPL #4 plants for 1 hour, followed by dip-inoculation with Pto WT. Bacterial titers were monitored at 2 dpi. The number of leaves (n) corresponds to collective values from three independent experiments.
D. The leaves treated with CV total RNAs displayed more necrotic symptoms as compared to the leaves treated with IR-CFA6/HRPL #4 total RNAs. The experiment was conducted as in C. but using five-week-old tomato (Solanum lycopersicum ‘Moneymaker’) plants. Representative pictures of infected leaves in the two conditions are depicted.
E. A reduced number of Pto DC3000-GFP foci was observed in tomato leaves treated with total RNA extracts from IR-CFA6 HRPL #4 versus CV plants. Infected-leaves were observed at 3 dpi under UV light to estimate the number of GFP loci. On the left: Dot plot representing the number of GFP loci analyzed using ImageJ software from 3-4 different leaves per condition with at least 4 pictures per leaf. The values used for the analysis are from two different independent experiments. Student's t-test was performed for the comparative analysis. On the right: Representative picture of the tomato leaves described in D.
F. Pto WT-GFP DNA content is decreased in tomato leaves treated with total RNA extracts from IR-CFA6/HRPL #4 versus CV plants. The level of bacterial DNA content was analyzed by qPCR using tomato Ubiquitin as a control. Student's t-test was performed for the comparative analysis.

Note: For A, B and C, statistically significant differences were assessed using ANOVA test (ns: p-value>0.05; **: p-value<0.01, ***: p-value<0.001).

FIG. 7. DCL-dependent antibacterial siRNAs, but not corresponding unprocessed dsRNA precursors, are the RNA entities responsible for AGS and for the suppression of stomatal reopening
A. Upper panel: Accumulation level of IR-CFA6/HRPL transcripts in Col-0, dcl2-1 dcl3-1 dcl4-2 (dcl234), IR-CFA6/HRPL #4 (#4) and IR-CFA6/HRPL #4 in dcl234 mutant background (#4×dcl234) was performed by RT-qPCR. Ubiquitin was used as a control. The graph represents the mean and standard deviation of three independent experiments. Lower panel: Accumulation level of anti-Cfa6 and anti-HrpL siRNAs was performed by low molecular weight Northern blot analyses in the same genotypes. U6 was used as a loading control.
B. Total RNA extract from #4×dcl234 plants does not alter the transcript accumulation levels of Cfa6 and HrpL. Pto WT cells were incubated in vitro for 8 hours with 20 ng/μl of total RNAs extracted from the same genotypes described in A. Accumulation levels of Cfa6 and HrpL transcripts was assessed by RT-qPCR analysis using GyrA as a control. Error bars indicate the standard deviations of values from three independent experiments. Statistically significant differences were assessed using ANOVA test (ns: p-value>0.05; *: p-value<0.05, **: p-value<0.01).
C. Total RNA extract from #4×dcl234 plants does not suppress Pto DC3000-induced stomatal reopening response. Col-0 leaves were treated with water or 20 ng/μl of total RNA extracts from the same genotypes than the ones used in A. for 1 hour and incubated with Pto WT for 3 hours. Stomatal aperture was measured and analyzed as described in FIG. 2A. Two other biological replicates are presented in Supplementary FIG. 4B.
D. Upper panel: Electrogram profiles representing the RNA size distribution of total, long and small RNAs from IR-CFA6/HRPL #4 plants determined with an agilent Bioanalyzer 2100 equipped with an RNA Nano chip. Low molecular weight RNA fractions are encircled for each sample. 18S and 25S ribosomal peaks are highlighted. Lower panel: Agarose gel picture of ethidium bromide stained total, long and small RNAs used in A.
E. Small RNA species, but not the corresponding long RNA species, from IR-CFA6/HRPL plants suppress stomatal reopening to the same extent as total RNA extracts. The experiment was conducted as in D. but with total, long (>200 nt) or small (<200 nt) RNA fractions, which were separated from total RNAs of IR-CFA6/HRPL #4 plants. Note: For all the stomata experiments, statistical significance was assessed using the ANOVA test (ns: p-value>0.05; ****: p-value<0.0001).

FIG. 8. A bacterially expressed small RNA resilient version of HrpL is refractory to gene silencing directed by anti HrpL siRNAs and exhibits a normal stomatal reopening phenotype upon exogenous application of anti HrpL siRNAs
A. Schematic representation of the PtoΔhrpL strain along with the complementation strains generated upon transformation with the plasmids encoding WT HrpL or mut HrpL, respectively under the control of the constitutive promoter NptII. B. In vitro AGS assay showing that the PtoΔhrpL WT HrpL strain is sensitive to antibacterial RNAs while the PtoΔhrpL mut HrpL is refractory to these RNA entities.

Bacterial PtoΔhrpL WT HrpL and PtoΔhrpL mut HrpL strains were incubated with total RNAs extracted from CV or IR-CFA6/HRPL #4 plants for 8 hours. Accumulation level of WT HrpL and mut HrpL transcripts was analyzed by RT-qPCR (the mRNA levels were relative to the level of GyrA transcript). Error bars indicate the standard deviations of values from three independent experiments. Statistically significant differences were assessed using ANOVA test (ns: p-value>0.05; *: p-value<0.05, **: p-value<0.01).
C. Accumulation of anti-Cfa6 and anti-HrpL siRNAs was assessed by low molecular weight northern analysis using total RNA extracts from N. benthamiana plants transiently expressing 35Sₚᵣₒ:IR-HRPL, 35Sₚᵣₒ:IR-CFA6/HRPL and from non-transformed N. benthamiana leaves (Nb). U6 was used as a loading control.
D. The PtoΔhrpL mut HrpL strain is refractory to anti HrpL siRNA action. Col-0 leaves were treated with total RNAs extracted either from N. benthamiana alone or from N. benthamiana expressing the inverted repeat IR-HRPL. Stomatal reopening response was assessed as described previously.

Note: For all the stomata experiments, statistical significance was assessed using the ANOVA test (ns: p-value>0.05; ****: p-value<0.00001).

FIG. 9. The apoplastic fluid of IR-CFA6/HRPL plants is composed of functional antibacterial siRNAs that are either embedded into EVs, and protected from micrococcal nuclease action, or in a free form, and sensitive to micrococcal nuclease digestion
A. The ability of Pto WT to reopen stomata was also altered to similar levels upon exogenous application of Apoplastic fluid (APF) extract as compared to total RNAs derived from IR-CFA6 HRPL plants. Total RNAs and APF extracted from CV plants was used as negative control. Col-0 leaves were treated for 1 hour with water (Mock) or 20 ng/μl of total RNAs or 500 μl of APF extracted from CV or IR-CFA6/HRPL #4 plants and were incubated with Pto WT for 3 hours. Stomatal aperture was measured and analyzed as described in previous experiments.
B. The two different vesicular fractions, P40 and P100, as well as the free RNA population present in the supernatent (SN) carry the antibacterial siRNAs and thus are involved in AGS. Apoplastic fluid extracted from both CV and IR-CFA6/HRPL #4 plants was subjected to ultracentrifugation at 40,000 g to pellet the larger population of EVs (P40) and the remaining supernatent was further subjected to ultracentrifugation at 100,000 g to pellet the smaller EVs (P100). SN was also restored. Col-0 leaves were treated for 1 hour with water (Mock) or P40, P100 and SN extracted from CV or IR-CFA6/HRPL #4 plants and were incubated with Pto WT for 3 hours. The P40, P100 and SN of #4 were treated with 20 units of Mnase and the SN of #4 was also treated with 20 units of Proteinase K. Stomatal aperture was measured and analyzed as described in previous experiments.

Note: For all the stomata experiments, statistical significance was assessed using the ANOVA test (ns: p-value>0.05; ****: p-value<0.00001).

FIG. 10. Exogenous delivery of in vitro synthesized antibacterial siRNAs reduces the pathogenicity as well as the viability of Pto DC3000
A. 2% Agarose gel of ethidium bromide stained in vitro synthesized long dsRNAs and RNase III digested siRNAs corresponding to IR-CYP51 and IR-CFA6/HRPL are depicted.
B. The ability of Pto WT to reopen stomata was altered upon exogenous application of in vitro synthesized siRNAs, but not the long dsRNAs, corresponding to IR-CFA6/HRPL. Long dsRNAs and siRNAs from IR-CYP51 was used as negative control. Col-0 leaves were treated for 1 hour with water (Mock) or RNA presented in A. and then incubated with Pto WT for 3 hours. Stomatal aperture was measured and analyzed as described in previous experiments.
C. In vitro AGS assay using the in vitro synthesized siRNAs from IR-CFA6/HRPL triggers silencing of both Cfa6 and HrpL genes. Pto WT cells were incubated in vitro for 8 hours with 2 ng/μl of in vitro synthesized siRNAs from IR-CYP51 or IR-CFA6/HRPL #4 plants. Significant reduction of the bacterial transcripts Cfa6 and HrpL was observed by RT-qPCR, while accumulation of ProC and RpoB transcripts remained unaffected. GyrA was used as an internal control to quantify the accumulation of bacterial transcripts. Error bars indicate the standard deviations of values from three independent experiments.
D. and E. In vitro synthesized siRNAs against fusA or gyrB of Pto DC3000 have a significant impact on the growth of the Pto DC3000-GFP strain. siRNAs directed against secE, gyrB and fusA genes of Pto DC3000 were synthesized using in vitro transcription followed by RNaseIII digestion. The Pto DC3000-GFP strain was incubated with the indicated concentration of in vitro synthesized siRNAs. 96-well plate was set on the machine for the samples to be fractioned in droplets by the droplet-based microfluidic system (Millidrop). For each well, 10 droplets of ˜500 nl each were formed and incubated inside the instrument. For each droplet, measurements of biomass and of GFP fluorescence were acquired every ˜30 minutes.

FIG. 11. Impact of exogenously delivered plant-derived small RNAs on the human pathogenic bacterium Pseudomonas aeruginosa PAK strain
A. Quantification of luminescence of the P. aeruginosa PAK Luciferase (lux-tagged PAK) strain incubated with plant-derived total RNA extracts in a time course (mins) is depicted. A concentration of 10⁸ cfu ml⁻¹ of the lux-tagged PAK strain was incubated with either water (Mock) or specific total RNAs at 20 ng/μl that were extracted from N. benthamiana non-transformed leaves (NB) or N. benthamiana leaves expressing IR-GF/FG (negative control) or IR-LuxA/LuxB (IR-LuxAB) and luminescence was measured using the Berthold Luminometer. Mean of readings measured at every 30 mins over a period of 4 hours from 4 technical replicates/condition is plotted.
B. Bacterial count (OD₆₀₀) for the samples in A at 4-hour timepoint was measured using a plate reader and plotted in the dot plot.
C. Same as in A but by incubating the lux-tagged PAK strain with either water (Mock) or specific total RNAs at a concentration of 20 ng/μl extracted from N. benthamiana non-transformed leaves (NB) or N. benthamiana leaves transiently expressing IR-GF/FG (negative control), IR-DnaA/DnaN/GyrB (IT13) or IR-RpoC/SecE/SodB (IT14).
D. Same as in B but for the samples depicted in C.

Note: For B and C statistical significance was assessed using the ANOVA test (ns: p-value>0.05; **: p-value<0.001).

FIG. 12. In vitro synthesized siRNAs directed against SecE, trigger growth reduction of the Pseudomonas aeruginosa PAO1 strain in in vitro conditions

In vitro synthesized antibacterial siRNAs were tested against several essential genes of P. aeruginosa PAO1 strain and were screened for having a significant impact on the growth of the bacteria. siRNAs directed against SecE, GyrB, DnaN, DnaA, RpoB or SodB genes of P. aeruginosa were synthesized using in vitro transcription followed by RNaseIII digestion. PAO1 strain at 10⁸ cfu ml⁻¹ was treated with 5 ng/μl concentration of individual gene targeting siRNAs. 96-well plate was set on the machine for the samples to be fractioned in droplets by the Millidrop Analyzer. For each well, 10 droplets of ˜500 nL each were formed and incubated inside the instrument. For each droplet, measurements of biomass were acquired every ˜30 minutes for 14 hours. Median of scattering signal acquired from 30 droplets/condition at each time point is plotted.

### EXAMPLES

### Example 1: Materials and Methods

Generation of Transgenic Lines Carrying Inverted Repeats Constructs

The IR-HRPL/CFA6 chimeric hairpin was designed to produce artificial siRNAs targeting a 250 bp region of Cfa6 (from nucleotide 1 to 250) and a 250 bp region of HrpL from nucleotide 99 to 348 (SEQ ID NO: 1, 2 and 3). The IR-CFA6-A and IR-CFA6-B are two independent inverted repeats that specifically target the Cfa6 gene from nucleotide 1 to 250 (SEQ ID NO: 4, 2 and 5) and from nucleotide 1 to 472 (SEQ ID NO: 6, 2 and 7), respectively. The IR-HRPL-A and IR-HRPL-B are two independent inverted repeats that specifically target HrpL from nucleotide 99 to 348 (SEQ ID NO: 8, 2 and 9) and from nucleotide 1 to 348 (SEQ ID NO: 10, 2 and 11), respectively. The IR-HRCC hairpin was designed to specifically target the HrcC gene (SEQ ID NO: 12, 2 and 13) and the IR-AvrPto/AvrPtoB to concomitantly target the type III effector AvrPto and AvrPtoB genes (SEQ ID NO: 14, 2 and 15). The IR-CYP51 hairpin was designed to produce siRNAs against three cytochrome P450 lanosterol C-14α-demethylase genes of the fungus F. graminearum, namely FgCYP51A, FgCYP51B and FgCYP51C as previously performed (SEQ ID NO: 16, 2 and 17), (19). This hairpin was used as a negative control for all the in planta assays of the invention. Additional inverted repeats were designed and cloned as part of this study to target virulence factors or essential genes from different strains of Pseudomonas, Xanthomonas and Ralstonia. These hairpins are described as follows: the IR-HrpG/HrpB/HrcC hairpin designed to concomitantly target the HrpG, HrpB and HrcC genes from Ralstonia species (SEQ ID NO: 18, 2 and 19), the IR-HrpB/HrcC/TssB/XpsR hairpin designed to concomitantly target the HrpB, HrcC, TssB and XpsR genes from Ralstonia species (SEQ ID NO: 20, 2 and 21), the IR-HrpG/HrpX/RsmA hairpin designed to concomitantly target the HrpG, HrpX and Rsma genes from Xanthomonas campestris pv. campestris (SEQ ID NO: 22, 2 and 23), the IR-RpoB/RpoC/FusA hairpin designed to concomitantly target the essential genes RpoB, RpoC and FusA from Pto DC3000 and Pseudomonas syringae strain CC440 (SEQ ID NO: 24, 2 and 25), the IR-SecE-RpoA-RplQ hairpin designed to concomitantly target the essential genes SecE, RpoA and RplQ from Pto DC3000 and Pseudomonas syringae strain CC440 (SEQ ID NO: 26, 2 and 27), the IR-NadHb/NadHd/NadHe hairpin designed to concomitantly target the essential genes NadHb, NadHd and NadHe from different Xanthomonas species including Xanthomonas campestris pv. campestris (SEQ ID NO: 28, 2 and 29), the IR-DnaA/DnaE1/DnaE2 hairpin designed to concomitantly target the essential genes NadHb, NadHd and NadHe from different Xanthomonas species including Xanthomonas campestris pv. campestris (SEQ ID NO: 30, 2 and 31). Inverted repeats were designed and cloned as part of this study to target virulence factors or essential genes from different strains of Pseudomonas aeruginosa and Shigella. These hairpins are described as follows: the IT13 hairpin targeting the DnaA, DnaN and GyrB genes (SEQ ID NO: 108-109), the IT14 hairpin targeting the RpoC, SecE and SodB genes (SEQ ID NO: 110-111), the IT16 hairpin targeting the XcpQ, PscF and PscC genes (SEQ ID NO: 112-113), the IT18 hairpin XcpQ, ExsA and HphA genes of P. aeruginosa (SEQ ID NO: 114-115), the IT21 hairpin targeting the FtsA, Can and Tsf genes (SEQ ID NO: 116-117), the IT26 hairpin of targeting the AccD, Der and Psd genes (SEQ ID NO: 118-119), and the IT27 hairpin targeting the VirF, VirB and IcsA genes of Shigella flexneri (SEQ ID NO: 120-121). Furthermore, a chimeric inverted repeat was designed and cloned as part of this study to target the Photorhabdus luminescens luxCDABE operon chromosomally expressed in Pto DC3000 under the constitutive kanamycin promoter: the IR-LuxA/LuxB hairpin, designed to concomitantly target the LuxA and LuxB genes from Pto DC3000 luciferase strain as well as P. aeruginosa luciferase strain (SEQ ID NO: 248, 2 and 249). All the above-described hairpins contain a specific intron sequence from the Petunia Chalcone synthase gene CHSA (SEQ ID NO: 2) and were cloned into a vector carrying the Cauliflower Mosaic Virus (CaMV) 35S constitutive promoter. More specifically, the following hairpin sequences: IR-HRPL/CFA6, IR-CYP51, IR-CFA6-B, IR-HRPL-B, IR-HrpG/HrpB/HrcC, IR-HrpB/HrcC TssB XpsR, IR-AvrPto/AvrPtoB, IR-HRCC, IR-HrpG HrpX/RsmA and IR-LuxA LuxB were cloned into a modified pDON221-P5-P2 vector carrying additional EcoRI and SalI restriction sites to facilitate the insertion of these long inverted-repeats into this vector. A double recombination between pDON221-P5-P2 carrying the hairpin sequence and pDON221-P1-P5r (Life Technologies, 12537-32), carrying the constitutive 35S promoter sequence, was conducted in the pB7WG GATEWAY compatible destination vector (binary vector carrying a BAR selection marker and gateway recombination sites). The remaining hairpins, namely the IR-CFA6-A, IR-HRPL-A, IR-RpoB/RpoC/FusA, IR-SecE-RpoA-RplQ, IR-NadHb/NadHd/NadHe and IR-DnaA/DnaE1/DnaE2 sequences were generated by PCR amplifications of the sense and antisense regions of the target genes using the bacterial genomic DNA as template and followed by the generation of modules required for the cloning into a final GreenGate destination vector pGGZ003. All the plasmids were then introduced into the Agrobacterium tumefaciens strains GV3101 or C58C1 and further used for either transient expression in Nicotiana benthamiana or stable expression in the Arabidopsis thaliana Columbia-0 (Col-0) reference accession.

Plant Material and Growth Conditions

Stable transgenic lines of IR-CFA6/HRPL and CV were generated by transforming Arabidopsis WT (accession Col-0) plants using Agrobacterium mediated-floral dip method. Three independent transgenic lines, #4, #5 and #10 expressing equal amount of anti-Cfa6 and anti-HrpL siRNAs were selected and propagated until T4 generation. Similarly, selected homozygous line of CV expresses abundant level of siRNAs against F. graminearum CYP51A/B/C genes was propagated until T4 generation for experimentation. Similarly, transgenic lines expressing IR-LuxA LuxB and IR-HrpG/HrpX/RsmA were selected on the basis of siRNA production and propagated further. For genetic analysis, dcl2 dcl3 dcl4 (dcl234) triple mutant plant was crossed with the reference IR-CFA6/HRPL #4 line and the F3 plants were genotyped to select homozygous dcl234 mutant containing homozygous IR-CFA6/HRPL transgene. Sterilized seeds of Arabidopsis Col-0 and the selected homozygous transgenic lines were first grown for 12-14 days at 22° C. on plates containing 12×MS medium (Duchefa), 1% sucrose and 0.8% agar (with or without antibiotic selection) in 8 h photoperiod. Seedlings were then pricked out to soil pots and grown in environmentally controlled conditions at 22° C./19° C. with an 8 h photoperiod under light intensity of 100 μE/m2/s. Four- to five-week-old plants were used for all the experiments. Seeds of tomato (Solanum lycopersicum ‘Moneymaker’) and N. benthamiana were directly sown on soil pots and grown in environmentally controlled conditions at 22° C./19° C. (day/night) with a 16 h photoperiod under light intensity of 100 μE/m2/s. Four- to five-week old plants were used for all the experiments.

Bacterial Strains

The GFP expressing Pto DC3000-GFP and the Pto DC3000Δcfa6-GFP (Pto DC3118) strains were a gift from Dr. S. Y. He, while the Pto DC3000ΔhrpL strain was a gift from Dr. Cayo Ramos. The Pto DC3000 luciferase strain was a gift from Dr. Chris Lamb. The Pto DC3000 ΔhrpL and Pto DC3000ΔhrcC strains expressing the GFP reporter gene were generated by transforming them with the same plasmid as in Pto DC3000-GFP by electroporation and then plated at 28° C. on NYGB medium (5 g/L bactopeptone, 3 g/L yeast extract, 20 ml/L glycerol) containing gentamycin (1 μg/ml) for selection. To generate the Pto DC3000-WT-HrpL and -mut-HrpL strains, the Pto DC3000ΔhrpL strain was transformed with the plasmids NPTIIₚᵣₒ:WT-HrpL and NPTIIₚᵣₒ:mut-HrpL, respectively, by electroporation and then plated in NYGB medium with gentamycin. The PAK and PAO1 strains of P. aeruginosa were availed from other labs in collaboration.

RNA Gel Blot Analyses

To perform northern blot analyses of low molecular weight RNAs, total RNA was extracted using TriZOL reagent and stabilized in 50% formamide. Around 30 μg of total RNA from the specified conditions were used to perform Northern blot analyses as previously described (51). Regions of 150 bp to 300 bp were amplified from the plasmids using gene specific primers and the amplicons were further used to generate specific 32P-radiolabelled probes synthesized by random priming. U6 probe was used as a control for equal loading of small RNAs.

Separation of Long and Small RNA Fractions

Total RNAs were extracted from Arabidopsis leaves of IR-CFA6/HRPL #4 using Tri-Reagent (Sigma, St. Louis, Mo.) according to the manufacturer's instructions. Using 100 μg of total RNA, long and small RNA fractions were separated using the mirVana miRNA isolation kit (Ambion, Life technologies) according to the manufacturer's instructions. The separation of long and small RNAs from the total RNAs was visualized using agarose gel electrophoresis and further analyzed using microfluidic based approach (Bioanalyzer 2100; Agilent Technologies, http://www.agilent.com). The total, long and small RNAs were further used to perform the stomatal reopening assay.

Bacterial Infection Assays in Plants

(a) Bacterial growth assay: Plants for this experiment were specifically used after three hours of beginning of the night cycle in growth chamber. Three plants per condition were dip-inoculated using the bacterium at 5×10⁷ cfu/ml with 0.02% Silwet L-77 (Lehle seeds). Plants upon bacterial dipping were immediately placed in chambers with high humidity to facilitate proper infection. Water-soaking symptoms upon dip-inoculation were observed 24 hours post-infection and pictures of leaves from three plants per condition were taken. Two days post-inoculation, bacterial titer for each mentioned condition was measured for individual infected leaf as described in (51). To quantify bacterial transcripts in infected plants, pool of infected leaf samples was collected three days post-inoculation.

(b) Wound-inoculation assay: To monitor the propagation of bacteria in the midveins, around 15 leaves from three plants per condition were manually inoculated with a toothpick dipped in GFP-tagged bacteria at a concentration of 5×10⁶ cfu/ml and then the plants were placed in chambers with high humidity for 3 days. Bacterial propagation was then analyzed by monitoring GFP signal under a UV light using an Olympus MV 10×macrozoom and pictures were taken with a CCD camera AxioCam MVrc Zeiss with a GFP filter.

(c) Plant protection assay: Prior to bacterial infection, four rosette leaves of three Arabidopsis plants per condition were individually treated by repeatedly soaking with mock solution or RNA solutions at a concentration of 20 ng/μl of specific total RNAs, both supplemented with Silwett L-77 (0.02%). One hour after pretreatment, leaves were dip-inoculated with Pto DC3000 WT or Pto DC3000Δcfa6 at a concentration of 5×10⁷ cfu/ml in similar way as that of RNAs. Bacterial titers were monitored two days post-inoculation, as specified earlier. In tomato, two leaves of three plants per condition were pretreated with a suspension having 20 ng/μl of specific total RNA supplemented with Silwett L-77 (0.02%) and then were dipped one hour after with GFP-tagged Pto DC3000 at 5×10⁷ cfu/ml. The plants were then placed in controlled conditions at 24° C./19° C. (day/night) with a 16 h photoperiod without lid cover for 3 days. Bacterial infection was then analyzed by monitoring GFP signal under a UV light using an Olympus MV 10× macrozoom and pictures were taken. Individual leaf samples were collected to quantify the amount of bacteria in each condition using ImageJ software.

In Vitro Synthesis of dsRNAs and sRNAs

In vitro synthesis of RNAs was generated following the instruction of the MEGAscript® RNAi Kit (Life Technologies, Carlsbad, Calif.). Templates like were amplified by PCR introducing the T7 promotor at both 5′ and 3′ end of the sequence. PCR amplification was done in two steps with two different annealing temperature to rise the specificity of primers annealing. After the amplification step, PCR products were purified by gel extraction thanks to the NucleoSpin® Gel and PCR Clean-up kit (Macherey-Nagel) to eliminate any parasite amplification. Those purified PCR products were then used as templates for in vitro transcription: 2 μg was incubated for five hours at 37° C. with 2 μL of T7 polymerase (T7 enzyme Mix), 2 μL of 10× T7 Reaction Buffer and 2 μL of each 75 mM ATP, CTP, GTP and UTP. The total volume is adjusted to 20 μL with Nuclease free water. After the transcription reaction, dsRNAs were treated with 2 μL of DNaseI, 2 μL of RNase, 5 μL of 10× reaction buffer to eliminate DNA templates and single stranded RNAs. Then, dsRNAs are purified with the filter cartridges provided with the kit. Long dsRNA obtained at this step are used for the following experiments. siRNAs were obtained thanks to ShortCut® RNase III (NEB, Ipswich, Mass.). DsRNAs were digested for 20 minutes with RNaseIII and then purified thanks to the mirVana™ miRNA Isolation Kit (Life Technologies, Carlsbad, Calif.). After purification, siRNAs are used for the following experiments. Each steps of the process were followed by gel electrophoresis (TAE 1X, 1% agarose gel for DNA amplification and 2% agarose gel for RNAs) to check the quality of RNAs.

Bacterial Luminescence Quantification

Three plants per condition were syringe-infiltrated with Pto DC3000 Luciferase (Pto Luc) strain at 1×10⁶ cfu/ml. Plants were placed in chambers with high humidity to facilitate proper infection. Leaf discs were placed in individual wells of a 96 well plate to quantify the luminescence using Berthold Centro LB 960 Microplate Luminometer. Four leaves per plant were taken into consideration. Leaf discs from individual leaves were pooled after to perform bacterial titer quantification as mentioned above. Luminiscence quantification assay with lux-tagged PAK strain was performed in LB medium with an inoculum of 1×10⁷ cfu/ml incubated with specific RNA extracts to obtain a final concentration of 20 ng/μl in at four individual wells per condition. The 96-well plate was set on the Berthold Centro LB 960 Microplate Luminometer and the luminescence was recorded every 30 minutes for a period of 4 hours.

Tomato Infection Quantification

(a) GFP loci quantification: Tomato leaves infected with Pto DC3000-GFP strain were subjected to GFP quantification under a UV light using an Olympus MV 10× macrozoom and pictures were taken with a CCD camera AxioCam Mrc Zeiss with a GFP filter. Number of GFP loci was quantified with ImageJ software for at least 10 pictures per condition.

(b) Bacterial genomic DNA Quantification

To quantify bacterial infection in the infected tomato plants (Ross et al., 2006), the amount of bacterial genomic DNA (gDNA) was measured relative to plant gDNA. Genomic DNA was isolated from tomato leaf samples infected with Pto DC3000-GFP using the DNeasy plant mini kit (QIAGEN, Germany) according to the manufacturer's instructions. Using 1 ng of gDNA, qPCR was performed using Takyon SYBR Green Supermix (Eurogentec®) and GFP gene-specific primers. Amount of bacterial gDNA was normalized to that of tomato using Ubiquitin-specific primers.

Agrobacterium-Mediated Transient Expression of Inverted Repeats in N. benthamiana

To produce single hairpins, IR-CFA6 and IR-HRPL, and the chimeric hairpin IR-CFA6/HRPL, the A. tumefaciens strain carrying the plasmids were grown overnight in LB medium at 28° C. Cells were harvested by centrifugation and resuspended in a solution containing 10 mM MES, pH 5.6, 10 mM MgCl2 and 200 μM acetosyringone at a final density of 0.5 OD₆₀₀. Cultures were incubated in the dark at room temperature for 5-6 hours before Agrobacterium-mediated infiltration in four-week old N. benthamiana. After 3 days of infiltration, leaf tissue was harvested and Northern blot analysis was performed to confirm the production of anti Cfa6 and HrpL siRNAs. The leaf samples were then used for total RNA extraction.

In Vitro Antibacterial Gene Silencing Assay

To assess whether the bacterial transcripts Cfa6 and HrpL can be directly targeted by the dsRNA and/or the siRNAs generated by the hairpin IR-CFA6/HRPL, 2 ml culture of Pto DC3000 WT, Pto DC3000-WT-HrpL and Pto DC3000-mut-HrpL at 10⁷ cfu/ml was treated for 4 and/or 8 hours, with 20 ng/μl of specified total RNA extracted from CV or IR-CFA6/HRPL #4 transgenic plants in a six-well plate, respectively. Similarly, to quantify the silencing of bacterial genes upon treatments with in vitro synthesized siRNAs, 2 ml of Pto DC3000-GFP at 10⁷ cfu/ml was treated for 6 hours with 2 ng/μl of in vitro synthesized IR-CYP51 siRNAs or IR-CFA6/HRPL siRNAs in a six-well plate, respectively. Bacteria were collected for each condition and further processed for molecular analyses.

Apoplastic Fluid (AF) and Extracellular Vesicles (EVs) Extraction

Extraction were done as previously described (46). Sixty leaves of 5 week-old CV or IR-CFA6/HRPL plants were infiltrated with Vesicle Isolation Buffer (VIB; 20 mM MES, 2 mM 324 CaCl2), 0.01 M NaCl, pH 6.0) with a syringe without needle. Leaves were then placed inside a 20 ml needless syringe. Syringe was then placed in 50 ml Falcon and centrifuged at 900 g for 15 minutes. The apoplastic fluid (APF) was collected and centrifuged subsequently at 2,000 g and 10,000 g for 30 minutes to get rid of any cell debris and then passed through a 0.45 μm filter. The APF was further subjected to ultracentrifugation step at 40,000 g to pellet EV fraction (P40). The pellet was resuspended in 2 ml of 20 μM Tris buffer pH=7.5. The supernatant was then subjected to ultracentrifugation step at 100,000 g to pellet EV fraction (P100). The supernatant from this step was restored (SN).

Stomatal Aperture Measurements

Plants were kept under light (100 μE/m²/s) for at least 3 hours before subjecting to any treatment to assure full expansion of stomata. Intact leaf sections from three four-week-old plants were dissected and immersed in water (Mock) or bacterial suspension at a concentration of 10⁸ cfu/ml. After 3 hours of treatment, unpeeled leaf abaxial surface was observed under SP5 laser scanning confocal microscope and the pictures were taken from different regions. The stomatal aperture (width/length) was measured using ImageJ software for 30-70 stomata per condition. In case of RNA pretreatments, the leaf sections were incubated with total RNAs extracted from specified genotypes for one hour before incubation with the bacteria. When required in specified experiments, 1 μM of exogenous Coronatine (COR) (Sigma) (52) was supplemented to the bacterial suspension.

Real-Time RT-PCR Analyses

To monitor plant-encoded transcripts, total RNA was extracted from plant samples using RNeasy Plant Mini kit (Qiagen). 0.5 μg of DNA-free RNA was reverse transcribed using qScript cDNA Supermix (Quanta Biosciences). cDNA was then amplified by real time PCR reactions using Takyon SYBR Green Supermix (Eurogentec®) and transcript-specific primers. Expression was normalized to that of Ubiquitin. To monitor bacterial transcripts, total RNA was extracted from bacteria-infected plant samples or from in vitro treated bacteria as described previously. After DNAse treatment, 250 ng of total RNA was reverse transcribed using random hexamer primers and qScript Flex cDNA kit (Quanta Biosciences). cDNA was then amplified by real time PCR reactions using Takyon SYBR Green Supermix (Eurogentec®) and transcript-specific primers. Expression was normalized to that of GyrA. PCR was performed in 384-well optical reaction plates heated at 95° C. for 10 min, followed by 45 cycles of denaturation at 95° C. for 15 s, annealing at 60° C. for 20 s, and elongation at 72° C. for 40 s. A melting curve was performed at the end of the amplification by steps of 1° C. (from 95° C. to 50° C.).

Droplet-Based Microfluidic Assay for the Monitoring of In Vitro Pto DC3000-GFP or P. aeruginosa PAO Growth

Droplet-based microfluidic experiments with Pto DC3000 were performed in NYGB medium at a temperature of 28° C., while the same experiments with P. aeruginosa PAO were performed in LB medium at a temperature of 37° C. RNAi assays were prepared by pipetting directly in the 96 well plate the different solutions to obtain 200 μl final: 100 μl of medium, 20 μl of bacteria at 10⁷ cfu/ml, 20 μl of in vitro synthesized candidate siRNAs to obtain the final concentration wanted or sterile water for the control sample followed by 60 μl of medium. The 96-well plate was set on the machine for the samples to be fractioned in droplets by the Millidrop Analyzer (http://www.millidrop.com). For each well, 10 droplets of ˜500 nl each were formed and incubated inside the instrument for the 24 hours. For each droplet, measurements of biomass (and GFP fluorescence for Pto DC3000-GFP) were acquired every ˜30 minutes.

### Example 2. Arabidopsis-Encoded siRNAs Directed Against Either Endogenous Virulence Factors or Artificial Reporter Genes from Pto DC3000 Trigger their Silencing in the Context of Bacterial Infection

To test whether host-encoded small RNAs could alter bacterial gene expression, we have generated Arabidopsis stable transgenic plants that constitutively express a chimeric inverted repeat bearing sequence homology to the ECF-family sigma factor HrpL gene and the coronatine (COR) biosynthesis, Cfa6 gene, both of which encode key virulent determinants of Pto DC3000 (FIG. 1A, (53, 54)). As negative controls, we have also generated transgenic lines overexpressing an inverted repeat bearing sequence homology to three cytochrome P450 lanosterol C-14α-demethylase (CYP51) genes of the fungus F. graminearum, which was previously shown to confer full protection against this fungal phytopathogen in both Arabidopsis and barley (20,21). These stable transgenic lines are referred to as IR-CFA6/HRPL and IR-CYP51 (or CV, Control Vector plants), respectively; and do not exhibit any developmental defect (FIG. 1B), despite high accumulation of artificial siRNAs (FIG. 1C). To investigate whether artificial siRNAs directed against Cfa6 and HrpL could interfere with the expression of these virulence factors during bacterial infection, we dip-inoculated the above transgenic plants with Pto DC3000 and further monitored Cfa6 and HrpL mRNA levels by RT-qPCR analyses. While the Cfa6 mRNA levels were moderately altered in two out of three independent IR-CFA6/HRPL lines compared to Col-0 plants, the levels of HrpL transcripts were reproducibly reduced in all the three IR-CFA6/HRPL lines compared to Col-0 plants at this timepoint (FIG. 1D). By contrast, the down-regulation of Cfa6 or HrpL mRNAs was not observed in IR-CYP51- versus Col-0-infected plants (FIG. 1D), supporting a specific effect of these antibacterial RNAs in this regulatory process. Similarly, the mRNA level of the non-targeted ProC gene was unchanged in both IR-CFA6/HRPL- and IR-CYP51-infected lines compared to Col-0-infected plants (FIG. 1D). Collectively, these data indicate that the Arabidopsis-encoded IR-CFA6/HRPL inverted repeat can at least trigger sequence-specific silencing of the bacterial HrpL transcript in the context of infection.

Because the expression of HrpL and Cfa6 virulence factors is known to be regulated by various environmental cues (54, 55), we also tested whether AGS could be effective against the Photorhabdus luminescens luxCDABE operon chromosomally expressed in Pto DC3000 under the constitutive kanamycin promoter (56). This lux-tagged Pto DC3000 strain spontaneously emits luminescence because it co-expresses the luciferase catalytic components luxA and luxB genes along with the genes required for substrate production, namely luxC, luxD and luxE (57). Two independent Arabidopsis transgenic lines, IR-LuxA LuxB lines, overexpressing anti-luxA and anti-luxB siRNAs were selected and syringe-infiltrated with the lux-tagged Pto DC3000 strain (FIG. 2A/B). The levels of luxA and luxB mRNAs as well as the luminescence activity were further monitored at 24 hours post-inoculation (hpi). By doing so, we found a significant reduction in both luxA and luxB mRNA abundance as well as in luminescence activity in IR-LuxA LuxB-compared to Col-0-infected plants (FIG. 2C). By contrast, the growth of the bacterial reporter strain was unchanged in IR-LuxA LuxB lines compared to Col-0 plants in those conditions (FIG. 2D), indicating that the above effects were not due to a decreased bacterial titer in these transgenic plants. Altogether, these data indicate that AGS is effective against both endogenous stress-responsive bacterial genes and exogenous constitutive bacterial reporter genes during Pto DC3000 infection.

### Example 3. Host-Encoded siRNAs Directed Against Cfa6 and HrpL Prevent Pto DC3000-Induced Stomatal Reopening Presumably by Suppressing Coronatine Biosynthesis

Because Cfa6 and HrpL are known to regulate each other (55) and because HrpL and Cfa6 are both essential for coronatine (COR) biosynthesis (54, 55), we next investigated whether IR-CFA6/HRPL plants could be protected from COR-dependent virulence responses. For this purpose, we monitored Pto DC3000-triggered stomatal reopening at 3 hours post-inoculation (3 hpi), a phenotype that is fully dependent on COR biosynthesis and thus abolished upon inoculation with Pto DC3000 mutants that are either deleted in Cfa6 or HrpL genes (FIG. 3A, (52)). It is noteworthy that this phenotype is not dependent on type III effectors at this timepoint of infection because a normal stomatal reopening response was observed upon treatment with the Pto DC3000 hrcC mutant (FIG. 3A, (50)), which is impaired in the assembly of the type III secretion system. Significantly, we found that Pto DC3000-induced stomatal reopening was fully abolished in the three independent IR-CFA6/HRPL transgenic lines infected with the virulent Pto DC3000 strain as compared to Col-0-infected leaves (FIG. 3B), thereby mimicking the phenotype observed on Col-0 leaves inoculated with the Pto DC3000 cfa6- or hrpl-deleted strains (FIG. 3A). By contrast, a normal Pto DC3000-induced stomatal reopening was observed in IR-CYP51-infected plants (FIG. 3C), indicating that the observed effect is specific to siRNAs directed against Cfa6 and HrpL genes. Furthermore, the compromised stomatal reopening phenotype detected in IR-CFA6/HRPL-infected transgenic plants was fully rescued upon exogenous application of COR (FIG. 3B). These data provide thus pharmacological evidence that the reduced Pto DC3000 pathogenesis manifested at infected IR-CFA6/HRPL stomata is likely caused by an altered ability of the associated and/or surrounding bacterial cells to produce COR.

### Example 4. Arabidopsis Stable Transgenic Plants Expressing Small RNAs Against Key Virulence Factors from Pto DC3000 or Xanthomonas campestris pv. Campestris are Protected from Bacterial Infections

To further monitor the possible effects that anti-Cfa6 and anti-HrpL siRNAs could have on Pto DC3000 pathogenicity, we next monitored the ability of this bacterium to spread in the leaf vasculature of Arabidopsis IR-CFA6/HRPL transgenic plants. For this purpose, we scored the number of bacterial spreads occurring at three sites from the midvein of individual leaves wound-inoculated with a virulent GFP-tagged Pto DC3000 (Pto DC3000-GFP) strain. Using this quantification method, we observed an index of bacterial propagation that was significantly decreased in the three independent IR-CFA6/HRPL transgenic lines as compared to Col-0 plants (FIG. 4A). This suggests that siRNAs directed against Cfa6 and HrpL can reach xylem vessels and further dampen the virulence activity of Pto DC3000 in Arabidopsis leaf vasculature. By contrast, a normal Pto DC3000 vascular spreading was observed in the IR-CYP51 transgenic line compared to Col-0-infected leaves (FIG. 4A), arguing for a specific effect of anti-Cfa6 and anti-HrpL siRNAs in this process. Collectively, these results indicate that siRNAs directed against the pathogenicity determinants Cfa6 and HrpL can specifically restrict the spreading of Pto DC3000 in Arabidopsis leaf vasculature. An enhanced vascular disease protection effect towards the Gram-negative bacterium Xanthomonas campestris pv. campestris (Xcc) was also found in Arabidopsis transgenic plants overexpressing siRNAs against the virulence factors HrpX, HrpG and RsmA (FIG. 5, data not shown, (58-62)). This demonstrates that AGS can additionally be used to protect plants against this well-characterized vascular bacterial pathogen of Arabidopsis, which is the causal agent of black rot, one of the most devastating diseases of crucifer crops worldwide (25, 63).

We next investigated whether stable expression of siRNAs against Cfa6 and HrpL could also impact growth of Pto DC3000 in planta, a phenotype known to be dependent on both COR and on a functional type III secretion system (54). To this end, we dip-inoculated IR-CFA6/HRPL, IR-CYP51 and WT plants with Pto DC3000 and further monitored bacterial titer at 48 hpi. Using this assay, we found a significant reduction in Pto DC3000 titer in the three independent IR-CFA6/HRPL transgenic lines compared to Col-0-infected plants, and this phenotype was reminiscent to the one observed in WT plants infected with a cfa6-deleted strain (FIG. 4C). Interestingly, we additionally observed a reduced Pto DC3000-induced water soaking disease symptoms in the three independent IR-CFA6/HRPL plants compared to WT-infected plants at 24 hpi, which resemble the phenotype observed in WT leaves dip-inoculated with the cfa6 mutant strain (FIG. 4D). By contrast, the bacterial growth and water soaking disease symptoms were unaltered in IR-CYP51 transgenic plants dip-inoculated with Pto DC3000 (FIG. 4C/D), indicating that the above effects are specific to siRNAs directed against Cfa6 and HrpL genes. Altogether, these data further support a major role for anti-Cfa6 and anti-HrpL siRNAs in dampening the virulence activity of Pto DC3000 in the context of infection. They also provide compelling evidence that AGS is an effective strategy that can be used to control bacterial pathogenicity in stable transgenic plants.

### Example 5. Exogenous Delivery of Total RNAs Derived from IR-CFA6/HRPL Plants Protect WT Arabidopsis and Tomato Plants Against Pto DC3000

Environmental RNAi is a phenomenon by which (micro)organisms can uptake external RNAs from the environment, resulting in the silencing of genes containing sequence homologies to the RNA triggers (24). This RNA-based process has been initially characterized in C. elegans (30-34), and was further found to operate in other nematodes but also in insects, plants and fungi (30, 35). However, this approach has never been used against a bacterial phytopathogen that lacks a canonical eukaryotic-like RNAi machinery such as Pto DC3000. To test this possibility, we first assessed whether RNAs expressed from IR-CFA6/HRPL plants could trigger silencing of Cfa6 and HrpL genes in in vitro conditions. For this purpose, we extracted total RNAs from CV and IR-CFA6/HRPL plants, incubated them with Pto DC3000 cells, and further analyzed by RT-qPCR the levels of Cfa6 and HrpL mRNAs at 4 and 8 hours after RNA treatments. Results from these analyses revealed a reduced accumulation of both virulence factor mRNAs upon treatment with RNA extracts from IR-CFA6/HRPL plants, a molecular effect that was not observed with RNA extracts derived from CV plants (FIG. 6A). By contrast, the level of the non-targeted ProC and RpoB mRNAs remained unaltered in the same conditions (FIG. 6A). These data therefore imply that plant antibacterial RNAs are likely taken-up by Pto DC3000 cells and subsequently trigger sequence-specific silencing of Cfa6 and HrpL genes. It also suggests that exogenous application of these antibacterial RNAs could be used as a strategy to dampen Pto DC3000 pathogenesis in Col-0 plants. To test this intriguing hypothesis, we pre-treated Arabidopsis Col-0 leaf tissues with total RNA extracts from IR-CFA6 HRPL plants for one hour, subsequently challenged them with Pto DC3000 for 3 hours, and further monitored bacterial-induced stomatal reopening events. Strikingly, we found that RNA extracts from IR-CFA6/HRPL plants fully suppressed the ability of Pto DC3000 to reopen stomata (FIG. 6B), thereby mimicking the phenotype observed in infected IR-CFA6/HRPL transgenic plants (FIG. 3). We additionally investigated whether this approach could be used to control the growth of Pto DC3000 in planta. For this purpose, we first pre-treated for one hour Col-0 Arabidopsis plants with total RNA extracts from IR-CFA6/HRPL plants and further dip-inoculated them with Pto DC3000. We found that these RNA extracts triggered a decreased Pto DC3000 titer at 2 dpi (FIG. 6C), a phenotype that was comparable to the ones observed in infected IR-CFA6/HRPL transgenic plants (FIG. 4C), as well as in Col-0 plants inoculated with the PtoΔcfa6 strain (FIG. 6C). By contrast, application of total RNA extracts from CV plants did not alter growth of Pto DC3000 in the same conditions (FIG. 6C), supporting a specific effect of antibacterial RNAs in this process. To assess whether such RNA-based biocontrol approach could also be effective in cultivated plants, we repeated the same assay on tomato (Solanum lycopersicum, cultivar Moneymaker), which is the natural host of Pto DC3000. Pre-treatment of WT tomato leaves for one hour with RNA extracts from IR-CFA6 HRPL plants led to compromised Pto DC3000-induced necrotic disease symptoms and also to a reduction in bacterial content compared to leaves pre-treated with RNA extracts derived from CV plants (FIG. 6D-F). Collectively, these data provide evidence that external application of plant-derived antibacterial RNAs can trigger AGS and disease protection against Pto DC3000 in both Arabidopsis and tomato plants.

### Example 6. Small RNA Species, but not their dsRNA Precursors, are Causal for the Compromised Stomatal Reopening Phenotype Observed Upon Exogenous Application of Total RNAs Derived from the IR-CFA6/HRPL Hairpin

Next, we interrogated which RNA entities are responsible for AGS and pathogenesis reduction upon external application of antibacterial RNAs. To address this question, we first crossed the IR-CFA6/HRPL #4 reference line with the dcl2-1 dcl3-1 dcl4-2 (dcl234) triple mutant and subsequently selected F₃ plants that were homozygous for the three dcl mutations and for the IR-CFA6/HRPL transgene. Molecular characterization of these IR-CFA6/HRPL #4×dcl234 plants revealed an enhanced accumulation of IR-CFA6/HRPL inverted repeat transcripts (i.e. unprocessed dsRNAs) compared to the level detected in IR-CFA6/HRPL #4 parental line (FIG. 7A). Furthermore, this effect was associated with undetectable levels of anti-Cfa6 and anti-HrpL siRNAs (FIG. 7A). These data are thus consistent with a role of DCL2, DCL3 and DCL4 in the biogenesis of these siRNAs through the processing of the IR-CFA6/HRPL inverted repeat. We subsequently extracted total RNAs from these plants, incubated them with Pto DC3000 cells for 8 hours, and further monitored Cfa6 and HrpL mRNA levels by RT-qPCR analysis. Using this in vitro assay, we found that RNA extracts from IR-CFA6 HRPL #4×dcl234 plants were no longer able to trigger down-regulation of Cfa6 and HrpL mRNAs (FIG. 7B), despite high accumulation of artificial dsRNA precursors (FIG. 7A). By contrast, RNA extracts from the IR-CFA6/HRPL #4 parental line, which contain high levels of anti-Cfa6 and anti-HrpL siRNAs (FIG. 7A), triggered reduced accumulation of both targeted virulence factors (FIG. 7B). Moreover, while RNA extracts from IR-CFA6/HRPL #4 plants suppressed Pto DC3000-induced stomatal reopening events, we found that RNA extracts from IR-CFA6/HRPL #4×dcl234 plants were inactive in this process, such as control RNA extracts derived from Col-0 or dcl234 plants (FIG. 7C, data not shown). Collectively, these data provide compelling evidence that dsRNAs produced from the IR-CFA6/HRPL inverted repeat are neither involved in AGS nor in pathogenesis reduction. They rather suggested that small RNAs are likely the antibacterial RNA entities responsible for these molecular and physiological phenotypes. To verify this assumption, we further purified small RNA species from IR-CFA6/HRPL plant total RNAs using a glass fiber filter-based method (FIG. 7D), and subjected them to stomatal reopening assay. By doing so, we found that these small RNA species suppressed Pto DC3000-triggered stomatal reopening, to the same extent as IR-CFA6/HRPL plant total RNA extracts (FIG. 7E). By contrast, long RNA species (above 200 bp), which were not filtered through the above columns, were inactive (FIG. 7E), further supporting that antibacterial plant dsRNAs are not involved in this response. Altogether, these data provide solid evidence that DCL-dependent siRNAs produced from the inverted repeat IR-CFA6/HRPL are critical for AGS and pathogenesis reduction, while cognate dsRNA precursors are ineffective for both processes.

### Example 7. A Bacterially Expressed Small RNA Resilient Version of HrpL is Insensitive to siRNA-Directed Silencing and Exhibits a Normal Stomatal Reopening Phenotype Indicating that Anti-HrpL siRNAs are Causal for AGS and Pathogenesis Reduction

Although the above findings indicate that external application of antibacterial siRNAs can trigger AGS and antibacterial activity, they do not firmly demonstrate that these RNA entities are causal for these phenomena. To address this issue, we decided to generate and characterize recombinant bacteria expressing a siRNA-resilient version of the HrpL gene, which was found to be subjected to AGS regulation in both in vitro and in planta conditions (FIGS. 1 and 6). To this end, we complemented the PtoΔhrpL mutant with either a WT HrpL transgene or a mutated version, mut HrpL that contains as many silent mutations as possible in the siRNA targeted region, which are predicted to alter the binding of siRNAs with the HrpL mRNA but to produce the same protein sequence. Furthermore, to assess the post-transcriptional regulatory control that anti-HrpL siRNAs might exert over these bacterial transgenes, we expressed them under the constitutive neomycin phosphotransferase II (NPTII) promoter. The two resulting recombinant bacteria are referred to as PtoΔhrpL WT HrpL and PtoΔhrpL mut HrpL, respectively, and were found to restored ability to reopen stomata when inoculated on Col-0 plants (FIG. 8A, data not shown), indicating that both transgenes are functional. We further assessed the sensitivity of each recombinant bacterium to AGS. For this purpose, we incubated PtoΔhrpL WT HrpL and PtoΔhrpL mut HrpL strains with total RNA extracts from CV and IR-CFA6 HRPL #4 plants for 8 hours and further monitored HrpL transgene mRNA levels by RT-qPCR analysis. We found a significant decrease in the accumulation of HrpL mRNAs expressed from the PtoΔhrpL WT HrpL strain, which was not detected upon treatment with control RNA extracts from CV plants (FIG. 8B). These data indicate that the WT HrpL transgene expressed from the PtoΔhrpL WT HrpL strain is fully sensitive to AGS despite its constitutive expression driven by the NPTII promoter. By contrast, the accumulation of HrpL mRNAs expressed from the PtoΔhrpL mut HrpL strain was unaltered in response to RNA extracts from IR-CFA6 HRPL #4 plants (FIG. 8B), indicating that siRNAs no longer exert their AGS effect towards this recombinant bacterium. Collectively, these findings demonstrate that anti-HrpL siRNAs are causal for the post-transcriptional silencing of the HrpL virulence factor gene within Pto DC3000 cells. Next, we investigated the responsiveness of each recombinant bacterial strain to siRNA-directed pathogenesis reduction by exploiting the Pto DC3000-induced stomatal reopening assay, which is highly sensitive to small RNA action. To assess the specific effect of siRNAs towards suppression of HrpL-mediated stomatal reopening function, we first cloned an IR-HRPL inverted repeat targeting the same HrpL sequence region than the one targeted by the IR-CFA6 HRPL hairpin, and further validated its capacity to produce HrpL siRNAs upon Agrobacterium-mediated transient transformation in Nicotiana benthamiana leaves (FIG. 8C). N. benthamiana total RNA extracts containing anti-HrpL siRNAs were found to fully suppress the ability of Pto DC3000 to reopen stomata (FIG. 8D). Importantly, similar results were obtained when N. benthamiana RNA extracts containing anti-HrpL siRNAs were incubated with the PtoΔhrpL WT HrpL strain (FIG. 8D), supporting a sensitivity of this bacterial strain to siRNA action. By contrast, the PtoΔhrpL mut HrpL strain was fully competent in reopening stomata in the same conditions (FIG. 8D), indicating that anti-HrpL siRNAs no longer exert their antibacterial effects towards this recombinant bacterial strain. These data provide thus evidence that anti-HrpL siRNAs are causal for the suppression of HrpL-mediated stomatal reopening function. They also further validate a novel role of HrpL in bacterial-induced stomatal reopening, indicating that AGS can be employed as a tool to characterize bacterial gene function.

### Example 8. The Apoplastic Fluid of IR-CFA6/HRPL Plants is Composed of Functional Antibacterial siRNAs that are Either Embedded into EVs, and Protected from Micrococcal Nuclease Action, or in a Free Form, and Sensitive to Micrococcal Nuclease Digestion

The results from the phenotypical analyses described in EXAMPLES 3 and 4 imply that small RNA species that are constitutively expressed in IR-CFA6/HRPL transgenic lines, must be externalized from plant cells towards the leaf surface, the apoplastic environment and xylem vessels in order to reach epiphytic and endophytic bacterial populations. To get some insights into the small RNA trafficking mechanisms that could be implicated in this phenomenon, we have first extracted the apoplastic fluid (APF) from IR-CFA6/HRPL plants and tested its ability to dampen bacterial pathogenesis by monitoring its impact on Pto DC3000-induced stomatal reopening. We found that this extracellular fluid triggered a full suppression of stomatal reopening during infection, thereby mimicking the effect triggered by IR-CFA6/HRPL-derived total RNAs (FIG. 9A). By contrast, the APF from IR-CYP51 plants was inactive, supporting a specific effect of anti-Cfa6 and anti-HrpL siRNAs from the AFP of IR-CFA6/HRPL plants in this process (FIG. 9A). We further tested whether EVs from IR-CFA6/HRPL plants could contribute to AGS. For this end, we recovered APF from IR-CFA6/HRPL plants and further performed differential ultracentrifugation at 40,000 g or 40,000 g followed by 100,000 g, which allowed us to collect two fractions, named P40 and P100, respectively. Interestingly, we found that both fractions were capable of suppressing stomatal reopening, although P100 was moderately less effective in this process (FIG. 9B). Importantly, both fractions remained active in the presence of micrococcal nuclease (Mnase), indicating that small RNAs are protected from external degradation when embedded into EVs. Intriguingly, we also noticed that the supernatant fraction (SN), recovered after the sequential centrifugation at 40,000 g and 100,000 g, exhibited strong antibacterial activity, despite a lack of canonical EVs detected in this fraction (FIG. 9B, data not shown). This suggests that EV-free small RNAs that are either associated with proteins and/or in a free-form could additionally be competent for AGS. To determine which of the two small RNA entities could possess such antibacterial activity, we treated SN fractions from IR-CFA6/HRPL plants with Mnase or proteinase K and further subjected them to stomatal reopening assay. Interestingly, we found that the Mnase treatment abrogated the antibacterial effect triggered by the IR-CFA6 HRPL-derived SN fraction, while an unaltered antibacterial activity was detected in the presence of proteinase K, which globally degraded proteins (FIG. 9B, data not shown). Collectively, these data indicate that functional EV-free antibacterial small RNAs are unlikely associated with proteins and are thus referred to here as Extracellular Free Small RNAs or “efsRNAs”. Our results also indicate that efsRNAs are sensitive to Mnase action because they lost their antibacterial effect upon treatment with this nuclease (FIG. 9B). Based on these findings, we propose that the APF from IR-CFA6/HRPL plants is composed of at least three populations of functional antibacterial small RNAs, which are 1) embedded into large EVs (P40 fraction), 2) embedded into EVs of smaller size (P100 fraction), or 3) in a free form.

### Example 9. The In Vitro Synthesis of Small RNAs is an Easy, Rapid and Reliable Approach to Screen for Candidate Small RNAs Possessing Antibacterial Activities

In order to develop a screening platform for the identification of candidate small RNAs with antibacterial activities, we aimed to produce in vitro synthesized siRNAs against specific bacterial gene transcripts and further test their activities on bacterial pathogenicity or survival. For this end, we first decided to generate in vitro synthesized anti-Cfa6 and anti-HrpL siRNAs targeting the same sequences than the plant siRNAs produced from the DCL-dependent processing of IR-CFA6/HRPL. To do so, we used primers carrying T7 promoter sequences to amplify either CYP51 or CFA6 HRPL DNA from plasmids containing the IR-CYP51 or IR-CFA6/HRPL sequences. The resulting PCR products were gel-purified and subsequently used as templates for in vitro RNA transcription using a T7 RNA polymerase, which led to the production of CYP51 or CFA6HRPL dsRNAs of expected size (FIG. 10A). Small RNAs were further obtained by digesting these dsRNAs into 18-25 bp siRNAs using the ShortCut® RNase III, although other non-commercial RNase III can also be used for this process (data not shown). As revealed by agarose gel electrophoresis, these siRNAs were deprived of dsRNA (FIG. 10A), indicating that the RNase III used in these experiments fully processed the initial pool of dsRNA molecules. We next analyzed the ability of synthetic dsRNA and siRNAs to suppress stomatal reopening. Consistent with our previous data showing that plant dsRNAs are inactive in triggering AGS (FIG. 7), we found that in vitro synthesized CFA6/HRPL dsRNAs did not interfere with Pto DC3000-induced stomatal reopening, nor did in vitro synthesized CYP51 dsRNAs, which were used as negative controls (FIG. 10B). By contrast, in vitro synthesized siRNAs directed against Cfa6 and HrpL fully prevented Pto DC3000-induced stomatal reopening, while in vitro synthesized anti-CYP51 siRNAs were inactive in this process (FIG. 10B). The latter result suggested that in vitro synthesized anti-Cfa6 and anti-HrpL siRNAs were likely capable of triggering silencing of Cfa6 and HrpL genes. To test this hypothesis, we further incubated the in vitro synthesized CYP51 and CFA6/HRPL siRNAs at a concentration of 2 ng/ul with 1×10⁷ cfu/ml of Pto DC3000 for 6 hours and further monitored Cfa6 and HrpL mRNAs by RT-qPCR analyses. By doing so, we found that anti-Cfa6HrpL siRNAs triggered a significant reduced accumulation of Cfa6 and HrpL mRNAs compared to anti-CYP51 siRNAs (FIG. 10B), a molecular effect which was comparable to the one observed in response to plant-derived total RNAs containing anti-Cfa6 and anti-HrpL siRNAs (FIG. 6A, 7B). By contrast, the levels of the non-targeted ProC and RpoB mRNAs remained unchanged in response to anti-Cfa6 and anti-HrpL siRNAs compared to anti-CYP51 siRNAs (FIG. 10B). Collectively, these data indicate that in vitro synthesized siRNAs can trigger AGS and antibacterial activity to the same extent as plant-derived anti-Cfa6 and anti-HrpL siRNAs.

We next decided to determine whether this approach could be instrumental for the identification of candidate siRNAs with bactericidal activities. To test this idea, we performed in vitro synthesis of siRNAs directed against three conserved and housekeeping genes from Pto DC3000, namely SecE (PSPTO_0613, preprotein translocase SecE subunit), FusA (PSPTO_0623, translation elongation factor G) and GyrB (PSPTO_0004, DNA gyrase subunit B) and further monitor their impact on the in vitro growth of this bacterium. To do so, we took advantage of an established droplet-based microfluidic system, which is suitable for the accurate measurements of bacterial biomass and bacterially-expressed fluorescence reporter activity. By using this approach, we found that 0.33 ng/μl of in vitro synthesized siRNAs directed against FusA was capable of reducing both the biomass and the GFP signal from a GFP-tagged Pto DC3000 (Pto DC3000-GFP), compared to the conditions in the absence of siRNAs or in the presence of anti-SecE siRNAs (FIG. 10E, F). Strikingly, we did not detect any GFP signal nor bacterial biomass when the Pto DC3000-GFP strain was incubated with 1 ng/ul of in vitro synthesized anti-FusA siRNAs, nor when in vitro synthesized siRNAs directed against GyrB were applied at concentrations of either 0.33 ng/μl or 1 ng/μl (FIG. 10E, F). These data indicate that siRNAs directed against FusA and GyrB possess a potent bactericidal activity that mimics the effect that would be detected in the presence of an antibiotic. Based on these proof-of-concept experiments, we conclude that the in vitro synthesis of siRNAs is an easy, rapid and reliable approach to screen for novel candidate small RNAs with antibacterial activities. They also unveil a role for FusA and GyrB in the survival of Pto DC3000, which has not previously been reported for this bacterium. These results therefore further support the fact that AGS can be employed as a tool to characterize bacterial gene function.

### Example 10. Plant Small RNAs and In Vitro Synthesized Small RNAs can Trigger AGS in Pseudomonas aeruginosa, and this Regulatory Process can be Exploited to Reduce the Growth of this Bacterium by Targeting Some of its Essential Genes

The above findings, along with the fact that long dsRNAs expressed from mammalian cells are known to trigger potent antiviral interferon response (37), which is not the case in plant cells, prompted us to further assess whether plants could be employed to produce small RNAs against animal pathogenic bacteria. For this end, we have first transiently expressed the inverted repeat IR-LuxA LuxB construct described in the EXAMPLE 2 in N. benthamiana leaves using Agrobacterium-mediated transformation. As a negative control, we have also transiently expressed in N. benthamiana leaves an inverted repeat carrying sequence homologies with the GFP reporter gene. Total RNAs, containing either anti-LuxA B siRNAs or anti-GFP siRNAs, were incubated with a previously described Pseudomonas aeruginosa (PAK) strain expressing a lux reporter system (64), and the bioluminescence activity was further monitored in in vitro conditions on a microplate reader. Using this approach, we detected a specific decrease in bioluminescence activity in the presence of anti-LuxA and anti-LuxB plant siRNAs, which was not observed with anti-GFP siRNAs (FIG. 11A). By contrast, the growth of the lux-tagged PAK strain was unchanged in the presence of either small RNA species, as revealed by the absorbance measurement at 600 nm (OD₆₀₀) (FIG. 111B). This result indicates that the above detected effect was not due to a decreased bacterial titer in the presence of anti-LuxA and anti-LuxB siRNAs. It rather indicates that plant-derived siRNAs directed against the luxA and luxB reporter genes can trigger AGS in the lux-tagged PAK strain.

To further determine whether AGS could additionally be detected against PAK endogenous genes, we have further generated chimeric inverted repeats designed to concomitantly target DnaA, DnaN and GyrB genes, or RpoC, SecE and SodB genes. It is noteworthy that these P. aeruginosa targets were chosen because their individual deletion was known to alter the survival of this bacterium (38-40). Both inverted repeat constructs were found to overexpress small RNAs against these bacterial genes upon Agrobacterium-mediated transformation in N. benthamiana leaves (data not shown). Interestingly, when 20 ng/ul of each total RNA extracts were incubated with the lux-tagged PAK strain, we found a decrease in bioluminescence activity compared to total RNAs extracts derived from non-transformed N. benthamiana leaves (FIG. 11C). Furthermore, these phenotypes were also associated with a decrease in the growth of the lux-tagged PAK strain, as revealed by a reduction in the absorbance at 600 nm (OD₆₀₀) (FIG. 11D). By contrast, RNA extracts containing anti-GFP siRNAs did not alter bioluminescence activity nor bacterial titer in the same conditions (FIG. 11C/D). These results indicate that plant artificial siRNAs concomitantly targeting essential genes from the PAK strain are effective in triggering AGS and bacterial growth reduction in in vitro conditions.

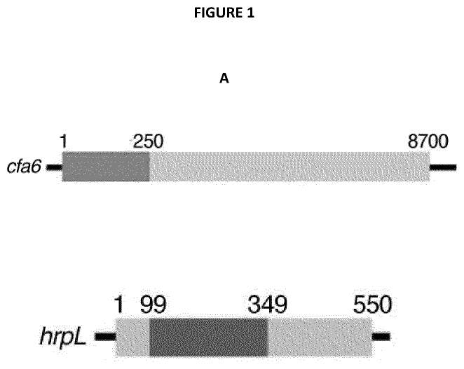

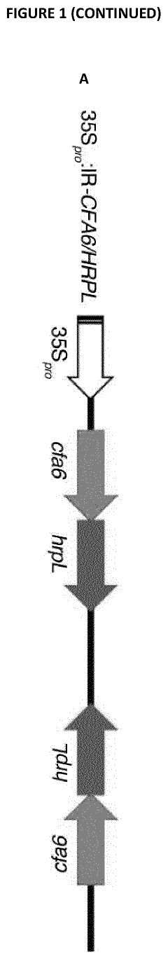

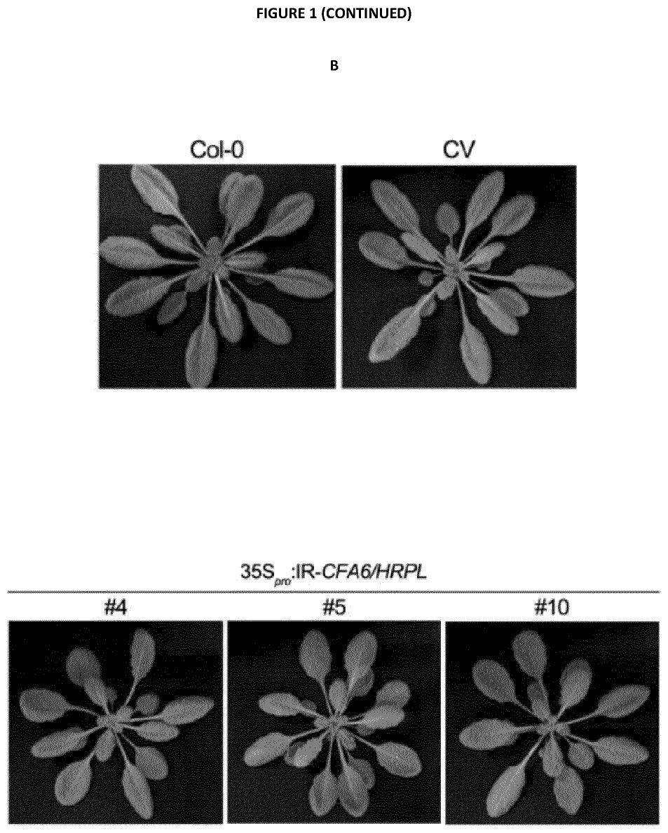

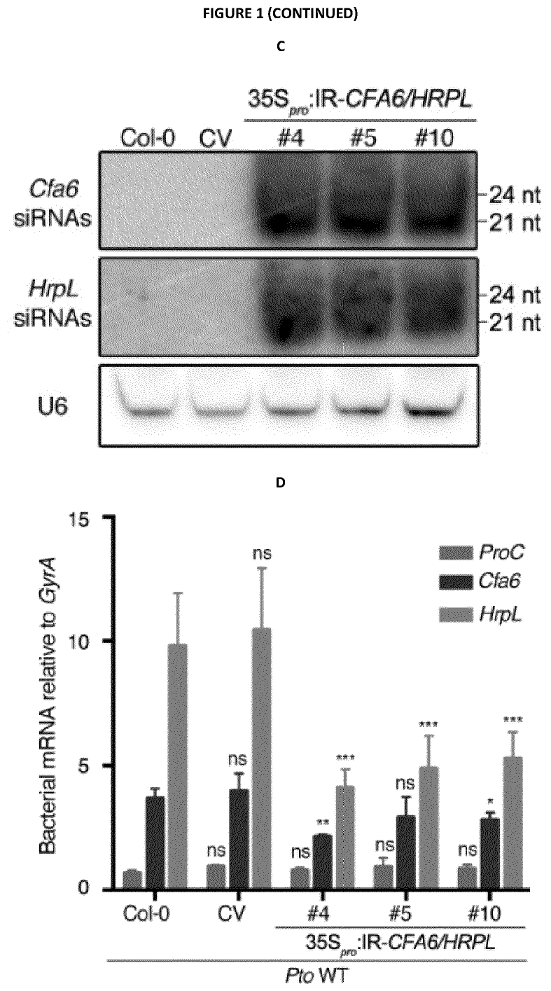

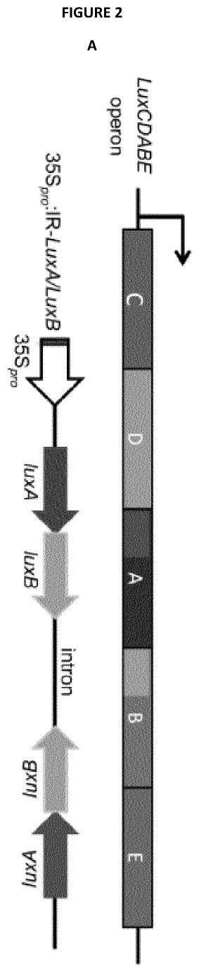

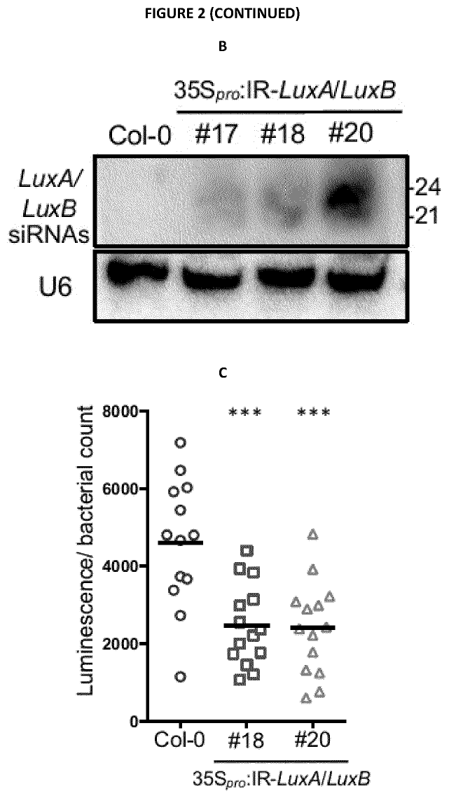

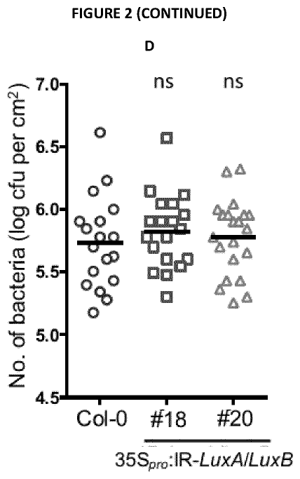

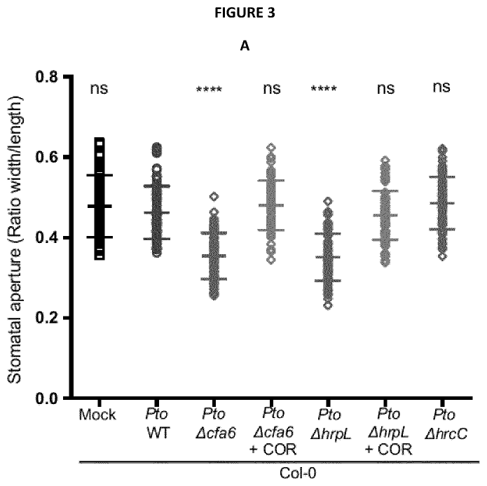

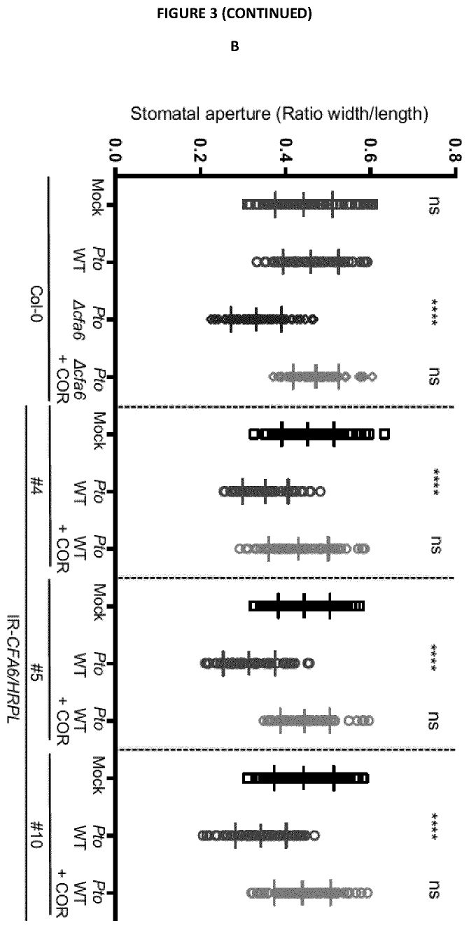

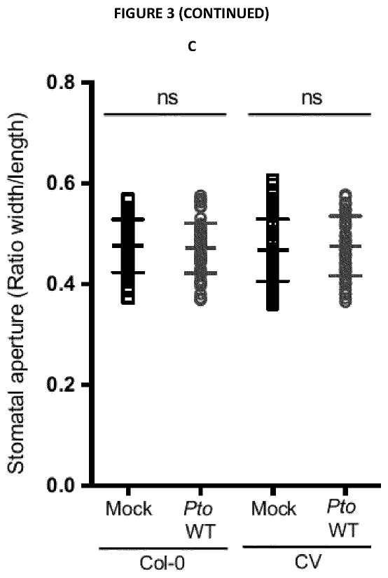

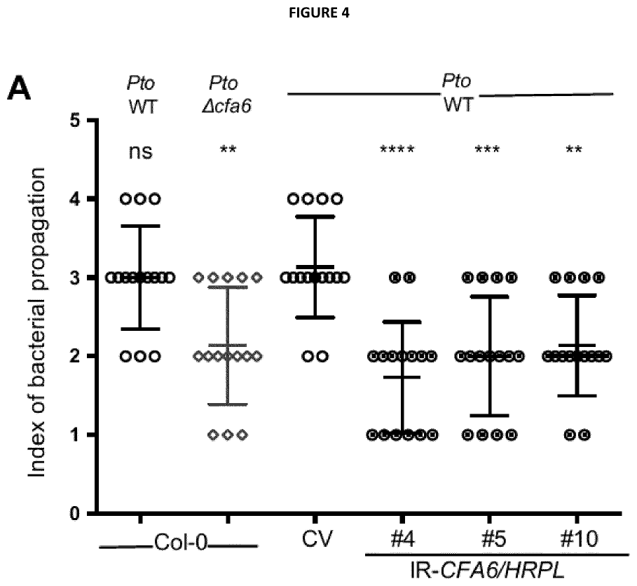

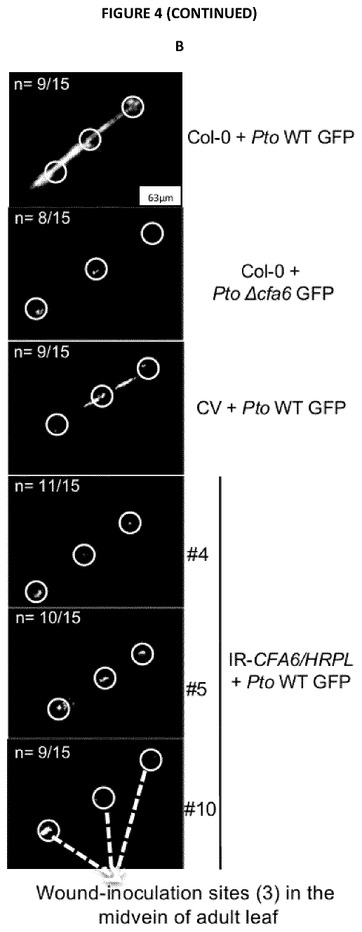

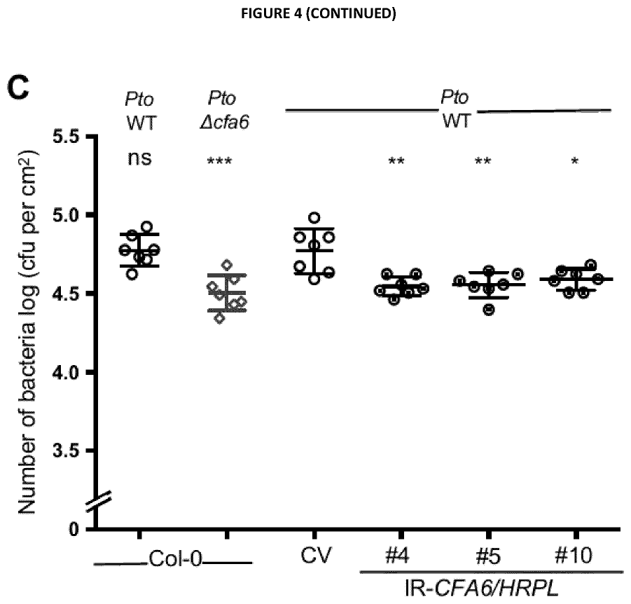

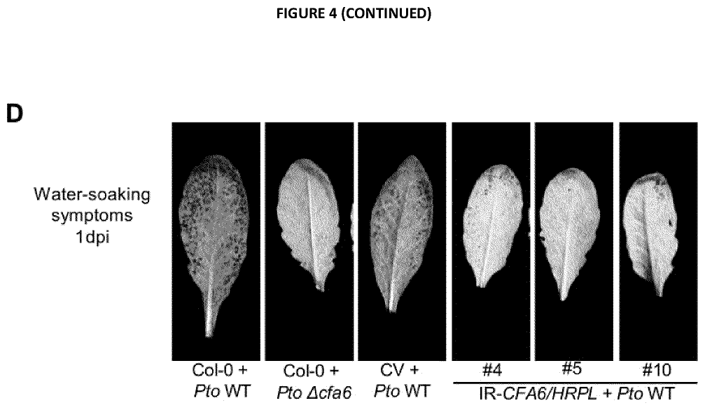

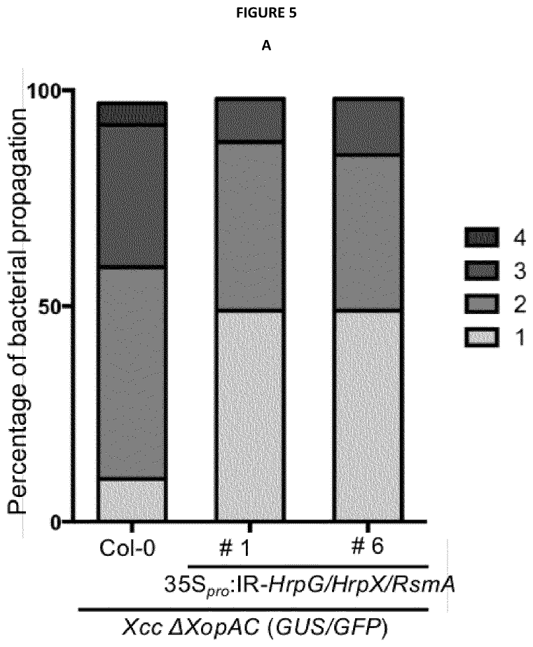

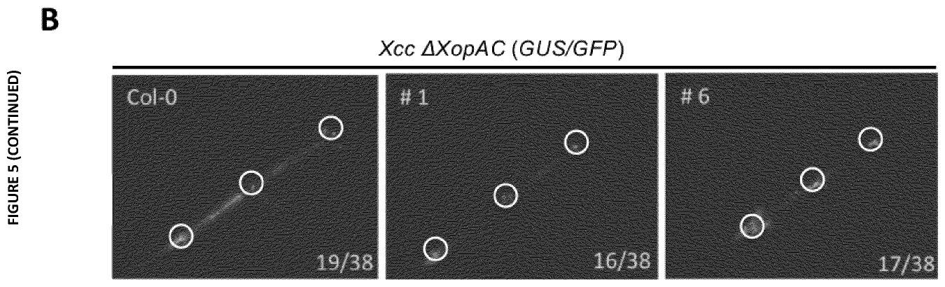

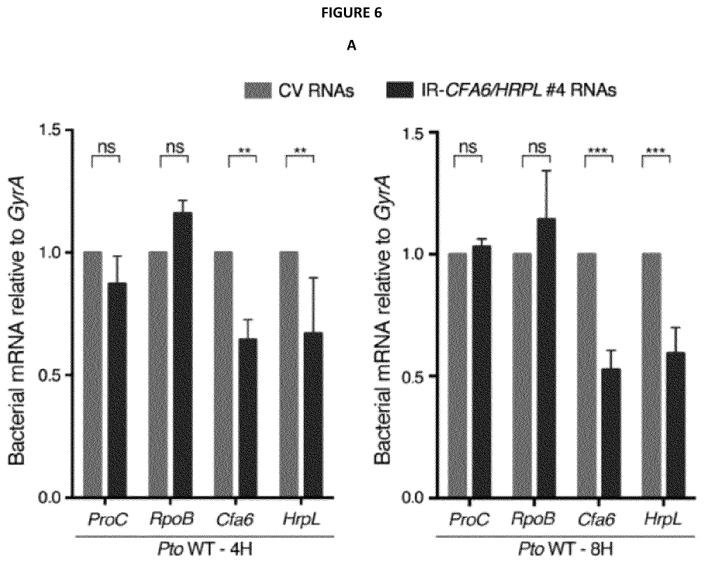

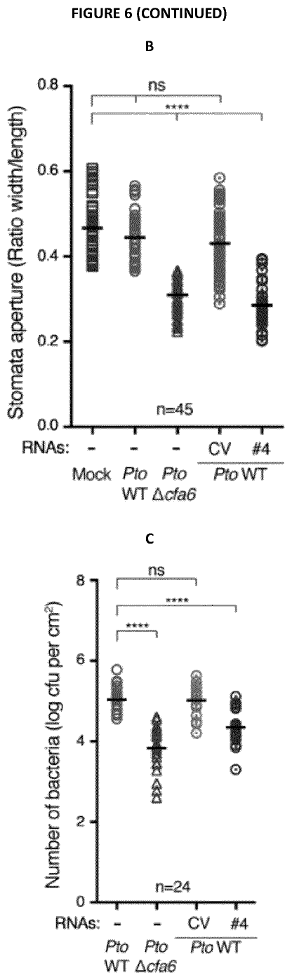

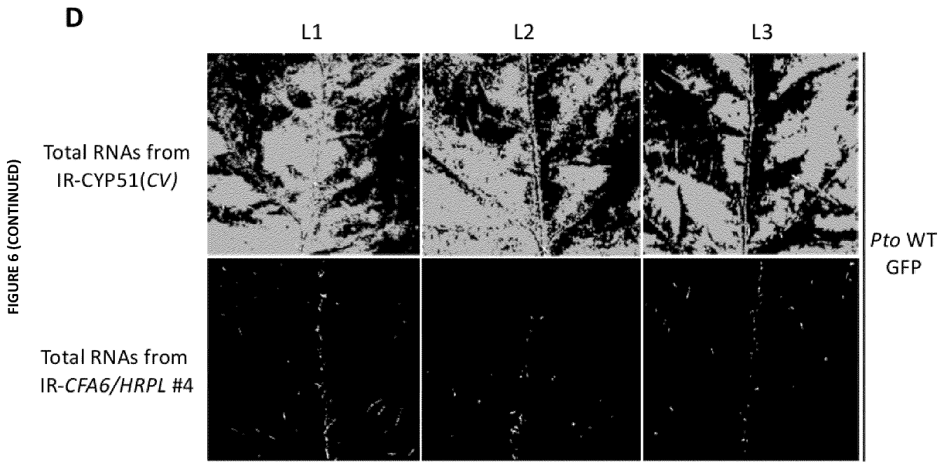

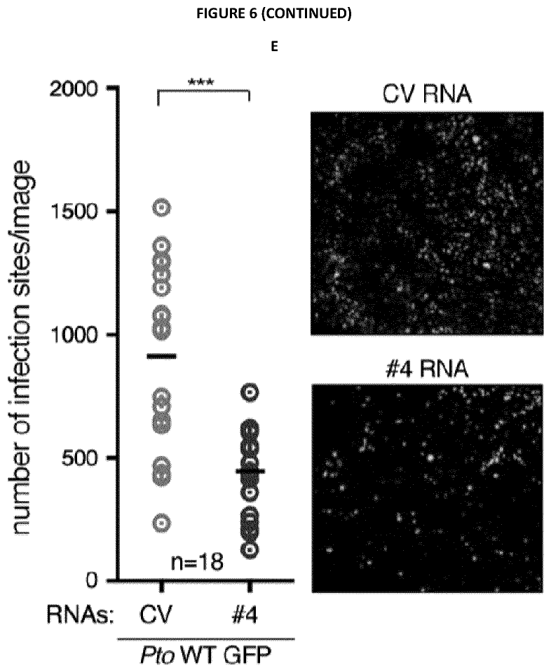

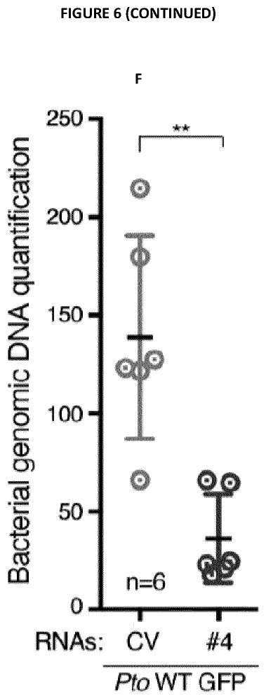

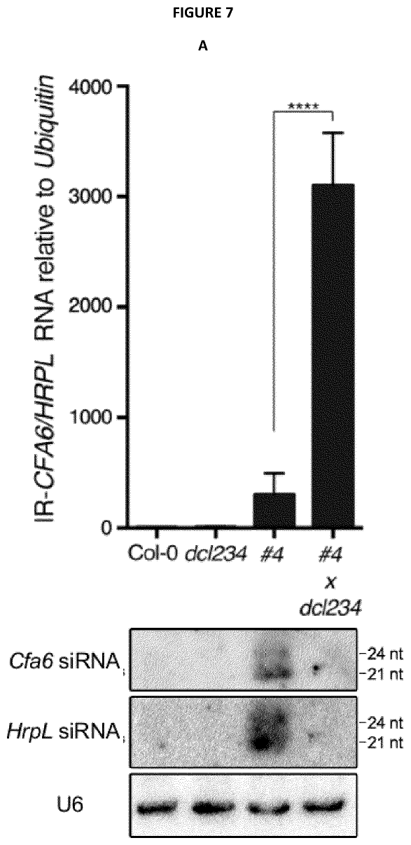

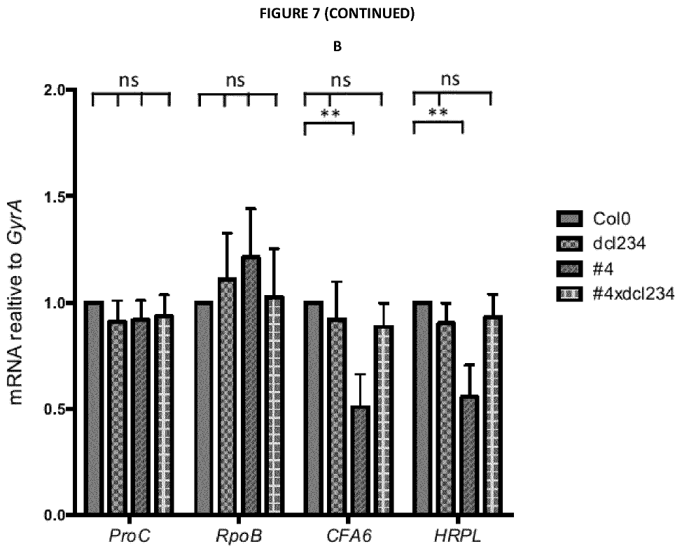

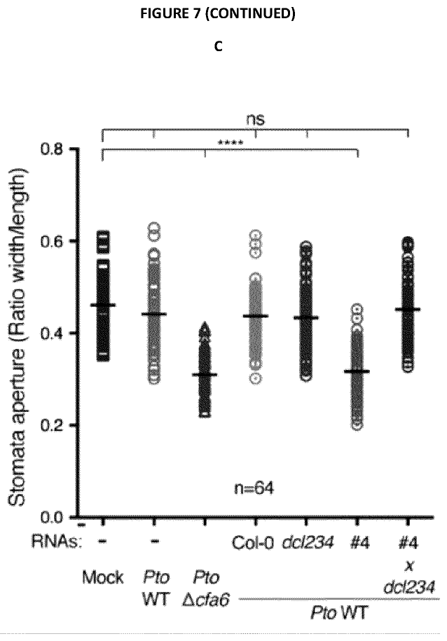

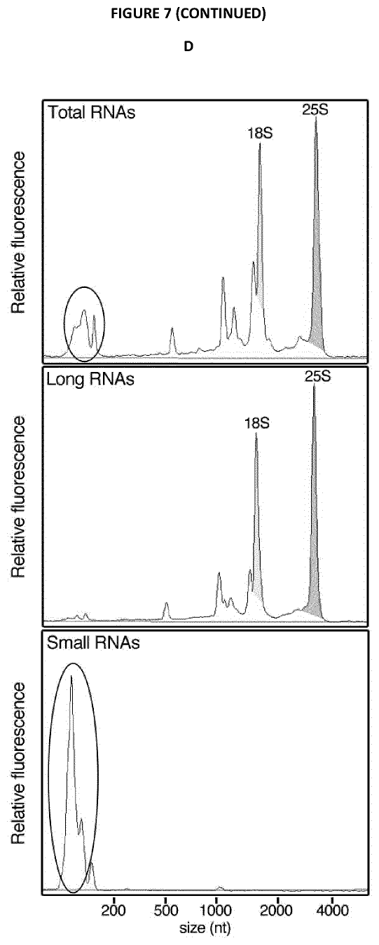

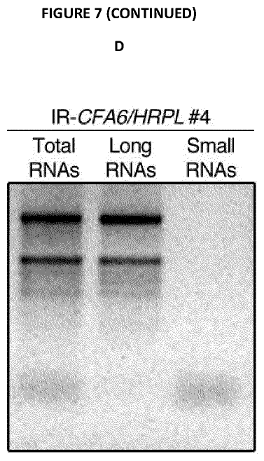

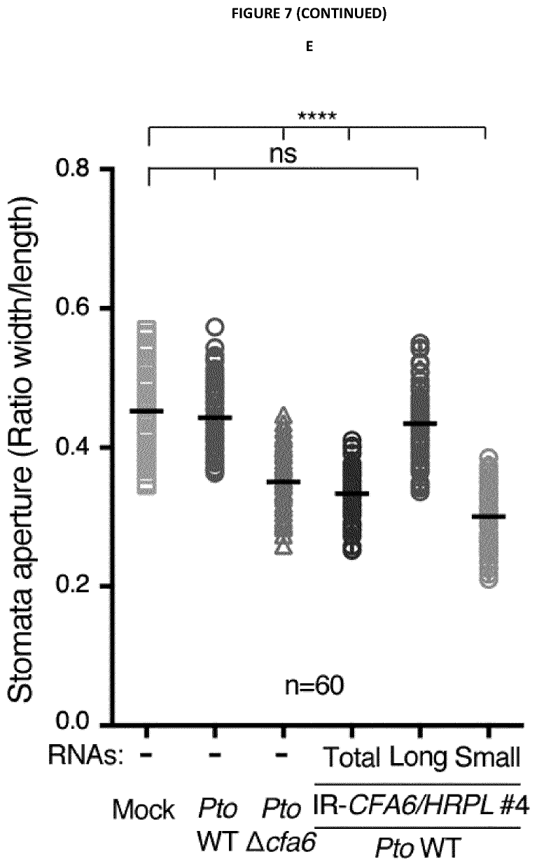

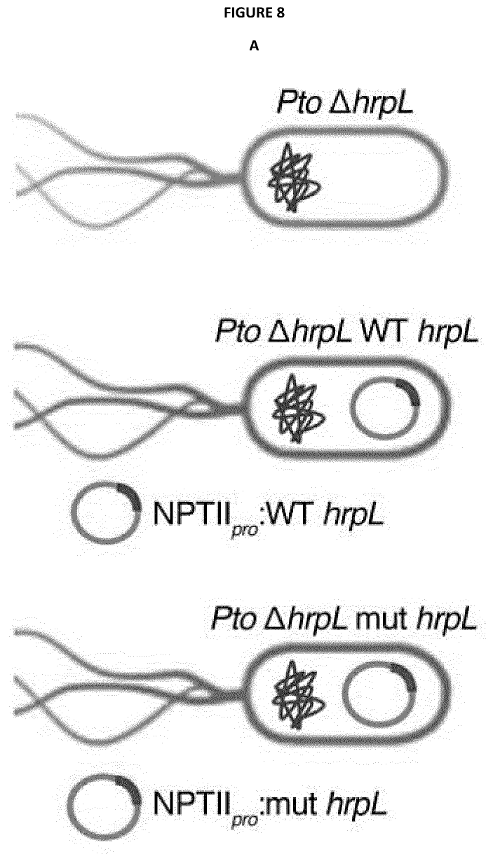

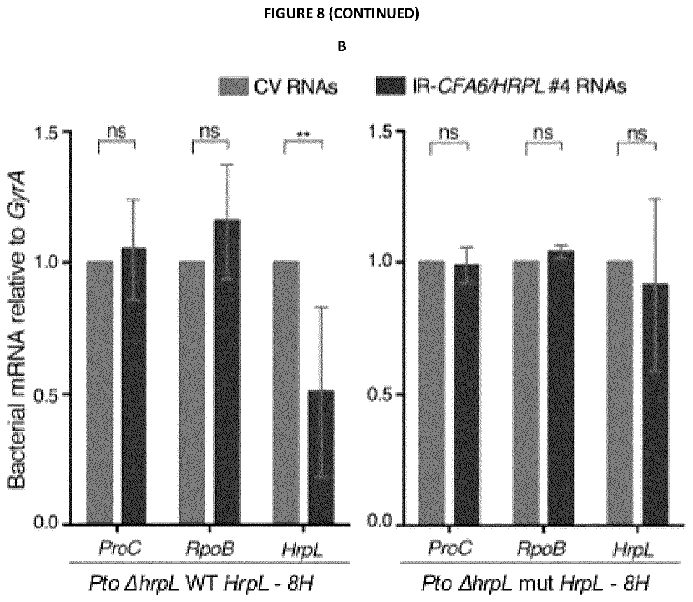

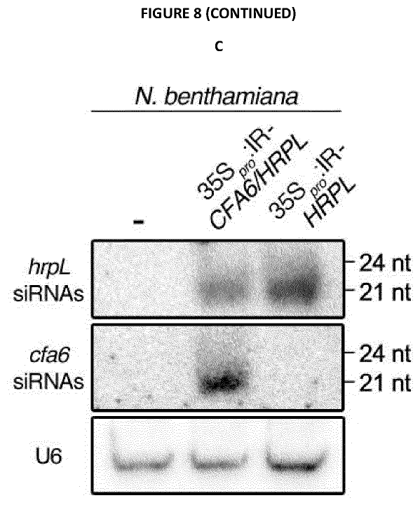

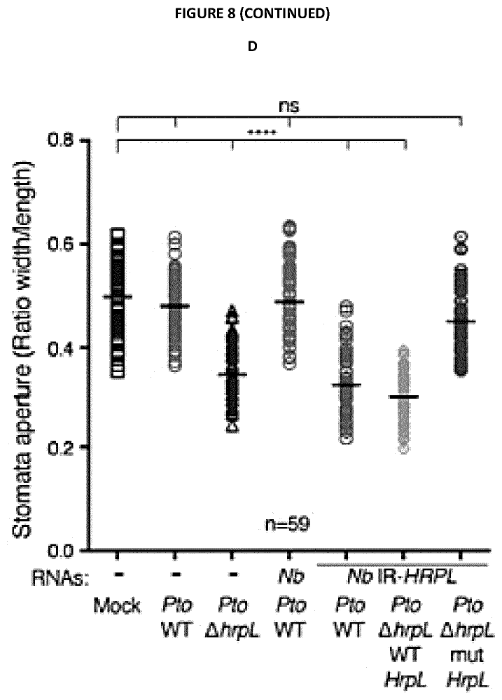

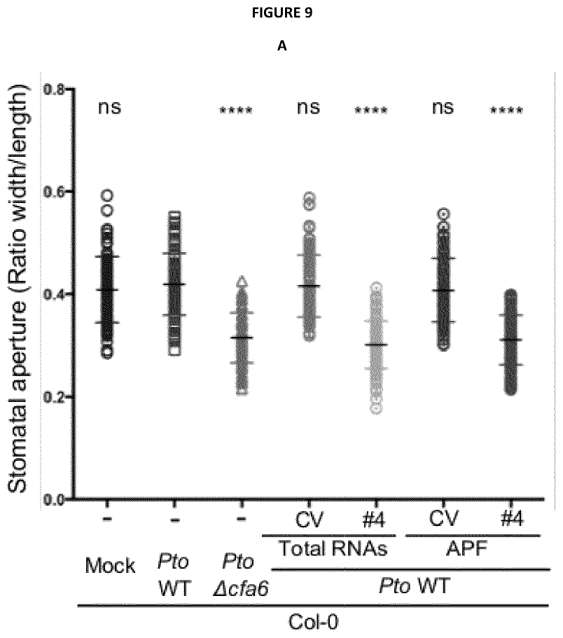

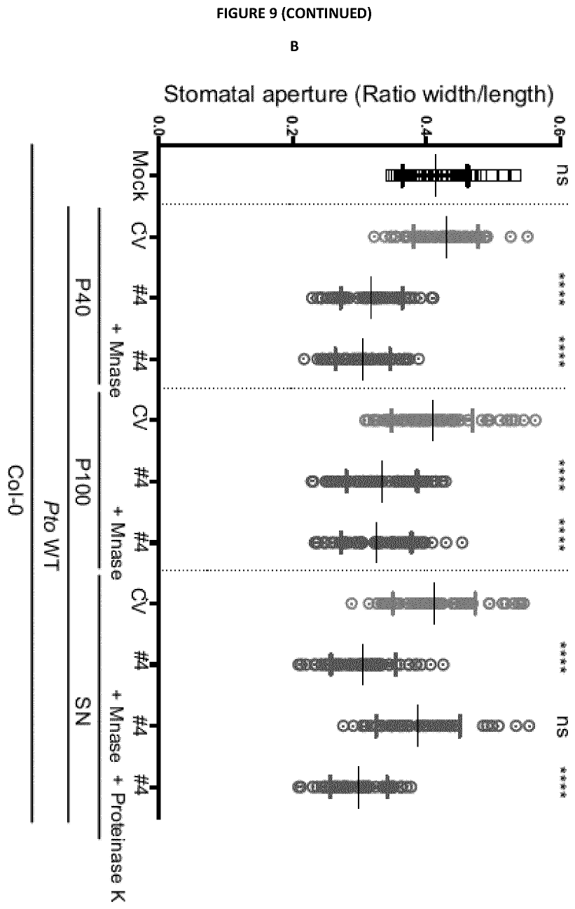

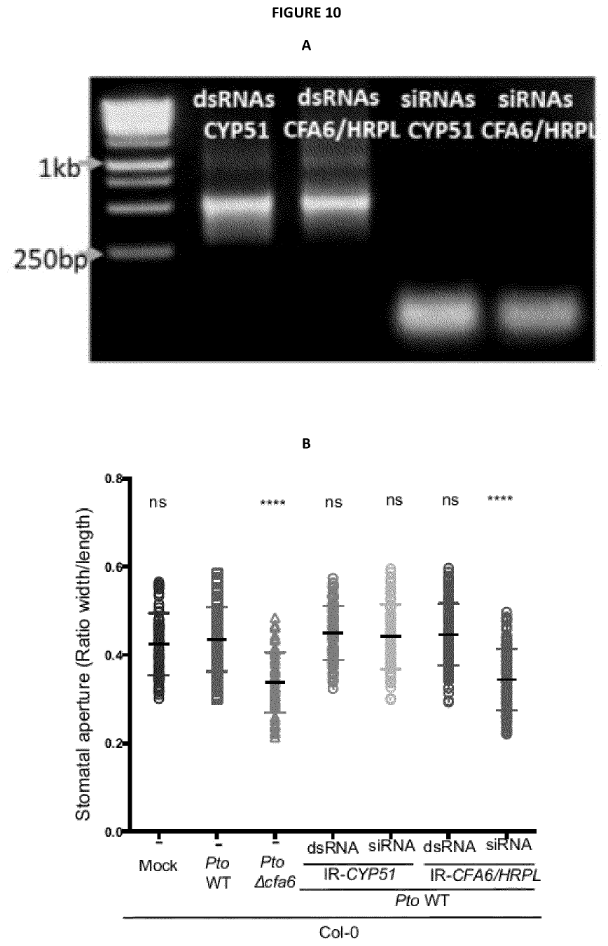

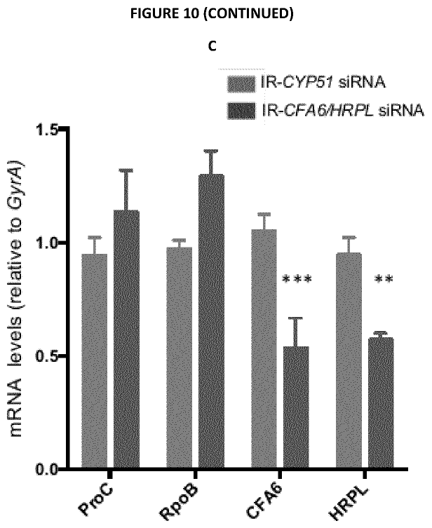

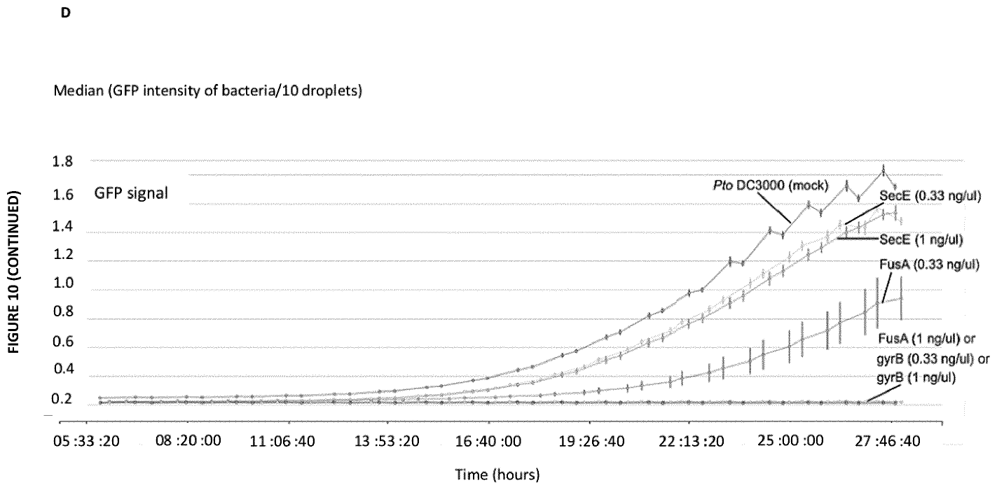

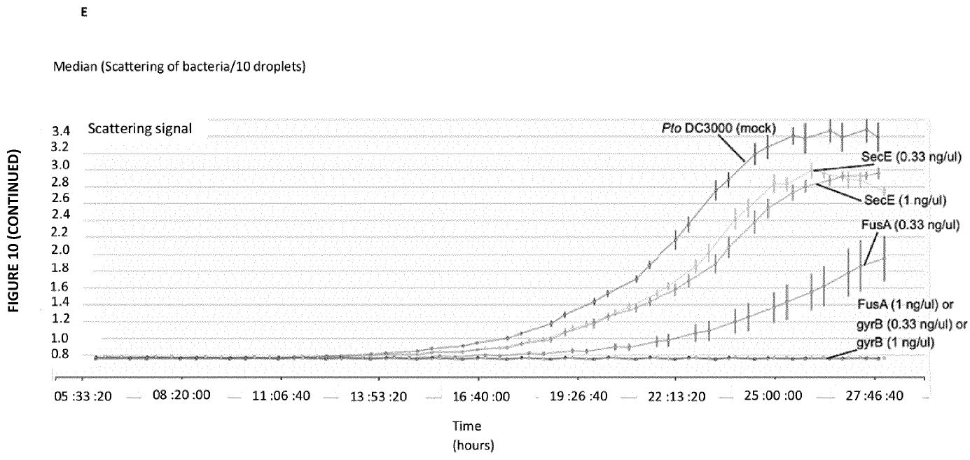

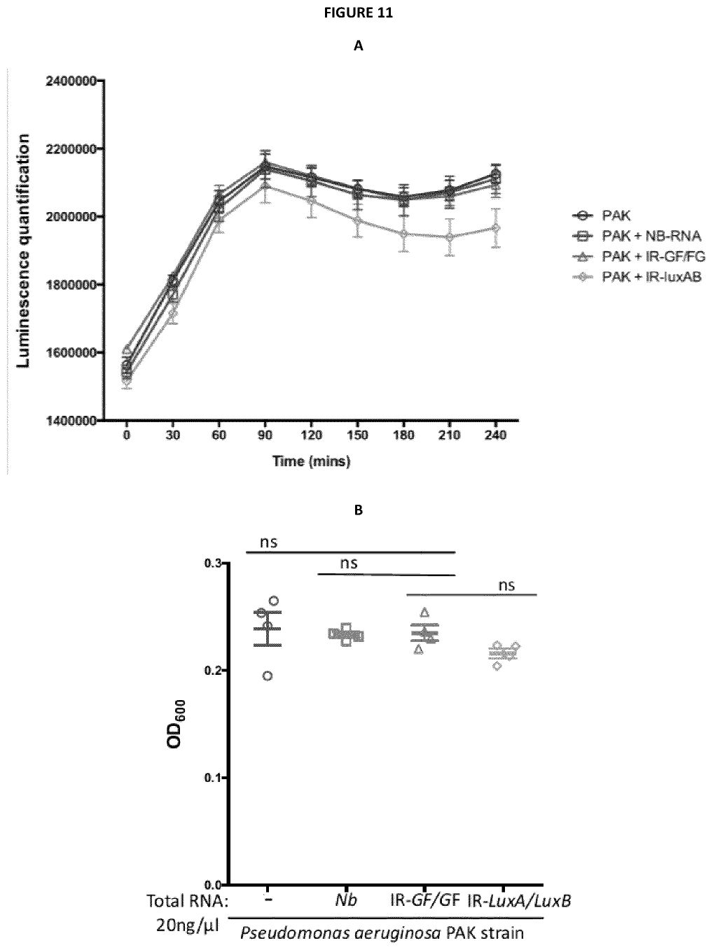

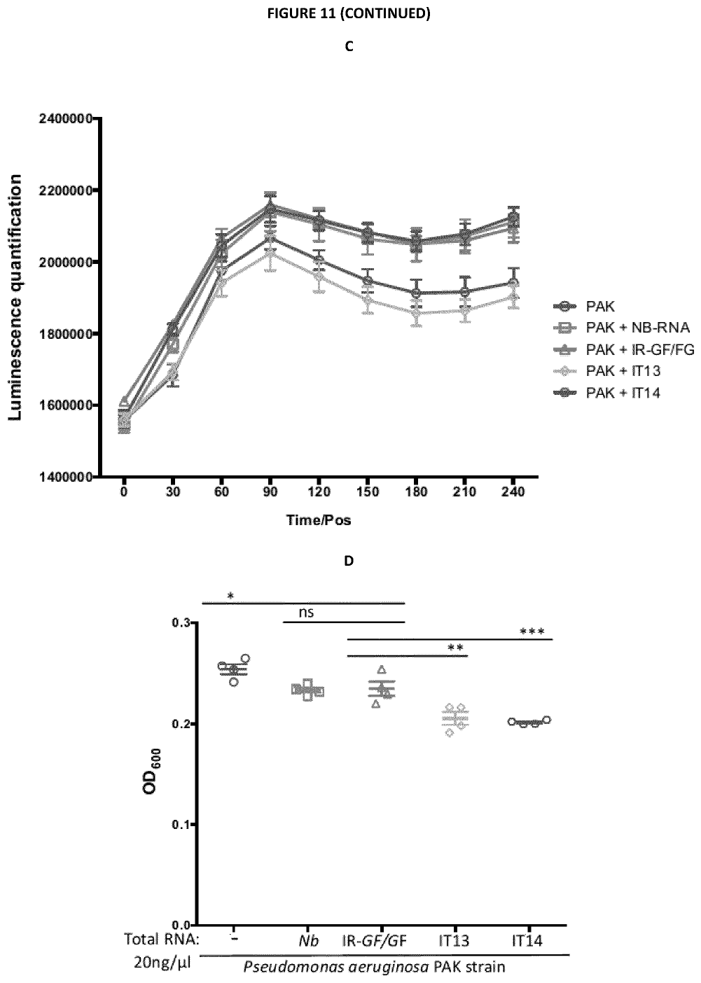

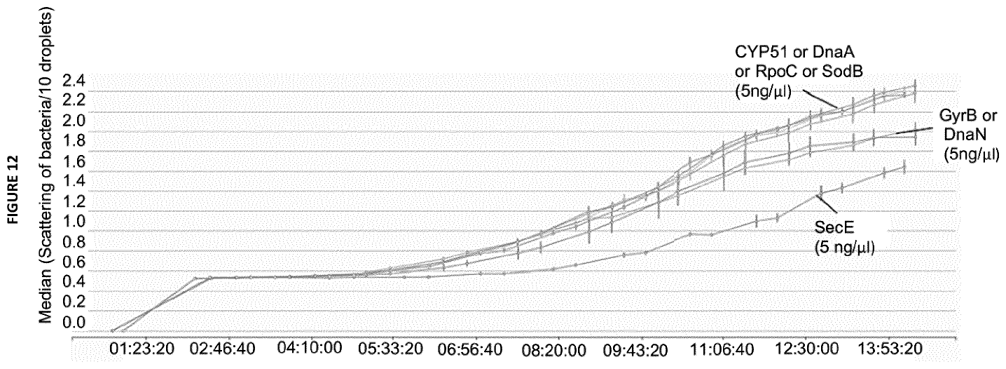

Finally, we investigated whether in vitro synthesized siRNAs could also be active in these prokaryotic cells, as observed in the phytopathogenic bacterium Pto DC3000 (EXAMPLE 10, FIG. 10). To test this hypothesis, we performed in vitro synthesis of siRNAs directed against either DnaA, DnaN, GyrB, RpoC, SecE or SodB. As negative control, we also synthesized siRNAs targeting the Fusarium CYP51 genes described in EXAMPLE 2. These siRNAs were incubated with the P. aeruginosa PAO strain at a concentration of 5 ng/ul and the growth of this bacterium was further analyzed using a droplet-based microfluidic system. Results from these analyses revealed that in vitro synthesized siRNAs directed against DnaA, RpoC or SodB genes did not alter the in vitro growth of the P. aeruginosa PAO strain compared to the control anti-CYP51 siRNAs (FIG. 12). By contrast, siRNAs directed against GyrB, DnaN or SecE genes triggered a decrease in the growth of this bacterium compared to anti-CYP51 siRNAs, with a stronger growth reduction effect being detected with anti-SecE siRNAs (FIG. 12). Based on these results, we conclude that the use of specific in vitro synthesized siRNAs can not only be effective in a phytopathogenic bacterial strain (EXAMPLE 9), but also in a typical Gram-negative human bacterial pathogen (EXAMPLE 10). This supports the idea that unrelated bacterial cells can uptake passively or activity external small RNAs, and subsequently trigger sequence-specific silencing of the targeted bacterial genes. These results also show that the in vitro synthesis of siRNAs, coupled with a screening system such as the droplet-based microfluid device used in this EXAMPLE, is a powerful approach to screen in an easy, rapid and reliable manner for small RNAs with antibacterial activities against animal bacterial pathogens.

### Example 11. In Planta Production of EV-Embedded siRNAs Directed Against Essential or Virulence Genes from Pseudomonas aeruginosa, Shigella Flexneri and Staphylococcus aureus

To produce plant EV-embedded small RNAs that might be ultimately used as RNAi-based prophylactic or therapeutic agents, we generate inverted repeat constructs and express them in planta (preferentially in tobacco by using transient and/or stable Agrobacterium-mediated transformation methods).

More specifically, we have targeted the essential genes from P. aeruginosa, including LptH, LolA, TolB, LpxA, LpxD, dnaA, dnaN, gyrB, rpoC, secE and sodB, using the following constructs (all of them containing the intron of SEQ ID NO:2, apart from the target sequences):
IR-LptH/LolA/TolB, SEQ ID NO: 250-251;
IR-LpxA/LpxD/TolB, SEQ ID NO: 252-253;
IR-dnaA/dnaB/gyrB, SEQ ID NO:108-109;
IR-rpoC/secE/SodB, SEQ ID NO:110-111; and
IR-secE/dnaN/gyrB, SEQ ID NO: 254-255.

We have also targeted the essential genes of Shigella flexneri, including FtsA, Can, Tsf AccD, Der, Psd using the constructs:
IR-FtsA/Can/Tsf SEQ ID NO: 116-117; and
IR-AccD/Der/Psd, SEQ ID NO: 118-119.

The same approach has been also used for the production of plant EV-embedded small RNAs directed against key virulence genes from P. aeruginosa, including genes involved in the regulation and/or assembly of type II or type III secretion systems, XcpQ, PscC, PcrV, PcrR, ExoS, ExoU, ExsA, Vrf the quorum sensing signaling factors LasR, RhlR, MvfR, VqsM, the GAC signaling components GacA, RsmA, by using the following constructs:
IR-XcpQ ExsA/PcrV/LasR/RhlR/VqsM/RmsA, SEQ ID NO: 256-257;
IR-XcpQ/PscF/PscC, SEQ ID NO: 258-259;
IR-ExoS/ExsA/Vrf, SEQ ID NO: 260-261;
IR-ExoU/ExsA/Vrf, SEQ ID NO: 262-263;
IR-LasR/RhlR/VqsM, SEQ ID NO: 264-265; and
IR-GacA/RmsA/MvfR, SEQ ID NO: 266-267.

We have also targeted the virulence genes of Shigella flexneri, including VirF, VirB, IcsA using the constructs IR-VirF/VirB IcsA, SEQ ID NO: 268-269, and the virulence genes of Staphylococcus aureus, including the genes encoding surface bound proteins fnbA, clfA, clfB, spa, atl, the leukotoxins lukF-PV, lukS-PV, lukE, lukD, HlgB, the alpha hemolysin hla, and the toxic shock syndrome toxin-1 tsst-1, by using the constructs
IR-fnbA/clfA/clfB/spa, SEQ ID NO: 270-271;
IR-lukF-PV/lukS-PV/lukE/lukD, SEQ ID NO: 272-273; and
IR-HlgB/hla tsst-1/atl, SEQ ID NO: 274-275.

### REFERENCES

1. Couto, D., Zipfel, C. (2016). Regulation of pattern recognition receptor signaling in plants. Nat Rev Immunol. 16, 537-552.
2. Jones, J D., and Dangl, J L. (2006). The plant immune system. Nature. 444, 323-329.
3. Block, A., Alfano, J R. (2011). Plant targets for Pseudomonas syringae type III effectors: virulence targets or guarded decoys? Curr Opin Microbiol. 14, 39-46.
4. Xin X F., Kvitko B., He S Y. (2018). Pseudomonas syringae: what it takes to be a pathogen. Nat Rev Microbiol. 16:316-328.
5. Jones, J D., Vance, R E., Dangl, J L. (2016). Intracellular innate immune surveillance devices in plants and animals. Science. 354, DOI: 10.1126/science.aaf6395.
6. Navarro, L., Zipfel, C., Rowland, O., Keller, I., Robatzek, S., Boller, T., Jones, J D. (2004). The transcriptional innate immune response to flg22. Interplay and overlap with Avr gene-dependent defense responses and bacterial pathogenesis. Plant Physiol. 135, 1113-28.
7. Tsuda, K and Katagiri, F. (2010). Comparing signaling mechanisms engaged in pattern-triggered and effector-triggered immunity. Curr Opin Plant Biol. 13: 459-65.
8. Hamilton A. J., Baulcombe, D C. A species of small antisense RNA in posttranscriptional gene silencing in plants. Science. 1999; 286(5441):950-2.
9. Staiger, D., Korneli, C., Lummer, M., and Navarro, L. (2013). Emerging role for RNA-based regulation in plant immunity. New Phytol. 197, 394-404.
10. Melnyk, C W., Molnar, A., Baulcombe, D C. (2011). Intercellular and systemic movement of RNA silencing signals. EMBO J. 30, 3553-63.
11. Baulcombe D C. (2015). VIGS, HIGS and FIGS: small RNA silencing in the interactions of viruses or filamentous organisms with their plant hosts. Curr Opin Plant Biol. 26, 141-6.
12. Weiberg, A., Bellinger, M., Jin H. (2015). Conversations between kingdoms: small RNAs. Curr Opin Biotechnol. 32, 207-15.
13. Koch, A., Kogel, K H. (2014). New wind in the sails: improving the agronomic value of crop plants through RNAi-mediated gene silencing. Plant Biotechnol J. 12, 821-31.
14. Zhang, T., Zhao, Y L., Zhao, J H., Wang, S., Jin, Y., Chen, Z Q., Fang, Y Y., Hua, C L., Ding, S W., Guo, H S. (2016). Cotton plants export microRNAs to inhibit virulence gene expression in a fungal pathogen. Nature Plants. DOI: 10.1038/nplants.2016.153.
15. Wang, M., Weiberg, Arne., Lin F M., Thomma, B P H., Huang, H D., Jin, H. (2016). Bidirectional cross-kingdom RNAi and fungal uptake of external RNAs confer plant protection. Nature plants. 16151.
16. Weiberg, A., Wang, M., Lin, F M., Zhao, H., Zhang, Z., Kaloshian, I., Huang, H D., Jin, H. (2013). Fungal small RNAs suppress plant immunity by hijacking host RNA interference pathways. Science. 342, 118-23.
17. Cai, Q., Qiao, L., Wang, M., He, B., Lin, F M., Palmquist, J., Huang, S D., Jin, H. (2018). Plants send small RNAs in extracellular vesicles to fungal pathogen to silence virulence genes. Science. 360, 1126-1129.
18. Micali, C O., Neumann, U., Grunewald, D., Panstruga, R., O'Connell. (2011). Biogenesis of a specialized plant-fungal interface during host cell internalization of Golovinomyces orontii haustoria. Cell Microbiol. 13, 210-226.
19. Hou, Y., Zhai, Y., Feng, L., Karimi, H., Rutter, B., Zeng, L., Seok Choi, D., Zhang, B., Gu, W., Chen, X., Ye, W., Innes R. W., Zhai, J., Ma, W. (2019). A phytophthora effector suppresses trans-kingdom RNAi to promote disease susceptibility. Cell Host & Microbe. 25, 153-165.
20. Koch A., Kumar N., Weber L., Keller, H., Imani J., Kogel, K H. (2013) Host-induced gene silencing of cytochrome P450 lanosterol C14 α-demethylase-encoding genes confers strong resistance to Fusarium species. Proc Natl Acad Sci USA. 110: 19324-9.
21. Koch, A., Biedenkopf, D., Furch, A., Weber, L., Rossbach, O., Abdellatef, E., Linicus, L., Johannsmeier, J., Jelonek, L., Goesmann, A., Cardoza, V., McMillan, J., Mentzel, T., Kogel, K H. (2016). An RNAi-based control of Fusarium graminearum infections through spraying of long dsRNAs involves a plant passage and is controlled by the fungal silencing machinery. PLoS Pathog. 12: e1005901.
22. Ghag S. B., Host induced gene silencing, an emerging science to engineer crop resistance against harmful plant pathogens. (2017). Physiological and Molecular Plant Pathology. 100: 242-254.
23. Lacombe, S., Rougon-Cardoso, A., Sherwood, E., Peeters, N., Dahlbeck, D., van Esse, H P., Smoker, M., Rallapalli, G., Thomma, B P., Staskawicz, B., Jones, J D., Zipfel, C. (2010). Interfamily transfer of a plant pattern-recognition receptor confers broad-spectrum bacterial resistance. Nat. Biotechnol. 28:365-9.
24. Wang, M., Thomas, N., Jin, H. (2017). Cross-kingdom RNA trafficking and environmental RNAi for powerful innovative pre- and post-harvest plant protection. Curr Opin Plant Biol. 38:133-141.
25. Mansfield, J., Genin, S., Magori, S., Citovsky, V., Sriariyanum, M., Ronald, P., Dow, M., Verdier, V., Beer, S V., Machado, M. A., Toth, I., Salmond, G., Foster, G. D. (2012). Top 10 plant pathogenic bacteria in molecular plant pathology. Mol Plant Pathol. 13:614-29.
26. Guchi, B., and Ashenafi, M. (2010). Microbial load, prevalence and antibiograms of salmonella and Shigella in lettuce and green peppers. Ethiopian journal of health sciences 20, 41-48.
27. Semenov, A. M., Kuprianov, A. A., and van Bruggen, A. H. (2010). Transfer of enteric pathogens to successive habitats as part of microbial cycles. Microb Ecol 60, 239-249.
28. Jo, S H. et al. (2019) A human pathogenic bacterium Shigella proliferates in plants through adoption of type III effectors for shigellosis. Plant Cell Environ. 10.1111/13603
29. Schikora, A., Garcia, A. V., and Hirt, H. (2012). Plants as alternative hosts for Salmonella. Trends in plant science 17, 245-249.
30. Winston W M, Sutherlin M, Wright A J, Feinberg E H, Hunter C P. (2007). Caenorhabditis elegans SID-2 is required for environmental RNA interference. Proc Natl Acad Sci USA. 104(25):10565-70.
31. Whangbo, J. S. & Hunter, C. P. (2008). Environmental RNA interference. Trends Genet. 24, 297-305.
32. McEwan, D. L., Weisman, A. S. & Huntert, C. P. (2012). Uptake of extracellular double-Stranded RNA by SID-2. Mol. Cell 47, 746-754.
33. Feinberg, E. H. & Hunter, C. P. (2003). Transport of dsRNA into cells by the transmembrane protein SID-1. Science 301, 1545-1547.
34. Hinas, A., Wright, A. J. & Hunter, C. P. (2012). SID-5 Is an endosome-associated protein required for efficient systemic RNAi in C. elegans. Curr. Biol. 22, [symbol] 1938-1943.
35. Ivashuta, S. et al. (2015). Environmental RNAi in herbivorous insects. RNA 21, 840-850
36. Bolognesi, R., Ramaseshadri, P., Anderson, J., Bachman, P., Clinton, W., Flannagan, R., Ilagan, O., Lawrence, C., Levine, S., Moar, W., Mueller, G., Tan, J., Uffman, J., Wiggins, E., Heck, G., Segers, G. (2012). Characterizing the mechanism of action of double-stranded RNA activity against western corn rootworm (Diabrotica virgifera virgifera LeConte). PloS one, 7(10), e47534.
37. Gantier M P, Williams B R (2007) The response of mammalian cells to double-stranded RNA. Cytokine Growth Factor Rev 18:363-371.
38. Liberati N T, et al. (2006) An ordered, nonredundant library of Pseudomonas aeruginosa strain PA14 transposon insertion mutants. Proc Natl Acad Sci USA 103(8):2833-2838.
39. Lee S A, et al. (2015) General and condition-specific essential functions of Pseudomonas aeruginosa. Proc Natl Acad Sci USA 112:5189-5194.
40. Turner K H, Wessel A K, Palmer G C, Murray J L, Whiteley M (2015) Essential genome of Pseudomonas aeruginosa in cystic fibrosis sputum. Proc Natl Acad Sci USA 112:4110-4115.
41. Murphy, D. E., et al. Extracellular vesicle-based therapeutics: natural versus engineered targeting and trafficking. Exp. Mol. Med. (2018).
42. Zhang, R., Lin, Y. (2009). DEG 5.0, a database of essential genes in both prokaryotes and eukaryotes. Nucleic Acids Res., 37: D455-D458.
43. Mitter, N., Worrall, E. A., Robinson, K. E., Li, P., Jain, R. G., Taochy, C., Fletcher, S. J., Carroll, B. J., Lu, G. Q., Xu, Z. P. (2017). Clay nanosheets for topical delivery of RNAi for sustained protection against plant viruses. Nat Plants, 3:16207.
44. Draz M S, Fang B A, Zhang P, et al. Nanoparticle-Mediated Systemic Delivery of siRNA for Treatment of Cancers and Viral Infections. Theranostics. 2014; 4: 872-92.
45. Kowal, J., Arras, G., Colombo, M., Jouve, M., Morath, J. P., Primdal-Bengtson, B., Dingli, F., Loew, D., Tkach, M., Théry, C. (2016). Proteomic comparison defines novel markers to characterize heterogenous populations of extracellular vesicle subtypes. Proc Natl Acad Sci USA., 113: E968-77.
46. Rutter, B. D., Innes, R. W. (2017). Extracellular vesicles isolated from the leaf apoplast carry stress-response proteins. Plant Physiol., 173: 728-741.
47. Rutter, B. D., Innes, R. W. (2018). Extracellular vesicles are key mediators of plant-microbe interactions. Current opinion in Plant Biology., 44:16-22.
48. Regente, M., Corti-Monzon, G., Maldonado, A. M., Pinedo, M., Jorrin, J., de la Canal, L. (2009). Vesicular fractions of sunflower apoplastic fluids are associated with potential exosome marker proteins. FEBS Lett., 583:3363-3366. 34.
49. Mathieu, M., Martin-Jaular, L., Lavieu, G., Théry, C. (2019). Specificities of secretion and uptake of exosomes and other extracellular vesicles for cell-to-cell communication. Nature cell biology., 9-17.
50. Tkach, M., Théry, C. (2016). Communication by Extracellular Vesicles: Where We Are and Where We Need to Go. Cell: 1226-32.
51. Navarro, L., Jay, F., Nomura, K., He, S. Y. & Voinnet, O. Suppression of the microRNA pathway by bacterial effector proteins. Science 321, 964-7 (2008).
52. Melotto, M., Underwood, W., Koczan, J., Nomura, K., He, S. Y. (2006). Plant stomata function in innate immunity against bacterial invasion. Cell. 126:969-80.
53. Fouts, D. E., Abramovitch, R. B., Alfano, J. R., Baldo, A. M., Buell, C. R., Cartinhour, S., Chatterjee, A. K., D'Ascenzo, M., Gwinn, M. L., Lazarowitz, S. G., Lin, N. C., Martin, G. B., Rehm, A. H., Schneider, D. J., van Dijk, K., Tang, X., Collmer, A. (2002). Genome-wide identification of Pseudomonas syringae pv. tomato DC3000 promoters controlled by the HrpL alternative sigma factor. Proc. Natl. Acad Sci. U.S.A. 19:2275-80.
54. Zwiesler-Vollick, J., Plovanich-Jones, A. E., Nomura, K., Bandyopadhyay, S., Joardar, V., Kunkel, B. N., and He S. Y. 2002. Identification of novel hrp-regulated genes through functional genomic analysis of the Pseudomonas syringae pv. tomato DC3000 genome. Mol. Microbiol. 45:1207-1218.
55. Sreedharan, A., Penaloza-Vazquez, A., Kunkel, B. N. & Bender, C. L. CorR regulates multiple components of virulence in Pseudomonas syringae pv. tomato DC3000. Mol Plant Microbe Interact 19, 768-79 (2006).
56. Fan, J., Crooks, C., Lamb, C. High-throughput quantitative luminescence assay of the growth in planta of Pseudomonas syringae chromosomalens luxCDABE. Plant J. 53, 393-390 (2008).
57. E. A. Meighen. (1991). Molecular biology of bacterial bioluminescence Microbiol. Rev., 55 pp. 123-142
58. W. Ma, Andrade, M. O., Farah, C. S. & Wang, N., 2014. The Post-transcriptional Regulator rsmA/csrA Activates T3 S by Stabilizing the 5′UTR of hrpG, the Master Regulator of hrp/hrc Genes, in Xanthomonas W. Ma, ed. PLoS Pathogens, 10(2), p.e1003945.
59. Oku, T., A. M. Alvarez, and C. I. Kado. (1995). Conservation of the hypersensitivity-pathogenicity regulatory gene hrpX of Xanthomonas campestris and X. oryzae. DNA Sequence 5:245-249.
60. Tang, X., Xiao, Y., Zhou, J M. (2006) Regulation of the type III secretion system in phytopathogenic bacteria. Mol Plant Microbe Interact 19: 1159-1166
61. Wengelnik, K., Van den Ackerveken, G., Bonas, U. (1996) HrpG, a key hrp regulatory protein of Xanthomonas campestris pv. vesicatoria is homologous to two-component response regulators. Molecular plant-microbe interactions 9: 704-712.
62. Wengelnik, K., Rossier, O., Bonas, U. (1999) Mutations in the regulatory gene hrpG of Xanthomonas campestris pv. vesicatoria result in constitutive expression of all hrp genes. Journal of bacteriology 181: 6828-6831.
63. Vicente J G, Holub E B. (2013) Xanthomonas campestris pv. campestris (cause of black rot of crucifers) in the genomic era is still a worldwide threat to brassica crops. Mol Plant Pathol. 14(1):2-18.
64. Ramphal R, Balloy V, Jyot J, Verma A, Si-Tahar M, Chignard M: Control of Pseudomonas aeruginosa in the lung requires the recognition of either lipopolysaccharide or flagellin. J Immunol 2008, 181:586-592.
65. EARS-NET. Surveillance report: “Antimicrobial resistance surveillance in Europe” (2014);
66. EMA/ECDC. Technical report: “The bacterial challenge: time to react” (2009)
67. Jia et al. (2017). CARD 2017: expansion and model-centric curation of the Comprehensive Antibiotic Resistance Database. Nucleic Acids Research, 45, D566-573.

1-43. (canceled)

44. An in vitro method for inhibiting the expression of at least one gene in a target bacterial cell, said method comprising the step of contacting said target bacterial cell with small RNAs, or with compositions containing small RNAs, said small RNAs having a length comprised between 15 and 30 base pairs.

45. The method of claim 1, wherein said bacteria are animal pathogenic bacteria.

46. The method of claim 1, wherein said bacteria are chosen from the group consisting of:
Actinomyces israelii, Bacillus anthracis, Bacillus cereus, Bacteroides fragilis, Bordetella pertussis, Borrelia sp. (burgdorferi, garinii, afzelii, recurrentis, crocidurae, duttonii, hermsii etc.), Brucella sp. (abortus, canis, melitensis, suis), Campylobacter jejuni, Chlamydia sp. (pneumoniae, trachomatis), Chlamydophila psittaci, Clostridium sp. (botulinum, difficile, perfringens, tetani), Corynebacterium diphtheriae, Ehrlichia sp. (canis, chaffeensis), Enterococcus (faecalis, faecium), Escherichia coli O157:H7, Francisella tularensis, Haemophilus influenza, Helicobacter pylori, Klebsiella pneumoniae, Legionella pneumophila, Leptospira sp., Listeria monocytogenes, Mycobacterium sp. (leprae, tuberculosis), Mycoplasma pneumoniae, Neisseria (gonorrhoeae, meningitidis), Pseudomonas aeruginosa, Porphyromonas gingivalis, Nocardia asteroides, Rickettsia rickettsii, Salmonella sp. (typhi, typhimurium), Shigella sp. (sonnei, dysenteriae), Staphylococcus (aureus, epidermidis, saprophyticus), Streptococcus sp. (agalactiae, mutans, pneumoniae, pyogenes, viridans), Tannerella forsythia, Treponema pallidum, Vibrio cholerae, and Yersinia pestis.

47. The method of claim 1, wherein said composition contains extracellular free small RNAs, or extracellular vesicles containing said small RNAs or apoplastic fluid containing said small RNAs or nanoparticles coupled to said small RNAs.

48. A therapeutical composition containing, as active principle, the small RNA as defined in claim 1.

49. The therapeutic composition according to claim 48, containing extracellular free small RNAs, or extracellular vesicles containing said small RNAs or apoplastic fluid containing said small RNAs or nanoparticles coupled said small RNAs, and a pharmaceutically acceptable excipient.

50. The therapeutic composition according to claim 48, wherein it is formulated for an oral, topical or systemic administration, preferably as a pill, a cream, or an oral spray.

51. A method for treating and/or preventing a bacterial infection in a subject in need thereof, said method comprising administering to said subject the therapeutic composition of claim 5.

52. The method according to claim 51, wherein said composition is administered orally, topically or systemically to said subject.

53. The method according to claim 51, wherein said bacterial infection is due to human pathogenic bacteria chosen from:
Actinomyces israelii, Bacillus anthracis, Bacillus cereus, Bacteroides fragilis, Bordetella pertussis, Borrelia sp. (burgdorferi, garinii, afzelii, recurrentis, crocidurae, duttonii, hermsii etc.), Brucella sp. (abortus, canis, melitensis, suis), Campylobacter jejuni, Chlamydia sp. (pneumoniae, trachomatis), Chlamydophila psittaci, Clostridium sp. (botulinum, difficile, perfringens, tetani), Corynebacterium diphtheriae, Ehrlichia sp. (canis, chaffeensis), Enterococcus (faecalis, faecium), Escherichia coli O157:H7, Francisella tularensis, Haemophilus influenza, Helicobacter pylori, Klebsiella pneumoniae, Legionella pneumophila, Leptospira sp., Listeria monocytogenes, Mycobacterium sp. (leprae, tuberculosis), Mycoplasma pneumoniae, Neisseria (gonorrhoeae, meningitidis), Pseudomonas aeruginosa, Porphyromonas gingivalis, Nocardia asteroides, Rickettsia rickettsii, Salmonella sp. (typhi, typhimurium), Shigella sp. (sonnei, dysenteriae), Staphylococcus (aureus, epidermidis, saprophyticus), Streptococcus sp. (agalactiae, mutans, pneumoniae, pyogenes, viridans), Tannerella forsythia, Treponema pallidum, Vibrio cholerae, and Yersinia pestis.

54. A method for promoting beneficial effects of commensal or symbiotic beneficial bacteria in a subject in need thereof, said method comprising administering to a subject in need thereof the composition as defined in claim 48.

55. The method according to claim 54, wherein said commensal or symbiotic beneficial bacteria is chosen from:
Actinomyces naeslundii, Veillonella dispar, Faecalibacterium prausnitzii, Enterobacteriaceae, Bacteroides thetaiotaomicron, Escherichia coli K2, Bifidobacterium sp. (longum, bifidum, adolescentis, dentium, breve, thermophilum), Eggerthella lenta, Bacteroides sp. (xylanisolvens, thetaiotaomicron, fragilis, vulgatus, salanitronis), Parabacteroides distasonis, Faecalibacterium prausnitzii, Ruminococcus sp. (bromii, champanellensis, SR1/5), Streptococcus (parasanguinis, salivarius, thermophilus, suis, pyogenes, anginosus), Lactococcus (lactis, garvieae), Enterococcus (faecium, faecalis, casselflavus, durans, hirae, Melissococcus plutonius, Tetragenococcus halophilus, Lactobacillus sp. (casei, ruminis, delbrueckii, buchneri, reuteri, fermentum, pentosus, amylovorus, salivarius), Pediococcus (pentosaceus, claussenii), Leuconostoc (mesenteroides, lactis, carnosum, gelidum, citreum), Weissella (thailandensis, koreensis), Oenococcus oeni, Paenibacillus sp. (terrae, polymyxa, mucilaginosus, Y412MCI0), Thermobacillus composti, Brevibacillus brevis, Bacillus (amyloliquefaciens, subtilis, lichenformis, atrophaeus, weihenstephanensis, cereus, thuringiensis, coagulans, megaterium, selenitireducens), Geobacillus thermodenitrificans, Lysinibacillus sphaericus, Halobacillus halophilus, Listeria sp., Streptomyces sp., Eubacterium (rectale, eligens, siraeum), Clostridium saccharolyticum, and butyrate-producing bacterium (SS3/4 and SSC/2).

56. A method for improving the efficiency of an antibiotic treatment in a subject in need thereof, said method comprising administering to said subject the therapeutic composition of claim 48, wherein said small RNA inhibits specifically the expression of at least one bacterial antibiotic resistance gene.

57. The method according to claim 56, wherein said antibiotic resistance gene is chosen from: VIM-1, VIM-2, VIM-3, VIM-5, Case, OXA-28, OXA-14, OXA-19, OXA-145, PER-4, TEM-116, and GES-9.

58. The method according to claim 56, wherein said antibiotic compound is chosen from: Aminoglycosides, Carbapenems, Ceftazidime (3rd generation), Cefepime (4th generation), Ceftobiprole (5th generation), Ceftolozane/tazobactam, Fluoroquinolones, Piperacillin/tazobactam, Ticarcillin/clavulanic acid, Amikacin, Gentamicin, Kanamycin, Neomycin, Netilmicin, Tobramycin, Paromomycin, Streptomycin, Spectinomycin, Geldenamycin, herbimycin, Rifaximin, Ertapenem, Doripenem, Imipenem, Meropenem, Cefadroxil, Cefazolin, Cephradine, Cephapirin, Cephalothin, Cefalexin, Cefaclor, Cefoxitin, Cefotetan, Cefamandole, Cefinetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cephalosporins, Cefepime, Cephalosporins, Ceftaroline fosamil, Ceftobiprole, Glycopeptides, Teicoplanin, Vancomycin, Telavancin, Dalbavancin, Oritavancin, Lincosamides(Bs), Clindamycin, Lincomycin, Lipopeptide, Daptomycin, Macrolides(Bs), Azithromycin, Clarithromycin, Erythromycin, Roxithromycin, Telithromycin, Spiramycin, Fidaxomicin, Monobactams, Aztreonam, Nitrofurans, Furazolidone, Nitrofurantoin(Bs), Oxazolidinones(Bs), Linezolid, Posizolid, Radezolid, Torezolid, Penicillins, Amoxicillin, Ampicillin, Azlocillin, Dicloxacillin, Flucloxacillin, Mezlocillin, Methicillin, Nafcillin, Oxacillin, Penicillin G, Penicillin, Piperacillin, Temocillin, Ticarcillin, Penicillin combinations, Amoxicillin/clavulanate, Ampicillin/sulbactam, Piperacillin/tazobactam, Ticarcillin/clavulanate, Polypeptides, Bacitracin, Colistin, Polymyxin B, Quinolones/Fluoroquinolones, Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, Temafloxacin, Sulfonamides(Bs), Mafenide, Sulfacetamide, Sulfadiazine, Silver sulfadiazine, Sulfadimethoxine, Sulfamethizole, Sulfamethoxazole, Sulfanilimide (archaic), Sulfasalazine, Sulfisoxazole, Trimethoprim-Sulfamethoxazole (Co-trimoxazole) (TMP-SMX), Sulfonamidochrysoidine (archaic), Tetracyclines(Bs), Demeclocycline, Doxycycline, Metacycline, Minocycline, Oxytetracycline, Tetracycline, Clofazimine, Dapsone, Capreomycin, Cycloserine, Ethambutol(Bs), Ethionamide, Isoniazid, Pyrazinamide, Rifampicin, Rifabutin, Rifapentine, Streptomycin, Arsphenamine, Chloramphenicol(Bs), Fosfomycin, Fusidic acid, Metronidazole, Mupirocin, Platensimycin, Quinupristin/Dalfopristin, Thiamphenicol, Tigecycline(Bs), Tinidazole, and Trimethoprim(Bs).

59. The method of claim 56 comprising:
a) administering to said subject the therapeutic composition of claim 5, and
b) administering to said subject, simultaneously or separately or in a staggered manner, an antibiotic compound.

60. An in vitro method to identify candidate genes involved in bacterial antibiotic resistance, or that affect the proliferation of human pathogenic bacterial cells, said method comprising the steps of:
a) generating small RNAs having a length comprised between 15 and 30 base pairs and inhibiting specifically the expression at least one bacterial gene,
b) incubating bacterial cells with said small RNA,
c) optionally, incubating said small RNA treated bacterial cells with an antibiotic compound,
d) assessing the viability, growth, metabolic activity, of said small RNA treated bacterial cells in the presence or absence of said antibiotic compound, and optionally compare same with the viability, growth, metabolic activity, of said small RNA treated bacterial cells in the absence of said antibiotic compound.

61. The method of claim 60, wherein the candidate gene is involved in bacterial antibiotic resistance if the viability, growth, metabolic activity, of said small RNA treated bacterial cells in the presence of said antibiotic compound is lower than the viability, growth, metabolic activity, of said small RNA treated bacterial cells in the absence of said antibiotic compound.

62. The method of claim 60, wherein said bacterial cells are chosen from:
Actinomyces israelii, Bacillus anthracis, Bacillus cereus, Bacteroides fragilis, Bordetella pertussis, Borrelia sp. (burgdorferi, garinii, afzelii, recurrentis, crocidurae, duttonii, hermsii etc.), Brucella sp. (abortus, canis, melitensis, suis), Campylobacter jejuni, Chlamydia sp. (pneumoniae, trachomatis), Chlamydophila psittaci, Clostridium sp. (botulinum, difficile, perfringens, tetani), Corynebacterium diphtheriae, Ehrlichia sp. (canis, chaffeensis), Enterococcus (faecalis, faecium), Escherichia coli O157:H7, Francisella tularensis, Haemophilus influenza, Helicobacter pylori, Klebsiella pneumoniae, Legionella pneumophila, Leptospira sp., Listeria monocytogenes, Mycobacterium sp. (leprae, tuberculosis), Mycoplasma pneumoniae, Neisseria (gonorrhoeae, meningitidis), Pseudomonas aeruginosa, Porphyromonas gingivalis, Nocardia asteroides, Rickettsia rickettsii, Salmonella sp. (typhi, typhimurium), Shigella sp. (sonnei, dysenteriae), Staphylococcus (aureus, epidermidis, saprophyticus), Streptococcus sp. (agalactiae, mutans, pneumoniae, pyogenes, viridans), Tannerella forsythia, Treponema pallidum, Vibrio cholerae, and Yersinia pestis.
